(19) United States
(12) Patent Application Publication
(10) Pub. No.: US 2015/0030615 A1
(43) Pub. Date: Jan. 29, 2015

(54) BIOMARKERS FOR CANCER STEM CELLS AND RELATED METHODS OF USE

(71) Applicant: VERASTEM, INC., Cambridge, MA (US)

(72) Inventors: Alan G. Derr, Westford, MA (US); David T. Weaver, Cambridge, MA (US); Irina Shapiro, Cambridge, MA (US); Daniel W. Paterson, Cambridge, MA (US); Jonathan A. Pachter, Cambridge, MA (US)

(21) Appl. No.: 14/384,026

(22) PCT Filed: Mar. 8, 2013
(86) PCT No.: PCT/US13/29895

§ 371 Date: Sep. 9, 2014

Related U.S. Application Data
(60) Provisional application No. 61/609,031, filed on Mar. 9, 2012.

(51) Int. Cl. C12Q 1/68

(52) U.S. Cl. CPC C12Q 1/6886; C12Q 2600/158
USPC 424/174.1; 506/9; 506/16

(57) ABSTRACT

Novel methods of classifying subjects as candidates for treatment with a cancer associated mesenchymal cell, tumor initiating cancer cell, or cancer stem cell inhibitor treatment and subsequent administration of the cancer associated mesenchymal cell, tumor initiating cancer cell, or cancer stem cell inhibitor are disclosed within.

### CROSS-REFERENCE TO RELATED APPLICATIONS

This application claims the priority of U.S. provisional application Ser. No. 61/609,031, filed Mar. 9, 2012, which is incorporated by reference herein in its entirety.

### BACKGROUND

Currently available therapeutic regimens are ineffective in treating many cancers. Cancer stem cells (CSCs), cancer associated mesenchymal cells, or tumor initiating cancer cells, comprise a unique subpopulation of a tumor and have been identified in a large variety of cancer types. Although this subpopulation of cells constitutes only a small fraction of a tumor, they are thought to be the main cancer cells responsible for tumor initiation, growth, and recurrence. Given that current cancer treatments have, in large part, been designed to target rapidly proliferating cells, this subpopulation of cells, which is often slow growing, may be relatively more resistant to these treatments. Therefore, methods to identify cancer patients likely to respond positively to a treatment comprising an agent that inhibits or kills cancer associated mesenchymal cells, tumor initiating cancer cells, or cancer stem cells are needed; and can provide the basis for subsequent administration of a treatment comprising an agent that inhibits or kills cancer associated mesenchymal cells, tumor initiating cancer cells, or cancer stem cells; to this candidate group of cancer patients.

### SUMMARY OF INVENTION

The present invention provides a method for classifying subjects likely to respond to a particular therapeutic regimen for treating cancer. The method is based, at least in part, on the characterization of signals (e.g., the level of gene expression) possessed by a candidate subject population for treatment with a preselected drug. In general, the method involves identifying differences in candidate and non-candidate subject populations, where for example, a subject population has a gene expression profile associated with a candidate or non-candidate classification. The method can further comprise administration of the therapeutic regimen to the candidate population based on the characterized gene expression profile.

In an aspect, the invention features a method of evaluating or treating a subject, comprising: (a) optionally, acquiring a subject sample, e.g., a tissue sample, such as a biopsy; bodily fluids, such as blood or plasma (b) acquiring a value or values that is a function of the level of gene expression for each of a plurality of genes selected from a first and/or second and/or third and/or fourth and/or fifth and/or sixth and/or seventh and/or eighth and/or ninth and/or tenth and/or eleventh and/or twelfth and/or thirteenth and/or fourteenth set of genes; (c) and responsive to said value or values (i) classifying the subject, e.g., classifying the subject as a candidate or non-candidate for treatment with a preselected drug, and/or treating, or withholding treatment from, the subject with a preselected drug; or (ii) administering a treatment comprising an agent that inhibits or kills cancer associated mesenchymal cells, tumor initiating cancer cells, or cancer stem cells, to the subject; provided that, if (c)(ii) is not performed the acquisition in (a) or (b) comprises directly acquiring; thereby evaluating or treating the subject.

In an embodiment, (a) optionally, acquiring a subject sample, e.g., a tissue sample, such as a biopsy; bodily fluids, such as blood or plasma, is performed. In an embodiment, (b) acquiring a value or values that is a function of the level of gene expression for each of a plurality of genes selected from a first and/or second and/or third and/or fourth and/or fifth and/or sixth and/or seventh and/or eighth and/or ninth and/or tenth and/or eleventh and/or twelfth and/or thirteenth and/or fourteenth set of genes, is performed. In an embodiment, (a) optionally, acquiring a subject sample, e.g., a tissue sample, such as a biopsy; bodily fluids, such as blood or plasma, is performed; and (b) acquiring a value or values that is a function of the level of gene expression for each of a plurality of genes selected from a first and/or second and/or third and/or fourth and/or fifth and/or sixth and/or seventh and/or eighth and/or ninth and/or tenth and/or eleventh and/or twelfth and/or thirteenth and/or fourteenth set of genes, is performed are performed.

In an embodiment, (c) responsive to said value or values (i) classifying the subject, e.g., classifying the subject as a candidate or non-candidate for treatment with a preselected drug, and/or treating, or withholding treatment from, the subject with a preselected drug; is performed. In an embodiment, (c) responsive to said value or values (ii) administering a treatment comprising an agent that inhibits or kills cancer associated mesenchymal cells, tumor initiating cancer cells, or cancer stem cells, to the subject; is performed. In an embodiment, (c) responsive to said value or values (i) classifying the subject, e.g., classifying the subject as a candidate or non-candidate for treatment with a preselected drug, and/or treating, or withholding treatment from, the subject with a preselected drug; is performed and (c) responsive to said value or values (ii) administering a treatment comprising an agent that inhibits or kills cancer associated mesenchymal cells, tumor initiating cancer cells, or cancer stem cells, to the subject; are performed.

In an embodiment, the invention features, responsive to said value or values, classifying the subject, e.g., classifying the subject as a candidate or non-candidate for treatment with a preselected drug, and/or treating, or withholding treatment from, the subject with a preselected drug, wherein the subject sample is directly acquired; thereby evaluating the subject.

In an embodiment, the invention features, responsive to said value or values, classifying the subject, e.g., classifying the subject as a candidate or non-candidate for treatment with a preselected drug, and/or treating, or withholding treatment from, the subject with a preselected drug, wherein said value or values is directly acquired; thereby evaluating the subject.

In an embodiment, the invention features, responsive to said value or values, classifying the subject, e.g., classifying the subject as a candidate or non-candidate for treatment with a preselected drug, and/or treating, or withholding treatment from, the subject with a preselected drug, wherein the subject sample and said value or values are directly acquired; thereby evaluating the subject.

In an embodiment, the invention features, responsive to said value or values, administering a treatment comprising an agent that inhibits or kills cancer associated mesenchymal cells, tumor initiating cancer cells, or cancer stem cells to said subject.

In an embodiment, the invention features, responsive to said value or values, classifying the subject, e.g., classifying the subject as a candidate or non-candidate for treatment with a preselected drug, and/or treating, or withholding treatment from, the subject with a preselected drug; and administering a treatment comprising an agent that inhibits or kills cancer associated mesenchymal cells, tumor initiating cancer cells, or cancer stem cells to said subject.

In an embodiment, the first set of genes are related to the delineation of a cancer stem cell, cancer associated mesenchymal cell, or a tumor initiating cancer cell phenotype and the determination of the attributes of the epithelial to mesenchymal transition (EMT) in cancers. For example,

(i) said first set of genes comprises or consists of:
KLF13, ANTXR2, IFNAR1, LIX1L, CHST11, AKAP2, PALM2-AKAPT, LOC80298, C7orf2, NUCKS, DKFZP566D1346, LOC388279, FLJ31795, 6orf107, FLJ12439, ENPP2, CITED2, ZEB1, NID2, SEMA5A, DAB2, KCNMA1, PTX3, PCDH9, CREB3L1, BGN, ROR1, DLC1, GNG11, CDH11, NR2F1, PRR16, MYL9, DOCK10, LRIG1, IER3, EML1, NEBL, RGL1, MLPH, DNAJB4, FBLN5, RGS4, HAS2, ITGBL1, IGFBP4, DPT, PCOLCE, GREM1, PPAP2B, CDH2, PMP22, LUM, CHN1, CYP1B1, MME, WNT5A, POSTN, MMP2, CTGF, FBLN1, IGFBP3, DCN, PRRX1, ANXA6, PVRL3, MAP1B, TNFAIP6, CYBRD1, FBN1, NID1, OLFML3, SNAI1, SNAI2, TAGLN, SYNC, TUBA1A, PPM1D, TWIST1, FN1, TGFBR3, SERPINF1, UGDH, SRGN, FAP, PTGER4, PRKCA, FSTL1, MMP1, NRP1, FILIP1L, SCCPDH, LTBP2, XYLT1, HS3ST2, SYT11, TSHZ1, THY1, Sep9, S100A4, TNS3, ENOX1, TGFB1I1, ZEB2, LMCD1, PDGFC, ECM1, TFPI, TBX3, DDR2, PTGER2, CDKN2C, VCAN, CD44, STARD13, SNED1, ZBTB38, SDC2, TPM1, COPZ2, STC1, CDH1, KRT5, KRT6B, CD24, EPCAM, ESRP1, CLDN3, CLDN4, ERBB3, SPOCK1, FERMT2, GLYR1, LTBP1, FADS2, KANK2, PTGFR, COL11A2, KLK3, EIF2C2, ZFP41, FAM49B, PSORS1C2, MRPL42, MRPL54, MRPL47, MRPS23, EIF3S9, ALG5, DNAJC19, TPRXL, NOTCH2, RBM15, ST3GAL3, NFYA, PCNX, FBXO21, WWOX, CAMK2B, PNPLA2, CLIC5, UGCGL1, FBXL18, ADRBK1, SLC38A2, IL8RA, TAS2R14, CD300LB, GIPC3, RAD51L1, EPPK1, COL1A1, MMP9, SERPINE1, SPARC, TGFB1, TGFB3, TGFBI, TGFBR1, TGIF, TGIF2, THBS1, ANXA5, ACTG1, ARF3, ATP1B3, BAT3, CALD1, CENTD2, CLIC1, CTBS, DPYSL3, DVL3, EXT1, FGFR1, FTL, GNB2L1, GPRC5A, H2AFZ, HIF1A, IL13RA1, KDELR2, LARP1, LPIN2, MARS, MMP10, MMP14, MT2A, MYO10, NUP62, PFKFB3, PLOD2, PSMB7, PSMD8, RIN2, RYBP, SDF4, SETD5, SPP1, STAU1, TUBB3, UBE2S, XPNPEP1, CDKN1A, CHRD, H19, ID3, ID4, IGFBP7, LRP1, MSX1, NOTCH3, PROCR, GBX2, KI67, CCNB1, BUB1, KNTC2, USP22, HCFC1, RNF2, ANK3, FGFR2, CES1, COL1A2, COL3A1, COL5A2, COL6A1, ANKRD25, C10ORF56, C5ORF13, KRT81, NPAC, PLEKHC1, SEPT9, SYNC1, MBP, ABLIM1, ALDH1A3, ALOX15B, ANXA3, AP1M2, ARTN, CA2, CA9, CDH3, CDS1, COL17A1, CORO1A, CTSL2, CXADR, CYP27B1, DSC2, DSG3, DST, EPB41L4B, FGFBP1, FGFR3, FST, GJB3, GRHL2, HBEGF, HOOK1, IL18, IL1B, IRF6, ITGB4, JAG2, KLK10, KLK5, KLK7, KLK8, KRT15, KRT16, KRT17, LEPREL1, MYO5C, NDRG1, NMU, PI3, RAB25, RLN2, RNF128, S100A14, S100A7, S100A8, SERPINB1, SERPINB2, SLC2A9, SLPI, SNX10, SPINT2, SPRR1A, SPRR1B, ST14, SYK, TMEM40, TRIM29, TSPAN1, UCHL1, VSNL1, ZBED2, AQP3, CBARA1, CLCA2, CNTNAP2, CSTA, DSC3, ESRP2, FXYD3, GJB5, GPR87, IL1A, LAMA3, LAMB3, MAP7, MAPK13, MPZL2, NRG1, SCNN1A, SERPINB13, SLC7A5, SPINT1, ST6GALNAC2, TGFA, TP63, TPD52L1, VGLL1, SLC6A8, ABCA12, ANXA8, ANXA8L1, ARHGAP25, ARHGAP8, BDKRB2, BIK, C10ORF10, C10ORF116, C1ORF116, C20ORF19, C6ORF105, CELSR2, CKMT1A, CKMT1B, CLDN1, CST6, CYP4F11, E2F5, ELMO3, EPHA1, EVA1, EXPH5, F11R, FAT2, FLJ12684, FLJ20366, FZD3, GLS2, GNAL, IFI30, IGFBP2, IL1RN, IL4R, IRX4, JUP, KCNK1, KIAA0040, KIAA0888, KIAA1815, KRT14, KRT18, LAD1, LGALS7, LOC553158, LOC653562, LOC728113, LOC729884, LRRC1, LSR, MST1R, MYO1D, NAIP, OCLN, NEFM, NUP62CL, PERP, PLS1, POLR3G, PRKCH, PRRG4, PRSS8, PTPN3, RBM35A, RBM35B, RHBDF2, RHOD, RPS6KA1, RTEL1, SAA1, SAA1, SAA2, SH2D3A, SLC6A10P, SMPDL3B, SNCA, STAC, STAP2, TACSTD1, THBD, TMEM30B, TMPRSS11E, TMPRSS4, TNFRSF6B, TP73L, WWC1, XDH, ZNF165, AXL, GPR30, PAX2, PI3KA, CD133, ALDH1, BMI1, KRT19, MSI1, JAG1, p38, ESA1, FOXC2, FOXC1, TWIST2, GSC, TCF3, MLPH1, ARHGEF11a, DPF2, CASP8, BCL2, SCGN, SWAP70, KIAA0276, C10orf9, C10orf7, ALKBH, TOB2, XPR1, CD59, LRP2, PLP2, MAPK14, CXCL2, MMP7, MGP, MLF1, FLNB, SCNM1, HSPC163, C5orf18, MGC4399, CDW92, TMC4, ZDHHC2, TICAM2, KDELR3, GNPDA1, THEM2, DBR1, FLJ90709, FLJ10774, C16orf33, GAPD, LDHA, MR-1, LARS, GTPBP1, PRSS16, WFDC2, AIM1, DHRS6, DHRS4, GC15429, MGC45840, ECHDC2, GOLGIN-67, AFURS1, KIAA0436, CYP4V2, JTV1, ICMT, DNMT3A, HNMT, METTL7A, METTL2, VIL2, TPD52, ARPC5, NOL8, NSF, RAD23B, SRP54, HSPA2, PBP, THAP2, CIRBP, SNRPN, KIAA0052, DUSP10, SSR1, ERBB4, EMP1, CHPT1, LRPAP1, FLJ11752, CSTF1, KLHL20, DNAJC13, APLP2, ARGBP2, DNAJB1, NEBL, SH3BGRL, NUDT5, GABARAPL1, MAPT, DCBLD1, STK39, PAK2, CSNK2A1, PILRB, ERN1, SGKL, WEE1, MAST4, C11orf17, NUP37, CLTC, COPB2, SLC25A25, ECOP, PDE8A, STAM, TUBB, SNX6, RAB23, PLAA, STC2, LTF, ISGF3G, ATXN3, GTF3C3, GSK3B, KLF10, ELL2, ZBTB20, IRX3, ETS1, SERTAD1, MGC4251, MAFF, SFPQ, CITED4, CEBPD, EIF4E2, HS2ST1, AGPS, PGK1, ATIC, ETNK1, LG2, NCE2, MARCH8, CNOT4, RNF8, PSMA5, DPF2, AMMECR1, KIAAI287, LOC144233, LOC286505, PNAS-4, FLJ20530, HUMPD3, GC45564, CAP350, ETAA16, HAN11, DNAPTP6, C7orf25, FLJ37953, FLJ10587, C7orf36, ELP4, NDEL1, NPD014, KFZP564D172, FAM53C, IER5, LOC255783, KIAA0146, KIAA0792, LOC439994, LOC283481, CG018, LOC130576, NGFRAP1L1, KIAAI217, 4orf7, C21 orf86, C9orf64, FLJ13456, FLJ12806, FLJ39370, GATS, CCDC92, FMNL2, ARID1B, ZFHX1B, SSBP2, ARID5B, LOC157381, KPNA3, ARHGAP24, CCND2, VIM, MYCBP2, GAS7, TRAM2, BASP1, FOXO1A, POLR2A, PER1, DDIT4, CD97, BIN1, SH2B3, DDB2, EMP3, NDST2, CHST2, NT5E, PDE4A, CPS1, PTGS1, GGCX, IRF5, ZBTB16, MAP4K4, CHST7, KLF12, NFRKB, PSD, FKSG49, NIFUN, FYN, ZMYM2, CACNA1G, SLC25A16, FLII, EIF1, SEPT6, NPAC, PHF15, NUP188, ABR, CNR1, LOC283824, FSTL4, DNM1, APOBEC3G, ATP2B1, SMPD1, SLC11A1, FXYD5, C14orf139, SH3BGRL3, LUZP1, ZNF335, SH3 KBP1, MST150, PRO1073, LOC388397, FKBP5, HIPK2, DTX1, ST3GAL2, ADAMTS7, TNRC6B, CYGB, SDHAL1, LOC572558, TRIO, FRAS1, KIAA1632, POLS, EBF, MAML2, PTPRA, PLEKHG2, DYM, SOX6, ARHGEF2, ZCCHC6, PPP3CA, FAM70B, TMED5, FLJ43663, HPS1, MEF2A, ST3GAL5, SMYD3, KLF7, LOC200230, RERE, QKI, BICD1, CTNNB1, POU2F2, EIF4ENIF1, BTG1, TCHP, FLJ90709, PCTK2, KIAA1600, B7-H4, PDE4DIP, KIAA0194, HOM-TES; and
(a) BGN, BIN1, CDH11, CDH2, CHN1, COL1A2, COL3A1, COL5A2, CREB3L1, CTGF, CYBRD1, DCN, DLC1, DNAJB4, DPT, ENPP2, FBLN1, FBLN5, FBN1, GREM1, HAS2, IGFBP3, IGFBP4, MLPH, MME, MMP2, MYL9, NEBL, NID1, NR2F1, OLFML3, PCOLCE, PMP22, POSTN, PPAP2B, PRR16, PRRX1, PTX3, PVRL3, RGL1, RGS4, ROR1, SEMA5A, TNFAIP6, TRAM2, WNT5A, ZEB1, ANXA6, CITED2, COL1A1, COL6A1, CYP1B1, DAB2, DOCK10, EML1, GNG11, IER3, ITGBL1, KCNMA1, LRIG1, LUM, MAP1B, NID2, PCDH9, PRKCA, PTGER4, PTGFR, SDC2, SERPINF1, SNED1, SYT11, TFPI, TGFBR3, VCAN, ANKRD25, C10ORF56, C5ORF13, CCDC92, CDKN2C, COPZ2, DDR2, ECM1, ENOX1, FADS2, FAP, FILIP1L, FSTL1, HS3ST2, KRT81, LMCD1, LTBP1, LTBP2, MMP1, NPAC, NRP1, PDGFC, PLEKHC1, PPM1D, PTGER2, SCCPDH, SEPT9, SPOCK1, SRGN, STARD13, STC1, SYNC1, TAGLN, TBX3, TGFB1I1, THY1, TNS3, TPM1, TSHZ1, TUBA1A, UGDH, VIM, XYLT1, ZBTB38, MBP, ABLIM1, ALDH1A3, ALOX15B, ANXA3, AP1M2, ARTN, CA2, CA9, CAMK2B, CCND2, CDH1, CDH3, CDS1, COL17A1, CORO1A, CTSL2, CXADR, CYP27B1, DSC2, DSG3, DST, EPB41L4B, FGFBP1, FGFR3, FST, GJB3, GRHL2, HBEGF, HOOK1, IL18, IL1B, IRF6, ITGB4, JAG2, KLK10, KLK5, KLK7, KLK8, KRT15, KRT16, KRT17, KRT5, KRT6B, LEPREL1, MYO5C, NDRG1, NMU, PI3, RAB25, RLN2, RNF128, S100A14, S100A7, S100A8, SERPINB1, SERPINB2, SLC2A9, SLPI, SNX10, SPINT2, SPRR1A, SPRR1B, ST14, SYK, TMEM40, TRIM29, TSPAN1, UCHL1, VSNL1, ZBED2, AQP3, CBARA1, CD24, CLCA2, CNTNAP2, CSTA, DSC3, EPCAM, ESRP2, FXYD3, GJB5, GPR87, IL1A, LAMA3, LAMB3, MAP7, MAPK13, MMP9, MPZL2, NRG1, SCNN1A, SERPINB13, SLC7A5, SPINT1, ST6GALNAC2, TGFA, TP63, TPD52L1, VGLL1, SLC6A8, ABCA12, ANK3, ANXA8, ANXA8L1, ARHGAP25, ARHGAP8, BDKRB2, BIK, C10ORF10, C10ORF116, C1ORF116, C20ORF19, C6ORF105, CELSR2, CKMT1A, CKMT1B, CLDN1, CST6, CYP4F11, E2F5, ELMO3, EPHA1, EVA1, EXPH5, F11R, FAT2, FGFR2, FLJ12684, FLJ20366, FZD3, GLS2, GNAL, IFI30, IGFBP2, IL1RN, IL4R, IRX4, JUP, KCNK1, KIAA0040, KIAA0888, KIAA1815, KRT14, KRT18, LAD1, LGALS7, LOC553158, LOC653562, LOC728113, LOC729884, LRRC1, LSR, MST1R, MYO1D, NAIP, OCLN, NEFM, NUP62CL, PERP, PLS1, POLR3G, PRKCH, PRRG4, PRSS8, PTPN3, RBM35A, RBM35B, RHBDF2, RHOD, RPS6KA1, RTEL1, SAA1, SAA1, SAA2, SH2D3A, SLC6A10P, SMPDL3B, SNCA, STAC, STAP2, TACSTD1, THBD, TMEM30B, TMPRSS11E, TMPRSS4, TNFRSF6B, TP73L, WWC1, XDH, ZNF165; and
(b) BGN, BIN1, CDH11, CDH2, CHN1, COL1A2, COL3A1, COL5A2, CREB3L1, CTGF, CYBRD1, DCN, DLC1, DNAJB4, DPT, ENPP2, FBLN1, FBLN5, FBN1, GREM1, HAS2, IGFBP3, IGFBP4, MLPH, MME, MMP2, MYL9, NEBL, NID1, NR2F1, OLFML3, PCOLCE, PMP22, POSTN, PPAP2B, PRR16, PRRX1, PTX3, PVRL3, RGL1, RGS4, ROR1, SEMA5A, TNFAIP6, TRAM2, WNT5A, ZEB1, ANXA6, CITED2, COL1A1, COL6A1, CYP1B1, DAB2, DOCK10, EML1, GNG11, IER3, ITGBL1, KCNMA1, LRIG1, LUM, MAP1B, NID2, PCDH9, PRKCA, PTGER4, PTGFR, SDC2, SERPINF1, SNED1, SYT11, TFPI, TGFBR3, VCAN, ANKRD25, C10ORF56, C5ORF13, CCDC92, CDKN2C, COPZ2, DDR2, ECM1, ENOX1, FADS2, FAP, FILIP1L, FSTL1, HS3ST2, KRT81, LMCD1, LTBP1, LTBP2, MMP1, NPAC, NRP1, PDGFC, PLEKHC1, PPM1D, PTGER2, SCCPDH, SEPT9, SPOCK1, SRGN, STARD13, STC1, SYNC1, TAGLN, TBX3, TGFB1I1, THY1, TNS3, TPM1, TSHZ1, TUBA1A, UGDH, VIM, XYLT1, ZBTB38; and
(c) ENPP2, CITED2, ZEB1, NID2, SEMA5A, DAB2, KCNMA1, PTX3, PCDH9, CREB3L1, BGN, ROR1, DLC1, GNG11, CDH11, NR2F1, NR2F2, PRR16, MYL9, DOCK10, LRIG1, IER3, EML1, NEBL, RGL1, MLPH, TRAM2, DNAJB4, FBLN5, RGS4, HAS2, ITGBL1, IGFBP4, BIN1, DPT, PCOLCE, GREM1, PPAP2B, CDH2, PMP22, LUM, CHN1, CYP1B1, MME, WNT5A, POSTN, MMP2, CTGF, FBLN1, IGFBP3, DCN, PRRX1, ANXA6, PVRL3, MAP1B, TNFAIP6, CYBRD1, FBN1, NID1, OLFML3, SNAI1, SNAI2, TAGLN, SYNC, TUBA1A, PPM1D, TWIST1, FN1, TGFBR3, SERPINF1, UGDH, SRGN, FAP, PTGER4, VIM, PRKCA, FSTL1, MMP1, NRP1, FILIP1L, SCCPDH, LTBP2, XYLT1, HS3ST2, SYT11, TSHZ1, THY1, SEPT9, S100A4, CCDC92, TNS3, ENOX1, TGFB1I1, ZEB2, LMCD1, PDGFC, ECM1, TFPI, TBX3, DDR2, PTGER2, CDKN2C, VCAN, CD44, STARD13, SNED1, ZBTB38, SDC2, TPM1, COPZ2, STC1, CDH1, KRT5, KRT6B, CD24, EPCAM, ESRP1, CLDN3, CLDN4, ERBB3, SPOCK1, FERMT2, GLYR1, LTBP1, FADS2, KANK2, PTGFR, ARTN, SLC7A5, EPB41L4B, KRT15, CDH3, TPD52L1, ANXA3, PI3, NDRG1, DSG3, S100A14, S100S7, KLK8, S100A8, PTX3, SPINT2, FXYD3, KRT16, RAB25, LGALS7, DSC3, COL5A2, ITGB4, TMEM40, SPRR1A, AP1M2, SERPINB13, TP73L; and
(d) CYBRD1, ARTN, CITED2, SLC7A5, CHN1, CTGF, CSTA, MYL9, EPB41L4B, KRT15, LRIG1, CDH3, TPD52L1, ANXA3, OLFML3, CORO1A, PI3, NDRG1, DSG3, PMP22, S100A14, S100S7, KLK8, S100A8, PTX3, SPINT2, IER3, FXYD3, KRT16, TGFBR3, KRT5, RAB25, SDC2, LUM, LGALS7, KRT6B, CD24, COL1A2, GNG11, DSC3, COL5A2, RGS4, ITGB4, TMEM40, SPRR1A, AP1M2, SERPINB13, SNED1, LEPREL1, TRAM2, TP73L, and PAP2B; and
(e) CYBRD1, ARTN, CITED2, SLC7A5, CHN1, CTGF, CSTA, MYL9, EPB41L4B, KRT15, LRIG1, CDH3, TPD52L1, ANXA3, OLFML3, CORO1A; and
(f) PI3, NDRG1, DSG3, PMP22, S100A14, S100S7, KLK8, S100A8, PTX3, SPINT2, IER3, FXYD3, KRT16, TGFBR3; and
(g) KRT5, RAB25, SDC2, LUM, LGALS7, KRT6B, CD24, COL1A2, GNG11, DSG3, COL5A2; and
(h) RGS4, ITGB4, TMEM40, SPRR1A, AP1M2, AERPINB13, SNED1, LEPREL1, TRAM2, TP73L, and PAB2B; and
(i) CYBRD1, ARTN, KRT15, ITGB4, RGS4, SPRR1B, SLC7A5, ANXA3, IL18, AP1M2, CHN1, OLFML3, ZBED2, LEPREL1, ABLIM1, CDH3, DNAJB4, TRAM2, UCHL1, CTGF; and
   (i) CYBRD1, ARTN, KRT15, ITGB4, RGS4, SPRR1B, SLC7A5, ANXA3, IL18, AP1M2; and
   (ii) CYBRD1, ARTN, KRT15, ITGB4, RGS4; and
(j) CYBRD1, RGS4, CDH3, CHN1, SLC7A5, PRRX1, CITED2, COL5A2, ARTN, ZBED2, SNED1, ITGB4, LUM, PVRL3, OLFML3, BIN1, CCND2, DAB2, ANXA3, IL18; and
   (i) CYBRD1, RGS4, CDH3, CHN1, SLC7A5, PRRX1, CITED2, COL5A2, ARTN, ZBED2; and
   (ii) CYBRD1, RGS4, CDH3, CHN1, SLC7A5; and
(k) CYBRD1, ARTN, RGS4, CITED2, SLC7A5, ITGB4, CHN1, PI3, TMEM40, NDRG1, SPRR1A, PCDH9, CTGF, MAP1B, DSG3, AP1M2, FBLN5, SERPINB13, PMP22, CSTA; and
   (i) CYBRD1, ARTN, RGS4, CITED2, SLC7A5, ITGB4, CHN1, PI3, TMEM40, NDRG1; and
   (ii) CYBRD1, ARTN, RGS4, CITED2, SLC7A5; and

(ii) said second set of genes comprises or consists of:

AXL, NOTCH3, GPR30, PROCR, PAX2, PI3KA, CD44, CD133, ALDH1, BMI1, KRT19, NRP1, MSI1, MTOR, JAG1, p38, ESA1, ZEB1, TWIST1, FOXC2, CDH2, ZEB2, FOXC1, SNAI1, SNAI2, TWIST2, GSC, TCF3, CD44, VIM, FN1, MLPH1, ARHGEF11; and

(iii) said third set of genes comprises or consists of:

At least one gene from said first set of genes and at least one of:

AXL, NOTCH3, GPR30, PROCR, PAX2, PI3KA, CD44, CD133, ALDH1, BMI1, KRT19, NRP1, MSI1, MTOR, JAG1, p38, ESA1, ZEB1, TWIST1, FOXC2, CDH2, ZEB2, FOXC1, SNAI1, SNAI2, TWIST2, GSC, TCF3, CD44, VIM, FN1, MLPH1, ARHGEF11; and

(iv) said fourth set of genes comprises or consists of:

at least two genes from said first set of genes and at least one of:

AXL, NOTCH3, GPR30, PROCR, PAX2, PI3KA, CD44, CD133, ALDH1, BMI1, KRT19, NRP1, MSI1, MTOR, JAG1, p38, ESA1, ZEB1, TWIST1, FOXC2, CDH2, ZEB2, FOXC1, SNAI1, SNAI2, TWIST2, GSC, TCF3, CD44, VIM, FN1, MLPH1, and ARHGEF11; and

(v) said fifth set of genes comprises or consists of:

beta catenin, FZD1, FZD2, FZD3, FZD4, FZD5, FZD6, FZD7, FZD8, FZD9, FZD10, LRP5, LRP6, WNT1, WNT2, WNT2B, WNT3, WNT3A, WNT4 WNT5A, WNT5B, WNT6, WNT7A, WNT7B, WNT8A, WNT8B, WNT9A, WNT9B, WNT10A, WNT10B, WNT11, WNT16, SRFP1, SRFP2, FRZB, SFRP4, SFRP5, DKK1, DKK2, DKK3, DKK4, WIF1, AKT2, PI3KA, Disheveled, Smoothened, Axin1, Axin2; and

(vi) said sixth set of genes comprises or consists of:

At least one gene from said first set of genes and at least one of:

beta catenin, FZD1, FZD2, FZD3, FZD4, FZD5, FZD6, FZD7, FZD8, FZD9, FZD10, LRP5, LRP6, WNT1, WNT2, WNT2B, WNT3, WNT3A, WNT4 WNT5A, WNT5B, WNT6, WNT7A, WNT7B, WNT8A, WNT8B, WNT9A, WNT9B, WNT10A, WNT10B, WNT11, WNT16, SRFP1, SRFP2, FRZB, SFRP4, SFRP5, DKK1, DKK2, DKK3, DKK4, WIF1, AKT2, PI3KA, Disheveled, Smoothened, Axin1, Axin2; and

(vii) said seventh set of genes comprises or consists of:

TGFbeta1, TGFbeta2, TGFbeta3, ALK1, ALK2, ALK3, ALK4, ALK5, ALK6, ALK7, TGFbetaR-II, BMPR-II, ACTR-IIA, ACTR-IIB, AMHR-II, SMAD2, SMAD3; and

(viii) said eighth set of genes comprises or consists of:

at least one gene from said first set of genes and at least one of:

TGFbeta1, TGFbeta2, TGFbeta3, ALK1, ALK2, ALK3, ALK4, ALK5, ALK6, ALK7, TGFbetaR-II, BMPR-II, ACTR-IIA, ACTR-IIB, AMHR-II, SMAD2, SMAD3; and

(ix) said ninth set of genes comprises or consists of:

S100A4, SDC2, CYP1B1, FERMT2, CYBRD1, SNAI2, CD44, BIN1, PMP22, CTGF, IGFBP3, ANXA6, MAP1B, TAGLN, ENPP2, FBLN5, PCOLCE, FN1, VIM, PPAP2B, SEM5A, VCAN, NID2, NID1, HAS2, PRRX1, TNFAIP6, CLDN4, FADS2, DDIT4, GNG11, PER1, ISCU, KPNA3, SLC44A1, FKBP5, NR2F1, MLPH, DOCK10, RGS4, IGFBP4; and

(x) said tenth set of genes comprises or consists of:

S100A4, SDC2, CYP1B1, FERMT2, CYBRD1, SNAI2, CD44, BIN1, ENPP2, FBLN5, PCOLCE, FN1, VIM, PPAP2B, SEM5A, VCAN, CLDN4, FADS2, DDIT4, GNG11, PER1, ISCU, KPNA3, SLC44A1, FKBP5, IGFBP4; and

(xi) said eleventh set of genes comprises or consists of:

SDC2, CYBRD1, SNAI2, PMP22, CTGF, IGFBP3, ANXA6, MAP1B, TAGLN, PPAP2B, HAS2, NRF2, MLPH, DOCK10, RGS4; and

(xii) said twelfth set of genes comprises or consists of:

FERMT2, BIN1, PMP22, CHN1, CTGF, IGFBP3, NID2, CLDN4, CYBRD1, PRR16, ANXA6, PCOLCE, PPAP2B, HAS2, CDH11, MLPH, DOCK10, RGS4, PVRL3, NID2, SLC44A1, FKBP5; and

(xiii) said thirteenth set of genes comprises or consists of:

FERMT2, BIN1, PMP22, CHN1, CTGF, IGFBP3, NID2, CLDN4; and

(xiv) said fourteenth set of genes comprises or consists of:

HAS2, BIN1, PCOLCE, FERMT2, CTGF, IGFBP3, NID2, SLC44A1, FKBP5, and MLPH.

In an embodiment, said plurality of genes is elected from gene sets one, i.e. (i); two i.e., (ii); three, i.e. (iii); four, i.e. (iv); five, i.e. (v); six, i.e. (vi); seven, i.e. (vii); or eight, i.e. (viii); or nine (ix); or ten (x); or eleven (xi); or twelve (xii); or thirteen (xiii); or fourteen (xiv). In an embodiment, said plurality of genes is elected from gene set (i). In an embodiment, said plurality of genes is elected from gene sets (i)a; (i)b; (i)c; (i)d; (i)e; (i)f; (i)g; (i)h; (i)i; (i)j; or (i)k. In an embodiment, said plurality of genes is elected from gene sets (i)i(i); or (i)i(ii). In an embodiment, said plurality of genes is elected from gene sets i(j)i; or (i)j(ii). In an embodiment, said plurality of genes is elected from gene sets (i)k(i); or (i)k(ii). In an embodiment, said plurality of genes is elected from gene set (ii). In an embodiment, said plurality of genes is elected from gene set (iii). In an embodiment, said plurality of genes is elected from gene set (iv). In an embodiment, said plurality of genes is elected from gene set (v). In an embodiment, said plurality of genes is elected from gene set (vi). In an embodiment, said plurality of genes is elected from gene set (vii). In an embodiment, said plurality of genes is elected from gene set (viii). In an embodiment, said plurality of genes is elected from gene set (ix). In an embodiment, said plurality of genes is elected from gene set (x). In an embodiment, said plurality of genes is elected from gene set (xi). In an embodiment, said plurality of genes is elected from gene set (xii). In an embodiment, said plurality of genes is elected from gene set (xiii). In an embodiment, said plurality of genes is elected from gene set (xiv). In an embodiment, said plurality of genes is any subset of genes in gene sets one, two, three, four, five, six, seven, eight, nine, ten, eleven, twelve, thirteen, or fourteen. In an embodiment, said plurality of genes is any subset of genes in gene set one. In an embodiment, said plurality of genes is any subset of genes in gene set two. In an embodiment, said plurality of genes is any subset of genes in gene set three. In an embodiment, said plurality of genes is any subset of genes in gene set four. In an embodiment, said plurality of genes is any subset of genes in gene set five. In an embodiment, said plurality of genes is any subset of genes in gene set six. In an embodiment, said plurality of genes is any subset of genes in gene set seven. In an embodiment, said plurality of genes is any subset of genes in gene set eight. In an embodiment, said plurality of genes is any subset of genes in gene set nine. In an embodiment, said plurality of genes is any subset of genes in gene set ten. In an embodiment, said plurality of genes is any subset of genes in gene set eleven. In an embodiment, said plurality of genes is any subset of genes in gene set twelve. In an embodiment, said plurality of genes is any subset of genes in gene set thirteen. In an embodiment, said plurality of genes is any subset of genes in gene set fourteen.

In an embodiment, said plurality of genes comprises at least two genes; four genes; six genes; eight genes; ten genes; twelve genes; fourteen genes; sixteen genes; eighteen genes; twenty genes; twenty five genes; thirty genes; forty genes; or any range intervening there between. In an embodiment, said plurality comprises more than forty genes.

In an embodiment, said plurality of genes comprises or consists of, a first gene and a second gene. In an embodiment, said plurality of genes further comprises, or consists of, a third gene; a third and fourth gene; a third, fourth, and fifth gene; a third, fourth, fifth, and sixth gene; a third, fourth, fifth, sixth, and seventh; a third, fourth, fifth, sixth, seventh, and eighth gene; a third, fourth, fifth, sixth, seventh, eighth and ninth gene; a third, fourth, fifth, sixth, seventh, eighth, ninth, and tenth gene. In an embodiment, said plurality of genes comprises of more than ten genes.

In an embodiment, said value or values is a function of the level of gene expression of a first gene and the level of gene expression of a second gene. In an embodiment, said value or values is a function of the level of gene expression of said first and second gene, and a third gene; a third and fourth gene; a third, fourth, and fifth gene; a third, fourth, fifth, and sixth gene; a third, fourth, fifth, sixth, and seventh gene; a third, fourth, fifth, sixth, seventh, and eighth gene; a third, fourth, fifth, sixth, seventh, eighth and ninth gene; a third, fourth, fifth, sixth, seventh, eighth, ninth, and tenth gene. In an embodiment, said value or values is a function of the level of gene expression of more than ten genes.

In an embodiment, a first value that is a function of the level of gene expression of said first gene and a second value that is a function of the level of gene expression of said second gene are acquired. In an embodiment, a first value that is a function of the level of gene expression of said first gene, a second value that is a function of the level of gene expression of said second gene, a third value that is a function of the level of gene expression of said third gene, a fourth value that is a function of the level of gene expression of said fourth gene, a fifth value that is a function of the level of gene expression of said fifth gene, a sixth value that is a function of the level of gene expression of said sixth gene, a seventh value that is a function of the level of gene expression of said seventh gene, an eighth value that is a function of the level of gene expression of said eighth gene, a ninth value that is a function of the level of gene expression of said ninth gene, and a tenth value that is a function of the level of gene expression of said tenth gene is acquired. In an embodiment, a plurality of values that is each a function of the level of gene expression of each of a plurality of genes is acquired. In an embodiment, more than ten values that is each a function of the level of gene expression of each of a plurality of genes is acquired.

In an embodiment, a first value that is a function of the level of gene expression of two or more genes of a plurality of genes and a second value that is a function of the level of gene expression of one of the genes of the plurality are acquired. In an embodiment, the invention further features the acquisition of a value or values that is a function of the level of gene expression of a gene not in said first, second, third, fourth, fifth, sixth, seventh, eighth, ninth, tenth, eleventh, twelfth, thirteenth, or fourteenth gene sets. In an embodiment, the invention further features the acquisition of a plurality of value or values that is a function of the level of gene expression of a plurality of genes not in said first, second, third, fourth, fifth, sixth, seventh, eighth, ninth, tenth, eleventh, twelfth, thirteenth, or fourteenth gene sets.

In an embodiment, the invention features the acquisition of a value, e.g., a composite value, that is a function of the level of gene expression of said first gene, the level of gene expression of said second gene, and a weighting factor. In an embodiment, one of said first value or said second value is a function of a weighting factor. In an embodiment, said first value is a function of a first weighting factor and said second value is a function of a second weighting factor. In an embodiment, said first weighting factor and said second weighting factor are different. In an embodiment, the invention features the acquisition of a value, e.g., a composite value, that is a function of the level of gene expression of each of a plurality of genes, and a weighting factor. In an embodiment, the invention features the acquisition of a value or values that is a function of the level of gene expression of each gene of said plurality of genes, wherein each gene in said plurality of genes is a function of a weighting factor. In an embodiment, the invention features the acquisition of a value or values that is the function of the level of gene expression of each gene of a plurality of genes that is further a function of a weighting factor. In an embodiment, the invention features the acquisition of a value or values that is the function of the level of gene expression of each gene of a plurality of genes that is further a function of a weighting factor, wherein each weighting factor for each gene of said plurality of genes is different.

In an embodiment, the invention features the acquisition of a value or values that is a function of the level of gene expression of each gene of said plurality of genes, wherein said value or values is a function of a comparison with a reference criterion. In an embodiment, said value or values is a function of the determination of whether the level of gene expression has a preselected relationship with a reference criterion, e.g., comparing said level of expression, with a preselected reference. In an embodiment, said value or values is a function of said determination; and in another embodiment, the invention features determining if said value or values has a preselected relationship with a reference criterion.

In an embodiment, the invention features the acquisition of a value or values that is a function of the level of gene expression of said plurality of genes, at a predetermined interval, e.g., a first point in time and at least a subsequent point in time.

### Gene Set Score

In an embodiment, the invention features the acquisition of a gene set score. In an embodiment, the gene set score is a function of a value or values that is a function of the level of gene expression of said plurality of genes in said gene sets one and/or two and/or three and/or four and/or five and/or six and/or seven and/or eight and/or nine and/or ten and/or eleven and/or twelve and/or thirteen and/or fourteen. In an embodiment, the gene set score is a function of a value or values that is a function of the level of gene expression of said plurality of genes in said gene sets one and/or two and/or three and/or four and/or five and/or six and/or seven and/or eight and/or nine and/or ten and/or eleven and/or twelve and/or thirteen and/or fourteen and further a function of the level of gene expression of a gene or plurality of genes in Table 1.

TABLE 1
Genes of tumor initiation, EMT, and Cancer Stem Cell classifiers

| | | | | | |
|---|---|---|---|---|---|
| DPF2 | KIAA0436 | CLTC | RAD51L1 | STAU1 | CTSL2 |
| CASP8 | CYP4V2 | COPB2 | EPPK1 | TUBB3 | CXADR |
| BCL2 | JTV1 | SLC25A25 | COL1A1 | UBE2S | CYP27B1 |
| SCGN | ICMT | ECOP | MMP9 | XPNPEP1 | DSC2 |
| SWAP70 | DNMT3A | PDE8A | SERPINE1 | CDKN1A | DSG3 |
| KIAA0276 | HNMT | STAM | SPARC | CHRD | DST |
| C10orf9 | METTL7A | TUBB | TGFB1 | H19 | EPB41L4B |
| C10orf7 | METTL2 | SNX6 | TGFB3 | ID3 | FGFBP1 |
| ALKBH | VIL2 | RAB23 | TGFB1 | ID4 | FGFR3 |
| TOB2 | TPD52 | PLAA | TGFBR1 | IGFBP7 | FST |
| XPR1 | ARPC5 | STC2 | TGIF | LRP1 | GJB3 |
| CD59 | NOL8 | LTF | TGIF2 | MSX1 | GRHL2 |
| LRP2 | NSF | ISGF3G | THBS1 | NOTCH3 | HBEGF |
| PLP2 | RAD23B | ATXN3 | ANXA5 | PROCR | HOOK1 |
| MAPK14 | SRP54 | GTF3C3 | ACTG1 | GBX2 | IL18 |
| CXCL2 | HSPA2 | GSK3B | ARF3 | KI67 | IL1B |
| MMP7 | PBP | KLF10 | ATP1B3 | CCNB1 | IRF6 |
| MGP | THAP2 | ELL2 | BAT3 | BUB1 | ITGB4 |
| MLF1 | CIRBP | ZBTB20 | CALD1 | KNTC2 | JAG2 |
| FLNB | SNRPN | IRX3 | CENTD2 | USP22 | KLK10 |
| SCNM1 | KIAA0052 | ETS1 | CLIC1 | HCFC1 | KLK5 |
| HSPC163 | DUSP10 | SERTAD1 | CTBS | RNF2 | KLK7 |
| C5orf18 | SSR1 | MGC4251 | DPYSL3 | ANK3 | KLK8 |
| MGC4399 | ERBB4 | MAFF | DVL3 | FGFR2 | KRT15 |
| CDW92 | EMP1 | SFPQ | EXT1 | CES1 | KRT16 |
| TMC4 | CHPT1 | CITED4 | FGFR1 | COL1A2 | KRT17 |
| ZDHHC2 | LRPAP1 | CEBPD | FTL | COL3A1 | LEPREL1 |
| TICAM2 | FLJ11752 | EIF4E2 | GNB2L1 | COL5A2 | MYO5C |
| KDELR3 | CSTF1 | HS2ST1 | GPRC5A | COL6A1 | NDRG1 |
| GNPDA1 | KLHL20 | AGPS | H2AFZ | ANKRD25 | NMU |
| THEM2 | DNAJC13 | PGK1 | HIF1A | C10ORF56 | PI3 |
| DBR1 | APLP2 | ATIC | IL13RA1 | C5ORF13 | RAB25 |
| FLJ90709 | ARGBP2 | ETNK1 | KDELR2 | KRT81 | RLN2 |
| FLJ10774 | DNAJB1 | LG2 | LARP1 | N-PAC | RNF128 |
| C16orf33 | NEBL | NCE2 | LPIN2 | PLEKHC1 | S100A14 |
| GAPD | SH3BGRL | 8-Mar | MARS | 9-Sep | S100A7 |
| LDHA | NUDT5 | CNOT4 | MMP10 | SYNC1 | S100A8 |
| MR-1 | GABARAPL1 | RNF8 | MMP14 | MBP | SERPINB1 |
| LARS | MAPT | PSMA5 | MT2A | ABLIM1 | SERPINB2 |
| GTPBP1 | DCBLD1 | DPF2 | MYO10 | ALDH1A3 | SLC2A9 |
| PRSS16 | STK39 | AMMECR1 | NUP62 | ALOX15B | SLPI |
| WFDC2 | PAK2 | KIAA1287 | ROR1 | TUBA1A | ESRP1 |
| AIM1 | CSNK2A1 | LOC144233 | DLC1 | PPM1D | CLDN3 |
| DHRS6 | PILRB | LOC286505 | GNG11 | TWIST1 | CLDN4 |
| DHRS4 | ERN1 | PNAS-4 | CDH11 | FN1 | ERBB3 |
| GC15429 | SGKL | FLJ20530 | NR2F1 | TGFBR3 | SPOCK1 |
| MGC45840 | WEE1 | HUMPD3 | PRR16 | SERPINF1 | FERMT2 |
| ECHDC2 | MAST4 | GC45564 | MYL9 | UGDH | GLYR1 |
| GOLGIN-67 | C11orf17 | CAP350 | DOCK10 | SRGN | LTBP1 |
| AFURS1 | NUP37 | ETAA16 | LRIG1 | FAP | FADS2 |
| HAN11 | GAS7 | ZNF335 | IER3 | PTGER4 | KANK2 |
| DNAPTP6 | TRAM2 | SH3KBP1 | EML1 | PRKCA | PTGFR |
| C7orf25 | BASP1 | MST150 | NEBL | FSTL1 | COL11A2 |
| FLJ37953 | FOXO1A | PRO1073 | RGL1 | MMP1 | KLK3 |
| FLJ10587 | POLR2A | LOC388397 | MLPH | NRP1 | EIF2C2 |
| C7orf36 | PER1 | FKBP5 | DNAJB4 | FILIP1L | ZFP41 |
| ELP4 | DDIT4 | HIPK2 | FBLN5 | SCCPDH | FAM49B |
| NDEL1 | CD97 | KLF13 | RGS4 | LTBP2 | PSORS1C2 |
| NPD014 | BIN1 | ANTXR2 | HAS2 | XYLT1 | MRPL42 |
| KFZP564D172 | SH2B3 | IFNAR1 | ITGBL1 | HS3ST2 | MRPL54 |
| FAM53C | DDB2 | LIX1L | IGFBP4 | SYT11 | MRPL47 |
| IER5 | EMP3 | CHST11 | DPT | TSHZ1 | MRPS23 |
| LOC255783 | NDST2 | AKAP2 | PCOLCE | THY1 | EIF3S9 |
| KIAA0146 | CHST2 | DTX1 | GREM1 | 9-Sep | ALG5 |
| KIAA0792 | NT5E | ST3GAL2 | PPAP2B | S100A4 | DNAJC19 |
| LOC439994 | PDE4A | ADAMTS7 | CDH2 | TNS3 | TPRXL |
| LOC283481 | CPS1 | TNRC6B | PMP22 | ENOX1 | NOTCH2 |
| CG018 | PTGS1 | CYGB | LUM | TGFB1I1 | RBM15 |
| LOC130576 | GGCX | SDHAL1 | CHN1 | ZEB2 | ST3GAL3 |
| NGFRAP1L1 | IRF5 | LOC572558 | CYP1B1 | LMCD1 | NFYA |
| KIAA1217 | ZBTB16 | TRIO | MME | PDGFC | PCNX |
| 4orf7 | MAP4K4 | FRAS1 | WNT5A | ECM1 | FBXO21 |
| C21orf86 | CHST7 | KIAA1632 | POSTN | TFPI | WWOX |
| C9orf64 | KLF12 | POLS | MMP2 | TBX3 | CAMK2B |
| FLJ13456 | NFRKB | EBF | CTGF | DDR2 | PNPLA2 |
| KIAA1600 | PSD | MAML2 | CLIC5 | PFKFB3 | ANXA3 |
| B7-H4 | FKSG49 | PTPRA | UGCGL1 | PLOD2 | AP1M2 |
| LOC80298 | NIFUN | PLEKHG2 | FBXL18 | PSMB7 | ARTN |
| C7orf2 | FYN | DYM | ADRBK1 | PSMD8 | CA2 |
| NUCKS | ZMYM2 | SOX6 | SLC38A2 | RIN2 | CA9 |
| DKFZP566D1346 | CACNA1G | ARHGEF2 | IL8RA | RYBP | CDH3 |
| LOC388279 | SLC25A16 | ZCCHC6 | TAS2R14 | SDF4 | CDS1 |
| FLJ31795 | FLII | PPP3CA | CD300LB | SETD5 | COL17A1 |
| 6orf107 | EIF1 | FAM70B | GIPC3 | SPP1 | CORO1A |
| FLJ12439 | SEPT6 | TMED5 | MYCBP2 | LUZP1 | TCHP |
| FLJ12806 | PHF15 | FLJ43663 | FLJ90709 | FBLN1 | CDKN2C |
| FLJ39370 | NUP188 | HPS1 | PCTK2 | IGFBP3 | VCAN |
| GATS | ABR | MEF2A | PDE4DIP | DCN | CD44 |
| CCDC92 | CNR1 | ST3GAL5 | KIAA0194 | PRRX1 | STARD13 |
| FMNL2 | LOC283824 | SMYD3 | HOM-TES-103 | ANXA6 | SNED1 |
| ARID1B | FSTL4 | KLF7 | ENPP2 | PVRL3 | ZBTB38 |
| ZFHX1B | DNM1 | LOC200230 | CITED2 | MAP1B | SDC2 |
| SSBP2 | APOBEC3G | RERE | ZEB1 | TNFAIP6 | TPM1 |
| ARID5B | ATP2B1 | QKI | NID2 | CYBRD1 | COPZ2 |
| LOC157381 | SMPD1 | BICD1 | SEMA5A | FBN1 | STC1 |
| KPNA3 | SLC11A1 | CTNNB1 | DAB2 | NID1 | CDH1 |
| ARHGAP24 | FXYD5 | POU2F2 | KCNMA1 | OLFML3 | KRT5 |
| CCND2 | C14orf139 | EIF4ENIF1 | PTX3 | SNAI1 | KRT6B |
| VIM | SH3BGRL3 | BTG1 | PCDH9 | SNAI2 | EPCAM |
| CREB3L1 | TAGLN | CD24 | BGN | SYNC | GLYR1 |
| PALM2 | | | | | |

### Level of RNA Expression

In an embodiment, the invention features acquiring a value or values that is a function of the level of gene expression of said plurality of genes in said gene sets one and/or two and/or three and/or four and/or five and/or six and/or seven and/or eight and/or nine and/or ten and/or eleven and/or twelve and/or thirteen and/or fourteen. In an embodiment, the level of gene expression is a function of the level of RNA expression of said plurality of genes. In an embodiment, the level of gene expression is a function of the level of RNA expression of each gene of said plurality of genes. In an embodiment, the level of RNA expression is acquired. In an embodiment, the level of RNA expression of said plurality of genes is assayed. In an embodiment, the level of RNA expression is assayed by detecting an RNA product, e.g., mRNA of said sample. In an embodiment, the level of RNA expression is assayed by a hybridization based method, e.g., hybridization with a probe that is specific for said RNA product. In an embodiment, the level of RNA expression is assayed by; applying said sample, or the mRNA isolated from, or amplified from; said sample, to a nucleic acid microarray, or chip array. In an embodiment, the level of RNA expression is assayed by microarray. In an embodiment, the level of RNA expression is assayed by a polymerase chain reaction (PCR) based method, e.g., qRT-PCR. In an embodiment, the level of RNA expression is assayed by a sequencing based method. In an embodiment, the level of RNA expression is assayed by quantitative RNA sequencing. In an embodiment, the level of RNA expression is assayed by RNA in situ hybridization. In an embodiment, the level of RNA expression is assayed in the whole subject sample. In an embodiment, the level of RNA expression is assayed in a subregion of the subject sample, e.g., subregions of a tissue sample.

In an embodiment, the level of gene expression is a function of the level of protein expression of a plurality of genes in said gene sets one and/or two and/or three and/or four and/or five and/or six and/or seven and/or eight and/or nine and/or ten and/or eleven and/or twelve and/or thirteen and/or fourteen. In an embodiment, the level of gene expression is a function of the level of protein expression of said plurality of genes. In an embodiment, the level of gene expression is a function of the level of protein expression of each gene of said plurality of genes. In an embodiment, the level of protein expression is acquired. In an embodiment, the level of protein expression is assayed. In an embodiment, the level of protein expression is assayed by detecting a protein product. In an embodiment, the level of protein expression is assayed using antibodies selective for said protein product. In an embodiment, the level of protein expression is assayed by an immunohistochemistry technique. In an embodiment, the level of protein expression is assayed by an immunohistochemistry technique, using antibodies specific for said protein product. In an embodiment, the level of protein expression is assayed by an immunoassay, e.g., Western blot, enzyme linked immunosorbant assay (ELISA). In an embodiment, the level of protein expression is assayed by an immunoassay specific for said protein. In an embodiment, levels of gene expression are assessed using protein activity assays, such as functional assays. In an embodiment, the level of protein expression is assayed in the whole subject sample. In an embodiment, the level of protein expression is assayed in a subregion of the subject sample, e.g., subregions of a tissue sample.

### Subject Sample

In an embodiment, the method of the invention features acquiring a subject sample, e.g., blood, urine, or tissue sample. In an embodiment, the subject sample is a tissue sample, e.g., biopsy. In an embodiment, the subject sample is a bodily fluid, e.g., blood, plasma, urine, saliva, sweat, tears, semen, or cerebrospinal fluid. In an embodiment, the subject sample is a bodily product, e.g., exhaled breath. In an embodiment, said subject sample is a tissue sample, wherein said tissue sample is derived from fixed tissue, paraffin embedded tissue, fresh tissue, or frozen tissue. In an embodiment, said subject sample is a tissue sample, wherein said tissue sample is fixed tissue, paraffin embedded tissue, fresh tissue, or frozen tissue.

In an embodiment, said subject sample is derived from a tumor. In an embodiment, said subject sample is obtained from a tumor sample. In an embodiment, said subject sample is a tumor sample. In an embodiment, said subject sample is obtained from tumor tissue. In an embodiment, the subject sample is tumor tissue. In an embodiment, said subject sample is obtained from tumor tissue, wherein said subject sample is fixed tumor tissue, paraffin embedded tumor tissue, fresh tumor tissue, or frozen tumor tissue. In an embodiment, said subject sample is a tissue sample, wherein said tissue sample is fixed, paraffin embedded, fresh, or frozen. In an embodiment, said subject sample is fixed, paraffin embedded, fresh, frozen, or fixed paraffin embedded tumor tissue.

In an embodiment, the subject sample is derived from a biopsy. In an embodiment, said subject sample derived from said biopsy is fresh tissue. In an embodiment, said subject sample derived from said biopsy is tumor tissue. In an embodiment, said subject sample derived from said biopsy is non-tumor tissue. In an embodiment, said subject sample is derived from a fine needle aspirate biopsy; large core needle biopsy; or directional vacuum assisted biopsy. In an embodiment, the subject sample is a tissue sample, wherein said tissue sample is derived from a fine needle aspirate; large core needle biopsy; or directional vacuum assisted biopsy.

In an embodiment, the subject sample is blood. In an embodiment, the subject sample is blood in which circulating tumor cells have been captured or isolated. In an embodiment, the subject sample is said circulating tumor cells that have been captured or isolated from said blood.

### Location Specific Acquisition of the Level of Gene Expression

In an embodiment, the invention features, acquiring a value or values for locations in a subject sample. In an embodiment, a value or values is acquired for a plurality of locations in a subject sample. In an embodiment, a first value or values is acquired for a first location in said subject sample. In an embodiment, a second value or values is acquired for a second location in said subject sample. In an embodiment, said first value or values is different from said second value or values. In an embodiment, the invention features, determining if said first value or values and said second value or values has a preselected relationship with a reference criterion. In an embodiment, determination of whether said first value or values and/or said second value or values has a preselected relationship with a reference criterion includes comparing said first value or values with said second value or values.

In an embodiment, said first value or values is associated with an increased likelihood of comprising a cancer stem cell, cancer associated mesenchymal cell, or tumor initiating cancer cell; than is said second value or values. In an embodiment, said first value or values is associated with a higher likelihood of comprising a cancer stem cell than is said second value or values. In an embodiment, said first value or values is associated with a higher likelihood of comprising a cancer associated mesenchymal cell than is said second value or values. In an embodiment, said first value or values is associated with a higher likelihood of comprising a tumor initiating cancer cell than is said second value or values. In an embodiment, said first value or values is indicative of a cancer stem cell, cancer associated mesenchymal cell, or tumor initiating cancer cell. In an embodiment, said first value or values is indicative of a cancer stem cell. In an embodiment, said first value or values is indicative of a cancer associated mesenchymal cell. In an embodiment, said first value or values is indicative of a tumor initiating cancer cell.

In an embodiment, the invention features, classifying a location in a subject sample as a cancer stem cell, cancer associated mesenchymal cell, or tumor initiating cancer cell. In an embodiment, the invention features, classifying said location as a cancer stem cell or non-cancer stem cell. In an embodiment, the invention features, classifying said location as a cancer stem cell. In an embodiment, the invention features, classifying said location as a non-cancer stem cell. In an embodiment, the invention features, classifying said location as a cancer associated mesenchymal cell. In an embodiment, the invention features, classifying said location as a tumor initiating cancer cell. In an embodiment, the invention features, acquiring a first value or values for a first location in said subject sample, wherein responsive to said first value or values, classifying said first location as comprising a cancer stem cell or non-cancer stem cell. In an embodiment, the invention features, acquiring a first value or values for a first location in said subject sample, wherein responsive to said first value or values, classifying said first location as comprising a cancer stem cell, cancer associated mesenchymal cell, or tumor initiating cancer cell.

In an embodiment, the invention features, acquiring a first value or values for a first location in a subject sample, wherein responsive to said first value or values, classifying said first location as comprising a cancer stem cell. In an embodiment, the invention features, acquiring a first value or values for a first location in said subject sample, wherein responsive to said first value or values, classifying said first location as comprising a non-cancer stem cell. In an embodiment, the invention features, acquiring a first value or values for a first location in a subject sample, wherein responsive to said first value or values, classifying said first location as comprising a cancer associated mesenchymal cell. In an embodiment, the invention features, acquiring a first value or values for a first location in a subject sample, wherein responsive to said first value or values, classifying said first location as comprising a tumor initiating cancer cell.

In an embodiment, said first location is classified as a cancer stem cell, cancer associated mesenchymal cell, or tumor initiating cancer cell. In an embodiment, said first location is classified as a cancer stem cell. In an embodiment, said first location is classified as a cancer associated mesenchymal cell. In an embodiment, said first location is classified as a tumor initiating cancer cell. In an embodiment, said first location is classified as a non-cancer stem cell. In an embodiment, said first location comprises a cancer stem cell, cancer associated mesenchymal cell, or tumor initiating cancer cell. In an embodiment, said first location comprises a cancer stem cell. In an embodiment, said first location comprises a cancer associated mesenchymal cell. In an embodiment, said first location comprises a tumor initiating cancer cell. In an embodiment, said first location comprises a non-cancer stem cell. In an embodiment, said first location is indicative of a cancer stem cell, cancer associated mesenchymal cell, or tumor initiating cancer cell. In an embodiment, said first location is indicative of a cancer stem cell. In an embodiment, said first location is indicative of a cancer associated mesenchymal cell. In an embodiment, said first location is indicative of a tumor initiating cancer cell. In an embodiment, said first location is indicative of a non-cancer stem cell.

In an embodiment, said first location comprises a subject sample. In an embodiment, said first location comprises a whole subject sample. In an embodiment, said first location comprises a sub-region of the subject sample. In an embodiment, said first location and said second location are separated by zero microns, i.e., said first location and second location are adjoining. In an embodiment, said first location and said second location are separated by more than zero microns; by more than ten microns; by more than twenty microns; by more than thirty microns; by more than forty microns; by more than fifty microns; by more than sixty microns; by more than seventy microns; by more than eighty microns; by more than ninety microns; or by more than one hundred microns. In an embodiment, said first location and said second location are separated by more than one thousand microns. In an embodiment, said first location and said second location are separated by at least ten microns; in an embodiment, said first location and said second location are separated by at least twenty microns; by at least thirty microns; by at least forty microns; by at least fifty microns; by at least sixty microns; by at least seventy microns; by at least eighty microns; by at least ninety microns; or by at least one hundred microns. In an embodiment, said first location and said second location are separated by more than one hundred microns. In an embodiment, said first location and said second location are separated by more than two hundred microns; three hundred microns; four hundred microns; five hundred microns; six hundred microns; seven hundred microns; eight hundred microns; nine hundred microns; or one thousand microns. In an embodiment, said first location and said second location are separated by at least one thousand microns. In an embodiment, said first location and said second location are separated by the maximum distance two locations of said subject sample can be separated. In an embodiment, said first location and said second location are separated by a distance between and including, zero and the maximum distance two locations of said subject sample can be separated.

In an embodiment, the average distance between said first location and said second location is more than zero microns; in an embodiment, the average distance between said first location and said second location is approximately ten microns; approximately twenty microns; approximately thirty microns; approximately forty micron; approximately fifty microns; approximately sixty microns; approximately seventy microns; approximately eighty microns; approximately ninety microns; or approximately one hundred microns. In an embodiment, the average distance between said first location and said second location is more than approximately fifty microns.

In an embodiment, the average distance between said first location and said second location is zero microns; in an embodiment, the average distance between said first location and said second location is more than ten microns; more than twenty microns; more than thirty microns; more than forty micron; more than fifty microns; more than sixty microns; more than seventy microns; more than eighty microns; more than ninety microns; or more than one hundred microns.

In an embodiment, the average distance between said first location and said second location is more than approximately one hundred microns. In an embodiment, the average distance between said first location and said second location is more than approximately two hundred; more than approximately three hundred; more than approximately four hundred; more than approximately five hundred; more than approximately six hundred; more than approximately seven hundred; more than approximately eight hundred; more than approximately nine hundred; or more than approximately one thousand microns. In an embodiment, the average distance between said first location and said second location is more than one thousand microns.

In an embodiment, the average distance between said first location and said second location is at least approximately ten microns; at least approximately twenty microns; at least approximately thirty microns; at least approximately forty microns; at least approximately fifty microns; at least approximately sixty microns; at least approximately seventy microns; at least approximately eighty microns; at least approximately ninety microns; at least approximately one hundred microns; at least approximately two hundred microns.

In an embodiment, said first value or values of said first location is a function of the level of gene expression of a gene at said first location. In embodiment, said first value or values is a function of the level of gene expression of a plurality of genes at said first location. In an embodiment, said first value or values is a function of the level of gene expression of each gene isoform of a plurality of genes at said first location. In an embodiment, the invention features the first value or values of said first location is a function of the level of gene expression of a gene or a plurality of genes at said first location, and responsive to said first value or values classifying said first location as a cancer stem cell or non cancer stem cell. In an embodiment, the invention features the first value or values of said first location is a function of the level of gene expression of a gene or a plurality of genes at said first location, and responsive to said first value or values classifying said first location as a cancer stem cell, cancer associated mesenchymal cell, or tumor initiating cancer cell. In an embodiment, said gene or said plurality of genes is in Table 1. In an embodiment, the level of gene expression is a function of the level of RNA expression of said gene or said plurality of genes. In an embodiment, the level of RNA expression of said gene or plurality of genes is assayed. In an embodiment, the level of RNA expression is assayed by detecting an RNA product. In an embodiment, the level of RNA expression is assayed by RNA in situ hybridization. In an embodiment, the level of gene expression is a function of the level of protein expression of said gene or said plurality of genes. In an embodiment, the level of protein expression is acquired. In an embodiment, the level of protein expression is assayed. In an embodiment, the level of protein expression is assayed by detecting a protein product. In an embodiment, the level of protein expression is assayed using antibodies selective for said protein product. In an embodiment, the level of protein expression is assayed by immunohistochemistry.

### Administration

In an embodiment, the invention features administering an agent that inhibits or kills cancer associated mesenchymal cells, tumor initiating cancer cells, or cancer stem cells. In an embodiment, said agent that inhibits or kills cancer associated mesenchymal cells, tumor initiating cancer cells or cancer stem cells is administered to said subject. In an embodiment, the agent that inhibits or kills cancer associated mesenchymal cells, tumor initiating cancer cells, or cancer stem cells is selected from, e.g., salinomycin; a gamma secretase inhibitor; a DLL4 inhibitor, e.g., a therapeutic antibody targeting DLL4; a TRAIL inhibitor, e.g., a therapeutic antibody targeting TRAIL; a Hedgehog inhibitor, e.g., a therapeutic antibody targeting Hedgehog; a NOTCH3 inhibitor, e.g., a therapeutic antibody targeting NOTCH3; a NOTCH4 inhibitor, e.g., a therapeutic antibody targeting NOTCH4; a panNOTCH inhibitor, e.g., a therapeutic antibody targeting panNOTCH; a FGFR1 inhibitor, e.g., a therapeutic antibody targeting FGR1; a FGFR2 inhibitor, e.g., a therapeutic antibody targeting FGR2; a FGFR3 inhibitor, e.g., a therapeutic antibody targeting FGR3; a FGFR4 inhibitor, e.g., a therapeutic antibody targeting FGR4; a RON inhibitor, e.g., a therapeutic antibody targeting RON; Wnt pathway inhibitor, e.g., therapeutic antibodies targeting the Wnt pathway; a PI3Kinase inhibitor; a mTOR inhibitor; sodium meta arsenite; verapail; reserpine; a perifosen inhibitor of FAK1; a FAK inhibitor; a p38 inhibitor.

In an embodiment, the method features selecting a regimen, e.g., dosage, formulation, route of administration, number of dosages, or adjunctive therapies, of the agent that inhibits or kills cancer associated mesenchymal cells, tumor initiating cancer cells, or cancer stem cells. In an embodiment, said selecting is responsive to said value or values that is a function of the level of gene expression of said plurality of genes selected from said first and/or second and/or third and/or fourth and/or fifth and/or sixth and/or seven and/or eighth gene sets. In an embodiment, the invention features administering an agent that inhibits or kills cancer associated mesenchymal cells, tumor initiating cancer cells, or cancer stem cells to the subject according to the selected regimen. In an embodiment, said administration is provided responsive to acquiring knowledge or information of said value or values from another party. In an embodiment, said administration is provided responsive to an identification of said value or values, wherein said identification arises from collaboration with another party. In an embodiment, the invention features receiving a communication of the presence of said value or values that is a function of the level of gene expression for each of a plurality of genes selected from said first and/or second and/or third and/or fourth and/or fifth and/or sixth and/or seventh and/or eighth and/or ninth and/or tenth and/or eleventh and/or twelfth and/or thirteenth and/or fourteenth set of genes in a subject. In an embodiment, the acquisition of said value or values is at the time of or after diagnosis of cancer in said subject. In an embodiment, the acquisition of said value or values is post diagnosis of said cancer in the subject. In an embodiment, said subject has cancer. In an embodiment, the cancer is characterized as comprising cancer associated mesenchymal cells, tumor initiating cancer cells, or cancer stem cells. In an embodiment, the cancer is characterized as comprising cancer associated mesenchymal cells. In an embodiment, the cancer is characterized as comprising tumor initiating cancer cells. In an embodiment, the cancer is characterized as comprising cancer stem cells. In an embodiment, the cancer is characterized as being enriched with cancer associated mesenchymal cells, tumor initiating cancer cells, or cancer stem cells. In an embodiment, the cancer is characterized as being enriched with cancer associated mesenchymal cells. In an embodiment, the cancer is characterized as being enriched with tumor initiating cancer cells. In an embodiment, the cancer is characterized as being enriched with cancer stem cells.

In an embodiment, said cancer is an epithelial cell cancer. In an embodiment, said cancer is breast, lung, pancreatic, colorectal, prostate, head and neck, melanoma, acute myelogenous leukemia, glioblastoma, triple negative breast cancer, basal-like breast cancer, or claudin-low breast cancer. In another embodiment, said cancer is breast cancer. In an embodiment, said cancer is triple negative breast cancer. In an embodiment, the cancer is basal-like breast cancer. In an embodiment, the cancer is claudin-low breast cancer. In an embodiment, said cancer is recurrent, i.e., cancer that returns following treatment, and after a period of time in which said cancer was undetectable. In another embodiment, said cancer is a primary tumor, i.e., located at the anatomical site of tumor growth initiation. In an embodiment, said cancer is metastatic, i.e., appearing at a second anatomical site other than the anatomical site of tumor growth initiation.

In an embodiment of the invention, the value or values that is a function of the level of gene expression of said plurality of genes selected from said first and/or second and/or third and/or fourth and/or fifth and/or sixth and/or seventh and/or eighth and/or ninth and/or tenth and/or eleventh and/or twelfth and/or thirteenth and/or fourteenth set of genes; is acquired prior to, during, or after administration of a treatment to said subject. In an embodiment, said value or values is acquired prior to the administration of a treatment to said subject. In an embodiment, said value or values is acquired during the administration of a treatment to said subject. In an embodiment, said value or values is acquired after the administration of a treatment to said subject. In an embodiment, said subject is a non-responder, to said treatment. In an embodiment, said treatment is an anti-cancer treatment, e.g., chemotherapeutic agent, radiation treatment, surgery, etc. In an embodiment, said anti-cancer treatment is a chemotherapeutic agent. In an embodiment, said chemotherapeutic agent may include but is not limited to is one or more of the following chemotherapeutic agents: alkylating agents (e.g., nitrogen mustards such as chlorambucil, cyclophosphamide, isofamide, mechlorethamine, melphalan, and uracil mustard; aziridines such as thiotepa; methanesulphonate esters such as busulfan; nitroso ureas such as carmustine, lomustine, and streptozocin; platinum complexes such as cisplatin and carboplatin; bioreductive alkylators such as mitomycin, procarbazine, dacarbazine and altretamine); DNA strand-breakage agents (e.g., bleomycin); topoisomerase II inhibitors (e.g., amsacrine, dactinomycin, daunorubicin, idarubicin, mitoxantrone, doxorubicin, etoposide, and teniposide); DNA minor groove binding agents (e.g., plicamydin); antimetabolites (e.g., folate antagonists such as methotrexate and trimetrexate; pyrimidine antagonists such as fluorouracil, fluorodeoxyuridine, CB3717, azacitidine, cytarabine, and floxuridine; purine antagonists such as mercaptopurine, 6-thioguanine, fludarabine, pentostatin; asparginase; and ribonucleotide reductase inhibitors such as hydroxyurea); tubulin interactive agents (e.g., vincristine, vinblastine, and paclitaxel (Taxol)); hormonal agents (e.g., estrogens; conjugated estrogens; ethinyl estradiol; diethylstilbesterol; chlortrianisen; idenestrol; progestins such as hydroxyprogesterone caproate, medroxyprogesterone, and megestrol; and androgens such as testosterone, testosterone propionate, fluoxymesterone, and methyltestosterone); adrenal corticosteroids (e.g., prednisone, dexamethasone, methylprednisolone, and prednisolone); leutinizing hormone releasing agents or gonadotropin-releasing hormone antagonists (e.g., leuprolide acetate and goserelin acetate); and antihormonal antigens (e.g., tamoxifen, antiandrogen agents such as flutamide; and antiadrenal agents such as mitotane and aminoglutethimide). In an embodiment, said chemotherapeutic agent is selected from one or more of the following chemotherapeutic agents: Capecitabine, Carboplatin, Cisplatin, Cyclophosphamide, Docetaxel, Doxorubicin, Epirubicin, Eribulin, mesylate5-Fluorouracil, Gemcitabine, Ixabepilone, Liposomal doxorubicin, Methotrexate, Paclitaxel, or Vinorelbine; or any combination thereof.

In an embodiment, the invention features administering an agent that inhibits or kills cancer associated mesenchymal cells, tumor initiating cancer cells, or cancer stem cells and a second treatment. In an embodiment, said second treatment is an anti-cancer agent. In an embodiment, said second treatment is an agent that inhibits or kills cancer associated mesenchymal cells, tumor initiating cancer cells, or cancer stem cells. In an embodiment, said second treatment is not an agent that inhibits or kills cancer associated mesenchymal cells, tumor initiating cancer cells, or cancer stem cells. In an embodiment, said second treatment kills or inhibits growth of non-cancer stem cells in the subject. In an embodiment, the second treatment kills or inhibits growth of cancer cells that are not cancer stem cells, cancer associated mesenchymal cells, or tumor initiating cancer cells. In an embodiment, the second treatment is an anti-cancer treatment that does not target cancer stem cells, cancer associated mesenchymal cells, or cancer stem cells. In an embodiment, the second treatment is an anti-cancer treatment that does not primarily target cancer stem cells, cancer associated mesenchymal cells, or cancer stem cells. In an embodiment, said second treatment kills or inhibits growth of non-cancer associated mesenchymal cells, non-tumor initiating cancer cells, or non-cancer stem cells in the subject. In an embodiment, said second treatment is a chemotherapeutic agent. In an embodiment, said second treatment may include but is not limited to one or more of the following: alkylating agents (e.g., nitrogen mustards such as chlorambucil, cyclophosphamide, isofamide, mechlorethamine, melphalan, and uracil mustard; aziridines such as thiotepa; methanesulphonate esters such as busulfan; nitroso ureas such as carmustine, lomustine, and streptozocin; platinum complexes such as cisplatin and carboplatin; bioreductive alkylators such as mitomycin, procarbazine, dacarbazine and altretamine); DNA strand-breakage agents (e.g., bleomycin); topoisomerase II inhibitors (e.g., amsacrine, dactinomycin, daunorubicin, idarubicin, mitoxantrone, doxorubicin, etoposide, and teniposide); DNA minor groove binding agents (e.g., plicamydin); antimetabolites (e.g., folate antagonists such as methotrexate and trimetrexate; pyrimidine antagonists such as fluorouracil, fluorodeoxyuridine, CB3717, azacitidine, cytarabine, and floxuridine; purine antagonists such as mercaptopurine, 6-thioguanine, fludarabine, pentostatin; asparginase; and ribonucleotide reductase inhibitors such as hydroxyurea); tubulin interactive agents (e.g., vincristine, vinblastine, and paclitaxel (Taxol)); hormonal agents (e.g., estrogens; conjugated estrogens; ethinyl estradiol; diethylstilbesterol; chlortrianisen; idenestrol; progestins such as hydroxyprogesterone caproate, medroxyprogesterone, and megestrol; and androgens such as testosterone, testosterone propionate, fluoxymesterone, and methyltestosterone); adrenal corticosteroids (e.g., prednisone, dexamethasone, methylprednisolone, and prednisolone); leutinizing hormone releasing agents or gonadotropin-releasing hormone antagonists (e.g., leuprolide acetate and goserelin acetate); and antihormonal antigens (e.g., tamoxifen, antiandrogen agents such as flutamide; and antiadrenal agents such as mitotane and aminoglutethimide). In an embodiment, said second therapeutic agent is selected from Capecitabine, Carboplatin, Cisplatin, Cyclophosphamide, Docetaxel, Doxorubicin, Epirubicin, Eribulin, mesylate5-Fluorouracil, Gemcitabine, Ixabepilone, Liposomal doxorubicin, Methotrexate, Paclitaxel, or Vinorelbine; or any combination thereof. In an embodiment, the invention features further administering an agent that inhibits or kills cancer associated mesenchymal cells, tumor initiating cells, or cancer stem cells and more than one additional therapeutic agent.

In an embodiment, the invention includes, responsive to the acquisition of said value or values that is a function of the level of gene expression of said plurality of genes selected from said first and/or second and/or third and/or fourth and/or fifth and/or sixth and/or seventh and/or eighth and/or ninth and/or tenth and/or eleventh and/or twelfth and/or thirteenth and/or fourteenth set of genes; further stratifying a patient population. In an embodiment, the invention features, responsive to the acquisition of said value or values; further identifying or selecting said subject as likely or unlikely to respond positively to a treatment. In another embodiment, the invention features, responsive to the acquisition of said value or values; further selecting a treatment. In another embodiment, the invention features, responsive to the acquisition of said value or values; further prognosticating the time course of the disease in the subject. In an embodiment, said disease is a cancer. In an embodiment, the invention features, responsive to the acquisition of said value or values, one or more of the following: stratifying a patient population; identifying or selecting said subject as likely or unlikely to respond to a treatment; selecting a treatment option; prognosticating the time course of the disease in the subject; measuring the response at the end of therapy and predicting the long term outcome; and/or determining the cancer stem cell population as a predictor of response to a treatment or therapy.

### Genotype

In an embodiment, the method of the invention features the acquisition of a genotype of said subject sample. The subject sample can be any suitable subject sample including those subject samples previously mentioned. In an embodiment, said subject sample is a tumor sample. In an embodiment, at least one nucleotide of the subject sample is sequenced to determine the presence or absence of at least one genetic event associated with cancer. In an embodiment, at least one oncogene or tumor suppressor gene in the sample is sequenced. In an embodiment, the oncogene or oncogenes or tumor suppressor gene or tumor suppressor genes may include but is not limited to one or any combination of: Abl, Af4/hrx, akt-2, alk, alk/npm, aml 1, aml 1/mtg8, APC, axl, bcl-2, bcl-3, bcl-6, bcr/abl, brca-1, brca-2, beta-catenin, CDKN2, c-myc, c-sis, dbl, dek/can, E2A/pbx1, egfr, enl/hrx, erg/TLS, erbB, erbB-2, erk, ets-1, ews/fli-1, fms, fos, fps, gli, gsp, HER2/neu, hox11, hst, IL-3, int-2, jun, kit, KS3, K-sam, Lbc, lck, lmo1, lmo2, L-myc, li1-1, lyt-10, lyt-10/C alpha1, mas, mdm-2, mll, mos, mtg8/aml1, myb, myc, MYH11/CBFB, neu, nm23, N-myc, ost, p53, pax-5, pbx1/E2A, pdgfr, PI3-K, pim-1, PRAD-1, raf, RAR/PML, rash, rasK, rasN, Rb, rel/nrg, ret, rhom1, rhom2, ros, ski, sis, set/can, src, tal1, tal2, tan-1, telomerase, Tiam1, TSC2, trk, vegfr, or wnt.

### Reports

In an embodiment, the present invention features optionally providing a prediction of the likelihood that a subject will respond positively or will not respond positively to treatment with an agent that inhibits or kills cancer associated mesenchymal cells, tumor initiating cancer cells, or cancer stem cells. In an embodiment, said prediction is in the form of a report. In an embodiment, said predication includes a recommendation of whether said subject should be treated with a preselected drug, or treatment with a preselected drug should be withheld. In an embodiment, said preselected drug is an anti-cancer agent. In an embodiment, said preselected drug is an agent that inhibits or kills cancer associated mesenchymal cells, tumor initiating cancer cells, or cancer stem cells. In an embodiment, said agent that inhibits or kills cancer associated mesenchymal cells, tumor initiating cancer cells, or cancer stem cells is selected from: e.g., salinomycin; a gamma secretase inhibitor; a DLL4 inhibitor, e.g., a therapeutic antibody targeting DLL4; a TRAIL inhibitor, e.g., a therapeutic antibody targeting TRAIL; a Hedgehog inhibitor, e.g., a therapeutic antibody targeting Hedgehog; a NOTCH3 inhibitor, e.g., a therapeutic antibody targeting NOTCH3; a NOTCH4 inhibitor, e.g., a therapeutic antibody targeting NOTCH4; a panNOTCH inhibitor, e.g., a therapeutic antibody targeting panNOTCH; a FGFR1 inhibitor, e.g., a therapeutic antibody targeting FGR1; a FGFR2 inhibitor, e.g., a therapeutic antibody targeting FGR2; a FGFR3 inhibitor, e.g., a therapeutic antibody targeting FGR3; a FGFR4 inhibitor, e.g., a therapeutic antibody targeting FGR4; a RON inhibitor, e.g., a therapeutic antibody targeting RON; Wnt pathway inhibitor, e.g., therapeutic antibodies targeting the Wnt pathway; a PI3Kinase inhibitor; a mTOR inhibitor; sodium meta arsenite; verapail; reserpine; a perifosen inhibitor of FAK1; a FAK inhibitor; a p38 inhibitor.

### Kits or Products

In an aspect, the present invention includes a kit or product comprising a first agent capable of interacting with a gene expression product of a gene from a first and/or second and/or third and/or fourth and/or fifth and/or sixth and/or seventh and/or eighth and/or ninth and/or tenth and/or eleventh and/or twelfth and/or thirteenth and/or fourteenth set of genes.

In an embodiment, the first set of genes are related to the delineation of a cancer stem cell, cancer associated mesenchymal cell, or a tumor initiating cancer cell phenotype and the determination of the attributes of the EMT in cancers. For example,

(i) said first set of genes comprises or consists of:
KLF13, ANTXR2, IFNAR1, LIX1L, CHST11, AKAP2, PALM2-AKAPT, LOC80298, C7orf2, NUCKS, DKFZP566D1346, LOC388279, FLJ31795, 6orf107, FLJ12439, ENPP2, CITED2, ZEB1, NID2, SEMA5A, DAB2, KCNMA1, PTX3, PCDH9, CREB3L1, BGN, ROR1, DLC1, GNG11, CDH11, NR2F1, PRR16, MYL9, DOCK10, LRIG1, IER3, EML1, NEBL, RGL1, MLPH, DNAJB4, FBLN5, RGS4, HAS2, ITGBL1, IGFBP4, DPT, PCOLCE, GREM1, PPAP2B, CDH2, PMP22, LUM, CHN1, CYP1B1, MME, WNT5A, POSTN, MMP2, CTGF, FBLN1, IGFBP3, DCN, PRRX1, ANXA6, PVRL3, MAP1B, TNFAIP6, CYBRD1, FBN1, NID1, OLFML3, SNAI1, SNAI2, TAGLN, SYNC, TUBA1A, PPM1D, TWIST1, FN1, TGFBR3, SERPINF1, UGDH, SRGN, FAP, PTGER4, PRKCA, FSTL1, MMP1, NRP1, FILIP1L, SCCPDH, LTBP2, XYLT1, HS3ST2, SYT11, TSHZ1, THY1, Sep9, S100A4, TNS3, ENOX1, TGFB1I1, ZEB2, LMCD1, PDGFC, ECM1, TFPI, TBX3, DDR2, PTGER2, CDKN2C, VCAN, CD44, STARD13, SNED1, ZBTB38, SDC2, TPM1, COPZ2, STC1, CDH1, KRT5, KRT6B, CD24, EPCAM, ESRP1, CLDN3, CLDN4, ERBB3, SPOCK1, FERMT2, GLYR1, LTBP1, FADS2, KANK2, PTGFR, COL11A2, KLK3, EIF2C2, ZFP41, FAM49B, PSORS1C2, MRPL42, MRPL54, MRPL47, MRPS23, EIF3S9, ALG5, DNAJC19, TPRXL, NOTCH2, RBM15, ST3GAL3, NFYA, PCNX, FBXO21, WWOX, CAMK2B, PNPLA2, CLIC5, UGCGL1, FBXL18, ADRBK1, SLC38A2, IL8RA, TAS2R14, CD300LB, GIPC3, RAD51L1, EPPK1, COL1A1, MMP9, SERPINE1, SPARC, TGFB1, TGFB3, TGFBI, TGFBR1, TGIF, TGIF2, THBS1, ANXA5, ACTG1, ARF3, ATP1B3, BAT3, CALD1, CENTD2, CLIC1, CTBS, DPYSL3, DVL3, EXT1, FGFR1, FTL, GNB2L1, GPRC5A, H2AFZ, HIF1A, IL13RA1, KDELR2, LARP1, LPIN2, MARS, MMP10, MMP14, MT2A, MYO10, NUP62, PFKFB3, PLOD2, PSMB7, PSMD8, RIN2, RYBP, SDF4, SETD5, SPP1, STAU1, TUBB3, UBE2S, XPNPEP1, CDKN1A, CHRD, H19, ID3, ID4, IGFBP7, LRP1, MSX1, NOTCH3, PROCR, GBX2, KI67, CCNB1, BUB1, KNTC2, USP22, HCFC1, RNF2, ANK3, FGFR2, CES1, COL1A2, COL3A1, COL5A2, COL6A1, ANKRD25, C10ORF56, C5ORF13, KRT81, NPAC, PLEKHC1, SEPT9, SYNC1, MBP, ABLIM1, ALDH1A3, ALOX15B, ANXA3, AP1M2, ARTN, CA2, CA9, CDH3, CDS1, COL17A1, CORO1A, CTSL2, CXADR, CYP27B1, DSC2, DSG3, DST, EPB41L4B, FGFBP1, FGFR3, FST, GJB3, GRHL2, HBEGF, HOOK1, IL18, IL1B, IRF6, ITGB4, JAG2, KLK10, KLK5, KLK7, KLK8, KRT15, KRT16, KRT17, LEPREL1, MYO5C, NDRG1, NMU, PI3, RAB25, RLN2, RNF128, S100A14, S100A7, S100A8, SERPINB1, SERPINB2, SLC2A9, SLPI, SNX10, SPINT2, SPRR1A, SPRR1B, ST14, SYK, TMEM40, TRIM29, TSPAN1, UCHL1, VSNL1, ZBED2, AQP3, CBARA1, CLCA2, CNTNAP2, CSTA, DSC3, ESRP2, FXYD3, GJB5, GPR87, IL1A, LAMA3, LAMB3, MAP7, MAPK13, MPZL2, NRG1, SCNN1A, SERPINB13, SLC7A5, SPINT1, ST6GALNAC2, TGFA, TP63, TPD52L1, VGLL1, SLC6A8, ABCA12, ANXA8, ANXA8L1, ARHGAP25, ARHGAP8, BDKRB2, BIK, C10ORF10, C10ORF116, C1ORF116, C20ORF19, C6ORF105, CELSR2, CKMT1A, CKMT1B, CLDN1, CST6, CYP4F11, E2F5, ELMO3, EPHA1, EVA1, EXPH5, F11R, FAT2, FLJ12684, FLJ20366, FZD3, GLS2, GNAL, IFI30, IGFBP2, IL1RN, IL4R, IRX4, JUP, KCNK1, KIAA0040, KIAA0888, KIAA1815, KRT14, KRT18, LAD1, LGALS7, LOC553158, LOC653562, LOC728113, LOC729884, LRRC1, LSR, MST1R, MYO1D, NAIP, OCLN, NEFM, NUP62CL, PERP, PLS1, POLR3G, PRKCH, PRRG4, PRSS8, PTPN3, RBM35A, RBM35B, RHBDF2, RHOD, RPS6KA1, RTEL1, SAA1, SAA1, SAA2, SH2D3A, SLC6A10P, SMPDL3B, SNCA, STAC, STAP2, TACSTD1, THBD, TMEM30B, TMPRSS11E, TMPRSS4, TNFRSF6B, TP73L, WWC1, XDH, ZNF165, AXL, GPR30, PAX2, PI3KA, CD133, ALDH1, BMI1, KRT19, MSI1, JAG1, p38, ESA1, FOXC2, FOXC1, TWIST2, GSC, TCF3, MLPH1, ARHGEF11a, DPF2, CASP8, BCL2, SCGN, SWAP70, KIAA0276, C10orf9, C10orf7, ALKBH, TOB2, XPR1, CD59, LRP2, PLP2, MAPK14, CXCL2, MMP7, MGP, MLF1, FLNB, SCNM1, HSPC163, C5orf18, MGC4399, CDW92, TMC4, ZDHHC2, TICAM2, KDELR3, GNPDA1, THEM2, DBR1, FLJ90709, FLJ10774, C16orf33, GAPD, LDHA, MR-1, LARS, GTPBP1, PRSS16, WFDC2, AIM1, DHRS6, DHRS4, GC15429, MGC45840, ECHDC2, GOLGIN-67, AFURS1, KIAA0436, CYP4V2, JTV1, ICMT, DNMT3A, HNMT, METTL7A, METTL2, VIL2, TPD52, ARPC5, NOL8, NSF, RAD23B, SRP54, HSPA2, PBP, THAP2, CIRBP, SNRPN, KIAA0052, DUSP10, SSR1, ERBB4, EMP1, CHPT1, LRPAP1, FLJ11752, CSTF1, KLHL20, DNAJC13, APLP2, ARGBP2, DNAJB1, NEBL, SH3BGRL, NUDT5, GABARAPL1, MAPT, DCBLD1, STK39, PAK2, CSNK2A1, PILRB, ERN1, SGKL, WEE1, MAST4, C11orf17, NUP37, CLTC, COPB2, SLC25A25, ECOP, PDE8A, STAM, TUBB, SNX6, RAB23, PLAA, STC2, LTF, ISGF3G, ATXN3, GTF3C3, GSK3B, KLF10, ELL2, ZBTB20, IRX3, ETS1, SERTAD1, MGC4251, MAFF, SFPQ, CITED4, CEBPD, EIF4E2, HS2ST1, AGPS, PGK1, ATIC, ETNK1, LG2, NCE2, MARCH8, CNOT4, RNF8, PSMA5, DPF2, AMMECR1, KIAA1287, LOC144233, LOC286505, PNAS-4, FLJ20530, HUMPD3, GC45564, CAP350, ETAA16, HAN11, DNAPTP6, C7orf25, FLJ37953, FLJ10587, C7orf36, ELP4, NDEL1, NPD014, KFZP564D172, FAM53C, IER5, LOC255783, KIAA0146, KIAA0792, LOC439994, LOC283481, CG018, LOC130576, NGFRAP1L1, KIAA1217, 4orf7, C21orf86, C9orf64, FLJ13456, FLJ12806, FLJ39370, GATS, CCDC92, FMNL2, ARID1B, ZFHX1B, SSBP2, ARID5B, LOC157381, KPNA3, ARHGAP24, CCND2, VIM, MYCBP2, GAS7, TRAM2, BASP1, FOXO1A, POLR2A, PER1, DDIT4, CD97, BIN1, SH2B3, DDB2, EMP3, NDST2, CHST2, NT5E, PDE4A, CPS1, PTGS1, GGCX, IRF5, ZBTB16, MAP4K4, CHST7, KLF12, NFRKB, PSD, FKSG49, NIFUN, FYN, ZMYM2, CACNA1G, SLC25A16, FLII, EIF1, SEPT6, NPAC, PHF15, NUP188, ABR, CNR1, LOC283824, FSTL4, DNM1, APOBEC3G, ATP2B1, SMPD1, SLC11A1, FXYD5, C14orf139, SH3BGRL3, LUZP1, ZNF335, SH3 KBP1, MST150, PRO1073, LOC388397, FKBP5, HIPK2, DTX1, ST3GAL2, ADAMTS7, TNRC6B, CYGB, SDHAL1, LOC572558, TRIO, FRAS1, KIAA1632, POLS, EBF, MAML2, PTPRA, PLEKHG2, DYM, SOX6, ARHGEF2, ZCCHC6, PPP3CA, FAM70B, TMED5, FLJ43663, HPS1, MEF2A, ST3GAL5, SMYD3, KLF7, LOC200230, RERE, QKI, BICD1, CTNNB1, POU2F2, EIF4ENIF1, BTG1, TCHP, FLJ90709, PCTK2, KIAA1600, B7-H4, PDE4DIP, KIAA0194, HOM-TES; and
(a) BGN, BIN1, CDH11, CDH2, CHN1, COL1A2, COL3A1, COL5A2, CREB3L1, CTGF, CYBRD1, DCN, DLC1, DNAJB4, DPT, ENPP2, FBLN1, FBLN5, FBN1, GREM1, HAS2, IGFBP3, IGFBP4, MLPH, MME, MMP2, MYL9, NEBL, NID1, NR2F1, OLFML3, PCOLCE, PMP22, POSTN, PPAP2B, PRR16, PRRX1, PTX3, PVRL3, RGL1, RGS4, ROR1, SEMA5A, TNFAIP6, TRAM2, WNT5A, ZEB1, ANXA6, CITED2, COL1A1, COL6A1, CYP1B1, DAB2, DOCK10, EML1, GNG11, IER3, ITGBL1, KCNMA1, LRIG1, LUM, MAP1B, NID2, PCDH9, PRKCA, PTGER4, PTGFR, SDC2, SERPINF1, SNED1, SYT11, TFPI, TGFBR3, VCAN, ANKRD25, C10ORF56, C5ORF13, CCDC92, CDKN2C, COPZ2, DDR2, ECM1, ENOX1, FADS2, FAP, FILIP1L, FSTL1, HS3ST2, KRT81, LMCD1, LTBP1, LTBP2, MMP1, NPAC, NRP1, PDGFC, PLEKHC1, PPM1D, PTGER2, SCCPDH, SEPT9, SPOCK1, SRGN, STARD13, STC1, SYNC1, TAGLN, TBX3, TGFB1I1, THY1, TNS3, TPM1, TSHZ1, TUBA1A, UGDH, VIM, XYLT1, ZBTB38, MBP, ABLIM1, ALDH1A3, ALOX15B, ANXA3, AP1M2, ARTN, CA2, CA9, CAMK2B, CCND2, CDH1, CDH3, CDS1, COL17A1, CORO1A, CTSL2, CXADR, CYP27B1, DSC2, DSG3, DST, EPB41L4B, FGFBP1, FGFR3, FST, GJB3, GRHL2, HBEGF, HOOK1, IL18, IL1B, IRF6, ITGB4, JAG2, KLK10, KLK5, KLK7, KLK8, KRT15, KRT16, KRT17, KRT5, KRT6B, LEPREL1, MYO5C, NDRG1, NMU, PI3, RAB25, RLN2, RNF128, S100A14, S100A7, S100A8, SERPINB1, SERPINB2, SLC2A9, SLPI, SNX10, SPINT2, SPRR1A, SPRR1B, ST14, SYK, TMEM40, TRIM29, TSPAN1, UCHL1, VSNL1, ZBED2, AQP3, CBARA1, CD24, CLCA2, CNTNAP2, CSTA, DSC3, EPCAM, ESRP2, FXYD3, GJB5, GPR87, IL1A, LAMA3, LAMB3, MAP7, MAPK13, MMP9, MPZL2, NRG1, SCNN1A, SERPINB13, SLC7A5, SPINT1, ST6GALNAC2, TGFA, TP63, TPD52L1, VGLL1, SLC6A8, ABCA12, ANK3, ANXA8, ANXA8L1, ARHGAP25, ARHGAP8, BDKRB2, BIK, C10ORF10, C10ORF116, C1ORF116, C20ORF19, C6ORF105, CELSR2, CKMT1A, CKMT1B, CLDN1, CST6, CYP4F11, E2F5, ELMO3, EPHA1, EVA1, EXPH5, F11R, FAT2, FGFR2, FLJ12684, FLJ20366, FZD3, GLS2, GNAL, IFI30, IGFBP2, IL1RN, IL4R, IRX4, JUP, KCNK1, KIAA0040, KIAA0888, KIAA1815, KRT14, KRT18, LAD1, LGALS7, LOC553158, LOC653562, LOC728113, LOC729884, LRRC1, LSR, MST1R, MYO1D, NAIP, OCLN, NEFM, NUP62CL, PERP, PLS1, POLR3G, PRKCH, PRRG4, PRSS8, PTPN3, RBM35A, RBM35B, RHBDF2, RHOD, RPS6KA1, RTEL1, SAA1, SAA1, SAA2, SH2D3A, SLC6A10P, SMPDL3B, SNCA, STAC, STAP2, TACSTD1, THBD, TMEM30B, TMPRSS11E, TMPRSS4, TNFRSF6B, TP73L, WWC1, XDH, ZNF165; and
(b) BGN, BIN1, CDH11, CDH2, CHN1, COL1A2, COL3A1, COL5A2, CREB3L1, CTGF, CYBRD1, DCN, DLC1, DNAJB4, DPT, ENPP2, FBLN1, FBLN5, FBN1, GREM1, HAS2, IGFBP3, IGFBP4, MLPH, MME, MMP2, MYL9, NEBL, NID1, NR2F1, OLFML3, PCOLCE, PMP22, POSTN, PPAP2B, PRR16, PRRX1, PTX3, PVRL3, RGL1, RGS4, ROR1, SEMA5A, TNFAIP6, TRAM2, WNT5A, ZEB1, ANXA6, CITED2, COL1A1, COL6A1, CYP1B1, DAB2, DOCK10, EML1, GNG11, IER3, ITGBL1, KCNMA1, LRIG1, LUM, MAP1B, NID2, PCDH9, PRKCA, PTGER4, PTGFR, SDC2, SERPINF1, SNED1, SYT11, TFPI, TGFBR3, VCAN, ANKRD25, C10ORF56, C5ORF13, CCDC92, CDKN2C, COPZ2, DDR2, ECM1, ENOX1, FADS2, FAP, FILIP1L, FSTL1, HS3ST2, KRT81, LMCD1, LTBP1, LTBP2, MMP1, NPAC, NRP1, PDGFC, PLEKHC1, PPM1D, PTGER2, SCCPDH, SEPT9, SPOCK1, SRGN, STARD13, STC1, SYNC1, TAGLN, TBX3, TGFB1I1, THY1, TNS3, TPM1, TSHZ1, TUBA1A, UGDH, VIM, XYLT1, ZBTB38; and
(c) ENPP2, CITED2, ZEB1, NID2, SEMA5A, DAB2, KCNMA1, PTX3, PCDH9, CREB3L1, BGN, ROR1, DLC1, GNG11, CDH11, NR2F1, NR2F2, PRR16, MYL9, DOCK10, LRIG1, IER3, EML1, NEBL, RGL1, MLPH, TRAM2, DNAJB4, FBLN5, RGS4, HAS2, ITGBL1, IGFBP4, BIN1, DPT, PCOLCE, GREM1, PPAP2B, CDH2, PMP22, LUM, CHN1, CYP1B1, MME, WNT5A, POSTN, MMP2, CTGF, FBLN1, IGFBP3, DCN, PRRX1, ANXA6, PVRL3, MAP1B, TNFAIP6, CYBRD1, FBN1, NID1, OLFML3, SNAI1, SNAI2, TAGLN, SYNC, TUBA1A, PPM1D, TWIST1, FN1, TGFBR3, SERPINF1, UGDH, SRGN, FAP, PTGER4, VIM, PRKCA, FSTL1, MMP1, NRP1, FILIP1L, SCCPDH, LTBP2, XYLT1, HS3ST2, SYT11, TSHZ1, THY1, SEPT9, S100A4, CCDC92, TNS3, ENOX1, TGFB1I1, ZEB2, LMCD1, PDGFC, ECM1, TFPI, TBX3, DDR2, PTGER2, CDKN2C, VCAN, CD44, STARD13, SNED1, ZBTB38, SDC2, TPM1, COPZ2, STC1, CDH1, KRT5, KRT6B, CD24, EPCAM, ESRP1, CLDN3, CLDN4, ERBB3, SPOCK1, FERMT2, GLYR1, LTBP1, FADS2, KANK2, PTGFR, ARTN, SLC7A5, EPB41L4B, KRT15, CDH3, TPD52L1, ANXA3, PI3, NDRG1, DSG3, S100A14, S100S7, KLK8, S100A8, PTX3, SPINT2, FXYD3, KRT16, RAB25, LGALS7, DSC3, COL5A2, ITGB4, TMEM40, SPRR1A, AP1M2, SERPINB13, TP73L; and
(d) CYBRD1, ARTN, CITED2, SLC7A5, CHN1, CTGF, CSTA, MYL9, EPB41L4B, KRT15, LRIG1, CDH3, TPD52L1, ANXA3, OLFML3, CORO1A, PI3, NDRG1, DSG3, PMP22, S100A14, S100S7, KLK8, S100A8, PTX3, SPINT2, IER3, FXYD3, KRT16, TGFBR3, KRT5, RAB25, SDC2, LUM, LGALS7, KRT6B, CD24, COL1A2, GNG11, DSC3, COL5A2, RGS4, ITGB4, TMEM40, SPRR1A, AP1M2, SERPINB13, SNED1, LEPREL1, TRAM2, TP73L, and PAP2B; and
(e) CYBRD1, ARTN, CITED2, SLC7A5, CHN1, CTGF, CSTA, MYL9, EPB41L4B, KRT15, LRIG1, CDH3, TPD52L1, ANXA3, OLFML3, CORO1A; and
(f) PI3, NDRG1, DSG3, PMP22, S100A14, S100S7, KLK8, S100A8, PTX3, SPINT2, IER3, FXYD3, KRT16, TGFBR3; and
(g) KRT5, RAB25, SDC2, LUM, LGALS7, KRT6B, CD24, COL1A2, GNG11, DSC3, COL5A2; and
(h) RGS4, ITGB4, TMEM40, SPRR1A, AP1M2, AERPINB13, SNED1, LEPREL1, TRAM2, TP73L, and PAB2B; and
   (i) CYBRD1, ARTN, KRT15, ITGB4, RGS4, SPRR1B, SLC7A5, ANXA3, IL18, AP1M2, CHN1, OLFML3, ZBED2, LEPREL1, ABLIM1, CDH3, DNAJB4, TRAM2, UCHL1, CTGF; and
   (i) CYBRD1, ARTN, KRT15, ITGB4, RGS4, SPRR1B, SLC7A5, ANXA3, IL18, AP1M2; and
   (ii) CYBRD1, ARTN, KRT15, ITGB4, RGS4; and
(j) CYBRD1, RGS4, CDH3, CHN1, SLC7A5, PRRX1, CITED2, COL5A2, ARTN, ZBED2, SNED1, ITGB4, LUM, PVRL3, OLFML3, BIN1, CCND2, DAB2, ANXA3, IL18; and
   (i) CYBRD1, RGS4, CDH3, CHN1, SLC7A5, PRRX1, CITED2, COL5A2, ARTN, ZBED2; and
   (ii) CYBRD1, RGS4, CDH3, CHN1, SLC7A5; and
(k) CYBRD1, ARTN, RGS4, CITED2, SLC7A5, ITGB4, CHN1, PI3, TMEM40, NDRG1, SPRR1A, PCDH9, CTGF, MAP1B, DSG3, AP1M2, FBLN5, SERPINB13, PMP22, CSTA; and
   (i) CYBRD1, ARTN, RGS4, CITED2, SLC7A5, ITGB4, CHN1, PI3, TMEM40, NDRG1; and
   (ii) CYBRD1, ARTN, RGS4, CITED2, SLC7A5; and

(ii) said second set of genes comprises or consists of:

AXL, NOTCH3, GPR30, PROCR, PAX2, PI3KA, CD44, CD133, ALDH1, BMI1, KRT19, NRP1, MSI1, MTOR, JAG1, p38, ESA1, ZEB1, TWIST1, FOXC2, CDH2, ZEB2, FOXC1, SNAI1, SNAI2, TWIST2, GSC, TCF3, CD44, VIM, FN1, MLPH1, ARHGEF11; and

(iii) said third set of genes comprises or consists of:

At least one gene from said first set of genes and at least one of:

AXL, NOTCH3, GPR30, PROCR, PAX2, PI3KA, CD44, CD133, ALDH1, BMI1, KRT19, NRP1, MSI1, MTOR, JAG1, p38, ESA1, ZEB1, TWIST1, FOXC2, CDH2, ZEB2, FOXC1, SNAI1, SNAI2, TWIST2, GSC, TCF3, CD44, VIM, FN1, MLPH1, ARHGEF11; and

(iv) said fourth set of genes comprises or consists of:

At least two genes from said first set of genes and at least one of: AXL, NOTCH3, GPR30, PROCR, PAX2, PI3KA, CD44, CD133, ALDH1, BMI1, KRT19, NRP1, MSI1, MTOR, JAG1, p38, ESA1, ZEB1, TWIST1, FOXC2, CDH2, ZEB2, FOXC1, SNAI1, SNAI2, TWIST2, GSC, TCF3, CD44, VIM, FN1, MLPH1, and ARHGEF11; and

(v) said fifth set of genes comprises or consists of:

beta catenin, FZD1, FZD2, FZD3, FZD4, FZD5, FZD6, FZD7, FZD8, FZD9, FZD10, LRP5, LRP6, WNT1, WNT2, WNT2B, WNT3, WNT3A, WNT4 WNT5A, WNT5B, WNT6, WNT7A, WNT7B, WNT8A, WNT8B, WNT9A, WNT9B, WNT10A, WNT10B, WNT11, WNT16, SRFP1, SRFP2, FRZB, SFRP4, SFRP5, DKK1, DKK2, DKK3, DKK4, WIF1, AKT2, PI3KA, Disheveled, Smoothened, Axin1, Axin2; and

(vi) said sixth set of genes comprises or consists of:

At least one gene from said first set of genes and at least one of:

beta catenin, FZD1, FZD2, FZD3, FZD4, FZD5, FZD6, FZD7, FZD8, FZD9, FZD10, LRP5, LRP6, WNT1, WNT2, WNT2B, WNT3, WNT3A, WNT4 WNT5A, WNT5B, WNT6, WNT7A, WNT7B, WNT8A, WNT8B, WNT9A, WNT9B, WNT10A, WNT10B, WNT11, WNT16, SRFP1, SRFP2, FRZB, SFRP4, SFRP5, DKK1, DKK2, DKK3, DKK4, WIF1, AKT2, PI3KA, Disheveled, Smoothened, Axin1, Axin2; and

(vii) said seventh set of genes comprises or consists of:

TGFbeta1, TGFbeta2, TGFbeta3, ALK1, ALK2, ALK3, ALK4, ALK5, ALK6, ALK7, TGFbetaR-II, BMPR-II, ACTR-IIA, ACTR-IIB, AMHR-II, SMAD2, SMAD3; and

(viii) said eighth set of genes comprises or consists of:

At least one gene from said first set of genes and at least one of:

TGFbeta1, TGFbeta2, TGFbeta3, ALK1, ALK2, ALK3, ALK4, ALK5, ALK6, ALK7, TGFbetaR-II, BMPR-II, ACTR-IIA, ACTR-IIB, AMHR-II, SMAD2, SMAD3; and

(ix) said ninth set of genes comprises or consists of:

S100A4, SDC2, CYP1B1, FERMT2, CYBRD1, SNAI2, CD44, BIN1, PMP22, CTGF, IGFBP3, ANXA6, MAP1B, TAGLN, ENPP2, FBLN5, PCOLCE, FN1, VIM, PPAP2B, SEM5A, VCAN, NID2, NID1, HAS2, PRRX1, TNFAIP6, CLDN4, FADS2, DDIT4, GNG11, PER1, ISCU, KPNA3, SLC44A1, FKBP5, NR2F1, MLPH, DOCK10, RGS4, IGFBP4; and

(x) said tenth set of genes comprises or consists of:

S100A4, SDC2, CYP1B1, FERMT2, CYBRD1, SNAI2, CD44, BIN1, ENPP2, FBLN5, PCOLCE, FN1, VIM, PPAP2B, SEM5A, VCAN, CLDN4, FADS2, DDIT4, GNG11, PER1, ISCU, KPNA3, SLC44A1, FKBP5, IGFBP4; and

(xi) said eleventh set of genes comprises or consists of:

SDC2, CYBRD1, SNAI2, PMP22, CTGF, IGFBP3, ANXA6, MAP1B, TAGLN, PPAP2B, HAS2, NRF2, MLPH, DOCK10, RGS4; and

(xii) said twelfth set of genes comprises or consists of:

FERMT2, BIN1, PMP22, CHN1, CTGF, IGFBP3, NID2, CLDN4, CYBRD1, PRR16, ANXA6, PCOLCE, PPAP2B, HAS2, CDH11, MLPH, DOCK10, RGS4, PVRL3, NID2, SLC44A1, FKBP5; and

(xiii) said thirteenth set of genes comprises or consists of:

FERMT2, BIN1, PMP22, CHN1, CTGF, IGFBP3, NID2, CLDN4; and

(xiv) said fourteenth set of genes comprises or consists of:

HAS2, BIN1, PCOLCE, FERMT2, CTGF, IGFBP3, NID2, SLC44A1, FKBP5, and MLPH.

In an embodiment, said kit or product features a second agent capable of interacting with a gene expression product from said first and/or second and/or third and/or fourth and/or fifth and/or sixth and/or seventh and/or eighth and/or ninth and/or tenth and/or eleventh and/or twelfth and/or thirteenth and/or fourteenth set of genes. In an embodiment, said kit or product features a plurality of agents capable of interacting with a gene expression product from said first and/or second and/or third and/or fourth and/or fifth and/or sixth and/or seventh and/or eighth and/or ninth and/or tenth and/or eleventh and/or twelfth and/or thirteenth and/or fourteenth set of genes. In an embodiment, said kit or product features a plurality of agents capable of interacting with a plurality of gene expression products from said first and/or second and/or third and/or fourth and/or fifth and/or sixth and/or seventh and/or eighth and/or ninth and/or tenth and/or eleventh and/or twelfth and/or thirteenth and/or fourteenth set of genes. In an embodiment, said plurality of gene expression products is any subset of gene expression products s in gene sets one, two, three, four, five, six, seven, eight, nine, ten, eleven, twelve, thirteen, or fourteen. In an embodiment, said plurality of gene expression products s is any subset of gene expression products in gene set one. In an embodiment, said plurality of gene expression products s is any subset of gene expression products in gene set two. In an embodiment, said plurality of gene expression products s is any subset of gene expression products in gene set three. In an embodiment, said plurality of gene expression products s is any subset of gene expression products in gene set four. In an embodiment, said plurality of gene expression products s is any subset of gene expression products in gene set five. In an embodiment, said plurality of gene expression products s is any subset of gene expression products in gene set six. In an embodiment, said plurality of gene expression products s is any subset of gene expression products in gene set seven. In an embodiment, said plurality of gene expression products s is any subset of gene expression products in gene set eight. In an embodiment, said plurality of gene expression products s is any subset of gene expression products in gene set nine. In an embodiment, said plurality of gene expression products s is any subset of gene expression products in gene set ten. In an embodiment, said plurality of gene expression products s is any subset of gene expression products in gene set eleven. In an embodiment, said plurality of gene expression products s is any subset of gene expression products in gene set twelve. In an embodiment, said plurality of gene expression products s is any subset of gene expression products in gene set thirteen. In an embodiment, said plurality of gene expression products s is any subset of gene expression products in gene set fourteen.

In an embodiment, said agent is a plurality of antibodies. In an embodiment, said agent is a plurality of oligonucleotides. In an embodiment, said agent is a plurality of antibodies and oligonucleotides. In an embodiment, said gene expression product is a RNA product. In an embodiment, said gene expression product is a protein product.

In an embodiment, said kit or product features an agent capable of interacting with a gene expression product of a gene in Table 1. In an embodiment, said kit or product contains plurality of agents capable of interacting with a plurality of genes in Table 1. In an embodiment, said kit or product features an agent capable of interacting with a gene expression product of a gene not in said first and/or second and/or third and/or fourth and/or fifth and/or sixth and/or seventh and/or eighth and/or ninth and/or tenth and/or eleventh and/or twelfth and/or thirteenth and/or fourteenth set of genes. In an embodiment, said kit or product features a plurality of agents capable of interacting with a gene expression product of a plurality of genes not in said first and/or second and/or third and/or fourth and/or fifth and/or sixth and/or seventh and/or eighth and/or ninth and/or tenth and/or eleventh and/or twelfth and/or thirteenth and/or fourteenth set of genes.

In one aspect, kits or products described herein include kits or products comprising a first agent capable of interacting with a gene expression product of a plurality of genes from a first and/or second and/or third and/or fourth and/or fifth and/or sixth and/or seventh and/or eighth and/or ninth and/or tenth and/or eleventh and/or twelfth and/or thirteenth and/or fourteenth set of genes, wherein:

(i) said first set of genes comprises or consists of:
KLF13, ANTXR2, IFNAR1, LIX1L, CHST11, AKAP2, PALM2-AKAPT, LOC80298, C7 orf2, NUCKS, DKFZP566D1346, LOC388279, FLJ31795, 6orf107, FLJ12439, ENPP2, CITED2, ZEB1, NID2, SEMA5A, DAB2, KCNMA1, PTX3, PCDH9, CREB3L1, BGN, ROR1, DLC1, GNG11, CDH11, NR2F1, PRR16, MYL9, DOCK10, LRIG1, IER3, EML1, NEBL, RGL1, MLPH, DNAJB4, FBLN5, RGS4, HAS2, ITGBL1, IGFBP4, DPT, PCOLCE, GREM1, PPAP2B, CDH2, PMP22, LUM, CHN1, CYP1B1, MME, WNT5A, POSTN, MMP2, CTGF, FBLN1, IGFBP3, DCN, PRRX1, ANXA6, PVRL3, MAP1B, TNFAIP6, CYBRD1, FBN1, NID1, OLFML3, SNAI1, SNAI2, TAGLN, SYNC, TUBA1A, PPM1D, TWIST1, FN1, TGFBR3, SERPINF1, UGDH, SRGN, FAP, PTGER4, PRKCA, FSTL1, MMP1, NRP1, FILIP1L, SCCPDH, LTBP2, XYLT1, HS3ST2, SYT11, TSHZ1, THY1, Sep9, S100A4, TNS3, ENOX1, TGFB1I1, ZEB2, LMCD1, PDGFC, ECM1, TFPI, TBX3, DDR2, PTGER2, CDKN2C, VCAN, CD44, STARD13, SNED1, ZBTB38, SDC2, TPM1, COPZ2, STC1, CDH1, KRT5, KRT6B, CD24, EPCAM, ESRP1, CLDN3, CLDN4, ERBB3, SPOCK1, FERMT2, GLYR1, LTBP1, FADS2, KANK2, PTGFR, COL11A2, KLK3, EIF2C2, ZFP41, FAM49B, PSORS1C2, MRPL42, MRPL54, MRPL47, MRPS23, EIF3S9, ALG5, DNAJC19, TPRXL, NOTCH2, RBM15, ST3GAL3, NFYA, PCNX, FBXO21, WWOX, CAMK2B, PNPLA2, CLIC5, UGCGL1, FBXL18, ADRBK1, SLC38A2, IL8RA, TAS2R14, CD300LB, GIPC3, RAD51L1, EPPK1, COL1A1, MMP9, SERPINE1, SPARC, TGFB1, TGFB3, TGFBI, TGFBR1, TGIF, TGIF2, THBS1, ANXA5, ACTG1, ARF3, ATP1B3, BAT3, CALD1, CENTD2, CLIC1, CTBS, DPYSL3, DVL3, EXT1, FGFR1, FTL, GNB2L1, GPRC5A, H2AFZ, HIF1A, IL13RA1, KDELR2, LARP1, LPIN2, MARS, MMP10, MMP14, MT2A, MYO10, NUP62, PFKFB3, PLOD2, PSMB7, PSMD8, RIN2, RYBP, SDF4, SETD5, SPP1, STAU1, TUBB3, UBE2S, XPNPEP1, CDKN1A, CHRD, H19, ID3, ID4, IGFBP7, LRP1, MSX1, NOTCH3, PROCR, GBX2, KI67, CCNB1, BUB1, KNTC2, USP22, HCFC1, RNF2, ANK3, FGFR2, CES1, COL1A2, COL3A1, COL5A2, COL6A1, ANKRD25, C10ORF56, C5ORF13, KRT81, NPAC, PLEKHC1, SEPT9, SYNC1, MBP, ABLIM1, ALDH1A3, ALOX15B, ANXA3, AP1M2, ARTN, CA2, CA9, CDH3, CDS1, COL17A1, CORO1A, CTSL2, CXADR, CYP27B1, DSC2, DSG3, DST, EPB41L4B, FGFBP1, FGFR3, FST, GJB3, GRHL2, HBEGF, HOOK1, IL18, IL1B, IRF6, ITGB4, JAG2, KLK10, KLK5, KLK7, KLK8, KRT15, KRT16, KRT17, LEPREL1, MYO5C, NDRG1, NMU, PI3, RAB25, RLN2, RNF128, S100A14, S100A7, S100A8, SERPINB1, SERPINB2, SLC2A9, SLPI, SNX10, SPINT2, SPRR1A, SPRR1B, ST14, SYK, TMEM40, TRIM29, TSPAN1, UCHL1, VSNL1, ZBED2, AQP3, CBARA1, CLCA2, CNTNAP2, CSTA, DSC3, ESRP2, FXYD3, GJB5, GPR87, IL1A, LAMA3, LAMB3, MAP7, MAPK13, MPZL2, NRG1, SCNN1A, SERPINB13, SLC7A5, SPINT1, ST6GALNAC2, TGFA, TP63, TPD52L1, VGLL1, SLC6A8, ABCA12, ANXA8, ANXA8L1, ARHGAP25, ARHGAP8, BDKRB2, BIK, C10ORF10, C10ORF116, C1ORF116, C20ORF19, C6ORF105, CELSR2, CKMT1A, CKMT1B, CLDN1, CST6, CYP4F11, E2F5, ELMO3, EPHA1, EVA1, EXPH5, F11R, FAT2, FLJ12684, FLJ20366, FZD3, GLS2, GNAL, IFI30, IGFBP2, IL1RN, IL4R, IRX4, JUP, KCNK1, KIAA0040, KIAA0888, KIAA1815, KRT14, KRT18, LAD1, LGALS7, LOC553158, LOC653562, LOC728113, LOC729884, LRRC1, LSR, MST1R, MYO1D, NAIP, OCLN, NEFM, NUP62CL, PERP, PLS1, POLR3G, PRKCH, PRRG4, PRSS8, PTPN3, RBM35A, RBM35B, RHBDF2, RHOD, RPS6KA1, RTEL1, SAA1, SAA1, SAA2, SH2D3A, SLC6A10P, SMPDL3B, SNCA, STAC, STAP2, TACSTD1, THBD, TMEM30B, TMPRSS11E, TMPRSS4, TNFRSF6B, TP73L, WWC1, XDH, ZNF165, AXL, GPR30, PAX2, PI3KA, CD133, ALDH1, BMI1, KRT19, MSI1, JAG1, p38, ESA1, FOXC2, FOXC1, TWIST2, GSC, TCF3, MLPH1, ARHGEF11a, DPF2, CASP8, BCL2, SCGN, SWAP70, KIAA0276, C10orf9, C10orf7, ALKBH, TOB2, XPR1, CD59, LRP2, PLP2, MAPK14, CXCL2, MMP7, MGP, MLF1, FLNB, SCNM1, HSPC163, C5orf18, MGC4399, CDW92, TMC4, ZDHHC2, TICAM2, KDELR3, GNPDA1, THEM2, DBR1, FLJ90709, FLJ10774, C16orf33, GAPD, LDHA, MR-1, LARS, GTPBP1, PRSS16, WFDC2, AIM1, DHRS6, DHRS4, GC15429, MGC45840, ECHDC2, GOLGIN-67, AFURS1, KIAA0436, CYP4V2, JTV1, ICMT, DNMT3A, HNMT, METTL7A, METTL2, VIL2, TPD52, ARPC5, NOL8, NSF, RAD23B, SRP54, HSPA2, PBP, THAP2, CIRBP, SNRPN, KIAA0052, DUSP10, SSR1, ERBB4, EMP1, CHPT1, LRPAP1, FLJ11752, CSTF1, KLHL20, DNAJC13, APLP2, ARGBP2, DNAJB1, NEBL, SH3BGRL, NUDT5, GABARAPL1, MAPT, DCBLD1, STK39, PAK2, CSNK2A1, PILRB, ERN1, SGKL, WEE1, MAST4, C11orf17, NUP37, CLTC, COPB2, SLC25A25, ECOP, PDE8A, STAM, TUBB, SNX6, RAB23, PLAA, STC2, LTF, ISGF3G, ATXN3, GTF3C3, GSK3B, KLF10, ELL2, ZBTB20, IRX3, ETS1, SERTAD1, MGC4251, MAFF, SFPQ, CITED4, CEBPD, EIF4E2, HS2ST1, AGPS, PGK1, ATIC, ETNK1, LG2, NCE2, MARCH8, CNOT4, RNF8, PSMA5, DPF2, AMMECR1, KIAA1287, LOC144233, LOC286505, PNAS-4, FLJ20530, HUMPD3, GC45564, CAP350, ETAA16, HAN11, DNAPTP6, C7orf25, FLJ37953, FLJ10587, C7orf36, ELP4, NDEL1, NPD014, KFZP564D172, FAM53C, IER5, LOC255783, KIAA0146, KIAA0792, LOC439994, LOC283481, CG018, LOC130576, NGFRAP1L1, KIAA1217, 4orf7, C21 orf86, C9orf64, FLJ13456, FLJ12806, FLJ39370, GATS, CCDC92, FMNL2, ARID1B, ZFHX1B, SSBP2, ARID5B, LOC157381, KPNA3, ARHGAP24, CCND2, VIM, MYCBP2, GAS7, TRAM2, BASP1, FOXO1A, POLR2A, PER1, DDIT4, CD97, BIN1, SH2B3, DDB2, EMP3, NDST2, CHST2, NT5E, PDE4A, CPS1, PTGS1, GGCX, IRF5, ZBTB16, MAP4K4, CHST7, KLF12, NFRKB, PSD, FKSG49, NIFUN, FYN, ZMYM2, CACNA1G, SLC25A16, FLII, EIF1, SEPT6, NPAC, PHF15, NUP188, ABR, CNR1, LOC283824, FSTL4, DNM1, APOBEC3G, ATP2B1, SMPD1, SLC11A1, FXYD5, C14orf139, SH3BGRL3, LUZP1, ZNF335, SH3 KBP1, MST150, PRO1073, LOC388397, FKBP5, HIPK2, DTX1, ST3GAL2, ADAMTS7, TNRC6B, CYGB, SDHAL1, LOC572558, TRIO, FRAS1, KIAA1632, POLS, EBF, MAML2, PTPRA, PLEKHG2, DYM, SOX6, ARHGEF2, ZCCHC6, PPP3CA, FAM70B, TMED5, FLJ43663, HPS1, MEF2A, ST3GAL5, SMYD3, KLF7, LOC200230, RERE, QKI, BICD1, CTNNB1, POU2F2, EIF4ENIF1, BTG1, TCHP, FLJ90709, PCTK2, KIAA1600, B7-H4, PDE4DIP, KIAA0194, HOM-TES; and
(a) BGN, BIN1, CDH11, CDH2, CHN1, COL1A2, COL3A1, COL5A2, CREB3L1, CTGF, CYBRD1, DCN, DLC1, DNAJB4, DPT, ENPP2, FBLN1, FBLN5, FBN1, GREM1, HAS2, IGFBP3, IGFBP4, MLPH, MME, MMP2, MYL9, NEBL, NID1, NR2F1, OLFML3, PCOLCE, PMP22, POSTN, PPAP2B, PRR16, PRRX1, PTX3, PVRL3, RGL1, RGS4, ROR1, SEMA5A, TNFAIP6, TRAM2, WNT5A, ZEB1, ANXA6, CITED2, COL1A1, COL6A1, CYP1B1, DAB2, DOCK10, EML1, GNG11, IER3, ITGBL1, KCNMA1, LRIG1, LUM, MAP1B, NID2, PCDH9, PRKCA, PTGER4, PTGFR, SDC2, SERPINF1, SNED1, SYT11, TFPI, TGFBR3, VCAN, ANKRD25, C10ORF56, C5ORF13, CCDC92, CDKN2C, COPZ2, DDR2, ECM1, ENOX1, FADS2, FAP, FILIP1L, FSTL1, HS3ST2, KRT81, LMCD1, LTBP1, LTBP2, MMP1, NPAC, NRP1, PDGFC, PLEKHC1, PPM1D, PTGER2, SCCPDH, SEPT9, SPOCK1, SRGN, STARD13, STC1, SYNC1, TAGLN, TBX3, TGFB1I1, THY1, TNS3, TPM1, TSHZ1, TUBA1A, UGDH, VIM, XYLT1, ZBTB38, MBP, ABLIM1, ALDH1A3, ALOX15B, ANXA3, AP1M2, ARTN, CA2, CA9, CAMK2B, CCND2, CDH1, CDH3, CDS1, COL17A1, CORO1A, CTSL2, CXADR, CYP27B1, DSC2, DSG3, DST, EPB41L4B, FGFBP1, FGFR3, FST, GJB3, GRHL2, HBEGF, HOOK1, IL18, IL1B, IRF6, ITGB4, JAG2, KLK10, KLK5, KLK7, KLK8, KRT15, KRT16, KRT17, KRT5, KRT6B, LEPREL1, MYO5C, NDRG1, NMU, PI3, RAB25, RLN2, RNF128, S100A14, S100A7, S100A8, SERPINB1, SERPINB2, SLC2A9, SLPI, SNX10, SPINT2, SPRR1A, SPRR1B, ST14, SYK, TMEM40, TRIM29, TSPAN1, UCHL1, VSNL1, ZBED2, AQP3, CBARA1, CD24, CLCA2, CNTNAP2, CSTA, DSC3, EPCAM, ESRP2, FXYD3, GJB5, GPR87, IL1A, LAMA3, LAMB3, MAP7, MAPK13, MMP9, MPZL2, NRG1, SCNN1A, SERPINB13, SLC7A5, SPINT1, ST6GALNAC2, TGFA, TP63, TPD52L1, VGLL1, SLC6A8, ABCA12, ANK3, ANXA8, ANXA8L1, ARHGAP25, ARHGAP8, BDKRB2, BIK, C10ORF10, C10ORF116, C1ORF116, C20ORF19, C6ORF105, CELSR2, CKMT1A, CKMT1B, CLDN1, CST6, CYP4F11, E2F5, ELMO3, EPHA1, EVA1, EXPH5, F11R, FAT2, FGFR2, FLJ12684, FLJ20366, FZD3, GLS2, GNAL, IFI30, IGFBP2, IL1RN, IL4R, IRX4, JUP, KCNK1, KIAA0040, KIAA0888, KIAA1815, KRT14, KRT18, LAD1, LGALS7S, LOC553158, LOC653562, LOC728113, LOC729884, LRRC1, LSR, MST1R, MYO1D, NAIP, OCLN, NEFM, NUP62CL, PERP, PLS1, POLR3G, PRKCH, PRRG4, PRSS8, PTPN3, RBM35A, RBM35B, RHBDF2, RHOD, RPS6KA1, RTEL1, SAA1, SAA1, SAA2, SH2D3A, SLC6A10P, SMPDL3B, SNCA, STAC, STAP2, TACSTD1, THBD, TMEM30B, TMPRSS11E, TMPRSS4, TNFRSF6B, TP73L, WWC1, XDH, ZNF165; and
(b) BGN, BIN1, CDH11, CDH2, CHN1, COL1A2, COL3A1, COL5A2, CREB3L1, CTGF, CYBRD1, DCN, DLC1, DNAJB4, DPT, ENPP2, FBLN1, FBLN5, FBN1, GREM1, HAS2, IGFBP3, IGFBP4, MLPH, MME, MMP2, MYL9, NEBL, NID1, NR2F1, OLFML3, PCOLCE, PMP22, POSTN, PPAP2B, PRR16, PRRX1, PTX3, PVRL3, RGL1, RGS4, ROR1, SEMA5A, TNFAIP6, TRAM2, WNT5A, ZEB1, ANXA6, CITED2, COL1A1, COL6A1, CYP1B1, DAB2, DOCK10, EML1, GNG11, IER3, ITGBL1, KCNMA1, LRIG1, LUM, MAP1B, NID2, PCDH9, PRKCA, PTGER4, PTGFR, SDC2, SERPINF1, SNED1, SYT11, TFPI, TGFBR3, VCAN, ANKRD25, C10ORF56, C5ORF13, CCDC92, CDKN2C, COPZ2, DDR2, ECM1, ENOX1, FADS2, FAP, FILIP1L, FSTL1, HS3ST2, KRT81, LMCD1, LTBP1, LTBP2, MMP1, NPAC, NRP1, PDGFC, PLEKHC1, PPM1D, PTGER2, SCCPDH, SEPT9, SPOCK1, SRGN, STARD13, STC1, SYNC1, TAGLN, TBX3, TGFB1I1, THY1, TNS3, TPM1, TSHZ1, TUBA1A, UGDH, VIM, XYLT1, ZBTB38; and
(c) ENPP2, CITED2, ZEB1, NID2, SEMA5A, DAB2, KCNMA1, PTX3, PCDH9, CREB3L1, BGN, ROR1, DLC1, GNG11, CDH11, NR2F1, NR2F2, PRR16, MYL9, DOCK10, LRIG1, IER3, EML1, NEBL, RGL1, MLPH, TRAM2, DNAJB4, FBLN5, RGS4, HAS2, ITGBL1, IGFBP4, BIN1, DPT, PCOLCE, GREM1, PPAP2B, CDH2, PMP22, LUM, CHN1, CYP1B1, MME, WNT5A, POSTN, MMP2, CTGF, FBLN1, IGFBP3, DCN, PRRX1, ANXA6, PVRL3, MAP1B, TNFAIP6, CYBRD1, FBN1, NID1, OLFML3, SNAI1, SNAI2, TAGLN, SYNC, TUBA1A, PPM1D, TWIST1, FN1, TGFBR3, SERPINF1, UGDH, SRGN, FAP, PTGER4, VIM, PRKCA, FSTL1, MMP1, NRP1, FILIP1L, SCCPDH, LTBP2, XYLT1, HS3ST2, SYT11, TSHZ1, THY1, SEPT9, S100A4, CCDC92, TNS3, ENOX1, TGFB1I1, ZEB2, LMCD1, PDGFC, ECM1, TFPI, TBX3, DDR2, PTGER2, CDKN2C, VCAN, CD44, STARD13, SNED1, ZBTB38, SDC2, TPM1, COPZ2, STC1, CDH1, KRT5, KRT6B, CD24, EPCAM, ESRP1, CLDN3, CLDN4, ERBB3, SPOCK1, FERMT2, GLYR1, LTBP1, FADS2, KANK2, PTGFR, ARTN, SLC7A5, EPB41L4B, KRT15, CDH3, TPD52L1, ANXA3, PI3, NDRG1, DSG3, S100A14, S100S7, KLK8, S100A8, PTX3, SPINT2, FXYD3, KRT16, RAB25, LGALS7, DSG3, COL5A2, ITGB4, TMEM40, SPRR1A, AP1M2, SERPINB13, TP73L; and
(d) CYBRD1, ARTN, CITED2, SLC7A5, CHN1, CTGF, CSTA, MYL9, EPB41L4B, KRT15, LRIG1, CDH3, TPD52L1, ANXA3, OLFML3, CORO1A, PI3, NDRG1, DSG3, PMP22, S100A14, S100S7, KLK8, S100A8, PTX3, SPINT2, IER3, FXYD3, KRT16, TGFBR3, KRT5, RAB25, SDC2, LUM, LGALS7, KRT6B, CD24, COL1A2, GNG11, DSC3, COL5A2, RGS4, ITGB4, TMEM40, SPRR1A, AP1M2, SERPINB13, SNED1, LEPREL1, TRAM2, TP73L, and PAP2B; and
(e) CYBRD1, ARTN, CITED2, SLC7A5, CHN1, CTGF, CSTA, MYL9, EPB41L4B, KRT15, LRIG1, CDH3, TPD52L1, ANXA3, OLFML3, CORO1A; and
(f) PI3, NDRG1, DSG3, PMP22, S100A14, S100S7, KLK8, S100A8, PTX3, SPINT2, IER3, FXYD3, KRT16, TGFBR3; and
(g) KRT5, RAB25, SDC2, LUM, LGALS7, KRT6B, CD24, COL1A2, GNG11, DSC3, COL5A2; and
(h) RGS4, ITGB4, TMEM40, SPRR1A, AP1M2, AERPINB13, SNED1, LEPREL1, TRAM2, TP73L, and PAB2B; and
(i) CYBRD1, ARTN, KRT15, ITGB4, RGS4, SPRR1B, SLC7A5, ANXA3, IL18, AP1M2, CHN1, OLFML3, ZBED2, LEPREL1, ABLIM1, CDH3, DNAJB4, TRAM2, UCHL1, CTGF; and
   (i) CYBRD1, ARTN, KRT15, ITGB4, RGS4, SPRR1B, SLC7A5, ANXA3, IL18, AP1M2; and
   (ii) CYBRD1, ARTN, KRT15, ITGB4, RGS4; and
(j) CYBRD1, RGS4, CDH3, CHN1, SLC7A5, PRRX1, CITED2, COL5A2, ARTN, ZBED2, SNED1, ITGB4, LUM, PVRL3, OLFML3, BIN1, CCND2, DAB2, ANXA3, IL18; and
   (i) CYBRD1, RGS4, CDH3, CHN1, SLC7A5, PRRX1, CITED2, COL5A2, ARTN, ZBED2; and
   (ii) CYBRD1, RGS4, CDH3, CHN1, SLC7A5; and
(k) CYBRD1, ARTN, RGS4, CITED2, SLC7A5, ITGB4, CHN1, PI3, TMEM40, NDRG1, SPRR1A, PCDH9, CTGF, MAP1B, DSG3, AP1M2, FBLN5, SERPINB13, PMP22, CSTA; and
   (i) CYBRD1, ARTN, RGS4, CITED2, SLC7A5, ITGB4, CHN1, PI3, TMEM40, NDRG1; and
   (ii) CYBRD1, ARTN, RGS4, CITED2, SLC7A5; and

(ii) said second set of genes comprises or consists of:

AXL, NOTCH3, GPR30, PROCR, PAX2, PI3KA, CD44, CD133, ALDH1, BMI1, KRT19, NRP1, MSI1, MTOR, JAG1, p38, ESA1, ZEB1, TWIST1, FOXC2, CDH2, ZEB2, FOXC1, SNAI1, SNAI2, TWIST2, GSC, TCF3, CD44, VIM, FN1, MLPH1, ARHGEF11; and

(iii) said third set of genes comprises or consists of:

At least one gene from said first set of genes and at least one of:

AXL, NOTCH3, GPR30, PROCR, PAX2, PI3KA, CD44, CD133, ALDH1, BMI1, KRT19, NRP1, MSI1, MTOR, JAG1, p38, ESA1, ZEB1, TWIST1, FOXC2, CDH2, ZEB2, FOXC1, SNAI1, SNAI2, TWIST2, GSC, TCF3, CD44, VIM, FN1, MLPH1, ARHGEF11; and

(iv) said fourth set of genes comprises or consists of:

At least two genes from said first set of genes and at least one of: AXL, NOTCH3, GPR30, PROCR, PAX2, PI3KA, CD44, CD133, ALDH1, BMI1, KRT19, NRP1, MSI1, MTOR, JAG1, p38, ESA1, ZEB1, TWIST1, FOXC2, CDH2, ZEB2, FOXC1, SNAI1, SNAI2, TWIST2, GSC, TCF3, CD44, VIM, FN1, MLPH1, and ARHGEF11; and

(v) said fifth set of genes comprises or consists of:

beta catenin, FZD1, FZD2, FZD3, FZD4, FZD5, FZD6, FZD7, FZD8, FZD9, FZD10, LRP5, LRP6, WNT1, WNT2, WNT2B, WNT3, WNT3A, WNT4 WNT5A, WNT5B, WNT6, WNT7A, WNT7B, WNT8A, WNT8B, WNT9A, WNT9B, WNT10A, WNT10B, WNT11, WNT16, SRFP1, SRFP2, FRZB, SFRP4, SFRP5, DKK1, DKK2, DKK3, DKK4, WIF1, AKT2, PI3KA, Disheveled, Smoothened, Axin1, Axin2; and

(vi) said sixth set of genes comprises or consists of:

At least one gene from said first set of genes and at least one of:

beta catenin, FZD1, FZD2, FZD3, FZD4, FZD5, FZD6, FZD7, FZD8, FZD9, FZD10, LRP5, LRP6, WNT1, WNT2, WNT2B, WNT3, WNT3A, WNT4 WNT5A, WNT5B, WNT6, WNT7A, WNT7B, WNT8A, WNT8B, WNT9A, WNT9B, WNT10A, WNT10B, WNT11, WNT16, SRFP1, SRFP2, FRZB, SFRP4, SFRP5, DKK1, DKK2, DKK3, DKK4, WIF1, AKT2, PI3KA, Disheveled, Smoothened, Axin1, Axin2; and

(vii) said seventh set of genes comprises or consists of:

TGFbeta1, TGFbeta2, TGFbeta3, ALK1, ALK2, ALK3, ALK4, ALK5, ALK6, ALK7, TGFbetaR-II, BMPR-II, ACTR-IIA, ACTR-IIB, AMHR-II, SMAD2, SMAD3; and

(viii) said eighth set of genes comprises or consists of:

At least one gene from said first set of genes and at least one of:

TGFbeta1, TGFbeta2, TGFbeta3, ALK1, ALK2, ALK3, ALK4, ALK5, ALK6, ALK7, TGFbetaR-II, BMPR-II, ACTR-IIA, ACTR-IIB, AMHR-II, SMAD2, SMAD3; and

(ix) said ninth set of genes comprises or consists of:

S100A4, SDC2, CYP1B1, FERMT2, CYBRD1, SNAI2, CD44, BIN1, PMP22, CTGF, IGFBP3, ANXA6, MAP1B, TAGLN, ENPP2, FBLN5, PCOLCE, FN1, VIM, PPAP2B, SEM5A, VCAN, NID2, NID1, HAS2, PRRX1, TNFAIP6, CLDN4, FADS2, DDIT4, GNG11, PER1, ISCU, KPNA3, SLC44A1, FKBP5, NR2F1, MLPH, DOCK10, RGS4, IGFBP4; and

(x) said tenth set of genes comprises or consists of:

S100A4, SDC2, CYP1B1, FERMT2, CYBRD1, SNAI2, CD44, BIN1, ENPP2, FBLN5, PCOLCE, FN1, VIM, PPAP2B, SEM5A, VCAN, CLDN4, FADS2, DDIT4, GNG11, PER1, ISCU, KPNA3, SLC44A1, FKBP5, IGFBP4; and

(xi) said eleventh set of genes comprises or consists of:

SDC2, CYBRD1, SNAI2, PMP22, CTGF, IGFBP3, ANXA6, MAP1B, TAGLN, PPAP2B, HAS2, NRF2, MLPH, DOCK10, RGS4; and

(xii) said twelfth set of genes comprises or consists of:

FERMT2, BIN1, PMP22, CHN1, CTGF, IGFBP3, NID2, CLDN4, CYBRD1, PRR16, ANXA6, PCOLCE, PPAP2B, HAS2, CDH11, MLPH, DOCK10, RGS4, PVRL3, NID2, SLC44A1, FKBP5; and

(xiii) said thirteenth set of genes comprises or consists of:

FERMT2, BIN1, PMP22, CHN1, CTGF, IGFBP3, NID2, CLDN4; and

(xiv) said fourteenth set of genes comprises or consists of:

HAS2, BIN1, PCOLCE, FERMT2, CTGF, IGFBP3, NID2, SLC44A1, FKBP5, and MLPH.

In one embodiment, the kit or product comprises a second agent capable of interacting with a gene expression product from said first and/or second and/or third and/or fourth and/or fifth and/or sixth and/or seventh and/or eighth and/or ninth and/or tenth and/or eleventh and/or twelfth and/or thirteenth and/or fourteenth set of genes. In one embodiment, the kit or product comprises a plurality of agents capable of interacting with a gene expression product from said first and/or second and/or third and/or fourth and/or fifth and/or sixth and/or seventh and/or eighth and/or ninth and/or tenth and/or eleventh and/or twelfth and/or thirteenth and/or fourteenth set of genes. In one embodiment, the kit or product comprises a plurality of agents capable of interacting with a plurality of gene expression products from said first and/or second and/or third and/or fourth and/or fifth and/or sixth and/or seventh and/or eighth and/or ninth and/or tenth and/or eleventh and/or twelfth and/or thirteenth and/or fourteenth set of genes.

In one embodiment, said agent is a plurality of antibodies. In one embodiment, said agent is a plurality of oligonucleotides. In one embodiment, said gene expression product is a RNA product. In one embodiment, said gene expression product is a protein product.

In one embodiment, the gene expression products are derived from a tumor sample, e.g., a preparation of a primary tumor, metastatic tumor, lymph node, circulating tumor cells, ascites, or pleural effusion, plasma, serum, circulating, and interstitial fluid levels.

In one embodiment, a value for the level of gene expression product for each gene is determined. In one embodiment, a value that is a function of the level of gene expression for each gene is determined. In one embodiment, the value is compared to a reference standard, e.g., the level of expression of a control gene in the tumor sample.

In one embodiment, the kit or product further comprises the performance of an algorithm on a computer system to determine a value or values that is a function of a location of a gene expression product in the subject sample and/or a function of a level of a gene expression product of a gene in the subject sample. In one embodiment, the algorithm compares a ratio of the level of gene expression product of at least one of the genes selected from the group: HAS2, BIN1, PCOLCE, FERMT2, CTGF, IGFBP3, NID2, SLC44A1, FKBP5, and MLPH; to the level of gene expression product of at least one of the genes selected from the group: CDH1, and Cytokeratin.

In one embodiment, the kit or product further comprises a plurality of agents capable of interacting with at least one gene expression product selected from the group: CTGF, IGFBP3, TNFAIP6, NID2, HAS2, CCL2, MLPH, NID1, IGFBP4, FBLN5, and PCOLCE. In one embodiment, the kit or product further comprises a plurality of agents capable of interacting with a gene expression product of each gene from the set of genes consisting of: CTGF, IGFBP3, TNFAIP6, NID2, HAS2, CCL2, MLPH, NID1, IGFBP4, FBLN5, and PCOLCE.

In one aspect, kits or products described herein include kits or products comprising a first agent capable of interacting with a gene expression product of a gene from a first and/or second and/or third and/or fourth and/or fifth and/or sixth set of genes, wherein:

(i) said first set of genes comprises or consists of:
S100A4, SDC2, CYP1B1, FERMT2, CYBRD1, SNAI2, CD44, BIN1, PMP22, CTGF, IGFBP3, ANXA6, MAP1B, TAGLN, ENPP2, FBLN5, PCOLCE, FN1, VIM, PPAP2B, SEM5A, VCAN, NID2, NID1, HAS2, PRRX1, TNFAIP6, CLDN4, FADS2, DDIT4, GNG11, PER1, ISCU, KPNA3, SLC44A1, FKBP5, NR2F1, MLPH, DOCK10, RGS4, IGFBP4; and

(ii) said second set of genes comprises or consists of:
S100A4, SDC2, CYP1B1, FERMT2, CYBRD1, SNAI2, CD44, BIN1, ENPP2, FBLN5, PCOLCE, FN1, VIM, PPAP2B, SEM5A, VCAN, CLDN4, FADS2, DDIT4, GNG11, PER1, ISCU, KPNA3, SLC44A1, FKBP5, IGFBP4; and

(iii) said third set of genes comprises or consists of:
SDC2, CYBRD1, SNAI2, PMP22, CTGF, IGFBP3, ANXA6, MAP1B, TAGLN, PPAP2B, HAS2, NRF2, MLPH, DOCK10, RGS4; and

(iv) said fourth set of genes comprises or consists of:
FERMT2, BIN1, PMP22, CHN1, CTGF, IGFBP3, NID2, CLDN4, CYBRD1, PRR16, ANXA6, PCOLCE, PPAP2B, HAS2, CDH11, MLPH, DOCK10, RGS4, PVRL3, NID2, SLC44A1, FKBP5; and

(v) said fifth set of genes comprises or consists of:
FERMT2, BIN1, PMP22, CHN1, CTGF, IGFBP3, NID2, CLDN4; and

(vi) said sixth set of genes comprises or consists of:
HAS2, BIN1, PCOLCE, FERMT2, CTGF, IGFBP3, NID2, SLC44A1, FKBP5, and MLPH.

In one embodiment, the kit or product comprises a second agent capable of interacting with a gene expression product of a plurality of genes from said first and/or second and/or third and/or fourth and/or fifth and/or sixth set of genes. In one embodiment, the kit or product comprises a plurality of agents capable of interacting with a gene expression product of a plurality of genes from said first and/or second and/or third and/or fourth and/or fifth and/or sixth set of genes. In one embodiment, the kit or product comprises a plurality of agents capable of interacting with a plurality of gene expression products of a plurality of genes from said first and/or second and/or third and/or fourth and/or fifth and/or sixth set of genes.

In one embodiment, said agent is a plurality of antibodies. In one embodiment, said agent is a plurality of oligonucleotides. In one embodiment, said gene expression product is a RNA product. In one embodiment, said gene expression product is a protein product. In one embodiment, a value for the level of gene expression product for each gene isoform is assayed. In one embodiment, a value for the level of gene expression product for each gene isoform is assayed by detecting a protein product. In one embodiment, the protein product is detected by an immunoassay, e.g., immunohistochemistry. In one embodiment, a value for the level of gene expression product for each gene isoform is assayed by detecting a RNA product. In one embodiment, the RNA product is detected by a hybridization based method. In one embodiment, the RNA product is detected by microarray. In one embodiment, said microarray is an exon microarray. In one embodiment, the RNA product is detected by a polymerase chain reaction based method. In one embodiment, the RNA product is detected by a sequencing based method. In one embodiment, the RNA product is detected by a quantitative RNA sequencing.

In one embodiment, the gene expression products are derived from a tumor sample, e.g., a preparation of a primary tumor, metastatic tumor, lymph node, circulating tumor cells, ascites, or pleural effusion, plasma, serum, circulating, and interstitial fluid levels.

In one embodiment, a value for the level of gene expression product for each gene is determined. In one embodiment, a value that is a function of the level of gene expression for each gene is determined. In one embodiment, the value is compared to a reference standard, e.g., the level of expression of a control gene in the tumor sample.

In one embodiment, the kit or product further comprises the performance of an algorithm on a computer system to determine a value or values that is a function of a location of a gene expression product in the subject sample and/or a function of a level of a gene expression product of a gene in the subject sample. In one embodiment, the algorithm compares a ratio of the level of gene expression product of at least one of the genes selected from the group: HAS2, BIN1, PCOLCE, FERMT2, CTGF, IGFBP3, NID2, SLC44A1, FKBP5, and MLPH; to the level of gene expression product of at least one of the genes selected from the group: CDH1, and Cytokeratin.

In one embodiment, the kit or product further comprises a plurality of agents capable of interacting with at least one gene expression product selected from the group: CTGF, IGFBP3, TNFAIP6, NID2, HAS2, CCL2, MLPH, NID1, IGFBP4, FBLN5, and PCOLCE. In one embodiment, the kit or product further comprises a plurality of agents capable of interacting with a gene expression product of each gene from the set of genes consisting of: CTGF, IGFBP3, TNFAIP6, NID2, HAS2, CCL2, MLPH, NID1, IGFBP4, FBLN5, and PCOLCE.

Methods of Assaying

In one aspect, methods described herein include methods of assaying in a subject sample the level of gene expression product of a plurality of genes from a first and/or second and/or third and/or fourth and/or fifth and/or sixth and/or seventh and/or eighth and/or ninth and/or tenth and/or eleventh and/or twelfth and/or thirteenth and/or fourteenth set of genes, wherein:

(i) said first set of genes comprises or consists of:
KLF13, ANTXR2, IFNAR1, LIX1L, CHST11, AKAP2, PALM2-AKAPT, LOC80298, C7orf2, NUCKS, DKFZP566D1346, LOC388279, FLJ31795, 6orf107, FLJ12439, ENPP2, CITED2, ZEB1, NID2, SEMA5A, DAB2, KCNMA1, PTX3, PCDH9, CREB3L1, BGN, ROR1, DLC1, GNG11, CDH11, NR2F1, PRR16, MYL9, DOCK10, LRIG1, IER3, EML1, NEBL, RGL1, MLPH, DNAJB4, FBLN5, RGS4, HAS2, ITGBL1, IGFBP4, DPT, PCOLCE, GREM1, PPAP2B, CDH2, PMP22, LUM, CHN1, CYP1B1, MME, WNT5A, POSTN, MMP2, CTGF, FBLN1, IGFBP3, DCN, PRRX1, ANXA6, PVRL3, MAP1B, TNFAIP6, CYBRD1, FBN1, NID1, OLFML3, SNAI1, SNAI2, TAGLN, SYNC, TUBA1A, PPM1D, TWIST1, FN1, TGFBR3, SERPINF1, UGDH, SRGN, FAP, PTGER4, PRKCA, FSTL1, MMP1, NRP1, FILIP1L, SCCPDH, LTBP2, XYLT1, HS3ST2, SYT11, TSHZ1, THY1, Sep9, S100A4, TNS3, ENOX1, TGFB1I1, ZEB2, LMCD1, PDGFC, ECM1, TFPI, TBX3, DDR2, PTGER2, CDKN2C, VCAN, CD44, STARD13, SNED1, ZBTB38, SDC2, TPM1, COPZ2, STC1, CDH1, KRT5, KRT6B, CD24, EPCAM, ESRP1, CLDN3, CLDN4, ERBB3, SPOCK1, FERMT2, GLYR1, LTBP1, FADS2, KANK2, PTGFR, COL11A2, KLK3, EIF2C2, ZFP41, FAM49B, PSORS1C2, MRPL42, MRPL54, MRPL47, MRPS23, EIF3S9, ALG5, DNAJC19, TPRXL, NOTCH2, RBM15, ST3GAL3, NFYA, PCNX, FBXO21, WWOX, CAMK2B, PNPLA2, CLIC5, UGCGL1, FBXL18, ADRBK1, SLC38A2, IL8RA, TAS2R14, CD300LB, GIPC3, RAD51L1, EPPK1, COL1A1, MMP9, SERPINE1, SPARC, TGFB1, TGFB3, TGFBI, TGFBR1, TGIF, TGIF2, THBS1, ANXA5, ACTG1, ARF3, ATP1B3, BAT3, CALD1, CENTD2, CLIC1, CTBS, DPYSL3, DVL3, EXT1, FGFR1, FTL, GNB2L1, GPRC5A, H2AFZ, HIF1A, IL13RA1, KDELR2, LARP1, LPIN2, MARS, MMP10, MMP14, MT2A, MYO10, NUP62, PFKFB3, PLOD2, PSMB7, PSMD8, RIN2, RYBP, SDF4, SETD5, SPP1, STAU1, TUBB3, UBE2S, XPNPEP1, CDKN1A, CHRD, H19, ID3, ID4, IGFBP7, LRP1, MSX1, NOTCH3, PROCR, GBX2, KI67, CCNB1, BUB1, KNTC2, USP22, HCFC1, RNF2, ANK3, FGFR2, CES1, COL1A2, COL3A1, COL5A2, COL6A1, ANKRD25, C10ORF56, C5ORF13, KRT81, NPAC, PLEKHC1, SEPT9, SYNC1, MBP, ABLIM1, ALDH1A3, ALOX15B, ANXA3, AP1M2, ARTN, CA2, CA9, CDH3, CDS1, COL17A1, CORO1A, CTSL2, CXADR, CYP27B1, DSC2, DSG3, DST, EPB41L4B, FGFBP1, FGFR3, FST, GJB3, GRHL2, HBEGF, HOOK1, IL18, IL1B, IRF6, ITGB4, JAG2, KLK10, KLK5, KLK7, KLK8, KRT15, KRT16, KRT17, LEPREL1, MYO5C, NDRG1, NMU, PI3, RAB25, RLN2, RNF128, S100A14, S100A7, S100A8, SERPINB1, SERPINB2, SLC2A9, SLPI, SNX10, SPINT2, SPRR1A, SPRR1B, ST14, SYK, TMEM40, TRIM29, TSPAN1, UCHL1, VSNL1, ZBED2, AQP3, CBARA1, CLCA2, CNTNAP2, CSTA, DSC3, ESRP2, FXYD3, GJB5, GPR87, IL1A, LAMA3, LAMB3, MAP7, MAPK13, MPZL2, NRG1, SCNN1A, SERPINB13, SLC7A5, SPINT1, ST6GALNAC2, TGFA, TP63, TPD52L1, VGLL1, SLC6A8, ABCA12, ANXA8, ANXA8L1, ARHGAP25, ARHGAP8, BDKRB2, BIK, C10ORF10, C10ORF116, C1ORF116, C20ORF19, C6ORF105, CELSR2, CKMT1A, CKMT1B, CLDN1, CST6, CYP4F11, E2F5, ELMO3, EPHA1, EVA1, EXPH5, F11R, FAT2, FLJ12684, FLJ20366, FZD3, GLS2, GNAL, IFI30, IGFBP2, IL1RN, IL4R, IRX4, JUP, KCNK1, KIAA0040, KIAA0888, KIAA1815, KRT14, KRT18, LAD1, LGALS7, LOC553158, LOC653562, LOC728113, LOC729884, LRRC1, LSR, MST1R, MYO1D, NAIP, OCLN, NEFM, NUP62CL, PERP, PLS1, POLR3G, PRKCH, PRRG4, PRSS8, PTPN3, RBM35A, RBM35B, RHBDF2, RHOD, RPS6KA1, RTEL1, SAA1, SAA1, SAA2, SH2D3A, SLC6A10P, SMPDL3B, SNCA, STAC, STAP2, TACSTD1, THBD, TMEM30B, TMPRSS11E, TMPRSS4, TNFRSF6B, TP73L, WWC1, XDH, ZNF165, AXL, GPR30, PAX2, PI3KA, CD133, ALDH1, BMI1, KRT19, MSI1, JAG1, p38, ESA1, FOXC2, FOXC1, TWIST2, GSC, TCF3, MLPH1, ARHGEF11a, DPF2, CASP8, BCL2, SCGN, SWAP70, KIAA0276, C10orf9, C10orf7, ALKBH, TOB2, XPR1, CD59, LRP2, PLP2, MAPK14, CXCL2, MMP7, MGP, MLF1, FLNB, SCNM1, HSPC163, C5orf18, MGC4399, CDW92, TMC4, ZDHHC2, TICAM2, KDELR3, GNPDA1, THEM2, DBR1, FLJ90709, FLJ10774, C16orf33, GAPD, LDHA, MR-1, LARS, GTPBP1, PRSS16, WFDC2, AIM1, DHRS6, DHRS4, GC15429, MGC45840, ECHDC2, GOLGIN-67, AFURS1, KIAA0436, CYP4V2, JTV1, ICMT, DNMT3A, HNMT, METTL7A, METTL2, VIL2, TPD52, ARPC5, NOL8, NSF, RAD23B, SRP54, HSPA2, PBP, THAP2, CIRBP, SNRPN, KIAA0052, DUSP10, SSR1, ERBB4, EMP1, CHPT1, LRPAP1, FLJ11752, CSTF1, KLHL20, DNAJC13, APLP2, ARGBP2, DNAJB1, NEBL, SH3BGRL, NUDT5, GABARAPL1, MAPT, DCBLD1, STK39, PAK2, CSNK2A1, PILRB, ERN1, SGKL, WEE1, MAST4, C11orf17, NUP37, CLTC, COPB2, SLC25A25, ECOP, PDE8A, STAM, TUBB, SNX6, RAB23, PLAA, STC2, LTF, ISGF3G, ATXN3, GTF3C3, GSK3B, KLF10, ELL2, ZBTB20, IRX3, ETS1, SERTAD1, MGC4251, MAFF, SFPQ, CITED4, CEBPD, EIF4E2, HS2ST1, AGPS, PGK1, ATIC, ETNK1, LG2, NCE2, MARCH8, CNOT4, RNF8, PSMA5, DPF2, AMMECR1, KIAA1287, LOC144233, LOC286505, PNAS-4, FLJ20530, HUMPD3, GC45564, CAP350, ETAA16, HAN11, DNAPTP6, C7orf25, FLJ37953, FLJ10587, C7orf36, ELP4, NDEL1, NPD014, KFZP564D172, FAM53C, IER5, LOC255783, KIAA0146, KIAA0792, LOC439994, LOC283481, CG018, LOC130576, NGFRAP1L1, KIAA1217, 4orf7, C21 orf86, C9orf64, FLJ13456, FLJ12806, FLJ39370, GATS, CCDC92, FMNL2, ARID1B, ZFHX1B, SSBP2, ARID5B, LOC157381, KPNA3, ARHGAP24, CCND2, VIM, MYCBP2, GAS7, TRAM2, BASP1, FOXO1A, POLR2A, PER1, DDIT4, CD97, BIN1, SH2B3, DDB2, EMP3, NDST2, CHST2, NT5E, PDE4A, CPS1, PTGS1, GGCX, IRF5, ZBTB16, MAP4K4, CHST7, KLF12, NFRKB, PSD, FKSG49, NIFUN, FYN, ZMYM2, CACNA1G, SLC25A16, FLII, EIF1, SEPT6, NPAC, PHF15, NUP188, ABR, CNR1, LOC283824, FSTL4, DNM1, APOBEC3G, ATP2B1, SMPD1, SLC11A1, FXYD5, C14orf139, SH3BGRL3, LUZP1, ZNF335, SH3 KBP1, MST150, PRO1073, LOC388397, FKBP5, HIPK2, DTX1, ST3GAL2, ADAMTS7, TNRC6B, CYGB, SDHAL1, LOC572558, TRIO, FRAS1, KIAA1632, POLS, EBF, MAML2, PTPRA, PLEKHG2, DYM, SOX6, ARHGEF2, ZCCHC6, PPP3CA, FAM70B, TMED5, FLJ43663, HPS1, MEF2A, ST3GAL5, SMYD3, KLF7, LOC200230, RERE, QKI, BICD1, CTNNB1, POU2F2, EIF4ENIF1, BTG1, TCHP, FLJ90709, PCTK2, KIAA1600, B7-H4, PDE4DIP, KIAA0194, HOM-TES; and
(a) BGN, BIN1, CDH11, CDH2, CHN1, COL1A2, COL3A1, COL5A2, CREB3L1, CTGF, CYBRD1, DCN, DLC1, DNAJB4, DPT, ENPP2, FBLN1, FBLN5, FBN1, GREM1, HAS2, IGFBP3, IGFBP4, MLPH, MME, MMP2, MYL9, NEBL, NID1, NR2F1, OLFML3, PCOLCE, PMP22, POSTN, PPAP2B, PRR16, PRRX1, PTX3, PVRL3, RGL1, RGS4, ROR1, SEMA5A, TNFAIP6, TRAM2, WNT5A, ZEB1, ANXA6, CITED2, COL1A1, COL6A1, CYP1B1, DAB2, DOCK10, EML1, GNG11, IER3, ITGBL1, KCNMA1, LRIG1, LUM, MAP1B, NID2, PCDH9, PRKCA, PTGER4, PTGFR, SDC2, SERPINF1, SNED1, SYT11, TFPI, TGFBR3, VCAN, ANKRD25, C10ORF56, C5ORF13, CCDC92, CDKN2C, COPZ2, DDR2, ECM1, ENOX1, FADS2, FAP, FILIP1L, FSTL1, HS3ST2, KRT81, LMCD1, LTBP1, LTBP2, MMP1, NPAC, NRP1, PDGFC, PLEKHC1, PPM1D, PTGER2, SCCPDH, SEPT9, SPOCK1, SRGN, STARD13, STC1, SYNC1, TAGLN, TBX3, TGFB1I1, THY1, TNS3, TPM1, TSHZ1, TUBA1A, UGDH, VIM, XYLT1, ZBTB38, MBP, ABLIM1, ALDH1A3, ALOX15B, ANXA3, AP1M2, ARTN, CA2, CA9, CAMK2B, CCND2, CDH1, CDH3, CDS1, COL17A1, CORO1A, CTSL2, CXADR, CYP27B1, DSC2, DSG3, DST, EPB41L4B, FGFBP1, FGFR3, FST, GJB3, GRHL2, HBEGF, HOOK1, IL18, IL1B, IRF6, ITGB4, JAG2, KLK10, KLK5, KLK7, KLK8, KRT15, KRT16, KRT17, KRT5, KRT6B, LEPREL1, MYO5C, NDRG1, NMU, PI3, RAB25, RLN2, RNF128, S100A14, S100A7, S100A8, SERPINB1, SERPINB2, SLC2A9, SLPI, SNX10, SPINT2, SPRR1A, SPRR1B, ST14, SYK, TMEM40, TRIM29, TSPAN1, UCHL1, VSNL1, ZBED2, AQP3, CBARA1, CD24, CLCA2, CNTNAP2, CSTA, DSC3, EPCAM, ESRP2, FXYD3, GJB5, GPR87, IL1A, LAMA3, LAMB3, MAP7, MAPK13, MMP9, MPZL2, NRG1, SCNN1A, SERPINB13, SLC7A5, SPINT1, ST6GALNAC2, TGFA, TP63, TPD52L1, VGLL1, SLC6A8, ABCA12, ANK3, ANXA8, ANXA8L1, ARHGAP25, ARHGAP8, BDKRB2, BIK, C10ORF10, C10ORF116, C1ORF116, C20ORF19, C6ORF105, CELSR2, CKMT1A, CKMT1B, CLDN1, CST6, CYP4F11, E2F5, ELMO3, EPHA1, EVA1, EXPH5, F11R, FAT2, FGFR2, FLJ12684, FLJ20366, FZD3, GLS2, GNAL, IFI30, IGFBP2, IL1RN, IL4R, IRX4, JUP, KCNK1, KIAA0040, KIAA0888, KIAA1815, KRT14, KRT18, LAD1, LGALS7, LOC553158, LOC653562, LOC728113, LOC729884, LRRC1, LSR, MST1R, MYO1D, NAIP, OCLN, NEFM, NUP62CL, PERP, PLS1, POLR3G, PRKCH, PRRG4, PRSS8, PTPN3, RBM35A, RBM35B, RHBDF2, RHOD, RPS6KA1, RTEL1, SAA1, SAA1, SAA2, SH2D3A, SLC6A10P, SMPDL3B, SNCA, STAC, STAP2, TACSTD1, THBD, TMEM30B, TMPRSS11E, TMPRSS4, TNFRSF6B, TP73L, WWC1, XDH, ZNF165; and
(b) BGN, BIN1, CDH11, CDH2, CHN1, COL1A2, COL3A1, COL5A2, CREB3L1, CTGF, CYBRD1, DCN, DLC1, DNAJB4, DPT, ENPP2, FBLN1, FBLN5, FBN1, GREM1, HAS2, IGFBP3, IGFBP4, MLPH, MME, MMP2, MYL9, NEBL, NID1, NR2F1, OLFML3, PCOLCE, PMP22, POSTN, PPAP2B, PRR16, PRRX1, PTX3, PVRL3, RGL1, RGS4, ROR1, SEMA5A, TNFAIP6, TRAM2, WNT5A, ZEB1, ANXA6, CITED2, COL1A1, COL6A1, CYP1B1, DAB2, DOCK10, EML1, GNG11, IER3, ITGBL1, KCNMA1, LRIG1, LUM, MAP1B, NID2, PCDH9, PRKCA, PTGER4, PTGFR, SDC2, SERPINF1, SNED1, SYT11, TFPI, TGFBR3, VCAN, ANKRD25, C10ORF56, C5ORF13, CCDC92, CDKN2C, COPZ2, DDR2, ECM1, ENOX1, FADS2, FAP, FILIP1L, FSTL1, HS3ST2, KRT81, LMCD1, LTBP1, LTBP2, MMP1, NPAC, NRP1, PDGFC, PLEKHC1, PPM1D, PTGER2, SCCPDH, SEPT9, SPOCK1, SRGN, STARD13, STC1, SYNC1, TAGLN, TBX3, TGFB1I1, THY1, TNS3, TPM1, TSHZ1, TUBA1A, UGDH, VIM, XYLT1, ZBTB38; and
(c) ENPP2, CITED2, ZEB1, NID2, SEMA5A, DAB2, KCNMA1, PTX3, PCDH9, CREB3L1, BGN, ROR1, DLC1, GNG11, CDH11, NR2F1, NR2F2, PRR16, MYL9, DOCK10, LRIG1, IER3, EML1, NEBL, RGL1, MLPH, TRAM2, DNAJB4, FBLN5, RGS4, HAS2, ITGBL1, IGFBP4, BIN1, DPT, PCOLCE, GREM1, PPAP2B, CDH2, PMP22, LUM, CHN1, CYP1B1, MME, WNT5A, POSTN, MMP2, CTGF, FBLN1, IGFBP3, DCN, PRRX1, ANXA6, PVRL3, MAP1B, TNFAIP6, CYBRD1, FBN1, NID1, OLFML3, SNAI1, SNAI2, TAGLN, SYNC, TUBA1A, PPM1D, TWIST1, FN1, TGFBR3, SERPINF1, UGDH, SRGN, FAP, PTGER4, VIM, PRKCA, FSTL1, MMP1, NRP1, FILIP1L, SCCPDH, LTBP2, XYLT1, HS3ST2, SYT11, TSHZ1, THY1, SEPT9, S100A4, CCDC92, TNS3, ENOX1, TGFB1I1, ZEB2, LMCD1, PDGFC, ECM1, TFPI, TBX3, DDR2, PTGER2, CDKN2C, VCAN, CD44, STARD13, SNED1, ZBTB38, SDC2, TPM1, COPZ2, STC1, CDH1, KRT5, KRT6B, CD24, EPCAM, ESRP1, CLDN3, CLDN4, ERBB3, SPOCK1, FERMT2, GLYR1, LTBP1, FADS2, KANK2, PTGFR, ARTN, SLC7A5, EPB41L4B, KRT15, CDH3, TPD52L1, ANXA3, PI3, NDRG1, DSG3, S100A14, S100S7, KLK8, S100A8, PTX3, SPINT2, FXYD3, KRT16, RAB25, LGALS7, DSC3, COL5A2, ITGB4, TMEM40, SPRR1A, AP1M2, SERPINB13, TP73L; and
(d) CYBRD1, ARTN, CITED2, SLC7A5, CHN1, CTGF, CSTA, MYL9, EPB41L4B, KRT15, LRIG1, CDH3, TPD52L1, ANXA3, OLFML3, CORO1A, PI3, NDRG1, DSG3, PMP22, S100A14, S100S7, KLK8, S100A8, PTX3, SPINT2, IER3, FXYD3, KRT16, TGFBR3, KRT5, RAB25, SDC2, LUM, LGALS7, KRT6B, CD24, COL1A2, GNG11, DSC3, COL5A2, RGS4, ITGB4, TMEM40, SPRR1A, AP1M2, SERPINB13, SNED1, LEPREL1, TRAM2, TP73L, and PAP2B; and
(e) CYBRD1, ARTN, CITED2, SLC7A5, CHN1, CTGF, CSTA, MYL9, EPB41L4B, KRT15, LRIG1, CDH3, TPD52L1, ANXA3, OLFML3, CORO1A; and
(f) PI3, NDRG1, DSG3, PMP22, S100A14, S100S7, KLK8, S100A8, PTX3, SPINT2, IER3, FXYD3, KRT16, TGFBR3; and
(g) KRT5, RAB25, SDC2, LUM, LGALS7, KRT6B, CD24, COL1A2, GNG11, DSC3, COL5A2; and
(h) RGS4, ITGB4, TMEM40, SPRR1A, AP1M2, AERPINB13, SNED1, LEPREL1, TRAM2, TP73L, and PAB2B; and
(i) CYBRD1, ARTN, KRT15, ITGB4, RGS4, SPRR1B, SLC7A5, ANXA3, IL18, AP1M2, CHN1, OLFML3, ZBED2, LEPREL1, ABLIM1, CDH3, DNAJB4, TRAM2, UCHL1, CTGF; and
   (i) CYBRD1, ARTN, KRT15, ITGB4, RGS4, SPRR1B, SLC7A5, ANXA3, IL18, AP1M2; and
   (ii) CYBRD1, ARTN, KRT15, ITGB4, RGS4; and
(j) CYBRD1, RGS4, CDH3, CHN1, SLC7A5, PRRX1, CITED2, COL5A2, ARTN, ZBED2, SNED1, ITGB4, LUM, PVRL3, OLFML3, BIN1, CCND2, DAB2, ANXA3, IL18; and
   (i) CYBRD1, RGS4, CDH3, CHN1, SLC7A5, PRRX1, CITED2, COL5A2, ARTN, ZBED2; and
   (ii) CYBRD1, RGS4, CDH3, CHN1, SLC7A5; and
(k) CYBRD1, ARTN, RGS4, CITED2, SLC7A5, ITGB4, CHN1, PI3, TMEM40, NDRG1, SPRR1A, PCDH9, CTGF, MAP1B, DSG3, AP1M2, FBLN5, SERPINB13, PMP22, CSTA; and
   (i) CYBRD1, ARTN, RGS4, CITED2, SLC7A5, ITGB4, CHN1, PI3, TMEM40, NDRG1; and
   (ii) CYBRD1, ARTN, RGS4, CITED2, SLC7A5; and

(ii) said second set of genes comprises or consists of:

AXL, NOTCH3, GPR30, PROCR, PAX2, PI3KA, CD44, CD133, ALDH1, BMI1, KRT19, NRP1, MSI1, MTOR, JAG1, p38, ESA1, ZEB1, TWIST1, FOXC2, CDH2, ZEB2, FOXC1, SNAI1, SNAI2, TWIST2, GSC, TCF3, CD44, VIM, FN1, MLPH1, ARHGEF11; and

(iii) said third set of genes comprises or consists of:

At least one gene from said first set of genes and at least one of:

AXL, NOTCH3, GPR30, PROCR, PAX2, PI3KA, CD44, CD133, ALDH1, BMI1, KRT19, NRP1, MSI1, MTOR, JAG1, p38, ESA1, ZEB1, TWIST1, FOXC2, CDH2, ZEB2, FOXC1, SNAI1, SNAI2, TWIST2, GSC, TCF3, CD44, VIM, FN1, MLPH1, ARHGEF11; and

(iv) said fourth set of genes comprises or consists of:

At least two genes from said first set of genes and at least one of: AXL, NOTCH3, GPR30, PROCR, PAX2, PI3KA, CD44, CD133, ALDH1, BMI1, KRT19, NRP1, MSI1, MTOR, JAG1, p38, ESA1, ZEB1, TWIST1, FOXC2, CDH2, ZEB2, FOXC1, SNAI1, SNAI2, TWIST2, GSC, TCF3, CD44, VIM, FN1, MLPH1, and ARHGEF11; and

(v) said fifth set of genes comprises or consists of:

beta catenin, FZD1, FZD2, FZD3, FZD4, FZD5, FZD6, FZD7, FZD8, FZD9, FZD10, LRP5, LRP6, WNT1, WNT2, WNT2B, WNT3, WNT3A, WNT4 WNT5A, WNT5B, WNT6, WNT7A, WNT7B, WNT8A, WNT8B, WNT9A, WNT9B, WNT10A, WNT10B, WNT11, WNT16, SRFP1, SRFP2, FRZB, SFRP4, SFRP5, DKK1, DKK2, DKK3, DKK4, WIF1, AKT2, PI3KA, Disheveled, Smoothened, Axin1, Axin2; and

(vi) said sixth set of genes comprises or consists of:

At least one gene from said first set of genes and at least one of:

beta catenin, FZD1, FZD2, FZD3, FZD4, FZD5, FZD6, FZD7, FZD8, FZD9, FZD10, LRP5, LRP6, WNT1, WNT2, WNT2B, WNT3, WNT3A, WNT4 WNT5A, WNT5B, WNT6, WNT7A, WNT7B, WNT8A, WNT8B, WNT9A, WNT9B, WNT10A, WNT10B, WNT11, WNT16, SRFP1, SRFP2, FRZB, SFRP4, SFRP5, DKK1, DKK2, DKK3, DKK4, WIF1, AKT2, PI3KA, Disheveled, Smoothened, Axin1, Axin2; and

(vii) said seventh set of genes comprises or consists of:

TGFbeta1, TGFbeta2, TGFbeta3, ALK1, ALK2, ALK3, ALK4, ALK5, ALK6, ALK7, TGFbetaR-II, BMPR-II, ACTR-IIA, ACTR-IIB, AMHR-II, SMAD2, SMAD3; and

(viii) said eighth set of genes comprises or consists of:

At least one gene from said first set of genes and at least one of:

TGFbeta1, TGFbeta2, TGFbeta3, ALK1, ALK2, ALK3, ALK4, ALK5, ALK6, ALK7, TGFbetaR-II, BMPR-II, ACTR-IIA, ACTR-IIB, AMHR-II, SMAD2, SMAD3; and

(ix) said ninth set of genes comprises or consists of:

S100A4, SDC2, CYP1B1, FERMT2, CYBRD1, SNAI2, CD44, BIN1, PMP22, CTGF, IGFBP3, ANXA6, MAP1B, TAGLN, ENPP2, FBLN5, PCOLCE, FN1, VIM, PPAP2B, SEM5A, VCAN, NID2, NID1, HAS2, PRRX1, TNFAIP6, CLDN4, FADS2, DDIT4, GNG11, PER1, ISCU, KPNA3, SLC44A1, FKBP5, NR2F1, MLPH, DOCK10, RGS4, IGFBP4; and

(x) said tenth set of genes comprises or consists of:

S100A4, SDC2, CYP1B1, FERMT2, CYBRD1, SNAI2, CD44, BIN1, ENPP2, FBLN5, PCOLCE, FN1, VIM, PPAP2B, SEM5A, VCAN, CLDN4, FADS2, DDIT4, GNG11, PER1, ISCU, KPNA3, SLC44A1, FKBP5, IGFBP4; and

(xi) said eleventh set of genes comprises or consists of:

SDC2, CYBRD1, SNAI2, PMP22, CTGF, IGFBP3, ANXA6, MAP1B, TAGLN, PPAP2B, HAS2, NRF2, MLPH, DOCK10, RGS4; and

(xii) said twelfth set of genes comprises or consists of:

FERMT2, BIN1, PMP22, CHN1, CTGF, IGFBP3, NID2, CLDN4, CYBRD1, PRR16, ANXA6, PCOLCE, PPAP2B, HAS2, CDH11, MLPH, DOCK10, RGS4, PVRL3, NID2, SLC44A1, FKBP5; and

(xiii) said thirteenth set of genes comprises or consists of:

FERMT2, BIN1, PMP22, CHN1, CTGF, IGFBP3, NID2, CLDN4; and

(xiv) said fourteenth set of genes comprises or consists of:

HAS2, BIN1, PCOLCE, FERMT2, CTGF, IGFBP3, NID2, SLC44A1, FKBP5, and MLPH; comprising a first agent capable of interacting with a gene expression product of a plurality of genes selected from a first and/or second and/or third and/or fourth and/or fifth and/or sixth set of genes; and wherein the method comprises assaying the level of gene expression product of the plurality of genes.

In one embodiment, the method comprises a second agent capable of interacting with a gene expression product of a plurality of genes in said first and/or second and/or third and/or fourth and/or fifth and/or sixth and/or seventh and/or eighth and/or ninth and/or tenth and/or eleventh and/or twelfth and/or thirteenth and/or fourteenth set of genes. In one embodiment, the method comprises a plurality of agents capable of interacting with a gene expression product of a plurality of genes from said first and/or second and/or third and/or fourth and/or fifth and/or sixth and/or seventh and/or eighth and/or ninth and/or tenth and/or eleventh and/or twelfth and/or thirteenth and/or fourteenth set of genes. In one embodiment, the method comprises a plurality of agents capable of interacting with a plurality of gene expression products of a plurality of genes from said first and/or second and/or third and/or fourth and/or fifth and/or sixth and/or seventh and/or eighth and/or ninth and/or tenth and/or eleventh and/or twelfth and/or thirteenth and/or fourteenth set of genes.

In one embodiment, said agent is a plurality of antibodies. In one embodiment, said agent is a plurality of oligonucleotides. In one embodiment, said gene expression product is a RNA product. In one embodiment, said gene expression product is a protein product. In one embodiment, said agent is a plurality of antibodies. In one embodiment, said agent is a plurality of oligonucleotides. In one embodiment, said gene expression product is a RNA product. In one embodiment, said gene expression product is a protein product. In one embodiment, a value for the level of gene expression product for each gene isoform is assayed. In one embodiment, a value for the level of gene expression product for each gene isoform is assayed by detecting a protein product. In one embodiment, the protein product is detected by an immunoassay, e.g., immunohistochemistry. In one embodiment, a value for the level of gene expression product for each gene isoform is assayed by detecting a RNA product. In one embodiment, the RNA product is detected by a hybridization based method. In one embodiment, the RNA product is detected by microarray. In one embodiment, said microarray is an exon microarray. In one embodiment, the RNA product is detected by a polymerase chain reaction based method. In one embodiment, the RNA product is detected by a sequencing based method. In one embodiment, the RNA product is detected by a quantitative RNA sequencing.

In one embodiment, the gene expression products are derived from a tumor sample, e.g., a preparation of a primary tumor, metastatic tumor, lymph node, circulating tumor cells, ascites, or pleural effusion, plasma, serum, circulating, and interstitial fluid levels.

In one embodiment, a value for the level of gene expression product for each gene is determined. In one embodiment, a value that is a function of the level of gene expression for each gene is determined. In one embodiment, the value is compared to a reference standard, e.g., the level of expression of a control gene in the tumor sample.

In one embodiment, the method further comprises the performance of an algorithm on a computer system to determine a value or values that is a function of a location of a gene expression product in the subject sample and/or a function of a level of a gene expression product of a gene in the subject sample. In one embodiment, the algorithm compares a ratio of the level of gene expression product of at least one of the genes selected from the group: HAS2, BIN1, PCOLCE, FERMT2, CTGF, IGFBP3, NID2, SLC44A1, FKBP5, and MLPH; to the level of gene expression product of at least one of the genes selected from the group: CDH1, and Cytokeratin.

In one embodiment, the method further comprises a plurality of agents capable of interacting with at least one gene expression product selected from the group: CTGF, IGFBP3, TNFAIP6, NID2, HAS2, CCL2, MLPH, NID1, IGFBP4, FBLN5, and PCOLCE. In one embodiment, the method further comprises a plurality of agents capable of interacting with a gene expression product of each gene from the set of genes consisting of: CTGF, IGFBP3, TNFAIP6, NID2, HAS2, CCL2, MLPH, NID1, IGFBP4, FBLN5, and PCOLCE.

In one aspect, methods described herein include methods of assaying in a subject sample the level of gene expression product of a plurality of genes from a first and/or second and/or third and/or fourth and/or fifth and/or sixth set of genes, wherein:
(i) said first set of genes comprises or consists of:
S100A4, SDC2, CYP1B1, FERMT2, CYBRD1, SNAI2, CD44, BIN1, PMP22, CTGF, IGFBP3, ANXA6, MAP1B, TAGLN, ENPP2, FBLN5, PCOLCE, FN1, VIM, PPAP2B, SEM5A, VCAN, NID2, NID1, HAS2, PRRX1, TNFAIP6, CLDN4, FADS2, DDIT4, GNG11, PER1, ISCU, KPNA3, SLC44A1, FKBP5, NR2F1, MLPH, DOCK10, RGS4, IGFBP4; and
(ii) said second set of genes comprises or consists of:
S100A4, SDC2, CYP1B1, FERMT2, CYBRD1, SNAI2, CD44, BIN1, ENPP2, FBLN5, PCOLCE, FN1, VIM, PPAP2B, SEM5A, VCAN, CLDN4, FADS2, DDIT4, GNG11, PER1, ISCU, KPNA3, SLC44A1, FKBP5, IGFBP4; and
(iii) said third set of genes comprises or consists of:
SDC2, CYBRD1, SNAI2, PMP22, CTGF, IGFBP3, ANXA6, MAP1B, TAGLN, PPAP2B, HAS2, NRF2, MLPH, DOCK10, RGS4; and
(iv) said fourth set of genes comprises or consists of:
FERMT2, BIN1, PMP22, CHN1, CTGF, IGFBP3, NID2, CLDN4, CYBRD1, PRR16, ANXA6, PCOLCE, PPAP2B, HAS2, CDH11, MLPH, DOCK10, RGS4, PVRL3, NID2, SLC44A1, FKBP5; and
(v) said fifth set of genes comprises or consists of:
FERMT2, BIN1, PMP22, CHN1, CTGF, IGFBP3, NID2, CLDN4; and
(vi) said sixth set of genes comprises or consists of:
HAS2, BIN1, PCOLCE, FERMT2, CTGF, IGFBP3, NID2, SLC44A1, FKBP5, and MLPH; comprising a first agent capable of interacting with a gene expression product of a plurality of genes selected from a first and/or second and/or third and/or fourth and/or fifth and/or sixth set of genes; and wherein the method comprises assaying the level of gene expression product of the plurality of genes.

In one embodiment, the method comprises a second agent capable of interacting with a gene expression product from said first and/or second and/or third and/or fourth and/or fifth and/or sixth set of genes. In one embodiment, the method comprises a plurality of agents capable of interacting with a gene expression product from said first and/or second and/or third and/or fourth and/or fifth and/or sixth set of genes. In one embodiment, the method comprises a plurality of agents capable of interacting with a plurality of gene expression products from said first and/or second and/or third and/or fourth and/or fifth and/or sixth set of genes.

In one embodiment, said agent is a plurality of antibodies. In one embodiment, said agent is a plurality of oligonucleotides. In one embodiment, said gene expression product is a RNA product. In one embodiment, said gene expression product is a protein product. In one embodiment, the protein product is detected by an immunoassay, e.g., immunohistochemistry. In one embodiment, a value for the level of gene expression product for each gene isoform is assayed by detecting a RNA product. In one embodiment, the RNA product is detected by a hybridization based method. In one embodiment, the RNA product is detected by microarray. In one embodiment, said microarray is an exon microarray. In one embodiment, the RNA product is detected by a polymerase chain reaction based method. In one embodiment, the RNA product is detected by a sequencing based method. In one embodiment, the RNA product is detected by a quantitative RNA sequencing.

In one embodiment, the gene expression products are derived from a tumor sample, e.g., a preparation of a primary tumor, metastatic tumor, lymph node, circulating tumor cells, ascites, or pleural effusion, plasma, serum, circulating, and interstitial fluid levels.

In one embodiment, a value for the level of gene expression product for each gene is determined. In one embodiment, a value that is a function of the level of gene expression for each gene is determined. In one embodiment, the value is compared to a reference standard, e.g., the level of expression of a control gene in the tumor sample.

In one embodiment, the method further comprises the performance of an algorithm on a computer system to determine a value or values that is a function of a location of a gene expression product in the subject sample and/or a function of a level of a gene expression product of a gene in the subject sample. In one embodiment, the algorithm compares a ratio of the level of gene expression product of at least one of the genes selected from the group: HAS2, BIN1, PCOLCE, FERMT2, CTGF, IGFBP3, NID2, SLC44A1, FKBP5, and MLPH; to the level of gene expression product of at least one of the genes selected from the group: CDH1, and Cytokeratin.

In one embodiment, the method further comprises a plurality of agents capable of interacting with at least one gene expression product selected from the group: CTGF, IGFBP3, TNFAIP6, NID2, HAS2, CCL2, MLPH, NID1, IGFBP4, FBLN5, and PCOLCE. In one embodiment, the method further comprises a plurality of agents capable of interacting with a gene expression product of each gene from the set of genes consisting of: CTGF, IGFBP3, TNFAIP6, NID2, HAS2, CCL2, MLPH, NID1, IGFBP4, FBLN5, and PCOLCE.

Reaction Mixtures

In one aspect, reaction mixtures described herein include a reaction mixture comprising: a plurality of detection reagents; and a plurality of target nucleic acid molecules derived from a subject, wherein each of the plurality of detection reagents comprises a plurality probes to measure the level of gene expression product of a gene from a first and/or second and/or third and/or fourth and/or fifth and/or sixth and/or seventh and/or eighth and/or ninth and/or tenth and/or eleventh and/or twelfth and/or thirteenth and/or fourteenth set of genes, wherein:

(i) said first set of genes comprises or consists of:
KLF13, ANTXR2, IFNAR1, LIX1L, CHST11, AKAP2, PALM2-AKAPT, LOC80298, C7orf2, NUCKS, DKFZP566D1346, LOC388279, FLJ31795, 6orf107, FLJ12439, ENPP2, CITED2, ZEB1, NID2, SEMA5A, DAB2, KCNMA1, PTX3, PCDH9, CREB3L1, BGN, ROR1, DLC1, GNG11, CDH11, NR2F1, PRR16, MYL9, DOCK10, LRIG1, IER3, EML1, NEBL, RGL1, MLPH, DNAJB4, FBLN5, RGS4, HAS2, ITGBL1, IGFBP4, DPT, PCOLCE, GREM1, PPAP2B, CDH2, PMP22, LUM, CHN1, CYP1B1, MME, WNT5A, POSTN, MMP2, CTGF, FBLN1, IGFBP3, DCN, PRRX1, ANXA6, PVRL3, MAP1B, TNFAIP6, CYBRD1, FBN1, NID1, OLFML3, SNAI1, SNAI2, TAGLN, SYNC, TUBA1A, PPM1D, TWIST1, FN1, TGFBR3, SERPINF1, UGDH, SRGN, FAP, PTGER4, PRKCA, FSTL1, MMP1, NRP1, FILIP1L, SCCPDH, LTBP2, XYLT1, HS3ST2, SYT11, TSHZ1, THY1, Sep9, S100A4, TNS3, ENOX1, TGFB1I1, ZEB2, LMCD1, PDGFC, ECM1, TFPI, TBX3, DDR2, PTGER2, CDKN2C, VCAN, CD44, STARD13, SNED1, ZBTB38, SDC2, TPM1, COPZ2, STC1, CDH1, KRT5, KRT6B, CD24, EPCAM, ESRP1, CLDN3, CLDN4, ERBB3, SPOCK1, FERMT2, GLYR1, LTBP1, FADS2, KANK2, PTGFR, COL11A2, KLK3, EIF2C2, ZFP41, FAM49B, PSORS1C2, MRPL42, MRPL54, MRPL47, MRPS23, EIF3S9, ALG5, DNAJC19, TPRXL, NOTCH2, RBM15, ST3 GAL3, NFYA, PCNX, FBXO21, WWOX, CAMK2B, PNPLA2, CLIC5, UGCGL1, FBXL18, ADRBK1, SLC38A2, IL8RA, TAS2R14, CD300LB, GIPC3, RAD51L1, EPPK1, COL1A1, MMP9, SERPINE1, SPARC, TGFB1, TGFB3, TGFBI, TGFBR1, TGIF, TGIF2, THBS1, ANXA5, ACTG1, ARF3, ATP1B3, BAT3, CALD1, CENTD2, CLIC1, CTBS, DPYSL3, DVL3, EXT1, FGFR1, FTL, GNB2L1, GPRC5A, H2AFZ, HIF1A, IL13RA1, KDELR2, LARP1, LPIN2, MARS, MMP10, MMP14, MT2A, MYO10, NUP62, PFKFB3, PLOD2, PSMB7, PSMD8, RIN2, RYBP, SDF4, SETD5, SPP1, STAU1, TUBB3, UBE2S, XPNPEP1, CDKN1A, CHRD, H19, ID3, ID4, IGFBP7, LRP1, MSX1, NOTCH3, PROCR, GBX2, KI67, CCNB1, BUB1, KNTC2, USP22, HCFC1, RNF2, ANK3, FGFR2, CES1, COL1A2, COL3A1, COL5A2, COL6A1, ANKRD25, C10ORF56, C5ORF13, KRT81, NPAC, PLEKHC1, SEPT9, SYNC1, MBP, ABLIM1, ALDH1A3, ALOX15B, ANXA3, AP1M2, ARTN, CA2, CA9, CDH3, CDS1, COL17A1, CORO1A, CTSL2, CXADR, CYP27B1, DSC2, DSG3, DST, EPB41L4B, FGFBP1, FGFR3, FST, GJB3, GRHL2, HBEGF, HOOK1, IL18, IL1B, IRF6, ITGB4, JAG2, KLK10, KLK5, KLK7, KLK8, KRT15, KRT16, KRT17, LEPREL1, MYO5C, NDRG1, NMU, PI3, RAB25, RLN2, RNF128, S100A14, S100A7, S100A8, SERPINB1, SERPINB2, SLC2A9, SLPI, SNX10, SPINT2, SPRR1A, SPRR1B, ST14, SYK, TMEM40, TRIM29, TSPAN1, UCHL1, VSNL1, ZBED2, AQP3, CBARA1, CLCA2, CNTNAP2, CSTA, DSC3, ESRP2, FXYD3, GJB5, GPR87, IL1A, LAMA3, LAMB3, MAP7, MAPK13, MPZL2, NRG1, SCNN1A, SERPINB13, SLC7A5, SPINT1, ST6GALNAC2, TGFA, TP63, TPD52L1, VGLL1, SLC6A8, ABCA12, ANXA8, ANXA8L1, ARHGAP25, ARHGAP8, BDKRB2, BIK, C10ORF10, C10ORF116, C1ORF116, C20ORF19, C6ORF105, CELSR2, CKMT1A, CKMT1B, CLDN1, CST6, CYP4F11, E2F5, ELMO3, EPHA1, EVA1, EXPH5, F11R, FAT2, FLJ12684, FLJ20366, FZD3, GLS2, GNAL, IFI30, IGFBP2, IL1RN, IL4R, IRX4, JUP, KCNK1, KIAA0040, KIAA0888, KIAA1815, KRT14, KRT18, LAD1, LGALS7, LOC553158, LOC653562, LOC728113, LOC729884, LRRC1, LSR, MST1R, MYO1D, NAIP, OCLN, NEFM, NUP62CL, PERP, PLS1, POLR3G, PRKCH, PRRG4, PRSS8, PTPN3, RBM35A, RBM35B, RHBDF2, RHOD, RPS6KA1, RTEL1, SAA1, SAA1, SAA2, SH2D3A, SLC6A10P, SMPDL3B, SNCA, STAC, STAP2, TACSTD1, THBD, TMEM30B, TMPRSS11E, TMPRSS4, TNFRSF6B, TP73L, WWC1, XDH, ZNF165, AXL, GPR30, PAX2, PI3KA, CD133, ALDH1, BMI1, KRT19, MSI1, JAG1, p38, ESA1, FOXC2, FOXC1, TWIST2, GSC, TCF3, MLPH1, ARHGEF11a, DPF2, CASP8, BCL2, SCGN, SWAP70, KIAA0276, C10orf9, C10orf7, ALKBH, TOB2, XPR1, CD59, LRP2, PLP2, MAPK14, CXCL2, MMP7, MGP, MLF1, FLNB, SCNM1, HSPC163, C5orf18, MGC4399, CDW92, TMC4, ZDHHC2, TICAM2, KDELR3, GNPDA1, THEM2, DBR1, FLJ90709, FLJ10774, C16orf33, GAPD, LDHA, MR-1, LARS, GTPBP1, PRSS16, WFDC2, AIM1, DHRS6, DHRS4, GC15429, MGC45840, ECHDC2, GOLGIN-67, AFURS1, KIAA0436, CYP4V2, JTV1, ICMT, DNMT3A, HNMT, METTL7A, METTL2, VIL2, TPD52, ARPC5, NOL8, NSF, RAD23B, SRP54, HSPA2, PBP, THAP2, CIRBP, SNRPN, KIAA0052, DUSP10, SSR1, ERBB4, EMP1, CHPT1, LRPAP1, FLJ11752, CSTF1, KLHL20, DNAJC13, APLP2, ARGBP2, DNAJB1, NEBL, SH3BGRL, NUDT5, GABARAPL1, MAPT, DCBLD1, STK39, PAK2, CSNK2A1, PILRB, ERN1, SGKL, WEE1, MAST4, C11orf17, NUP37, CLTC, COPB2, SLC25A25, ECOP, PDE8A, STAM, TUBB, SNX6, RAB23, PLAA, STC2, LTF, ISGF3G, ATXN3, GTF3C3, GSK3B, KLF10, ELL2, ZBTB20, IRX3, ETS1, SERTAD1, MGC4251, MAFF, SFPQ, CITED4, CEBPD, EIF4E2, HS2ST1, AGPS, PGK1, ATIC, ETNK1, LG2, NCE2, MARCH8, CNOT4, RNF8, PSMA5, DPF2, AMMECR1, KIAA1287, LOC144233, LOC286505, PNAS-4, FLJ20530, HUMPD3, GC45564, CAP350, ETAA16, HAN11, DNAPTP6, C7orf25, FLJ37953, FLJ10587, C7orf36, ELP4, NDEL1, NPD014, KFZP564D172, FAM53C, IER5, LOC255783, KIAA0146, KIAA0792, LOC439994, LOC283481, CG018, LOC130576, NGFRAP1L1, KIAA1217, 4orf7, C21orf86, C9orf64, FLJ13456, FLJ12806, FLJ39370, GATS, CCDC92, FMNL2, ARID1B, ZFHX1B, SSBP2, ARID5B, LOC157381, KPNA3, ARHGAP24, CCND2, VIM, MYCBP2, GAS7, TRAM2, BASP1, FOXO1A, POLR2A, PER1, DDIT4, CD97, BIN1, SH2B3, DDB2, EMP3, NDST2, CHST2, NT5E, PDE4A, CPS1, PTGS1, GGCX, IRF5, ZBTB16, MAP4K4, CHST7, KLF12, NFRKB, PSD, FKSG49, NIFUN, FYN, ZMYM2, CACNA1G, SLC25A16, FLII, EIF1, SEPT6, NPAC, PHF15, NUP188, ABR, CNR1, LOC283824, FSTL4, DNM1, APOBEC3G, ATP2B1, SMPD1, SLC11A1, FXYD5, C14orf139, SH3BGRL3, LUZP1, ZNF335, SH3 KBP1, MST150, PRO1073, LOC388397, FKBP5, HIPK2, DTX1, ST3GAL2, ADAMTS7, TNRC6B, CYGB, SDHAL1, LOC572558, TRIO, FRAS1, KIAA1632, POLS, EBF, MAML2, PTPRA, PLEKHG2, DYM, SOX6, ARHGEF2, ZCCHC6, PPP3CA, FAM70B, TMED5, FLJ43663, HPS1, MEF2A, ST3GAL5, SMYD3, KLF7, LOC200230, RERE, QKI, BICD1, CTNNB1, POU2F2, EIF4ENIF1, BTG1, TCHP, FLJ90709, PCTK2, KIAA1600, B7-H4, PDE4DIP, KIAA0194, HOM-TES; and
(a) BGN, BIN1, CDH11, CDH2, CHN1, COL1A2, COL3A1, COL5A2, CREB3L1, CTGF, CYBRD1, DCN, DLC1, DNAJB4, DPT, ENPP2, FBLN1, FBLN5, FBN1, GREM1, HAS2, IGFBP3, IGFBP4, MLPH, MME, MMP2, MYL9, NEBL, NID1, NR2F1, OLFML3, PCOLCE, PMP22, POSTN, PPAP2B, PRR16, PRRX1, PTX3, PVRL3, RGL1, RGS4, ROR1, SEMA5A, TNFAIP6, TRAM2, WNT5A, ZEB1, ANXA6, CITED2, COL1A1, COL6A1, CYP1B1, DAB2, DOCK10, EML1, GNG11, IER3, ITGBL1, KCNMA1, LRIG1, LUM, MAP1B, NID2, PCDH9, PRKCA, PTGER4, PTGFR, SDC2, SERPINF1, SNED1, SYT11, TFPI, TGFBR3, VCAN, ANKRD25, C10ORF56, C5ORF13, CCDC92, CDKN2C, COPZ2, DDR2, ECM1, ENOX1, FADS2, FAP, FILIP1L, FSTL1, HS3ST2, KRT81, LMCD1, LTBP1, LTBP2, MMP1, NPAC, NRP1, PDGFC, PLEKHC1, PPM1D, PTGER2, SCCPDH, SEPT9, SPOCK1, SRGN, STARD13, STC1, SYNC1, TAGLN, TBX3, TGFB1I1, THY1, TNS3, TPM1, TSHZ1, TUBA1A, UGDH, VIM, XYLT1, ZBTB38, MBP, ABLIM1, ALDH1A3, ALOX15B, ANXA3, AP1M2, ARTN, CA2, CA9, CAMK2B, CCND2, CDH1, CDH3, CDS1, COL17A1, CORO1A, CTSL2, CXADR, CYP27B1, DSC2, DSG3, DST, EPB41L4B, FGFBP1, FGFR3, FST, GJB3, GRHL2, HBEGF, HOOK1, IL18, IL1B, IRF6, ITGB4, JAG2, KLK10, KLK5, KLK7, KLK8, KRT15, KRT16, KRT17, KRT5, KRT6B, LEPREL1, MYO5C, NDRG1, NMU, PI3, RAB25, RLN2, RNF128, S100A14, S100A7, S100A8, SERPINB1, SERPINB2, SLC2A9, SLPI, SNX10, SPINT2, SPRR1A, SPRR1B, ST14, SYK, TMEM40, TRIM29, TSPAN1, UCHL1, VSNL1, ZBED2, AQP3, CBARA1, CD24, CLCA2, CNTNAP2, CSTA, DSC3, EPCAM, ESRP2, FXYD3, GJB5, GPR87, IL1A, LAMA3, LAMB3, MAP7, MAPK13, MMP9, MPZL2, NRG1, SCNN1A, SERPINB13, SLC7A5, SPINT1, ST6GALNAC2, TGFA, TP63, TPD52L1, VGLL1, SLC6A8, ABCA12, ANK3, ANXA8, ANXA8L1, ARHGAP25, ARHGAP8, BDKRB2, BIK, C10ORF10, C10ORF116, C1ORF116, C20ORF19, C6ORF105, CELSR2, CKMT1A, CKMT1B, CLDN1, CST6, CYP4F11, E2F5, ELMO3, EPHA1, EVA1, EXPH5, F11R, FAT2, FGFR2, FLJ12684, FLJ20366, FZD3, GLS2, GNAL, IFI30, IGFBP2, IL1RN, IL4R, IRX4, JUP, KCNK1, KIAA0040, KIAA0888, KIAA1815, KRT14, KRT18, LAD1, LGALS7, LOC553158, LOC653562, LOC728113, LOC729884, LRRC1, LSR, MST1R, MYO1D, NAIP, OCLN, NEFM, NUP62CL, PERP, PLS1, POLR3G, PRKCH, PRRG4, PRSS8, PTPN3, RBM35A, RBM35B, RHBDF2, RHOD, RPS6KA1, RTEL1, SAA1, SAA1, SAA2, SH2D3A, SLC6A10P, SMPDL3B, SNCA, STAC, STAP2, TACSTD1, THBD, TMEM30B, TMPRSS11E, TMPRSS4, TNFRSF6B, TP73L, WWC1, XDH, ZNF165; and
(b) BGN, BIN1, CDH11, CDH2, CHN1, COL1A2, COL3A1, COL5A2, CREB3L1, CTGF, CYBRD1, DCN, DLC1, DNAJB4, DPT, ENPP2, FBLN1, FBLN5, FBN1, GREM1, HAS2, IGFBP3, IGFBP4, MLPH, MME, MMP2, MYL9, NEBL, NID1, NR2F1, OLFML3, PCOLCE, PMP22, POSTN, PPAP2B, PRR16, PRRX1, PTX3, PVRL3, RGL1, RGS4, ROR1, SEMA5A, TNFAIP6, TRAM2, WNT5A, ZEB1, ANXA6, CITED2, COL1A1, COL6A1, CYP1B1, DAB2, DOCK10, EML1, GNG11, IER3, ITGBL1, KCNMA1, LRIG1, LUM, MAP1B, NID2, PCDH9, PRKCA, PTGER4, PTGFR, SDC2, SERPINF1, SNED1, SYT11, TFPI, TGFBR3, VCAN, ANKRD25, C10ORF56, C5ORF13, CCDC92, CDKN2C, COPZ2, DDR2, ECM1, ENOX1, FADS2, FAP, FILIP1L, FSTL1, HS3ST2, KRT81, LMCD1, LTBP1, LTBP2, MMP1, NPAC, NRP1, PDGFC, PLEKHC1, PPM1D, PTGER2, SCCPDH, SEPT9, SPOCK1, SRGN, STARD13, STC1, SYNC1, TAGLN, TBX3, TGFB1I1, THY1, TNS3, TPM1, TSHZ1, TUBA1A, UGDH, VIM, XYLT1, ZBTB38; and
(c) ENPP2, CITED2, ZEB1, NID2, SEMA5A, DAB2, KCNMA1, PTX3, PCDH9, CREB3L1, BGN, ROR1, DLC1, GNG11, CDH11, NR2F1, NR2F2, PRR16, MYL9, DOCK10, LRIG1, IER3, EML1, NEBL, RGL1, MLPH, TRAM2, DNAJB4, FBLN5, RGS4, HAS2, ITGBL1, IGFBP4, BIN1, DPT, PCOLCE, GREM1, PPAP2B, CDH2, PMP22, LUM, CHN1, CYP1B1, MME, WNT5A, POSTN, MMP2, CTGF, FBLN1, IGFBP3, DCN, PRRX1, ANXA6, PVRL3, MAP1B, TNFAIP6, CYBRD1, FBN1, NID1, OLFML3, SNAI1, SNAI2, TAGLN, SYNC, TUBA1A, PPM1D, TWIST1, FN1, TGFBR3, SERPINF1, UGDH, SRGN, FAP, PTGER4, VIM, PRKCA, FSTL1, MMP1, NRP1, FILIP1L, SCCPDH, LTBP2, XYLT1, HS3ST2, SYT11, TSHZ1, THY1, SEPT9, S100A4, CCDC92, TNS3, ENOX1, TGFB1I1, ZEB2, LMCD1, PDGFC, ECM1, TFPI, TBX3, DDR2, PTGER2, CDKN2C, VCAN, CD44, STARD13, SNED1, ZBTB38, SDC2, TPM1, COPZ2, STC1, CDH1, KRT5, KRT6B, CD24, EPCAM, ESRP1, CLDN3, CLDN4, ERBB3, SPOCK1, FERMT2, GLYR1, LTBP1, FADS2, KANK2, PTGFR, ARTN, SLC7A5, EPB41L4B, KRT15, CDH3, TPD52L1, ANXA3, PI3, NDRG1, DSG3, S100A14, S100S7, KLK8, S100A8, PTX3, SPINT2, FXYD3, KRT16, RAB25, LGALS7, DSC3, COL5A2, ITGB4, TMEM40, SPRR1A, AP1M2, SERPINB13, TP73L; and
(d) CYBRD1, ARTN, CITED2, SLC7A5, CHN1, CTGF, CSTA, MYL9, EPB41L4B, KRT15, LRIG1, CDH3, TPD52L1, ANXA3, OLFML3, CORO1A, PI3, NDRG1, DSG3, PMP22, S100A14, S100S7, KLK8, S100A8, PTX3, SPINT2, IER3, FXYD3, KRT16, TGFBR3, KRT5, RAB25, SDC2, LUM, LGALS7, KRT6B, CD24, COL1A2, GNG11, DSC3, COL5A2, RGS4, ITGB4, TMEM40, SPRR1A, AP1M2, SERPINB13, SNED1, LEPREL1, TRAM2, TP73L, and PAP2B; and
(e) CYBRD1, ARTN, CITED2, SLC7A5, CHN1, CTGF, CSTA, MYL9, EPB41L4B, KRT15, LRIG1, CDH3, TPD52L1, ANXA3, OLFML3, CORO1A; and
(f) PI3, NDRG1, DSG3, PMP22, S100A14, S100S7, KLK8, S100A8, PTX3, SPINT2, IER3, FXYD3, KRT16, TGFBR3; and
(g) KRT5, RAB25, SDC2, LUM, LGALS7, KRT6B, CD24, COL1A2, GNG11, DSC3, COL5A2; and
(h) RGS4, ITGB4, TMEM40, SPRR1A, AP1M2, AERPINB13, SNED1, LEPREL1, TRAM2, TP73L, and PAB2B; and
(i) CYBRD1, ARTN, KRT15, ITGB4, RGS4, SPRR1B, SLC7A5, ANXA3, IL18, AP1M2, CHN1, OLFML3, ZBED2, LEPREL1, ABLIM1, CDH3, DNAJB4, TRAM2, UCHL1, CTGF; and
   (i) CYBRD1, ARTN, KRT15, ITGB4, RGS4, SPRR1B, SLC7A5, ANXA3, IL18, AP1M2; and
   (ii) CYBRD1, ARTN, KRT15, ITGB4, RGS4; and
(j) CYBRD1, RGS4, CDH3, CHN1, SLC7A5, PRRX1, CITED2, COL5A2, ARTN, ZBED2, SNED1, ITGB4, LUM, PVRL3, OLFML3, BIN1, CCND2, DAB2, ANXA3, IL18; and
   (i) CYBRD1, RGS4, CDH3, CHN1, SLC7A5, PRRX1, CITED2, COL5A2, ARTN, ZBED2; and
   (ii) CYBRD1, RGS4, CDH3, CHN1, SLC7A5; and
(k) CYBRD1, ARTN, RGS4, CITED2, SLC7A5, ITGB4, CHN1, PI3, TMEM40, NDRG1, SPRR1A, PCDH9, CTGF, MAP1B, DSG3, AP1M2, FBLN5, SERPINB13, PMP22, CSTA; and
   (i) CYBRD1, ARTN, RGS4, CITED2, SLC7A5, ITGB4, CHN1, PI3, TMEM40, NDRG1; and
   (ii) CYBRD1, ARTN, RGS4, CITED2, SLC7A5; and

(ii) said second set of genes comprises or consists of:

AXL, NOTCH3, GPR30, PROCR, PAX2, PI3KA, CD44, CD133, ALDH1, BMI1, KRT19, NRP1, MSI1, MTOR, JAG1, p38, ESA1, ZEB1, TWIST1, FOXC2, CDH2, ZEB2, FOXC1, SNAI1, SNAI2, TWIST2, GSC, TCF3, CD44, VIM, FN1, MLPH1, ARHGEF11; and

(iii) said third set of genes comprises or consists of:

At least one gene from said first set of genes and at least one of:

AXL, NOTCH3, GPR30, PROCR, PAX2, PI3KA, CD44, CD133, ALDH1, BMI1, KRT19, NRP1, MSI1, MTOR, JAG1, p38, ESA1, ZEB1, TWIST1, FOXC2, CDH2, ZEB2, FOXC1, SNAI1, SNAI2, TWIST2, GSC, TCF3, CD44, VIM, FN1, MLPH1, ARHGEF11; and

(iv) said fourth set of genes comprises or consists of:

At least two genes from said first set of genes and at least one of: AXL, NOTCH3, GPR30, PROCR, PAX2, PI3KA, CD44, CD133, ALDH1, BMI1, KRT19, NRP1, MSI1, MTOR, JAG1, p38, ESA1, ZEB1, TWIST1, FOXC2, CDH2, ZEB2, FOXC1, SNAI1, SNAI2, TWIST2, GSC, TCF3, CD44, VIM, FN1, MLPH1, and ARHGEF11; and

(v) said fifth set of genes comprises or consists of:

beta catenin, FZD1, FZD2, FZD3, FZD4, FZD5, FZD6, FZD7, FZD8, FZD9, FZD10, LRP5, LRP6, WNT1, WNT2, WNT2B, WNT3, WNT3A, WNT4 WNT5A, WNT5B, WNT6, WNT7A, WNT7B, WNT8A, WNT8B, WNT9A, WNT9B, WNT10A, WNT10B, WNT11, WNT16, SRFP1, SRFP2, FRZB, SFRP4, SFRP5, DKK1, DKK2, DKK3, DKK4, WIF1, AKT2, PI3KA, Disheveled, Smoothened, Axin1, Axin2; and

(vi) said sixth set of genes comprises or consists of:

At least one gene from said first set of genes and at least one of:

beta catenin, FZD1, FZD2, FZD3, FZD4, FZD5, FZD6, FZD7, FZD8, FZD9, FZD10, LRP5, LRP6, WNT1, WNT2, WNT2B, WNT3, WNT3A, WNT4 WNT5A, WNT5B, WNT6, WNT7A, WNT7B, WNT8A, WNT8B, WNT9A, WNT9B, WNT10A, WNT10B, WNT11, WNT16, SRFP1, SRFP2, FRZB, SFRP4, SFRP5, DKK1, DKK2, DKK3, DKK4, WIF1, AKT2, PI3KA, Disheveled, Smoothened, Axin1, Axin2; and

(vii) said seventh set of genes comprises or consists of:

TGFbeta1, TGFbeta2, TGFbeta3, ALK1, ALK2, ALK3, ALK4, ALK5, ALK6, ALK7, TGFbetaR-II, BMPR-II, ACTR-IIA, ACTR-IIB, AMHR-II, SMAD2, SMAD3; and

(viii) said eighth set of genes comprises or consists of:

At least one gene from said first set of genes and at least one of:

TGFbeta1, TGFbeta2, TGFbeta3, ALK1, ALK2, ALK3, ALK4, ALK5, ALK6, ALK7, TGFbetaR-II, BMPR-II, ACTR-IIA, ACTR-IIB, AMHR-II, SMAD2, SMAD3; and

(ix) said ninth set of genes comprises or consists of:

S100A4, SDC2, CYP1B1, FERMT2, CYBRD1, SNAI2, CD44, BIN1, PMP22, CTGF, IGFBP3, ANXA6, MAP1B, TAGLN, ENPP2, FBLN5, PCOLCE, FN1, VIM, PPAP2B, SEM5A, VCAN, NID2, NID1, HAS2, PRRX1, TNFAIP6, CLDN4, FADS2, DDIT4, GNG11, PER1, ISCU, KPNA3, SLC44A1, FKBP5, NR2F1, MLPH, DOCK10, RGS4, IGFBP4; and

(x) said tenth set of genes comprises or consists of:

S100A4, SDC2, CYP1B1, FERMT2, CYBRD1, SNAI2, CD44, BIN1, ENPP2, FBLN5, PCOLCE, FN1, VIM, PPAP2B, SEM5A, VCAN, CLDN4, FADS2, DDIT4, GNG11, PER1, ISCU, KPNA3, SLC44A1, FKBP5, IGFBP4; and

(xi) said eleventh set of genes comprises or consists of:

SDC2, CYBRD1, SNAI2, PMP22, CTGF, IGFBP3, ANXA6, MAP1B, TAGLN, PPAP2B, HAS2, NRF2, MLPH, DOCK10, RGS4; and

(xii) said twelfth set of genes comprises or consists of:

FERMT2, BIN1, PMP22, CHN1, CTGF, IGFBP3, NID2, CLDN4, CYBRD1, PRR16, ANXA6, PCOLCE, PPAP2B, HAS2, CDH11, MLPH, DOCK10, RGS4, PVRL3, NID2, SLC44A1, FKBP5; and

(xiii) said thirteenth set of genes comprises or consists of:

FERMT2, BIN1, PMP22, CHN1, CTGF, IGFBP3, NID2, CLDN4; and

(xiv) said fourteenth set of genes comprises or consists of:

HAS2, BIN1, PCOLCE, FERMT2, CTGF, IGFBP3, NID2, SLC44A1, FKBP5, and MLPH.

In one embodiment, each probe comprises a DNA, RNA or mixed DNA/RNA molecule, which is complementary to a nucleic acid sequence on each of the plurality of target nucleic acid molecules, wherein each target nucleic acid molecule is derived from a gene in said first and/or second and/or third and/or fourth and/or fifth and/or sixth and/or seventh and/or eighth and/or ninth and/or tenth and/or eleventh and/or twelfth and/or thirteenth and/or fourteenth set of genes. In one embodiment, each of the plurality of detection reagents comprises a probe to measure the expression at least 1, 2, 3, 4, 5, 6, 7, 8, 9, 10, 11, 12, 13, 14, 15, 16, 17, 18, 19, or 20 genes in said first and/or second and/or third and/or fourth and/or fifth and/or sixth and/or seventh and/or eighth and/or ninth and/or tenth and/or eleventh and/or twelfth and/or thirteenth and/or fourteenth set of genes. In one embodiment, each of the plurality of detection reagents comprises a probe to measure the expression 1, 2, 3, 4, 5, 6, 7, 8, 9, 10, 11, 12, 13, 14, 15, 16, 17, 18, 19, or genes in said first and/or second and/or third and/or fourth and/or fifth and/or sixth and/or seventh and/or eighth and/or ninth and/or tenth and/or eleventh and/or twelfth and/or thirteenth and/or fourteenth set of genes. In one embodiment, each of the plurality of detection reagents comprises a probe to measure the expression of no more than 1, 2, 3, 4, 5, 6, 7, 8, 9, 10, 11, 12, 13, 14, 15, 16, 17, 18, 19, or 20 genes in said first and/or second and/or third and/or fourth and/or fifth and/or sixth and/or seventh and/or eighth and/or ninth and/or tenth and/or eleventh and/or twelfth and/or thirteenth and/or fourteenth set of genes. In one embodiment, each of the plurality of detection reagents comprises a probe to measure the expression of only genes in said first and/or second and/or third and/or fourth and/or fifth and/or sixth and/or seventh and/or eighth and/or ninth and/or tenth and/or eleventh and/or twelfth and/or thirteenth and/or fourteenth set of genes.

In an embodiment, the probe is a nucleic acid molecule. In one embodiment, the plurality of target nucleic acid molecules is derived from a subject with cancer. Also described herein are kits comprising detection reagents described herein.

In one aspect, reaction mixtures described herein include a reaction mixture comprising:

a plurality of detection reagents, e.g., a plurality of substrates, e.g., a plurality of antibodies; and a plurality of target proteins derived from a cancer, wherein each of the plurality of target proteins is encoded by a gene in said first and/or second and/or third and/or fourth and/or fifth and/or sixth and/or seventh and/or eighth and/or ninth and/or tenth and/or eleventh and/or twelfth and/or thirteenth and/or fourteenth set of genes, and wherein each of the plurality of detection reagents is a probe specific for one of the plurality of target proteins, e.g., binds to the target protein.

In one embodiment, each of the plurality of detection reagents comprises a probe to measure the expression at least 1, 2, 3, 4, 5, 6, 7, 8, 9, 10, 11, 12, 13, 14, 15, 16, 17, 18, 19, or 20 genes in said first and/or second and/or third and/or fourth and/or fifth and/or sixth and/or seventh and/or eighth and/or ninth and/or tenth and/or eleventh and/or twelfth and/or thirteenth and/or fourteenth set of genes. In one embodiment, each of the plurality of detection reagents comprises a probe to measure the expression 1, 2, 3, 4, 5, 6, 7, 8, 9, 10, 11, 12, 13, 14, 15, 16, 17, 18, 19, or 20 genes in said first and/or second and/or third and/or fourth and/or fifth and/or sixth and/or seventh and/or eighth and/or ninth and/or tenth and/or eleventh and/or twelfth and/or thirteenth and/or fourteenth set of genes. In one embodiment, each of the plurality of detection reagents comprises a probe to measure the expression of no more than 1, 2, 3, 4, 5, 6, 7, 8, 9, 10, 11, 12, 13, 14, 15, 16, 17, 18, 19, or 20 genes in said first and/or second and/or third and/or fourth and/or fifth and/or sixth and/or seventh and/or eighth and/or ninth and/or tenth and/or eleventh and/or twelfth and/or thirteenth and/or fourteenth set of genes. In one embodiment, each of the plurality of detection reagents comprises a probe to measure the expression of only genes in said first and/or second and/or third and/or fourth and/or fifth and/or sixth and/or seventh and/or eighth and/or ninth and/or tenth and/or eleventh and/or twelfth and/or thirteenth and/or fourteenth set of genes.

In one embodiment, the plurality of target proteins is derived from a patient with a cancer. Also described herein are kits comprising detection reagents described herein.

Also described herein are methods of making a reaction mixture.

In one aspect, described herein are methods of making a reaction mixture comprising:

combining a plurality of detection reagents, with a plurality of target nucleic acid molecules derived from a patient with an ovarian cancer, wherein each target nucleic acid molecule is derived from a plurality of genes in Table 1, and wherein each of the plurality of detection reagents comprises a probe to measure the expression of a gene in said first and/or second and/or third and/or fourth and/or fifth and/or sixth and/or seventh and/or eighth and/or ninth and/or tenth and/or eleventh and/or twelfth and/or thirteenth and/or fourteenth set of genes.

In one aspect, described herein are methods of making a reaction mixture comprising:

combining a plurality of detection reagents, e.g., a plurality of substrates, e.g., a plurality of antibodies; and a plurality of target proteins derived from an ovarian cancer, wherein each of the plurality of target proteins is encoded by a gene in said first and/or second and/or third and/or fourth and/or fifth and/or sixth and/or seventh and/or eighth and/or ninth and/or tenth and/or eleventh and/or twelfth and/or thirteenth and/or fourteenth set of genes, and wherein each of the plurality of detection reagents is a probe specific for one of the plurality of target proteins, e.g., binds to the target protein.

In one aspect, reaction mixtures described herein include a reaction mixture comprising: a plurality of detection reagents; and a plurality of target nucleic acid molecules derived from a subject, wherein each of the plurality of detection reagents comprises a plurality probes to measure the level of gene expression product of a gene from a first and/or second and/or third and/or fourth and/or fifth and/or sixth set of genes, wherein:
(i) said first set of genes comprises or consists of:
   S100A4, SDC2, CYP1B1, FERMT2, CYBRD1, SNAI2, CD44, BIN1, PMP22, CTGF, IGFBP3, ANXA6, MAP1B, TAGLN, ENPP2, FBLN5, PCOLCE, FN1, VIM, PPAP2B, SEM5A, VCAN, NID2, NID1, HAS2, PRRX1, TNFAIP6, CLDN4, FADS2, DDIT4, GNG11, PER1, ISCU, KPNA3, SLC44A1, FKBP5, NR2F1, MLPH, DOCK10, RGS4, IGFBP4; and
(ii) said second set of genes comprises or consists of:
   S100A4, SDC2, CYP1B1, FERMT2, CYBRD1, SNAI2, CD44, BIN1, ENPP2, FBLN5, PCOLCE, FN1, VIM, PPAP2B, SEM5A, VCAN, CLDN4, FADS2, DDIT4, GNG11, PER1, ISCU, KPNA3, SLC44A1, FKBP5, IGFBP4; and
(iii) said third set of genes comprises or consists of:

### SDC2, CYBRD1, SNAI2, PMP22, CTGF, IGFBP3, ANXA6, MAP1B, TAGLN, PPAP2B, HAS2, NRF2, MLPH, DOCK10, RGS4; and

(iv) said fourth set of genes comprises or consists of:

### FERMT2, BIN1, PMP22, CHN1, CTGF, IGFBP3, NID2, CLDN4, CYBRD1, PRR16, ANXA6, PCOLCE, PPAP2B, HAS2, CDH11, MLPH, DOCK10, RGS4, PVRL3, NID2, SLC44A1, FKBP5; and

(v) said fifth set of genes comprises or consists of:

### FERMT2, BIN1, PMP22, CHN1, CTGF, IGFBP3, NID2, CLDN4; and

(vi) said sixth set of genes comprises or consists of:
HAS2, BIN1, PCOLCE, FERMT2, CTGF, IGFBP3, NID2, SLC44A1, FKBP5, and MLPH.

In one embodiment, each probe comprises a DNA, RNA or mixed DNA/RNA molecule, which is complementary to a nucleic acid sequence on each of the plurality of target nucleic acid molecules, wherein each target nucleic acid molecule is derived from a gene in said first and/or second and/or third and/or fourth and/or fifth and/or sixth set of genes. In one embodiment, each of the plurality of detection reagents comprises a probe to measure the expression at least 1, 2, 3, 4, 5, 6, 7, 8, 9, 10, 11, 12, 13, 14, 15, 16, 17, 18, 19, or 20 genes in said first and/or second and/or third and/or fourth and/or fifth and/or sixth set of genes. In one embodiment, each of the plurality of detection reagents comprises a probe to measure the expression 1, 2, 3, 4, 5, 6, 7, 8, 9, 10, 11, 12, 13, 14, 15, 16, 17, 18, 19, or 20 genes in said first and/or second and/or third and/or fourth and/or fifth and/or sixth set of genes. In one embodiment, each of the plurality of detection reagents comprises a probe to measure the expression of no more than 1, 2, 3, 4, 5, 6, 7, 8, 9, 10, 11, 12, 13, 14, 15, 16, 17, 18, 19, or 20 genes in said first and/or second and/or third and/or fourth and/or fifth and/or sixth set of genes. In one embodiment, each of the plurality of detection reagents comprises a probe to measure the expression of only genes in said first and/or second and/or third and/or fourth and/or fifth and/or sixth set of genes.

In an embodiment, the probe is a nucleic acid molecule. In one embodiment, the plurality of target nucleic acid molecules is derived from a subject with cancer. Also described herein are kits comprising detection reagents described herein.

In one aspect, reaction mixtures described herein include a reaction mixture comprising:

a plurality of detection reagents, e.g., a plurality of substrates, e.g., a plurality of antibodies; and a plurality of target proteins derived from a cancer, wherein each of the plurality of target proteins is encoded by a gene in a first and/or second and/or third and/or fourth and/or fifth and/or sixth set of genes, wherein:
(i) said first set of genes comprises or consists of:
S100A4, SDC2, CYP1B1, FERMT2, CYBRD1, SNAI2, CD44, BIN1, PMP22, CTGF, IGFBP3, ANXA6, MAP1B, TAGLN, ENPP2, FBLN5, PCOLCE, FN1, VIM, PPAP2B, SEM5A, VCAN, NID2, NID1, HAS2, PRRX1, TNFAIP6, CLDN4, FADS2, DDIT4, GNG11, PER1, ISCU, KPNA3, SLC44A1, FKBP5, NR2F1, MLPH, DOCK10, RGS4, IGFBP4; and
(ii) said second set of genes comprises or consists of:
S100A4, SDC2, CYP1B1, FERMT2, CYBRD1, SNAI2, CD44, BIN1, ENPP2, FBLN5, PCOLCE, FN1, VIM, PPAP2B, SEM5A, VCAN, CLDN4, FADS2, DDIT4, GNG11, PER1, ISCU, KPNA3, SLC44A1, FKBP5, IGFBP4; and
(iii) said third set of genes comprises or consists of:
SDC2, CYBRD1, SNAI2, PMP22, CTGF, IGFBP3, ANXA6, MAP1B, TAGLN, PPAP2B, HAS2, NRF2, MLPH, DOCK10, RGS4; and
(iv) said fourth set of genes comprises or consists of:
FERMT2, BIN1, PMP22, CHN1, CTGF, IGFBP3, NID2, CLDN4, CYBRD1, PRR16, ANXA6, PCOLCE, PPAP2B, HAS2, CDH11, MLPH, DOCK10, RGS4, PVRL3, NID2, SLC44A1, FKBP5; and
(v) said fifth set of genes comprises or consists of:
FERMT2, BIN1, PMP22, CHN1, CTGF, IGFBP3, NID2, CLDN4; and
(vi) said sixth set of genes comprises or consists of:

HAS2, BIN1, PCOLCE, FERMT2, CTGF, IGFBP3, NID2, SLC44A1, FKBP5, and MLPH;

and wherein each of the plurality of detection reagents is a probe specific for one of the plurality of target proteins, e.g., binds to the target protein.

In one embodiment, each of the plurality of detection reagents comprises a probe to measure the expression at least 1, 2, 3, 4, 5, 6, 7, 8, 9, 10, 11, 12, 13, 14, 15, 16, 17, 18, 19, or 20 genes in said first and/or second and/or third and/or fourth and/or fifth and/or sixth set of genes. In one embodiment, each of the plurality of detection reagents comprises a probe to measure the expression 1, 2, 3, 4, 5, 6, 7, 8, 9, 10, 11, 12, 13, 14, 15, 16, 17, 18, 19, or 20 genes in said first and/or second and/or third and/or fourth and/or fifth and/or sixth set of genes. In one embodiment, each of the plurality of detection reagents comprises a probe to measure the expression of no more than 1, 2, 3, 4, 5, 6, 7, 8, 9, 10, 11, 12, 13, 14, 15, 16, 17, 18, 19, or 20 genes in said first and/or second and/or third and/or fourth and/or fifth and/or sixth set of genes. In one embodiment, each of the plurality of detection reagents comprises a probe to measure the expression of only genes in said first and/or second and/or third and/or fourth and/or fifth and/or sixth set of genes.

In one embodiment, the plurality of target proteins is derived from a patient with a cancer. Also described herein are kits comprising detection reagents described herein.

Also described herein are methods of making a reaction mixture.

In one aspect, described herein are methods of making a reaction mixture comprising:

combining a plurality of detection reagents, with a plurality of target nucleic acid molecules derived from a patient with an ovarian cancer, wherein each target nucleic acid molecule is derived from a plurality of genes in a first and/or second and/or third and/or fourth and/or fifth and/or sixth set of genes, wherein:
(i) said first set of genes comprises or consists of:
S100A4, SDC2, CYP1B1, FERMT2, CYBRD1, SNAI2, CD44, BIN1, PMP22, CTGF, IGFBP3, ANXA6, MAP1B, TAGLN, ENPP2, FBLN5, PCOLCE, FN1, VIM, PPAP2B, SEM5A, VCAN, NID2, NID1, HAS2, PRRX1, TNFAIP6, CLDN4, FADS2, DDIT4, GNG11, PER1, ISCU, KPNA3, SLC44A1, FKBP5, NR2F1, MLPH, DOCK10, RGS4, IGFBP4; and
(ii) said second set of genes comprises or consists of:
S100A4, SDC2, CYP1B1, FERMT2, CYBRD1, SNAI2, CD44, BIN1, ENPP2, FBLN5, PCOLCE, FN1, VIM, PPAP2B, SEM5A, VCAN, CLDN4, FADS2, DDIT4, GNG11, PER1, ISCU, KPNA3, SLC44A1, FKBP5, IGFBP4; and
(iii) said third set of genes comprises or consists of:
SDC2, CYBRD1, SNAI2, PMP22, CTGF, IGFBP3, ANXA6, MAP1B, TAGLN, PPAP2B, HAS2, NRF2, MLPH, DOCK10, RGS4; and
(iv) said fourth set of genes comprises or consists of:
FERMT2, BIN1, PMP22, CHN1, CTGF, IGFBP3, NID2, CLDN4, CYBRD1, PRR16, ANXA6, PCOLCE, PPAP2B, HAS2, CDH11, MLPH, DOCK10, RGS4, PVRL3, NID2, SLC44A1, FKBP5; and
(v) said fifth set of genes comprises or consists of:
FERMT2, BIN1, PMP22, CHN1, CTGF, IGFBP3, NID2, CLDN4; and
(vi) said sixth set of genes comprises or consists of:
HAS2, BIN1, PCOLCE, FERMT2, CTGF, IGFBP3, NID2, SLC44A1, FKBP5, and MLPH;

and wherein each of the plurality of detection reagents comprises a probe to measure the expression of a gene in said first and/or second and/or third and/or fourth and/or fifth and/or sixth set of genes.

In one aspect, described herein are methods of making a reaction mixture comprising:

combining a plurality of detection reagents, e.g., a plurality of substrates, e.g., a plurality of antibodies; and a plurality of target proteins derived from an ovarian cancer, wherein each of the plurality of target proteins is encoded by a gene in a first and/or second and/or third and/or fourth and/or fifth and/or sixth set of genes, wherein:
(i) said first set of genes comprises or consists of:
S100A4, SDC2, CYP1B1, FERMT2, CYBRD1, SNAI2, CD44, BIN1, PMP22, CTGF, IGFBP3, ANXA6, MAP1B, TAGLN, ENPP2, FBLN5, PCOLCE, FN1, VIM, PPAP2B, SEM5A, VCAN, NID2, NID1, HAS2, PRRX1, TNFAIP6, CLDN4, FADS2, DDIT4, GNG11, PER1, ISCU, KPNA3, SLC44A1, FKBP5, NR2F1, MLPH, DOCK10, RGS4, IGFBP4; and
(ii) said second set of genes comprises or consists of:
S100A4, SDC2, CYP1B1, FERMT2, CYBRD1, SNAI2, CD44, BIN1, ENPP2, FBLN5, PCOLCE, FN1, VIM, PPAP2B, SEM5A, VCAN, CLDN4, FADS2, DDIT4, GNG11, PER1, ISCU, KPNA3, SLC44A1, FKBP5, IGFBP4; and
(iii) said third set of genes comprises or consists of:
SDC2, CYBRD1, SNAI2, PMP22, CTGF, IGFBP3, ANXA6, MAP1B, TAGLN, PPAP2B, HAS2, NRF2, MLPH, DOCK10, RGS4; and
(iv) said fourth set of genes comprises or consists of:
FERMT2, BIN1, PMP22, CHN1, CTGF, IGFBP3, NID2, CLDN4, CYBRD1, PRR16, ANXA6, PCOLCE, PPAP2B, HAS2, CDH11, MLPH, DOCK10, RGS4, PVRL3, NID2, SLC44A1, FKBP5; and
(v) said fifth set of genes comprises or consists of:
FERMT2, BIN1, PMP22, CHN1, CTGF, IGFBP3, NID2, CLDN4; and
(vi) said sixth set of genes comprises or consists of:
HAS2, BIN1, PCOLCE, FERMT2, CTGF, IGFBP3, NID2, SLC44A1, FKBP5, and MLPH;

and wherein each of the plurality of detection reagents is a probe specific for one of the plurality of target proteins, e.g., binds to the target protein.

In Vitro Assays

Also described herein are in vitro methods and assays. In one aspect described herein are in vitro methods and assays of determining if a subject is a potential candidate for treatment with an agent that inhibits or kills cancer associated mesenchymal cells, tumor initiating cancer cells, or cancer stem cells, the method comprising determining the level of gene expression product of a plurality of genes selected from a first and/or second and/or third and/or fourth and/or fifth and/or sixth and/or seventh and/or eighth and/or ninth and/or tenth and/or eleventh and/or twelfth and/or thirteenth and/or fourteenth set of genes, wherein:

(i) said first set of genes comprises or consists of:
KLF13, ANTXR2, IFNAR1, LIX1L, CHST11, AKAP2, PALM2-AKAPT, LOC80298, C7 orf2, NUCKS, DKFZP566D1346, LOC388279, FLJ31795, 6orf107, FLJ12439, ENPP2, CITED2, ZEB1, NID2, SEMA5A, DAB2, KCNMA1, PTX3, PCDH9, CREB3L1, BGN, ROR1, DLC1, GNG11, CDH11, NR2F1, PRR16, MYL9, DOCK10, LRIG1, IER3, EML1, NEBL, RGL1, MLPH, DNAJB4, FBLN5, RGS4, HAS2, ITGBL1, IGFBP4, DPT, PCOLCE, GREM1, PPAP2B, CDH2, PMP22, LUM, CHN1, CYP1B1, MME, WNT5A, POSTN, MMP2, CTGF, FBLN1, IGFBP3, DCN, PRRX1, ANXA6, PVRL3, MAP1B, TNFAIP6, CYBRD1, FBN1, NID1, OLFML3, SNAI1, SNAI2, TAGLN, SYNC, TUBA1A, PPM1D, TWIST1, FN1, TGFBR3, SERPINF1, UGDH, SRGN, FAP, PTGER4, PRKCA, FSTL1, MMP1, NRP1, FILIP1L, SCCPDH, LTBP2, XYLT1, HS3ST2, SYT11, TSHZ1, THY1, Sep9, S100A4, TNS3, ENOX1, TGFB1I1, ZEB2, LMCD1, PDGFC, ECM1, TFPI, TBX3, DDR2, PTGER2, CDKN2C, VCAN, CD44, STARD13, SNED1, ZBTB38, SDC2, TPM1, COPZ2, STC1, CDH1, KRT5, KRT6B, CD24, EPCAM, ESRP1, CLDN3, CLDN4, ERBB3, SPOCK1, FERMT2, GLYR1, LTBP1, FADS2, KANK2, PTGFR, COL11A2, KLK3, EIF2C2, ZFP41, FAM49B, PSORS1C2, MRPL42, MRPL54, MRPL47, MRPS23, EIF3S9, ALG5, DNAJC19, TPRXL, NOTCH2, RBM15, ST3 GAL3, NFYA, PCNX, FBXO21, WWOX, CAMK2B, PNPLA2, CLIC5, UGCGL1, FBXL18, ADRBK1, SLC38A2, IL8RA, TAS2R14, CD300LB, GIPC3, RAD51L1, EPPK1, COL1A1, MMP9, SERPINE1, SPARC, TGFB1, TGFB3, TGFBI, TGFBR1, TGIF, TGIF2, THBS1, ANXA5, ACTG1, ARF3, ATP1B3, BAT3, CALD1, CENTD2, CLIC1, CTBS, DPYSL3, DVL3, EXT1, FGFR1, FTL, GNB2L1, GPRC5A, H2AFZ, HIF1A, IL13RA1, KDELR2, LARP1, LPIN2, MARS, MMP10, MMP14, MT2A, MYO10, NUP62, PFKFB3, PLOD2, PSMB7, PSMD8, RIN2, RYBP, SDF4, SETD5, SPP1, STAU1, TUBB3, UBE2S, XPNPEP1, CDKN1A, CHRD, H19, ID3, ID4, IGFBP7, LRP1, MSX1, NOTCH3, PROCR, GBX2, KI67, CCNB1, BUB1, KNTC2, USP22, HCFC1, RNF2, ANK3, FGFR2, CES1, COL1A2, COL3A1, COL5A2, COL6A1, ANKRD25, C10ORF56, C5ORF13, KRT81, NPAC, PLEKHC1, SEPT9, SYNC1, MBP, ABLIM1, ALDH1A3, ALOX15B, ANXA3, AP1M2, ARTN, CA2, CA9, CDH3, CDS1, COL17A1, CORO1A, CTSL2, CXADR, CYP27B1, DSC2, DSG3, DST, EPB41L4B, FGFBP1, FGFR3, FST, GJB3, GRHL2, HBEGF, HOOK1, IL18, IL1B, IRF6, ITGB4, JAG2, KLK10, KLK5, KLK7, KLK8, KRT15, KRT16, KRT17, LEPREL1, MYO5C, NDRG1, NMU, PI3, RAB25, RLN2, RNF128, S100A14, S100A7, S100A8, SERPINB1, SERPINB2, SLC2A9, SLPI, SNX10, SPINT2, SPRR1A, SPRR1B, ST14, SYK, TMEM40, TRIM29, TSPAN1, UCHL1, VSNL1, ZBED2, AQP3, CBARA1, CLCA2, CNTNAP2, CSTA, DSC3, ESRP2, FXYD3, GJB5, GPR87, IL1A, LAMA3, LAMB3, MAP7, MAPK13, MPZL2, NRG1, SCNN1A, SERPINB13, SLC7A5, SPINT1, ST6GALNAC2, TGFA, TP63, TPD52L1, VGLL1, SLC6A8, ABCA12, ANXA8, ANXA8L1, ARHGAP25, ARHGAP8, BDKRB2, BIK, C10ORF10, C10ORF116, C1ORF116, C20ORF19, C6ORF105, CELSR2, CKMT1A, CKMT1B, CLDN1, CST6, CYP4F11, E2F5, ELMO3, EPHA1, EVA1, EXPH5, F11R, FAT2, FLJ12684, FLJ20366, FZD3, GLS2, GNAL, IFI30, IGFBP2, IL1RN, IL4R, IRX4, JUP, KCNK1, KIAA0040, KIAA0888, KIAA1815, KRT14, KRT18, LAD1, LGALS7, LOC553158, LOC653562, LOC728113, LOC729884, LRRC1, LSR, MST1R, MYO1D, NAIP, OCLN, NEFM, NUP62CL, PERP, PLS1, POLR3G, PRKCH, PRRG4, PRSS8, PTPN3, RBM35A, RBM35B, RHBDF2, RHOD, RPS6KA1, RTEL1, SAA1, SAA1, SAA2, SH2D3A, SLC6A10P, SMPDL3B, SNCA, STAC, STAP2, TACSTD1, THBD, TMEM30B, TMPRSS11E, TMPRSS4, TNFRSF6B, TP73L, WWC1, XDH, ZNF165, AXL, GPR30, PAX2, PI3KA, CD133, ALDH1, BMI1, KRT19, MSI1, JAG1, p38, ESA1, FOXC2, FOXC1, TWIST2, GSC, TCF3, MLPH1, ARHGEF11a, DPF2, CASP8, BCL2, SCGN, SWAP70, KIAA0276, C10orf9, C10orf7, ALKBH, TOB2, XPR1, CD59, LRP2, PLP2, MAPK14, CXCL2, MMP7, MGP, MLF1, FLNB, SCNM1, HSPC163, C5orf18, MGC4399, CDW92, TMC4, ZDHHC2, TICAM2, KDELR3, GNPDA1, THEM2, DBR1, FLJ90709, FLJ10774, C16orf33, GAPD, LDHA, MR-1, LARS, GTPBP1, PRSS16, WFDC2, AIM1, DHRS6, DHRS4, GC15429, MGC45840, ECHDC2, GOLGIN-67, AFURS1, KIAA0436, CYP4V2, JTV1, ICMT, DNMT3A, HNMT, METTL7A, METTL2, VIL2, TPD52, ARPC5, NOL8, NSF, RAD23B, SRP54, HSPA2, PBP, THAP2, CIRBP, SNRPN, KIAA0052, DUSP10, SSR1, ERBB4, EMP1, CHPT1, LRPAP1, FLJ11752, CSTF1, KLHL20, DNAJC13, APLP2, ARGBP2, DNAJB1, NEBL, SH3BGRL, NUDT5, GABARAPL1, MAPT, DCBLD1, STK39, PAK2, CSNK2A1, PILRB, ERN1, SGKL, WEE1, MAST4, C11orf17, NUP37, CLTC, COPB2, SLC25A25, ECOP, PDE8A, STAM, TUBB, SNX6, RAB23, PLAA, STC2, LTF, ISGF3G, ATXN3, GTF3C3, GSK3B, KLF10, ELL2, ZBTB20, IRX3, ETS1, SERTAD1, MGC4251, MAFF, SFPQ, CITED4, CEBPD, EIF4E2, HS2ST1, AGPS, PGK1, ATIC, ETNK1, LG2, NCE2, MARCH8, CNOT4, RNF8, PSMA5, DPF2, AMMECR1, KIAA1287, LOC144233, LOC286505, PNAS-4, FLJ20530, HUMPD3, GC45564, CAP350, ETAA16, HAN11, DNAPTP6, C7orf25, FLJ37953, FLJ10587, C7orf36, ELP4, NDEL1, NPD014, KFZP564D172, FAM53C, IER5, LOC255783, KIAA0146, KIAA0792, LOC439994, LOC283481, CG018, LOC130576, NGFRAP1L1, KIAA1217, 4orf7, C21 orf86, C9orf64, FLJ13456, FLJ12806, FLJ39370, GATS, CCDC92, FMNL2, ARID1B, ZFHX1B, SSBP2, ARID5B, LOC157381, KPNA3, ARHGAP24, CCND2, VIM, MYCBP2, GAS7, TRAM2, BASP1, FOXO1A, POLR2A, PER1, DDIT4, CD97, BIN1, SH2B3, DDB2, EMP3, NDST2, CHST2, NT5E, PDE4A, CPS1, PTGS1, GGCX, IRF5, ZBTB16, MAP4K4, CHST7, KLF12, NFRKB, PSD, FKSG49, NIFUN, FYN, ZMYM2, CACNA1G, SLC25A16, FLII, EIF1, SEPT6, NPAC, PHF15, NUP188, ABR, CNR1, LOC283824, FSTL4, DNM1, APOBEC3G, ATP2B1, SMPD1, SLC11A1, FXYD5, C14orf139, SH3BGRL3, LUZP1, ZNF335, SH3 KBP1, MST150, PRO1073, LOC388397, FKBP5, HIPK2, DTX1, ST3GAL2, ADAMTS7, TNRC6B, CYGB, SDHAL1, LOC572558, TRIO, FRAS1, KIAA1632, POLS, EBF, MAML2, PTPRA, PLEKHG2, DYM, SOX6, ARHGEF2, ZCCHC6, PPP3CA, FAM70B, TMED5, FLJ43663, HPS1, MEF2A, ST3GAL5, SMYD3, KLF7, LOC200230, RERE, QKI, BICD1, CTNNB1, POU2F2, EIF4ENIF1, BTG1, TCHP, FLJ90709, PCTK2, KIAA1600, B7-H4, PDE4DIP, KIAA0194, HOM-TES; and
(a) BGN, BIN1, CDH11, CDH2, CHN1, COL1A2, COL3A1, COL5A2, CREB3L1, CTGF, CYBRD1, DCN, DLC1, DNAJB4, DPT, ENPP2, FBLN1, FBLN5, FBN1, GREM1, HAS2, IGFBP3, IGFBP4, MLPH, MME, MMP2, MYL9, NEBL, NID1, NR2F1, OLFML3, PCOLCE, PMP22, POSTN, PPAP2B, PRR16, PRRX1, PTX3, PVRL3, RGL1, RGS4, ROR1, SEMA5A, TNFAIP6, TRAM2, WNT5A, ZEB1, ANXA6, CITED2, COL1A1, COL6A1, CYP1B1, DAB2, DOCK10, EML1, GNG11, IER3, ITGBL1, KCNMA1, LRIG1, LUM, MAP1B, NID2, PCDH9, PRKCA, PTGER4, PTGFR, SDC2, SERPINF1, SNED1, SYT11, TFPI, TGFBR3, VCAN, ANKRD25, C10ORF56, C5ORF13, CCDC92, CDKN2C, COPZ2, DDR2, ECM1, ENOX1, FADS2, FAP, FILIP1L, FSTL1, HS3ST2, KRT81, LMCD1, LTBP1, LTBP2, MMP1, NPAC, NRP1, PDGFC, PLEKHC1, PPM1D, PTGER2, SCCPDH, SEPT9, SPOCK1, SRGN, STARD13, STC1, SYNC1, TAGLN, TBX3, TGFB1I1, THY1, TNS3, TPM1, TSHZ1, TUBA1A, UGDH, VIM, XYLT1, ZBTB38, MBP, ABLIM1, ALDH1A3, ALOX15B, ANXA3, AP1M2, ARTN, CA2, CA9, CAMK2B, CCND2, CDH1, CDH3, CDS1, COL17A1, CORO1A, CTSL2, CXADR, CYP27B1, DSC2, DSG3, DST, EPB41L4B, FGFBP1, FGFR3, FST, GJB3, GRHL2, HBEGF, HOOK1, IL18, IL1B, IRF6, ITGB4, JAG2, KLK10, KLK5, KLK7, KLK8, KRT15, KRT16, KRT17, KRT5, KRT6B, LEPREL1, MYO5C, NDRG1, NMU, PI3, RAB25, RLN2, RNF128, S100A14, S100A7, S100A8, SERPINB1, SERPINB2, SLC2A9, SLPI, SNX10, SPINT2, SPRR1A, SPRR1B, ST14, SYK, TMEM40, TRIM29, TSPAN1, UCHL1, VSNL1, ZBED2, AQP3, CBARA1, CD24, CLCA2, CNTNAP2, CSTA, DSC3, EPCAM, ESRP2, FXYD3, GJB5, GPR87, IL1A, LAMA3, LAMB3, MAP7, MAPK13, MMP9, MPZL2, NRG1, SCNN1A, SERPINB13, SLC7A5, SPINT1, ST6GALNAC2, TGFA, TP63, TPD52L1, VGLL1, SLC6A8, ABCA12, ANK3, ANXA8, ANXA8L1, ARHGAP25, ARHGAP8, BDKRB2, BIK, C10ORF10, C10ORF116, C1ORF116, C20ORF19, C6ORF105, CELSR2, CKMT1A, CKMT1B, CLDN1, CST6, CYP4F11, E2F5, ELMO3, EPHA1, EVA1, EXPH5, F11R, FAT2, FGFR2, FLJ12684, FLJ20366, FZD3, GLS2, GNAL, IFI30, IGFBP2, IL1RN, IL4R, IRX4, JUP, KCNK1, KIAA0040, KIAA0888, KIAA1815, KRT14, KRT18, LAD1, LGALS7, LOC553158, LOC653562, LOC728113, LOC729884, LRRC1, LSR, MST1R, MYO1D, NAIP, OCLN, NEFM, NUP62CL, PERP, PLS1, POLR3G, PRKCH, PRRG4, PRSS8, PTPN3, RBM35A, RBM35B, RHBDF2, RHOD, RPS6KA1, RTEL1, SAA1, SAA1, SAA2, SH2D3A, SLC6A10P, SMPDL3B, SNCA, STAC, STAP2, TACSTD1, THBD, TMEM30B, TMPRSS11E, TMPRSS4, TNFRSF6B, TP73L, WWC1, XDH, ZNF165; and
(b) BGN, BIN1, CDH11, CDH2, CHN1, COL1A2, COL3A1, COL5A2, CREB3L1, CTGF, CYBRD1, DCN, DLC1, DNAJB4, DPT, ENPP2, FBLN1, FBLN5, FBN1, GREM1, HAS2, IGFBP3, IGFBP4, MLPH, MME, MMP2, MYL9, NEBL, NID1, NR2F1, OLFML3, PCOLCE, PMP22, POSTN, PPAP2B, PRR16, PRRX1, PTX3, PVRL3, RGL1, RGS4, ROR1, SEMA5A, TNFAIP6, TRAM2, WNT5A, ZEB1, ANXA6, CITED2, COL1A1, COL6A1, CYP1B1, DAB2, DOCK10, EML1, GNG11, IER3, ITGBL1, KCNMA1, LRIG1, LUM, MAP1B, NID2, PCDH9, PRKCA, PTGER4, PTGFR, SDC2, SERPINF1, SNED1, SYT11, TFPI, TGFBR3, VCAN, ANKRD25, C10ORF56, C5ORF13, CCDC92, CDKN2C, COPZ2, DDR2, ECM1, ENOX1, FADS2, FAP, FILIP1L, FSTL1, HS3ST2, KRT81, LMCD1, LTBP1, LTBP2, MMP1, NPAC, NRP1, PDGFC, PLEKHC1, PPM1D, PTGER2, SCCPDH, SEPT9, SPOCK1, SRGN, STARD13, STC1, SYNC1, TAGLN, TBX3, TGFB1I1, THY1, TNS3, TPM1, TSHZ1, TUBA1A, UGDH, VIM, XYLT1, ZBTB38; and
(c) ENPP2, CITED2, ZEB1, NID2, SEMA5A, DAB2, KCNMA1, PTX3, PCDH9, CREB3L1, BGN, ROR1, DLC1, GNG11, CDH11, NR2F1, NR2F2, PRR16, MYL9, DOCK10, LRIG1, IER3, EML1, NEBL, RGL1, MLPH, TRAM2, DNAJB4, FBLN5, RGS4, HAS2, ITGBL1, IGFBP4, BIN1, DPT, PCOLCE, GREM1, PPAP2B, CDH2, PMP22, LUM, CHN1, CYP1B1, MME, WNT5A, POSTN, MMP2, CTGF, FBLN1, IGFBP3, DCN, PRRX1, ANXA6, PVRL3, MAP1B, TNFAIP6, CYBRD1, FBN1, NID1, OLFML3, SNAI1, SNAI2, TAGLN, SYNC, TUBA1A, PPM1D, TWIST1, FN1, TGFBR3, SERPINF1, UGDH, SRGN, FAP, PTGER4, VIM, PRKCA, FSTL1, MMP1, NRP1, FILIP1L, SCCPDH, LTBP2, XYLT1, HS3ST2, SYT11, TSHZ1, THY1, SEPT9, S100A4, CCDC92, TNS3, ENOX1, TGFB1I1, ZEB2, LMCD1, PDGFC, ECM1, TFPI, TBX3, DDR2, PTGER2, CDKN2C, VCAN, CD44, STARD13, SNED1, ZBTB38, SDC2, TPM1, COPZ2, STC1, CDH1, KRT5, KRT6B, CD24, EPCAM, ESRP1, CLDN3, CLDN4, ERBB3, SPOCK1, FERMT2, GLYR1, LTBP1, FADS2, KANK2, PTGFR, ARTN, SLC7A5, EPB41L4B, KRT15, CDH3, TPD52L1, ANXA3, PI3, NDRG1, DSG3, S100A14, S100S7, KLK8, S100A8, PTX3, SPINT2, FXYD3, KRT16, RAB25, LGALS7, DSC3, COL5A2, ITGB4, TMEM40, SPRR1A, AP1M2, SERPINB13, TP73L; and
(d) CYBRD1, ARTN, CITED2, SLC7A5, CHN1, CTGF, CSTA, MYL9, EPB41L4B, KRT15, LRIG1, CDH3, TPD52L1, ANXA3, OLFML3, CORO1A, PI3, NDRG1, DSG3, PMP22, S100A14, S100S7, KLK8, S100A8, PTX3, SPINT2, IER3, FXYD3, KRT16, TGFBR3, KRT5, RAB25, SDC2, LUM, LGALS7, KRT6B, CD24, COL1A2, GNG11, DSC3, COL5A2, RGS4, ITGB4, TMEM40, SPRR1A, AP1M2, SERPINB13, SNED1, LEPREL1, TRAM2, TP73L, and PAP2B; and
(e) CYBRD1, ARTN, CITED2, SLC7A5, CHN1, CTGF, CSTA, MYL9, EPB41L4B, KRT15, LRIG1, CDH3, TPD52L1, ANXA3, OLFML3, CORO1A; and
(f) PI3, NDRG1, DSG3, PMP22, S100A14, S100S7, KLK8, S100A8, PTX3, SPINT2, IER3, FXYD3, KRT16, TGFBR3; and
(g) KRT5, RAB25, SDC2, LUM, LGALS7, KRT6B, CD24, COL1A2, GNG11, DSC3, COL5A2; and
(h) RGS4, ITGB4, TMEM40, SPRR1A, AP1M2, AERPINB13, SNED1, LEPREL1, TRAM2, TP73L, and PAB2B; and
(i) CYBRD1, ARTN, KRT15, ITGB4, RGS4, SPRR1B, SLC7A5, ANXA3, IL18, AP1M2, CHN1, OLFML3, ZBED2, LEPREL1, ABLIM1, CDH3, DNAJB4, TRAM2, UCHL1, CTGF; and
   (i) CYBRD1, ARTN, KRT15, ITGB4, RGS4, SPRR1B, SLC7A5, ANXA3, IL18, AP1M2; and
   (ii) CYBRD1, ARTN, KRT15, ITGB4, RGS4; and
(j) CYBRD1, RGS4, CDH3, CHN1, SLC7A5, PRRX1, CITED2, COL5A2, ARTN, ZBED2, SNED1, ITGB4, LUM, PVRL3, OLFML3, BIN1, CCND2, DAB2, ANXA3, IL18; and
   (i) CYBRD1, RGS4, CDH3, CHN1, SLC7A5, PRRX1, CITED2, COL5A2, ARTN, ZBED2; and
   (ii) CYBRD1, RGS4, CDH3, CHN1, SLC7A5; and
(k) CYBRD1, ARTN, RGS4, CITED2, SLC7A5, ITGB4, CHN1, PI3, TMEM40, NDRG1, SPRR1A, PCDH9, CTGF, MAP1B, DSG3, AP1M2, FBLN5, SERPINB13, PMP22, CSTA; and
   (i) CYBRD1, ARTN, RGS4, CITED2, SLC7A5, ITGB4, CHN1, PI3, TMEM40, NDRG1; and
   (ii) CYBRD1, ARTN, RGS4, CITED2, SLC7A5; and

(ii) said second set of genes comprises or consists of:

AXL, NOTCH3, GPR30, PROCR, PAX2, PI3KA, CD44, CD133, ALDH1, BMI1, KRT19, NRP1, MSI1, MTOR, JAG1, p38, ESA1, ZEB1, TWIST1, FOXC2, CDH2, ZEB2, FOXC1, SNAI1, SNAI2, TWIST2, GSC, TCF3, CD44, VIM, FN1, MLPH1, ARHGEF11; and

(iii) said third set of genes comprises or consists of:

At least one gene from said first set of genes and at least one of:

AXL, NOTCH3, GPR30, PROCR, PAX2, PI3KA, CD44, CD133, ALDH1, BMI1, KRT19, NRP1, MSI1, MTOR, JAG1, p38, ESA1, ZEB1, TWIST1, FOXC2, CDH2, ZEB2, FOXC1, SNAI1, SNAI2, TWIST2, GSC, TCF3, CD44, VIM, FN1, MLPH1, ARHGEF11; and

(iv) said fourth set of genes comprises or consists of:

At least two genes from said first set of genes and at least one of: AXL, NOTCH3, GPR30, PROCR, PAX2, PI3KA, CD44, CD133, ALDH1, BMI1, KRT19, NRP1, MSI1, MTOR, JAG1, p38, ESA1, ZEB1, TWIST1, FOXC2, CDH2, ZEB2, FOXC1, SNAI1, SNAI2, TWIST2, GSC, TCF3, CD44, VIM, FN1, MLPH1, and ARHGEF11; and

(v) said fifth set of genes comprises or consists of:

beta catenin, FZD1, FZD2, FZD3, FZD4, FZD5, FZD6, FZD7, FZD8, FZD9, FZD10, LRP5, LRP6, WNT1, WNT2, WNT2B, WNT3, WNT3A, WNT4 WNT5A, WNT5B, WNT6, WNT7A, WNT7B, WNT8A, WNT8B, WNT9A, WNT9B, WNT10A, WNT10B, WNT11, WNT16, SRFP1, SRFP2, FRZB, SFRP4, SFRP5, DKK1, DKK2, DKK3, DKK4, WIF1, AKT2, PI3KA, Disheveled, Smoothened, Axin1, Axin2; and

(vi) said sixth set of genes comprises or consists of:

At least one gene from said first set of genes and at least one of:

beta catenin, FZD1, FZD2, FZD3, FZD4, FZD5, FZD6, FZD7, FZD8, FZD9, FZD10, LRP5, LRP6, WNT1, WNT2, WNT2B, WNT3, WNT3A, WNT4 WNT5A, WNT5B, WNT6, WNT7A, WNT7B, WNT8A, WNT8B, WNT9A, WNT9B, WNT10A, WNT10B, WNT11, WNT16, SRFP1, SRFP2, FRZB, SFRP4, SFRP5, DKK1, DKK2, DKK3, DKK4, WIF1, AKT2, PI3KA, Disheveled, Smoothened, Axin1, Axin2; and

(vii) said seventh set of genes comprises or consists of:

TGFbeta1, TGFbeta2, TGFbeta3, ALK1, ALK2, ALK3, ALK4, ALK5, ALK6, ALK7, TGFbetaR-II, BMPR-II, ACTR-IIA, ACTR-IIB, AMHR-II, SMAD2, SMAD3; and

(viii) said eighth set of genes comprises or consists of:

At least one gene from said first set of genes and at least one of:

TGFbeta1, TGFbeta2, TGFbeta3, ALK1, ALK2, ALK3, ALK4, ALK5, ALK6, ALK7, TGFbetaR-II, BMPR-II, ACTR-IIA, ACTR-IIB, AMHR-II, SMAD2, SMAD3; and

(ix) said ninth set of genes comprises or consists of:

S100A4, SDC2, CYP1B1, FERMT2, CYBRD1, SNAI2, CD44, BIN1, PMP22, CTGF, IGFBP3, ANXA6, MAP1B, TAGLN, ENPP2, FBLN5, PCOLCE, FN1, VIM, PPAP2B, SEM5A, VCAN, NID2, NID1, HAS2, PRRX1, TNFAIP6, CLDN4, FADS2, DDIT4, GNG11, PER1, ISCU, KPNA3, SLC44A1, FKBP5, NR2F1, MLPH, DOCK10, RGS4, IGFBP4; and

(x) said tenth set of genes comprises or consists of:

S100A4, SDC2, CYP1B1, FERMT2, CYBRD1, SNAI2, CD44, BIN1, ENPP2, FBLN5, PCOLCE, FN1, VIM, PPAP2B, SEM5A, VCAN, CLDN4, FADS2, DDIT4, GNG11, PER1, ISCU, KPNA3, SLC44A1, FKBP5, IGFBP4; and

(xi) said eleventh set of genes comprises or consists of:

SDC2, CYBRD1, SNAI2, PMP22, CTGF, IGFBP3, ANXA6, MAP1B, TAGLN, PPAP2B, HAS2, NRF2, MLPH, DOCK10, RGS4; and

(xii) said twelfth set of genes comprises or consists of:

FERMT2, BIN1, PMP22, CHN1, CTGF, IGFBP3, NID2, CLDN4, CYBRD1, PRR16, ANXA6, PCOLCE, PPAP2B, HAS2, CDH11, MLPH, DOCK10, RGS4, PVRL3, NID2, SLC44A1, FKBP5; and

(xiii) said thirteenth set of genes comprises or consists of:

FERMT2, BIN1, PMP22, CHN1, CTGF, IGFBP3, NID2, CLDN4; and

(xiv) said fourteenth set of genes comprises or consists of:

HAS2, BIN1, PCOLCE, FERMT2, CTGF, IGFBP3, NID2, SLC44A1, FKBP5, and MLPH; and
optionally, administering the agent to the subject.

In some embodiments, the determining the level of gene expression product comprises determining the level of RNA expression of each gene of said plurality of genes. In an embodiment, the level of gene expression is a function of the level of RNA expression of each gene of said plurality of genes. In an embodiment, the level of RNA expression is acquired. In an embodiment, the level of RNA expression of said plurality of genes is assayed. In an embodiment, the level of RNA expression is assayed by detecting an RNA product, e.g., mRNA of said sample. In an embodiment, the level of RNA expression is assayed by a hybridization based method, e.g., hybridization with a probe that is specific for said RNA product. In an embodiment, the level of RNA expression is assayed by; applying said sample, or the mRNA isolated from, or amplified from; said sample, to a nucleic acid microarray, or chip array. In an embodiment, the level of RNA expression is assayed by microarray. In an embodiment, the level of RNA expression is assayed by a polymerase chain reaction (PCR) based method, e.g., qRT-PCR. In an embodiment, the level of RNA expression is assayed by a sequencing based method. In an embodiment, the level of RNA expression is assayed by quantitative RNA sequencing. In an embodiment, the level of RNA expression is assayed by RNA in situ hybridization. In an embodiment, the level of RNA expression is assayed in the whole subject sample. In an embodiment, the level of RNA expression is assayed in a subregion of the subject sample, e.g., subregions of a tissue sample.

In some embodiments, the determining the level of gene expression product comprises determining the level of protein expression of each gene of said plurality of genes. In an embodiment, the level of protein expression is acquired. In an embodiment, the level of protein expression is assayed. In an embodiment, the level of protein expression is assayed by detecting a protein product. In an embodiment, the level of protein expression is assayed using antibodies selective for said protein product. In an embodiment, the level of protein expression is assayed by an immunohistochemistry technique. In an embodiment, the level of protein expression is assayed by an immunohistochemistry technique, using antibodies specific for said protein product. In an embodiment, the level of protein expression is assayed by an immunoassay, e.g., Western blot, enzyme linked immunosorbant assay (ELISA). In an embodiment, the level of protein expression is assayed by an immunoassay specific for said protein. In an embodiment, levels of gene expression are assessed using protein activity assays, such as functional assays. In an embodiment, the level of protein expression is assayed in the whole subject sample. In an embodiment, the level of protein expression is assayed in a subregion of the subject sample, e.g., subregions of a tissue sample.

In some embodiments, the method further comprises determining the level of gene expression product in a cell. In some embodiments, the determining the level of gene expression product in a cell comprises: contacting the cell with an agent; determining the level of gene expression product; and comparing the level of gene expression product to an appropriate control.

In some embodiments, the subject sample is a sample described herein, e.g., blood, urine, or tissue sample. In an embodiment, the subject sample is a tissue sample, e.g., biopsy. In an embodiment, the subject sample is a bodily fluid, e.g., blood, plasma, urine, saliva, sweat, tears, semen, or cerebrospinal fluid. In an embodiment, the subject sample is a bodily product, e.g., exhaled breath. In an embodiment, said subject sample is a tissue sample, wherein said tissue sample is derived from fixed tissue, paraffin embedded tissue, fresh tissue, or frozen tissue. In an embodiment, said subject sample is a tissue sample, wherein said tissue sample is fixed tissue, paraffin embedded tissue, fresh tissue, or frozen tissue.

In some embodiments the subject has cancer, e.g., a cancer described herein, e.g., breast cancer. The cancer can include cancers characterized as comprising cancer stem cells, cancer associated mesenchymal cells, or tumor initiating cancer cells. The cancer can include cancers that have been characterized as being enriched with cancer stem cells, cancer associated mesenchymal cells, or tumor initiating cancer cells. Exemplary cancers include epithelial cancers, breast, lung, pancreatic, colorectal, prostate, head and neck, melanoma, acute myelogenous leukemia, and glioblastoma. Exemplary breast cancers include triple negative breast cancer, basal-like breast cancer, claudin-low breast cancer, invasive, inflammatory, metaplastic, and advanced Her-2 positive or ER-positive cancers resistant to therapy. Other cancers include but are not limited to, brain, abdominal, esophagus, gastrointestinal, glioma, liver, tongue, neuroblastoma, osteosarcoma, ovarian, retinoblastoma, Wilm's tumor, multiple myeloma, skin, lymphoma, blood, retinal, acute lymphoblastic leukemia, bladder, cervical, kidney, endometrial, meningioma, lymphoma, skin, uterine, lung, non small cell lung, nasopharyngeal carcinoma, neuroblastoma, solid tumor, hematologic malignancy, leukemia, squamous cell carcinoma, testicular, thyroid, mesothelioma, brain vulval, sarcoma, intestine, oral, T cell leukemia, endocrine, salivary, spermatocytic seminoma, sporadic medulalry thyroid carcinoma, non-proliferating testes cells, cancers related to malignant mast cells, non-Hodgkin's lymphoma, and diffuse large B cell lymphoma.

The cancer can be a primary tumor, i.e., located at the anatomical site of tumor growth initiation. The cancer can also be metastatic, i.e., appearing at least a second anatomical site other than the anatomical site of tumor growth initiation. The cancer can be a recurrent cancer, i.e., cancer that returns following treatment, and after a period of time in which the cancer was undetectable. The recurrent cancer can be anatomically located locally to the original tumor, e.g., anatomically near the original tumor; regionally to the original tumor, e.g., in a lymph node located near the original tumor; or distantly to the original tumor, e.g., anatomically in a region remote from the original tumor.

Also described herein are in vitro methods and assays. In one aspect described herein are in vitro methods and assays of determining if a subject is a potential candidate for treatment with an agent that inhibits or kills cancer associated mesenchymal cells, tumor initiating cancer cells, or cancer stem cells, the method comprising determining the level of gene expression product of a plurality of genes selected from a first and/or second and/or third and/or fourth and/or fifth and/or sixth set of genes, in a subject sample, wherein:
(i) said first set of genes comprises or consists of:
S100A4, SDC2, CYP1B1, FERMT2, CYBRD1, SNAI2, CD44, BIN1, PMP22, CTGF, IGFBP3, ANXA6, MAP1B, TAGLN, ENPP2, FBLN5, PCOLCE, FN1, VIM, PPAP2B, SEM5A, VCAN, NID2, NID1, HAS2, PRRX1, TNFAIP6, CLDN4, FADS2, DDIT4, GNG11, PER1, ISCU, KPNA3, SLC44A1, FKBP5, NR2F1, MLPH, DOCK10, RGS4, IGFBP4; and
(ii) said second set of genes comprises or consists of:
S100A4, SDC2, CYP1B1, FERMT2, CYBRD1, SNAI2, CD44, BIN1, ENPP2, FBLN5, PCOLCE, FN1, VIM, PPAP2B, SEM5A, VCAN, CLDN4, FADS2, DDIT4, GNG11, PER1, ISCU, KPNA3, SLC44A1, FKBP5, IGFBP4; and
(iii) said third set of genes comprises or consists of:
SDC2, CYBRD1, SNAI2, PMP22, CTGF, IGFBP3, ANXA6, MAP1B, TAGLN, PPAP2B, HAS2, NRF2, MLPH, DOCK10, RGS4; and
(iv) said fourth set of genes comprises or consists of:
FERMT2, BIN1, PMP22, CHN1, CTGF, IGFBP3, NID2, CLDN4, CYBRD1, PRR16, ANXA6, PCOLCE, PPAP2B, HAS2, CDH11, MLPH, DOCK10, RGS4, PVRL3, NID2, SLC44A1, FKBP5; and
(v) said fifth set of genes comprises or consists of:
FERMT2, BIN1, PMP22, CHN1, CTGF, IGFBP3, NID2, CLDN4; and
(vi) said sixth set of genes comprises or consists of:
HAS2, BIN1, PCOLCE, FERMT2, CTGF, IGFBP3, NID2, SLC44A1, FKBP5, and MLPH; and
optionally, administering the agent to the subject.

In some embodiments, the determining the level of gene expression product comprises determining the level of RNA expression of each gene of said plurality of genes. In an embodiment, the level of gene expression is a function of the level of RNA expression of each gene of said plurality of genes. In an embodiment, the level of RNA expression is acquired. In an embodiment, the level of RNA expression of said plurality of genes is assayed. In an embodiment, the level of RNA expression is assayed by detecting an RNA product, e.g., mRNA of said sample. In an embodiment, the level of RNA expression is assayed by a hybridization based method, e.g., hybridization with a probe that is specific for said RNA product. In an embodiment, the level of RNA expression is assayed by; applying said sample, or the mRNA isolated from, or amplified from; said sample, to a nucleic acid microarray, or chip array. In an embodiment, the level of RNA expression is assayed by microarray. In an embodiment, the level of RNA expression is assayed by a polymerase chain reaction (PCR) based method, e.g., qRT-PCR. In an embodiment, the level of RNA expression is assayed by a sequencing based method. In an embodiment, the level of RNA expression is assayed by quantitative RNA sequencing. In an embodiment, the level of RNA expression is assayed by RNA in situ hybridization. In an embodiment, the level of RNA expression is assayed in the whole subject sample. In an embodiment, the level of RNA expression is assayed in a subregion of the subject sample, e.g., subregions of a tissue sample.

In some embodiments, the determining the level of gene expression product comprises determining the level of protein expression of each gene of said plurality of genes. In an embodiment, the level of protein expression is acquired. In an embodiment, the level of protein expression is assayed. In an embodiment, the level of protein expression is assayed by detecting a protein product. In an embodiment, the level of protein expression is assayed using antibodies selective for said protein product. In an embodiment, the level of protein expression is assayed by an immunohistochemistry technique. In an embodiment, the level of protein expression is assayed by an immunohistochemistry technique, using antibodies specific for said protein product. In an embodiment, the level of protein expression is assayed by an immunoassay, e.g., Western blot, enzyme linked immunosorbant assay (ELISA). In an embodiment, the level of protein expression is assayed by an immunoassay specific for said protein. In an embodiment, levels of gene expression are assessed using protein activity assays, such as functional assays. In an embodiment, the level of protein expression is assayed in the whole subject sample. In an embodiment, the level of protein expression is assayed in a subregion of the subject sample, e.g., subregions of a tissue sample.

In some embodiments, the method further comprises determining the level of gene expression product in a cell. In some embodiments, the determining the level of gene expression product in a cell comprises: contacting the cell with an agent; determining the level of gene expression product; and comparing the level of gene expression product to an appropriate control.

In some embodiments, the subject sample is a sample described herein, e.g., blood, urine, or tissue sample. In an embodiment, the subject sample is a tissue sample, e.g., biopsy. In an embodiment, the subject sample is a bodily fluid, e.g., blood, plasma, urine, saliva, sweat, tears, semen, or cerebrospinal fluid. In an embodiment, the subject sample is a bodily product, e.g., exhaled breath. In an embodiment, said subject sample is a tissue sample, wherein said tissue sample is derived from fixed tissue, paraffin embedded tissue, fresh tissue, or frozen tissue. In an embodiment, said subject sample is a tissue sample, wherein said tissue sample is fixed tissue, paraffin embedded tissue, fresh tissue, or frozen tissue.

In some embodiments the subject has cancer, e.g., a cancer described herein, e.g., breast cancer. The cancer can include cancers characterized as comprising cancer stem cells, cancer associated mesenchymal cells, or tumor initiating cancer cells. The cancer can include cancers that have been characterized as being enriched with cancer stem cells, cancer associated mesenchymal cells, or tumor initiating cancer cells. Exemplary cancers include epithelial cancers, breast, lung, pancreatic, colorectal, prostate, head and neck, melanoma, acute myelogenous leukemia, and glioblastoma. Exemplary breast cancers include triple negative breast cancer, basal-like breast cancer, claudin-low breast cancer, invasive, inflammatory, metaplastic, and advanced Her-2 positive or ER-positive cancers resistant to therapy. Other cancers include but are not limited to, brain, abdominal, esophagus, gastrointestinal, glioma, liver, tongue, neuroblastoma, osteosarcoma, ovarian, retinoblastoma, Wilm's tumor, multiple myeloma, skin, lymphoma, blood, retinal, acute lymphoblastic leukemia, bladder, cervical, kidney, endometrial, meningioma, lymphoma, skin, uterine, lung, non small cell lung, nasopharyngeal carcinoma, neuroblastoma, solid tumor, hematologic malignancy, leukemia, squamous cell carcinoma, testicular, thyroid, mesothelioma, brain vulval, sarcoma, intestine, oral, T cell leukemia, endocrine, salivary, spermatocytic seminoma, sporadic medulalry thyroid carcinoma, non-proliferating testes cells, cancers related to malignant mast cells, non-Hodgkin's lymphoma, and diffuse large B cell lymphoma.

The cancer can be a primary tumor, i.e., located at the anatomical site of tumor growth initiation. The cancer can also be metastatic, i.e., appearing at least a second anatomical site other than the anatomical site of tumor growth initiation. The cancer can be a recurrent cancer, i.e., cancer that returns following treatment, and after a period of time in which the cancer was undetectable. The recurrent cancer can be anatomically located locally to the original tumor, e.g., anatomically near the original tumor; regionally to the original tumor, e.g., in a lymph node located near the original tumor; or distantly to the original tumor, e.g., anatomically in a region remote from the original tumor.

### BRIEF DESCRIPTION OF THE DRAWINGS

FIG. 1 depicts genes highly ranked from multiple statistical gene ranking methods. Genes listed are concordant between two of three statistical gene ranking methods: LASSO, STEPWISE, and CART. A one (1) indicates a gene that is one of the top 60 genes of the ranking algorithm. A zero (0) indicates a gene that is not one of the top 60 genes of the ranking algorithm, but is a top 60 gene of another ranking algorithm.

FIG. 2 depicts the concordance of the top 60 genes from three gene ranking methods. Three gene ranking methods were utilized to fit a gene rank order from EMT (epithelial-to-mesenchymal transition) and CSC genes. The three methods used were LASSO, STEPWISE, and CART. FIG. 2 illustrates the level of concordance between the three methods.

FIG. 3 illustrates the LASSO-20 gene model. The top twenty highly ranked genes from the LASSO gene ranking algorithm were used to distinguish ‘likely to recur’ and ‘no recurrence’ triple negative breast cancer patient subgroups. The left panel is a time to recurrence Kaplan-Meier plot for the Event-Free fraction from 0-7 years. As labeled, ‘Recurrence’ refers to ‘Likely to Recur’ and ‘No Recurrence’ refers to ‘Not Likely to Recur’. The right panel is a receiver operator characteristic (ROC) plot and calculation of Area-Under-Curve (AUC), as a projection of the relationship between the true positive rate and the false positive rate.

FIG. 4 illustrates the STEPWISE 20-gene model. The top twenty highly ranked genes from the STEPWISE gene ranking algorithm were used to distinguish ‘likely to recur’ and ‘no recurrence’ triple negative breast cancer patient subgroups. The left panel is a time to recurrence Kaplan-Meier plot for the Event-Free fraction from 0-7 years. As labeled, ‘Recurrence’ refers to ‘Likely to Recur’ and ‘No Recurrence’ refers to ‘Not Likely to Recur’. The right panel is a receiver operator characteristic (ROC) plot and calculation of Area-Under-Curve (AUC), as a projection of the relationship between the true positive rate and the false positive rate.

FIG. 5 illustrates the CART 20-gene model. The top twenty highly ranked genes from the CART gene ranking algorithm were used to distinguish ‘likely to recur’ and ‘no recurrence’ triple negative breast cancer patient subgroups. The left panel is a time to recurrence Kaplan-Meier plot for the Event-Free fraction from 0-7 years. As labeled, ‘Recurrence’ refers to ‘Likely to Recur’ and ‘No Recurrence’ refers to ‘Not Likely to Recur’. The right panel is a receiver operator characteristic (ROC) plot and calculation of Area-Under-Curve (AUC), as a projection of the relationship between the true positive rate and the false positive rate.

FIG. 6 illustrates the addition of driver genes to base gene models [Add-One-In]. The table lists candidate genes that are tested in a base gene model with 4 other genes (5-gene models). Genes are sorted by p-value improvement, when tested for their prediction of ‘recurrence’ versus ‘no recurrence’ sub grouping of triple negative breast cancer patients, where biopsies were isolated prior to treatment. High performing ‘add-in’ genes (p-value<1e−5) are shown as ̂̂genes, ** genes that are ‘positive control’ (Ranked top 5 genes via LASSO).

FIG. 7 illustrates recurrence plots for driver genes designated as Group A. Five gene models were tested for their prediction of ‘recurrence’ versus ‘no recurrence’ sub grouping of triple negative breast cancer patients, where biopsies were isolated prior to treatment. The figure depicts ‘time to recurrence’ Kaplan-Meier display for the ‘event-free fraction’ from 0-7 years. As labeled, ‘Recurrence’ refers to ‘Likely to Recur’ and ‘No Recurrence’ refers to ‘Not Likely to Recur’. The 5-gene models were constructed with a base model of 4 genes and addition of a single candidate driver gene. Driver genes were: MSI1, NRP1, TWIST1, BMI1, AXL, and ZEB1. Solid Lines depict the addition of a driver gene. Dotted Lines depict the addition of a positive control gene.

FIG. 8 illustrates recurrence plots for driver genes designated as Group B. Five gene models were tested for their prediction of ‘recurrence’ versus ‘no recurrence’ sub grouping of triple negative breast cancer patients, where biopsies were isolated prior to treatment. The figure depicts ‘time to recurrence’ Kaplan-Meier display for the ‘event-free fraction’ from 0-7 years. The 5-gene models were constructed with a base model of 4 genes and addition of a single candidate driver gene. Driver genes were: CD44, KRT19, PROCR, FOXC2, and NOTCH3. Solid Lines depict the addition of a driver gene. Dotted Lines depict the addition of a positive control gene.

FIG. 9 illustrates a recurrence plot for driver genes designated as a CSC group. A CSC gene model was tested for its prediction of ‘recurrence’ versus ‘no recurrence’ sub grouping of triple negative breast cancer patients, where biopsies were isolated prior to treatment. FIG. 9 depicts ‘time to recurrence’ Kaplan-Meier display for the ‘event-free fraction’ from 0-7 years. The nine CSC candidate genes evaluated were NOTCH3, PROCR, BMI1, KRT19, MSI1, NRP1, JAG1, ALDH1A1, and HTATIP. The p-value between the ‘recurrence’ and ‘no recurrence’ subgroups of patients was p=5.77e−4.

FIG. 10 illustrates a recurrence plot for a 5-gene CSC model. A five gene model was tested for its prediction of ‘recurrence’ versus ‘no recurrence’ subgroups of triple negative breast cancer patients, where biopsies were isolated prior to treatment. FIG. 10 depicts ‘time to recurrence’ Kaplan-Meier display for the ‘event-free fraction’ from 0-7 years. The genes evaluated in the model were NRP1, BMI1, JAG1, MSI1, and HTATIP. The p-value between the ‘recurrence’ and ‘no recurrence’ subgroups of patients was p=1.04e−3.

FIG. 11 illustrates the relative expression of candidate cancer stem cell and tumor initiating cancer cell genes, comparing tumorspheres with two-dimensional cell culture. The fold change in gene expression was measured by Q-PCR for RNA samples isolated from Tumorspheres or from two-dimensional cell culture of human breast cancer cell lines, MCF7, SUM159, and MDA-MB-231. The genes KRT5, SERPINF1, S100A4, RGL1, FAP, TGFBR3, FBLN5, MLPH, FADS2, TFPI, PPAP2B, SEPT9, CYBRD1, UGDH, TRAM2, PCDH9, and DLC1 were evaluated in this analysis. Plots show the fold change of Tumorsphere/2D culture on a log 2 scale for the three cell lines.

FIG. 12 illustrates a Time to Recurrence plot for 9 genes validated from tumorsphere culture. A Kaplan-Meier display for the ‘event-free fraction’ from 0-7 years is shown. As labeled, ‘Recurrence’ refers to ‘Likely to Recur’ and ‘No Recurrence’ refers to ‘Not Likely to Recur’. The genes in the model were CYBRD1, FAP, PPAP2B, TRAM2, TFPI, PCDH9, RGL1, FADS2, SERPINF1, DLC1, SEP9, MLPH, S100A4, FBLN5, and TGFBR3. A combined Gene Value was assigned that separates the patients into two groups. The p-value between the ‘Recurrence’ and ‘No recurrence’ subgroups of patients was p=6.83e−08.

FIG. 13 illustrates a recurrence plot for 5 genes validated from tumorsphere culture. The five gene model was tested for its prediction of ‘recurrence’ versus ‘no recurrence’ sub grouping of triple negative breast cancer patients, where biopsies were isolated prior to treatment. The figure depicts ‘time to recurrence’ Kaplan-Meier display for the ‘event-free fraction’ from 0-7 years. As labeled, ‘Recurrence’ refers to ‘Likely to Recur’ and ‘No Recurrence’ refers to ‘Not Likely to Recur’. The genes in the model were CYBRD1, FAP, PPAP2B, TRAM2, and TFPI. The p-value between the ‘Recurrence’ and ‘No recurrence’ subgroups of patients was p=2.19e−08.

FIG. 14 illustrates the upregulation of 11 cancer stem cell associated genes in sequential passaging of tumorspheres of SUM159 cells. The figure further illustrates the enhancement of the upregulation of the 11 cancer stem cell associated genes in paclitaxel treated SUM159 cell tumorspheres. The plot shows the percentage change in gene expression normalized to the expression level in the primary SUM159 tumorspheres.

FIG. 15 illustrates genes expressed in mesenchymal (M) tumor cells (i.e. basal breast cancer cell line), but are only expressed to a low or not expressed in epithelial (E) tumor cells (i.e. luminal breast cancer cell line) and fibroblast (F) non-tumor cells (i.e. fibroblasts). The values shown are the expression levels per gene per cell line, and the mean Expression level is indicated. These genes are designated as M-high, E-low, and Fibroblast-low. The example in the Box is one gene, EML1, where the Expression Level is shown for Basal, Luminal, and Fibroblast cell lines.

FIG. 16A illustrates a Recurrence plot for 19 genes designated as a M-high, E-low, and Fibroblast-low group. The 19 gene model was tested for its prediction of ‘recurrence’ versus ‘no recurrence’ sub grouping of triple negative breast cancer patients, where biopsies were isolated prior to treatment. The figure depicts ‘time to recurrence’ Kaplan-Meier display for the ‘event-free fraction’ from 0-7 years. As labeled, ‘Recurrence’ refers to ‘Likely to Recur’ and ‘No Recurrence’ refers to ‘Not Likely to Recur’. The genes in the model were CDH2, CHN1, CTGF, CYP1B1, DNAJB4, EML1, ENPP2, HAS2, MAP1B, NID1, PDGFC, PRR16, PRRX1, PVRL3, ROR1, SDC2, SNAI1, SNAI2, TNFAIP6, and VCAN. The p-value between the ‘Recurrence’ and ‘No recurrence’ subgroups of patients was p=1.9e−03. FIG. 16B is a receiver operator characteristic (ROC) plot and calculation of Area-Under-Curve (AUC), as a projection of the relationship between the true positive rate and the false positive rate, for the M-high, E-low, and Fibroblast-low 19 gene model. FIG. 16C illustrates a Recurrence plot for 6 genes designated as a M-high, E-low, and Fibroblast-low group. The 6 gene model was tested for its prediction of ‘recurrence’ versus ‘no recurrence’ sub grouping of triple negative breast cancer patients, where biopsies were isolated prior to treatment. The figure depicts ‘time to recurrence’ Kaplan-Meier display for the ‘event-free fraction’ from 0-7 years. As labeled, ‘Recurrence’ refers to ‘Likely to Recur’ and ‘No Recurrence’ refers to ‘Not Likely to Recur’. The genes in the 6 gene model were CHN1, CTGF, DNAJB4, PRRX1, PVRL3, and VCAN. The p-value between the ‘Recurrence’ and ‘No recurrence’ subgroups of patients was p=1.7e−04. FIG. 16D is a receiver operator characteristic (ROC) plot and calculation of Area-Under-Curve (AUC), as a projection of the relationship between the true positive rate and the false positive rate, for the M-high, E-low, and Fibroblast-low 6 gene model.

FIG. 17 illustrates the p-values associated with expression of significant genes (significance based on variance) over a large group of human breast cancer specimens. Twenty-five genes highly ranked by the variance criteria are shown to have a higher degree of variability as illustrated by p-value significance (below the indicated horizontal line, p<1.01).

FIG. 18 illustrates the polarity associated with expression of significant genes (significance based on polarity) over a large group of human breast cancer specimens. Genes with the highest and lowest Model Polarity are the ones that have the highest fraction of specimens that either upregulate or downregulate expression of the specified gene.

FIG. 19 illustrates a Time to Recurrence plot for an 18 gene model for candidate genes identified by Variance and Polarity. The 18 gene model was tested for its prediction of ‘recurrence’ versus ‘no recurrence’ subgroups of triple negative breast cancer patients, where biopsies were isolated prior to treatment. The left panel depicts ‘time to recurrence’ Kaplan-Meier display for the ‘event-free fraction’ from 0-7 years. The p-value between the ‘recurrence’ and ‘no recurrence’ subgroups of patients was p<0e+00. The right panel is a receiver operator characteristic (ROC) plot and calculation of Area-Under-Curve (AUC), as a projection of the relationship between the true positive rate and the false positive rate.

FIG. 20 illustrates a Time to Recurrence plot for a 12 gene model for candidate genes identified by Variance and Polarity. The 12 gene model was tested for its prediction of ‘recurrence’ versus ‘no recurrence’ subgroups of triple negative breast cancer patients, where biopsies were isolated prior to treatment. The left panel depicts ‘time to recurrence’ Kaplan-Meier display for the ‘event-free fraction’ from 0-7 years. The p-value between the ‘recurrence’ and ‘no recurrence’ subgroups of patients was p<0e+00. The right panel is a receiver operator characteristic (ROC) plot and calculation of Area-Under-Curve (AUC), as a projection of the relationship between the true positive rate and the false positive rate.

FIG. 21 illustrates a Time to Recurrence plot for a 6 gene model for candidate genes identified by Variance and Polarity. The 6 gene model was tested for its prediction of ‘recurrence’ versus ‘no recurrence’ subgroups of triple negative breast cancer patients, where biopsies were isolated prior to treatment. The left panel depicts ‘time to recurrence’ Kaplan-Meier display for the ‘event-free fraction’ from 0-7 years. The p-value between the ‘recurrence’ and ‘no recurrence’ subgroups of patients was p<1.22e−10. The right panel is a receiver operator characteristic (ROC) plot and calculation of Area-Under-Curve (AUC), as a projection of the relationship between the true positive rate and the false positive rate.

FIG. 22A illustrates a Time to Recurrence plot for a Tumorsphere gene model. The 6 gene model (CYBRD1, SERPINF1, FAP, PPAP2B, RGS4 and PRRX1) was tested for its prediction of ‘recurrence’ versus ‘no recurrence’ subgroups of triple negative breast cancer patients, where biopsies were isolated prior to treatment. The left panel depicts ‘time to recurrence’ Kaplan-Meier display for the ‘event-free fraction’ from 0-7 years. The p-value between the ‘recurrence’ and ‘no recurrence’ subgroups of patients was p=1.21e−07. FIG. 22B illustrates a receiver operator characteristic (ROC) plot and calculation of Area-Under-Curve (AUC), as a projection of the relationship between the true positive rate and the false positive rate, for the 6 gene model (CYBRD1, SERPINF1, FAP, PPAP2B, RGS4, and PRRX1). An AUC of 0.756 was observed. FIG. 22C illustrates a Time to Recurrence plot for a Tumorsphere 8-gene model combining CYBRD1, SERPINF1, FAP, PPAP2B with RGS4, PRRX1, FBXO21, and ID3. The 8 gene model was tested for its prediction of ‘recurrence’ versus ‘no recurrence’ subgroups of triple negative breast cancer patients where biopsies were isolated prior to chemotherapy treatment. The left panel depicts ‘time to recurrence’ Kaplan-Meier display for the ‘event-free fraction’ from 0-7 years. The p-value between the ‘recurrence’ and ‘no recurrence’ subgroups of patients was p=2.66e−12. FIG. 22B illustrates a receiver operator characteristic (ROC) plot and calculation of Area-Under-Curve (AUC), as a projection of the relationship between the true positive rate and the false positive rate, for the 8-gene model combining CYBRD1, SERPINF1, FAP, PPAP2B with RGS4, PRRX1, FBXO21, and ID3. An AUC was computed to 0.821 in Receiver Operator Curve analysis.

### DETAILED DESCRIPTION

Certain terms are first defined. Additional terms are defined throughout the specification.

“Acquire” or “acquiring” as the terms are used herein, refer to obtaining possession of a physical entity, or a value, e.g., a numerical value, by “directly acquiring” or “indirectly acquiring” the physical entity or value. “Directly acquiring” means performing a process (e.g., performing a synthetic or analytical method) to obtain the physical entity or value. “Indirectly acquiring” refers to receiving the physical entity or value from another party or source (e.g., a third party laboratory that directly acquired the physical entity or value). Directly acquiring a physical entity includes performing a process that includes a physical change in a physical substance, e.g., a starting material. Exemplary changes include making a physical entity from two or more starting materials, shearing or fragmenting a substance, separating or purifying a substance, combining two or more separate entities into a mixture, performing a chemical reaction that includes breaking or forming a covalent or non-covalent bond. Directly acquiring a value includes performing a process that includes a physical change in a sample or another substance, e.g., performing an analytical process which includes a physical change in a substance, e.g., a sample, analyte, or reagent (sometimes referred to herein as “physical analysis”), performing an analytical method, e.g., a method which includes one or more of the following: separating or purifying a substance, e.g., an analyte, or a fragment or other derivative thereof, from another substance; combining an analyte, or fragment or other derivative thereof, with another substance, e.g., a buffer, solvent, or reactant; or changing the structure of an analyte, or a fragment or other derivative thereof, e.g., by breaking or forming a covalent or non-covalent bond, between a first and a second atom of the analyte; or by changing the structure of a reagent, or a fragment or other derivative thereof, e.g., by breaking or forming a covalent or non-covalent bond, between a first and a second atom of the reagent.

“Acquiring a sample” as the term is used herein, refers to obtaining possession of a sample, e.g., a tissue sample or nucleic acid sample, by “directly acquiring” or “indirectly acquiring” the sample. “Directly acquiring a sample” means performing a process (e.g., performing a physical method such as a surgery or extraction) to obtain the sample. “Indirectly acquiring a sample” refers to receiving the sample from another party or source (e.g., a third party laboratory that directly acquired the sample). Directly acquiring a sample includes performing a process that includes a physical change in a physical substance, e.g., a starting material, such as a tissue, e.g., a tissue in a human patient or a tissue that has was previously isolated from a patient. Exemplary changes include making a physical entity from a starting material, dissecting or scraping a tissue; separating or purifying a substance (e.g., a sample tissue or a nucleic acid sample); combining two or more separate entities into a mixture; performing a chemical reaction that includes breaking or forming a covalent or non-covalent bond. Directly acquiring a sample includes performing a process that includes a physical change in a sample or another substance, e.g., as described above. As used herein, a subject who is a “candidate” is a one likely to respond to a particular therapeutic regimen, relative to a reference subject or group of subjects. A “non-candidate” subject is one not likely to respond to a particular therapeutic regimen, relative to a reference subject or group of subjects.

The term “cancer stem cell” refers to a cell or group of cells in a tumor having stem-like progenitor properties.

The term “tumor initiating cancer cell” refers to a cell with stem-like properties and the ability to initiate a tumor upon introduction into a tissue.

The term “cancer associated mesenchymal cell” refers to a cell or cells in a tumor that have acquired or retained mesenchymal properties.

The term “anti-cancer stem cell agent” refers to an inhibitor or killer of cancer stem cells causing a reduction or elimination of these cells or a reduction in the ability of these cells to proliferative or to survive the treatment.

The term “agent that inhibits or kills cancer associated mesenchymal cells” refers to an inhibitor or killer of cancer mesenchymal cells causing a reduction or elimination of these cells or a reduction in the ability of these cells to proliferative or to survive the treatment.

The term “agent that inhibits or kills tumor initiating cancer cells” refers to an inhibitor or killer of cells with stem-like properties and the ability to initiate a tumor upon introduction into a tissue.

The term “agent that kills or inhibits cancer stem cells” refers to an inhibitor or killer of cells or a group of cells in a tumor having stem-like progenitor properties.

The term “anti-cancer agent” refers to an inhibitor of cancer initiation, growth, progression, or metastasis

The terms “cancer” and “tumor” are used interchangeably herein. These terms refer to the presence of cells possessing characteristics typical of cancer-causing cells, such as uncontrolled proliferation, immortality, metastatic potential, rapid growth and proliferation rate, and certain characteristic morphological features. Cancer cells are often in the form of a tumor, but such cells can exist alone within an animal, or can be a non-tumorigenic cancer cell, such as a leukemia cell. These terms include a solid tumor, a soft tissue tumor, or a metastatic lesion.

“Chemotherapeutic agent” means a chemical substance, such as a cytotoxic or cytostatic agent, that is used to treat a condition, particularly cancer. As used herein, “chemotherapy” and “chemotherapeutic” and “chemotherapeutic agent” are synonymous terms.

“Likely to” or “increased likelihood,” as used herein, refers to an increased probability that an item, object, thing or person will occur. Thus, in one example, a subject that is likely to respond to treatment with an agent that inhibits or kills cancer associated mesenchymal cells, tumor initiating cancer cells, or cancer stem cells; alone or in combination, has an increased probability of responding to treatment with the agent that inhibits or kills cancer associated mesenchymal cells, tumor initiating cancer cells, or cancer stem cell; alone or in combination, relative to a reference subject or group of subjects.

The term “location”, as used herein, refers to a zone of a sample defined by preselected criteria, such as morphology, histopathology, and other attributes. A zone of a tumor can be defined by a unique gene expression pattern of a set of preselected genes. A zone may be classified as containing a specific cell type or multiple cell types, e.g., a zone may be classified as a nodule of cancer stem cells; a nodule of cancer associated mesenchymal cells; a nodule of tumor initiating cancer cells; a zone of transition, e.g., an area between epithelial and mesenchymal features of a tumor region; or it may be a niche indicated by the presence of a particular cell type or class, e.g., mesenchymal cells, stromal cells, inflammatory cells, endothelial cells, etc.

“Unlikely to” or “decreased likelihood” refers to a decreased probability that an event, item, object, thing or person will occur with respect to a reference. Thus, a subject that is unlikely to respond to treatment with an agent that inhibits or kills cancer associated mesenchymal cells, tumor initiating cancer cells, or cancer stem cells; alone or in combination, has a decreased probability of responding to treatment with the agent that inhibits or kills cancer associated mesenchymal cells, tumor initiating cancer cells, or cancer stem cells; alone or in combination, relative to a reference subject or group of subjects.

“Sequencing” a nucleic acid molecule requires determining the identity of at least one nucleotide in the molecule. The identity of less than all of the nucleotides in a molecule can be determined. The identity of a majority or all of the nucleotides in the molecule can be determined.

The terms “sample” and “subject sample” are used interchangeably herein. These terms refer to biological material obtained from a subject. The source of the sample can be solid tissue as from a fresh, frozen and/or preserved organ, tissue sample, biopsy, or aspirate; blood or any blood constituents; bodily fluids such as cerebral spinal fluid, amniotic fluid, peritoneal fluid or interstitial fluid; or cells from any time in gestation or development of the subject. The tissue sample can contain compounds that are not naturally intermixed with the tissue in nature such as preservatives, anticoagulants, buffers, fixatives, nutrients, antibiotics or the like. The sample can be preserved as a frozen sample or as formaldehyde- or paraformaldehyde-fixed paraffin-embedded (FFPE) tissue preparation. For example, the sample can be embedded in a matrix, e.g., an FFPE block or a frozen sample. The sample can also be a cell line, a cell line previously established, a cell line derived previously from a subject, etc.

The terms “treat” and “treatment” and “treatment regimen” and “therapeutic regimen” are used interchangeably herein. As used herein, the terms “treat” and “treatment” and “treatment regimen” and “therapeutic regimen” are defined as the application or administration of a compound, alone or in combination with, a second compound to a sample, e.g., a sample, or application or administration of the compound to an isolated tissue or cell, e.g., cell line, from a subject, e.g., a subject, who has a disorder (e.g., a disorder as described herein), a symptom of a disorder, or a predisposition toward a disorder, with the purpose to cure, heal, alleviate, relieve, alter, remedy, ameliorate, improve or affect the disorder, one or more symptoms of the disorder or the predisposition toward the disorder (e.g., to minimize at least one symptom of the disorder or to delay onset of at least one symptom of the disorder).

A “weighting factor” as used herein, refers to an element used as an adjustment factor for a specific value or group of similar values.

A subject that will “respond positively” or “respond favorably” as used herein, refers to a subject that will experience some degree of alleviation in one or more characteristics of a disease or disorder after receiving treatment with a therapeutic agent; and/or some degree of alleviation in one or more symptoms caused by a disease or disorder, after receiving treatment with a therapeutic agent.

A “responder” as used herein, is a subject that will experience some degree of alleviation in one or more characteristics of a disease or disorder; and/or some degree of alleviation in one or more symptoms caused by a disease or disorder, after receiving treatment with a therapeutic agent.

A “non-responder” as used herein, is a subject that will not experience some degree of alleviation in one or more characteristics of a disease or disorder after receiving treatment with a therapeutic agent; nor some degree of alleviation in one or more symptoms caused by a disease or disorder, after receiving treatment with the therapeutic agent.

A “reference criterion” as used herein, refers to a characteristic forming the basis of comparison for the evaluation or assessment of a measured characteristic.

### Cancer and Cancer Stem Cells

Cancer is one of the most significant health conditions and leading causes of death worldwide. Currently available treatments include chemotherapy, radiation, surgery, hormonal therapy, immunotherapy, epigenetic therapy, anti-angiogenesis inhibitors, and other modalities, including targeted therapies, such as tyrosine kinase inhibitors and antibody based therapies. However, these treatments are ineffective in treating many cancers, and/or preventing reoccurrence. This ineffectiveness or unsustainability may be due, at least in part, to the innate heterogenic nature of cancer.

Cancers are known to be heterogeneous entities, with subsets of cancer cells exhibiting distinct molecular characteristics, including distinct gene expression profiles. Furthermore, cells with different molecular characteristics within the same cancer can respond differently to a single treatment. Cancer stem cells, cancer associated mesenchymal cells, and tumor initiating cancer cells, comprise a unique subpopulation of a tumor and have been identified in a large variety of cancer types. Relative to the remaining portion of the tumor, i.e., the tumor bulk, this subset of cancer cells is more tumorigenic, more slow growing or quiescent, and often more resistant to chemotherapeutic agents. Although, this subpopulation of cells constitutes only a small fraction of a tumor, these cells are thought to be responsible for cancer initiation, growth, and recurrence.

Given that currently available cancer treatments have, in large part, been designed to attack rapidly proliferating cells (i.e. those cancer cells that comprise the tumor bulk); cancer stem cells, cancer associated mesenchymal cells, and tumor initiating cancer cells, which are often slow growing, may be relatively more resistant to these treatments. Therefore, methods to identify cancer patients likely to respond positively to a treatment comprising an agent that inhibits or kills cancer associated mesenchymal cells, tumor initiating cancer cells, or cancer stem cells are needed; and can provide the basis for subsequent administration of a treatment comprising an agent that inhibits or kills cancer associated mesenchymal cells, tumor initiating cancer cells, or cancer stem cells to this candidate group of cancer patients.

The present invention provides a method of classifying subjects likely to respond to a particular therapeutic regimen for treating cancer. The method is based, at least in part, on the characterization of signals (e.g., the level of gene expression) possessed by a candidate subject population for treatment with a preselected drug. In general, the method involves identifying differences in candidate and non-candidate subject populations, where for example, a subject population has a gene expression profile associated with a candidate or non-candidate classification. The method can further include administration of the therapeutic regimen to the candidate population based on the characterized gene expression profile.

Overall, the invention described herein methods of evaluating and/or treating a subject, including acquiring a value or values that is a function of the level of gene expression for (each of) a plurality of genes selected from a first and/or second and/or third and/or fourth and/or fifth and/or sixth and/or or seventh and/or eighth set of genes; responsive to the value or values, classifying the subject as a candidate or non-candidate for treatment with a preselected drug; optionally, further treating the subject by administering the agent that inhibits or kills cancer associated mesenchymal cells, tumor initiating cancer cells, or cancer stem cells; or withholding treatment from the subject; provided that if an agent that inhibits or kills cancer associated mesenchymal cells, tumor initiating cancer cells, or cancer stem cells is not administered, the acquisition of the subject sample or the acquisition of the value or values that is a function of the level of gene expression comprises directly acquiring; thereby evaluating or treating the subject. In response to the value or values, the invention also features: stratification of a subject population; identification or selection of the subject as likely or unlikely to respond positively to a treatment; selection of a treatment; or prognostication of the time course of the disease in the subject; measuring the response at the end of therapy and predicting the long term outcome; and/or determining the cancer stem cell population as a predictor of response to a treatment or therapy.

### Subject Sample

The present invention features methods including, acquiring a subject sample. The terms “subject sample” and “sample” are used interchangeably herein. The subject sample can be a tissue, or bodily fluid, or bodily product. Tissue samples can include fixed, paraffin embedded, fresh, or frozen samples. For example, the tissue sample can include a biopsy, cheek swab, fine needle aspirates, large core needle biopsy, or directional vacuum assisted biopsy. Exemplary tissues include breast, brain, lung, pancreas, colon, prostate, lymph node, skin, hair follicles and nails. The tissue sample can also include a blood sample in which circulating tumor cells have been captured or isolated. Exemplary bodily fluids include blood, plasma, urine, lymph, tears, sweat, saliva, semen, and cerebrospinal fluid. Exemplary bodily products include exhaled breath.

The sample tissue, fluid, or product can be analyzed for the level of gene expression of a gene or a plurality of genes. The sample tissue, fluid or product can be analyzed for the level of gene expression of a gene or plurality of genes of a preselected signaling pathway or phenotypic pathway, e.g., a cancer stem cell phenotype, cancer associated mesenchymal cell phenotype, tumor initiating cancer cell phenotype, the epithelial to mesenchymal transition pathway, the Wnt signaling pathway, Notch pathway, or the TGFbeta signaling pathway. The sample tissue, fluid or product can be analyzed for the level of gene expression of a combination of genes from a plurality of preselected signaling or phenotypic pathways.

The tissue, fluid or product can be removed from the patient and analyzed. The evaluation can include one or more of: performing the analysis of the tissue, fluid or product; requesting analysis of the tissue fluid or product; requesting results from analysis of the tissue, fluid or product; or receiving the results from analysis of the tissue, fluid or product.

Acquisition of a Value or Values that is a Function of the Level of Gene Expression

The present invention features methods including, acquiring a value or values that is a function of the level of gene expression of a plurality of genes in a subject sample. The acquired value or values can be a function of a comparison with a reference criterion. The value or values can also be a function of the determination of whether the level of gene expression has a preselected relationship with a reference criterion (e.g., comparing the level of gene expression, with a preselected reference criterion). The reference criterion, as used herein, refers to a characteristic forming the basis of comparison for the evaluation or assessment of a measured characteristic. The preselected reference criterion can include the level of gene expression of a reference gene or the level of gene expression of a group of reference genes (e.g., housekeeping genes). The preselected reference criterion can include the level of gene expression of a gene from a control sample, e.g., a non-cancer sample. The appropriate reference criterion will depend on the gene or genes of which the level of expression is being acquired and the sample from which the level of gene expression of the genes was acquired from, and can be determined by one skilled in the art.

At least one or both of, acquiring a value or values that is the function of the level of gene expression, and determining if the level of gene expression has a preselected relationship with a reference criterion; can include one or more of: analyzing the sample, requesting analysis of the sample, requesting results from analysis of the sample, or receiving the results from analysis of the sample. Generally, analysis can include one or both of performing the underlying method (e.g., analysis of the level of gene expression) or receiving data from another who has performed the underlying method.

The acquired value or values can also be a function of a weighting factor. A weighting factor as used herein, refers to an element used to give an adjustment factor to a value. The weighting factor can be a composite weighting factor for a group of genes. For example, a first value or values that is a function of the level of gene expression of a plurality of genes can be a function of a weighting factor. The weighting factor can also be a specific weighting factor for a specific gene that only applies to that specific gene. For example, a first value or values that is a function of the level of gene expression of a first gene can be a function of a weighting factor, and a second value or values that is a function of the level of gene expression of a second gene can be a function of a second weighting factor; the first and the second weighting factor can be different.

### Level of Gene Expression

The present invention features methods of acquiring a value or values that is a function of the level of gene expression of a plurality of genes. To acquire the level of gene expression in a subject sample, the level of gene expression can be assayed, such as by measuring the level of RNA or protein product produced by the relevant gene. Thus the level of gene expression can be a function of the level of a RNA product produced by the relevant gene; or the level of gene expression can be a function of the level of a protein product produced by the relevant gene. The level of gene expression can also be a function of the protein or RNA activity level, which can be assayed by determining the protein (or RNA, e.g., mRNA) activity levels, e.g., transcriptional activation activity, catalytic activity, gene silencing activity, kinase activity, etc. The level of RNA expression can be assayed by a PCR based method. For example, mRNA can be isolated from a tissue sample, and subjected to qRT-PCR, and, optionally, Southern blot analysis, or gene chip or microarray analysis or some variant thereof. The subject sample, or the mRNA isolated from, or amplified from, the subject sample, can be applied to a nucleic acid microarray, or chip array. The level of RNA expression can also be measured by, for example, RNA in situ hybridization, quantitative RNA sequencing, or Northern blot. The level of protein product expressed by the relevant gene can be assayed by various antibody based techniques, including but not limited to Western blot, immunohistochemistry, and immunoassays, e.g. ELISA. The levels of gene expression, e.g., level of RNA expression of the relevant gene, level of protein expression of the relevant gene; can be assayed by other molecular biology methods known to those skilled in the art.

Optionally, the level of gene expression data can be configured into a file, such as a data file, e.g., an image corresponding to the levels of gene expression. Optionally, the gene expression data can be stored in a tangible medium and/or transmitted to a second site. The evaluation of the data file or image can include one or more of, performing statistical data analysis or imaging analysis, requesting statistical data analysis or imaging analysis, requesting results from statistical data analysis or imaging analysis, or receiving the results from data statistical analysis or imaging analysis.

### Location Specific Acquisition of the Level of Gene Expression

The present invention features methods which include the acquisition of a value or values for locations in the subject sample. The value or values can be a function of the level of gene expression of a gene or plurality of genes at the location. This can include the acquisition of a first value or values for a first location in the subject sample, and a second value or values for a second location in the subject sample, in which the value or values are a function of the level of gene expression of a gene or plurality of genes at the location. The term, “location”, as used herein, refers to a zone of a sample defined by preselected criteria, such as morphology, histopathology, and other attributes. A zone of a tumor can be defined by a unique gene expression pattern of a set of preselected genes. A zone may be classified as containing specific cell type or multiple cell types, e.g., a zone may be classified as a nodule of cancer stem cells, a nodule of cancer associated mesenchymal cells, a nodule of tumor initiating cancer cells; a zone of transition, e.g., an area between epithelial and mesenchymal features of a tumor region; or a boundary between tumor regions of different types; or it may be a niche indicated by the presence of a particular cell type or class, e.g., mesenchymal cells, stromal cells, inflammatory cells, endothelial cells, cancer stem cells, cancer associated mesenchymal cells, tumor initiating cancer cells, etc.

The level of gene expression at a location can be measured by RNA in situ hybridization and/or antibody based immunohistochemistry techniques. These techniques also allow for the association of the levels of gene expression with specific cell types in a zone or region through further definition or identification of the cells. The definition or identification of these cells can be assayed using computational overlays of the cells with specific gene markers of interest, or for adjoining cells. For example, an overlay may be achieved by evaluation of serial sections of formalin-fixed or frozen tumor tissues that are sectioned 3-5 microns in thickness. Adjoining sections may be evaluated with different probes, and computational methods applied to condense into a single image file with pseudocoloring representative of the different probes. Alternatively, probes that may be identified in different wavelength channels may be used together. The definition or identification of these cells can be determined by assaying the level of expression of gene markers of interest; or assaying the level of expression of gene markers of interest in adjoining cells. The definition or identification of the cells can also be assayed by histopathology criteria, e.g., cell shape, cell size, shape of cell, nucleus shape, nucleus size, and nuclei morphology, e.g., fuzzy nuclei.

The location in the subject sample can be defined, for example, as a distance from a morphological region of the subject sample, e.g., distance from an endothelial cell or blood vessel. The location can be the whole subject sample, e.g., a tumor sample. A first location can be the whole subject sample; with subsequent acquisition of the level of gene expression of a subset of genes that define a specific zone, e.g., zones defined by biological criteria, such as detection of genes associated with a specific identity, e.g., cancer stem cell, EMT, vasculature, etc.

The acquired value or values of each location can be a function of a comparison with a reference criterion. The value or values can be a function of the level of expression of a single gene at the location or a function of a combination of the level of gene expression of multiple genes at the location. For example, the level of gene expression of a group of genes can be measured with a uniform technique so that the collective expression of a set of genes together is acquired. For example, RNA in situ hybridization techniques can be used in which probe sets are used for two or more genes of interest that may be combined for analysis of subject samples.

The acquired value or values can be a function of a comparison with a reference criterion. The value or values can also be a function of the determination of whether the level of gene expression has a preselected relationship with a reference criterion (e.g., comparing the level of gene expression, with a preselected reference criterion). The reference criterion, as used herein, refers to a characteristic forming the basis of comparison for the evaluation or assessment of measured characteristic. The preselected reference criterion can include the level of gene expression of a reference gene or the level of gene expression of a group of reference genes (e.g., housekeeping genes). The preselected reference criterion can include the level of gene expression of a gene from a control sample, e.g., a non-cancer sample. The determination of whether the level of gene expression has a preselected relationship with a reference criterion can also include comparing the acquired value or values of a first location with the acquired value or values of a second location.

At least one or both of acquiring a value or values that is the function of the level of gene expression at a first and/or second location, and determining if the level of gene expression has a preselected relationship with a reference criterion, can include one or more of the following: analyzing the sample; requesting analysis of the sample; requesting results from analysis of the sample; or receiving the results from analysis of the sample. Generally, analysis can include one or both of performing the underlying method (e.g., analysis of the level of gene expression) or receiving data from another who has performed the underlying method.

The value or values of a first location can be associated with a higher or lower likelihood of being a cancer stem cell, cancer associated mesenchymal cell, or tumor initiating cancer cell, than a second value or values of a second location. The value or values of a first location can be associated with a higher or lower likelihood of being a cancer stem cell than a second value or values of a second location. The value or values of a first location can be associated with a higher or lower likelihood of being a cancer associated mesenchymal cell than a second value or values of a second location. The value or values of a first location can be associated with a higher or lower likelihood of being a tumor initiating cancer cell than a second value or values of a second location. Responsive to the acquisition of the value or values acquired for each of a plurality of locations, each location can be classified as being indicative of a cancer stem cell or non-cancer stem cell. For example, a location indicative of a cancer stem cell or a tumor initiating cancer cell can exhibit a high level of CD44 gene expression (CD44(high)) and a concurrent low level of CD24 gene expression (CD24(low)) compared to a reference criterion; an increased level of gene expression compared to a reference criterion of an EMT (epithelial to mesenchymal transition) transcription factor, e.g., ZEB1, Twist, FoxC2; a decreased level of gene expression compared to a reference criterion of tight junction and adhesion genes, e.g., Claudin1-7, E-cadherin; an increased level of gene expression of mesenchymal adhesion proteins, e.g., N-cadherin. Responsive to the acquisition of the value or values acquired for each of a plurality of locations, each location can be classified as a cancer stem cell or non-cancer stem cell. Each location can also be classified as a cancer stem cell, a cancer associated mesenchymal cell, or a tumor initiating cancer cell.

Where the value or values of a location are a function of the level of gene expression of multiple genes, the value or values can be indicative of a cancer stem cell, cancer associated mesenchymal cell, or tumor initiating cancer cell. For example, the level of gene expression of a set of genes can be measured with a uniform technique as described above so that the collective level of expression of the genes identify cancer stem cells, cancer associated mesenchymal cells, or tumor initiating cancer cells. Where the value or values of a location are a function of the level of gene expression of multiple genes, the value or values can be indicative of a cancer stem cell. For example, the level of gene expression of a set of genes can be measured with a uniform technique as described above so that the collective level of expression of the genes identifies cancer stem cells. Where the value or values of a location are a function of the level of gene expression of multiple genes, the value or values can be indicative of a cancer associated mesenchymal cell. For example, the level of gene expression of a set of genes can be measured with a uniform technique as described above so that the collective level of expression of the genes identifies cancer associated mesenchymal cells. Where the value or values of a location are a function of the level of gene expression of multiple genes, the value or values can be indicative of a tumor initiating cancer cell. For example, the level of gene expression of a set of genes can be measured with a uniform technique as described above so that the collective level of expression of the genes identifies tumor initiating cancer cells.

The locations can be separated by no distance, i.e., adjoining locations, in the subject sample or separated by range of distances; up to the maximum distance allowed by the sample size. For example, the locations can be separated by zero microns, ten microns, twenty microns, thirty microns, forty microns, fifty microns, sixty microns, seventy microns, eighty microns, ninety microns, one hundred microns, one hundred and fifty microns, two hundred microns, or three hundred microns; the locations can be separated by more than zero microns, more than ten microns, more than twenty microns, more than thirty microns, more than forty microns, more than fifty microns, more than sixty microns, more than seventy microns, more than eighty microns, more than ninety microns, more than one hundred microns, more than one hundred and fifty microns, more than two hundred microns, or more than three hundred microns; separated by at least one micron but not over one hundred microns; separated by at least fifty microns but not over one hundred microns; separated by at least one hundred microns; separated by at least one hundred microns but not more than two hundred microns; separated by at least two hundred microns but not more than three hundred microns; separated by at least three hundred microns; separated by at least four hundred microns; separated by at least five hundred microns; separated by at least six hundred microns, separated by at least seven hundred microns, separated by at least eight hundred microns, separated by at least nine hundred microns; separated by at least one thousand microns; separated by a distance over one thousand microns; separated by a distance under one thousand microns. The distance between locations can be any distance between zero and the maximum distance two locations can be separated based on the size of the sample, including zero and the maximum distance two locations can be separated based on the size of the sample.

The average distance between the locations can be zero microns; ten microns; twenty microns; thirty microns; forty micron; fifty microns; sixty microns; seventy microns; eighty microns; ninety microns; or one hundred microns. The average distance between the locations can be more than zero microns; more than ten microns; more than twenty microns; more than thirty microns; more than forty micron; more than fifty microns; more than sixty microns; more than seventy microns; more than eighty microns; more than ninety microns; or more than one hundred microns. The average distance between the locations can be more than one thousand microns. The average distance between the locations can be more than one hundred microns; more than 200 hundred microns; more than three hundred microns; more than four hundred microns; more than five hundred microns, or more than one thousand microns. The average distance between locations can be any distance between zero and the maximum distance two locations can be separated based on the size of the sample, including zero and the maximum distance two locations can be separated based on the size of the sample.

### Gene Set Score

The present invention features methods of acquiring a gene set score. The gene set score can be a function of the level of gene expression of a plurality of genes. The level of gene expression can be acquired as described above. The gene set score can further be a function of the level expression of a gene isoform. The level of a gene isoform can be acquired as described above. The gene set score can be a function of both the level of gene expression and the level of expression of a gene isoform. The gene set score can be a function of both the level of gene expression and the level of expression of a plurality of gene isoforms of a gene. The gene set score can be a function of both the level of gene expression of a gene or plurality of genes; and the level of expression of a gene isoform of a gene. The gene set score can be a function of the level of gene expression of a gene or plurality of genes; and the level of expression of each gene isoform of a plurality of gene isoforms of a gene. The gene set score can be a function of both the level of gene expression of a gene or plurality of genes; and the level of expression of a plurality of gene isoforms of a gene. The set gene score can be a function of both the level of gene expression of a gene or plurality of genes; and the level of expression of a plurality of gene isoforms of a plurality of genes. The gene set score can be a function of both the level of gene expression of a gene or plurality of genes; and the level of expression of each gene isoform of a plurality of gene isoforms of a plurality of genes.

The gene set score can be acquired by mathematical computation. The gene set score can be computed using the following algorithm:

$${S}_{sig\_X} = \frac{1}{N} {\sum }_{i = 1}^{N} ( {e}_{i} - {\overline{e}}_{i} )$$

### Where:

S_{sig}_{_{—}}_{X}=the score for a subset of the genes in the signature gene set (i.e., S_{sig}_{_{—}}_{UP} or S_{sig}_{_{—}}_{DN})

N=number of genes in the gene set

eᵢ=the log 2 expression level of gene i in the gene set

ēᵢ=the mean log 2 expression level of gene i over all samples in the sample set Gene set score:

S_{sig}=S_{sig}_{_{—}}_{UP}−S_{sig}_{_{—}}_{DN}

### Where:

S_{sig}_{_{—}}_{UP}=gene set score over upregulated genes in the signature

S_{sig}_{_{—}}_{DN}=gene set score over downregulated genes in the signature.

### Genotype

The present invention features methods that include the acquisition of a genotype of the subject sample. The subject sample can be any sample type described herein, e.g., a tissue sample, bodily fluid, or bodily product. The genotype can be directly acquired or indirectly acquired. The genotype can be directly acquired through assaying. The genotype can be assayed using a sequencing based method. “Sequencing” a nucleic acid molecule as used herein, requires determining the identity of at least one nucleotide in the molecule. The identity of less than all of the nucleotides in a molecule can be determined. The identity of a majority or all of the nucleotides in the molecule can be determined. The genotype can be assayed using a sequencing based method, e.g., SNP (single nucleotide polymorphism) analysis, PCR based method, restriction fragment length polymorphism, terminal restriction fragment length polymorphism, amplified restriction fragment length polymorphism, multiplex restriction fragment length polymorphism, or other sequencing and molecular biology techniques known to those skilled in the art.

In genotyping, genetic events associated with cancer can be assayed. For example, nucleotides of the sample can be sequenced to determine the presence or absence of a genetic event associated with cancer; an oncogene or oncogenes and/or tumor suppressor genes can be sequenced, e.g., Abl, Af4/hrx, akt-2, alk, alk/npm, aml 1, aml 1/mtg8, APC, axl, bcl-2, bcl-3, bcl-6, bcr/abl, brca-1, brca-2, beta-catenin, CDKN2, c-myc, c-sis, dbl, dek/can, E2A/pbx1, egfr, enl/hrx, erg/TLS, erbB, erbB-2, erk, ets-1, ews/fli-1, fms, fos, fps, gli, gsp, HER2/neu, hox11, hst, IL-3, int-2, jun, kit, KS3, K-sam, Lbc, lck, lmo1, lmo2, L-myc, li1-1, lyt-10, lyt-10/C alpha1, mas, mdm-2, mll, mos, mtg8/aml1, myb, myc, MYH11/CBFB, neu, nm23, N-myc, ost, p53, pax-5, pbx1/E2A, pdgfr, PI3-K, pim-1, PRAD-1, raf, RAR/PML, rash, rasK, rasN, Rb, rel/nrg, ret, rhom1, rhom2, ros, ski, sis, set/can, src, tal1, tal2, tan-1, telomerase, Tiam1, TSC2, trk, vegfr, or wnt.

### Classification

The present invention features methods including, classifying the subject, e.g., classifying the subject as a candidate or a non-candidate for treatment with a preselected drug, e.g., an agent that inhibits or kills cancer associated mesenchymal cells, tumor initiating cancer cells, or cancer stem cells. As used herein, a subject who is a “candidate” is a one more likely to respond to a particular therapeutic regimen, relative to a reference subject or group of subjects. A “non-candidate” subject is one not more likely to respond to a particular therapeutic regimen, relative to a reference subject or group of subjects. The preselected drug can include but is not limited to, an agent that inhibits or kills cancer associated mesenchymal cells, tumor initiating cancer cells, or cancer stem cells; which can include but is not limited to, e.g., salinomycin; a gamma secretase inhibitor; a DLL4 inhibitor, e.g., a therapeutic antibody targeting DLL4; a TRAIL inhibitor, e.g., a therapeutic antibody targeting TRAIL; a Hedgehog inhibitor, e.g., a therapeutic antibody targeting Hedgehog; a NOTCH3 inhibitor, e.g., a therapeutic antibody targeting NOTCH3; a NOTCH4 inhibitor, e.g., a therapeutic antibody targeting NOTCH4; a panNOTCH inhibitor, e.g., a therapeutic antibody targeting panNOTCH; a FGFR1 inhibitor, e.g., a therapeutic antibody targeting FGR1; a FGFR2 inhibitor, e.g., a therapeutic antibody targeting FGR2; a FGFR3 inhibitor, e.g., a therapeutic antibody targeting FGR3; a FGFR4 inhibitor, e.g., a therapeutic antibody targeting FGR4; a RON inhibitor, e.g., a therapeutic antibody targeting RON; Wnt pathway inhibitor, e.g., therapeutic antibodies targeting the Wnt pathway; a PI3Kinase inhibitor; a mTOR inhibitor; sodium meta arsenite; verapail; reserpine; a perifosen inhibitor of FAK1; a FAK inhibitor; a p38 inhibitor. Classification as a candidate subject can also reflect an increased likelihood the subject will respond positively to treatment with an agent that inhibits or kills cancer associated mesenchymal cells, tumor initiating cancer cells, or cancer stem cells.

### Administration

The present invention features methods including, administering a treatment comprising an agent that inhibits or kills cancer associated mesenchymal cells, tumor initiating cancer cells, or cancer stem cells to the subject. The invention can further include selecting a regimen, e.g., dosage, formulation, route of administration, number of dosages, or adjunctive or combination therapies of an agent that inhibits or kills cancer associated mesenchymal cells, tumor initiating cancer cells, or cancer stem cells. The administration of an agent that inhibits or kills cancer associated mesenchymal cells, tumor initiating cancer cells, or cancer stem cells can be responsive to the acquisition of the value or values that is a function of the level of gene expression described herein, and/or classification of a subject as a candidate or non-candidate for treatment with an agent that inhibits or kills cancer associated mesenchymal cells, tumor initiating cancer cells, or cancer stem cells. The selection of the regimen can be responsive to the acquisition of the value or values that is a function of the level of gene expression described herein, and/or classification of a subject as a candidate or non-candidate for treatment with an agent that inhibits or kills cancer associated mesenchymal cells, tumor initiating cancer cells, or cancer stem cells. The invention can further include the administration of the selected regimen. The administration can be provided responsive to acquiring knowledge or information of the value or values that is a function of the level of gene expression described herein, from another party; receiving communication of the presence of the value or values that is a function of the level of gene expression in a subject; or responsive to the acquisition of the value or values that is a function of the level of gene expression in a subject, wherein the acquisition arises from collaboration with another party.

An agent that inhibits or kills cancer associated mesenchymal cells, tumor initiating cancer cells, or cancer stem cells, e.g., salinomycin; a gamma secretase inhibitor; a DLL4 inhibitor, e.g., a therapeutic antibody targeting DLL4; a TRAIL inhibitor, e.g., a therapeutic antibody targeting TRAIL; a Hedgehog inhibitor, e.g., a therapeutic antibody targeting Hedgehog; a NOTCH3 inhibitor, e.g., a therapeutic antibody targeting NOTCH3; a NOTCH4 inhibitor, e.g., a therapeutic antibody targeting NOTCH4; a panNOTCH inhibitor, e.g., a therapeutic antibody targeting panNOTCH; a FGFR1 inhibitor, e.g., a therapeutic antibody targeting FGR1; a FGFR2 inhibitor, e.g., a therapeutic antibody targeting FGR2; a FGFR3 inhibitor, e.g., a therapeutic antibody targeting FGR3; a FGFR4 inhibitor, e.g., a therapeutic antibody targeting FGR4; a RON inhibitor, e.g., a therapeutic antibody targeting RON; Wnt pathway inhibitor, e.g., therapeutic antibodies targeting the Wnt pathway; a PI3Kinase inhibitor; a mTOR inhibitor; sodium meta arsenite; verapail; reserpine; a perifosen inhibitor of FAK1; a FAK inhibitor; a p38 inhibitor; can be administered to a subject using any amount and any route of administration effective for treating cancer, or symptoms associated with cancer. The exact dosage required will vary from subject to subject, depending on subject specific factors, e.g., the age and general condition of the subject, concurrent treatments, concurrent diseases or conditions; cancer specific factors, e.g., the type of cancer, whether the cancer is recurrent, whether the cancer is metastatic, the severity of the disease; and agent specific factors., e.g., its composition, its mode of administration, its mode of activity, and the like. For example, the dosage may vary depending on whether the subject is currently receiving or had previously received a treatment regimen prior to the administration of an agent that inhibits or kills cancer associated mesenchymal cells, tumor initiating cancer cells, or cancer stem cells; whether the subject is a non-responder to such current or previous treatment; whether the subject's cancer is recurrent; or whether the subject's cancer has metastasized to a second tissue site.

The total daily usage of a therapeutic composition of an agent that inhibits or kills cancer associated mesenchymal cells, tumor initiating cancer cells, or cancer stem cells can be decided by an attending physician within the scope of sound medical judgment. The specific therapeutically effective, dose level for any particular subject will depend upon a variety of factors including the type of cancer being treated; the severity of the cancer; the metastatic state of the cancer; the recurrence state of the cancer; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

The agent that inhibits or kills cancer associated mesenchymal cells, tumor initiating cancer cells, or cancer stem cells may be administered by any route, including by those routes currently accepted and approved for known products. Exemplary routes of administration include, e.g., oral, intraventricular, transdermal, rectal, intravaginal, topical (e.g. by powders, ointments, creams, gels, lotions, and/or drops), mucosal, nasal, buccal, enteral, vitreal, sublingual; by intratracheal instillation, bronchial instillation, and/or inhalation; as an oral spray, nasal spray, and/or aerosol, and/or through a portal vein catheter. An agent may be administered in a way, which allows the agent to cross the blood-brain barrier, vascular barrier, or other epithelial barrier.

Other exemplary routes include administration by a parenteral mode (e.g., intravenous, subcutaneous, intraperitoneal, or intramuscular injection). The phrases “parenteral administration” and “administered parenterally” as used herein mean modes of administration other than enteral and topical administration, usually by injection, and include, without limitation, intravenous, intramuscular, intraarterial, intrathecal, intracapsular, intramedullary, intratumoral, intraorbital, intracardiac, intradermal, intraperitoneal, transtracheal, subcutaneous, subcuticular, intraarticular, subcapsular, subarachnoid, intraspinal, epidural and intrasternal injection and infusion.

Pharmaceutical compositions can be formulated in a variety of different forms, such as liquid, semi-solid and solid dosage forms, such as liquid solutions (e.g., injectable and infusible solutions), dispersions or suspensions, tablets, pills, powders, liposomes and suppositories. The preferred form can depend on the intended mode of administration and therapeutic application. A pharmaceutical composition comprising an agent that inhibits or kills cancer associated mesenchymal cells, tumor initiating cancer cells, or cancer stem cells may be administered on various dosing schedules. The dosing schedule will be dependent on several factors including, the type of cancer being treated; the severity of the cancer; the metastatic state of the cancer; the recurrence state of the cancer; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

Exemplary dosing schedules of an agent that inhibits or kills cancer associated mesenchymal cells, tumor initiating cancer cells, or cancer stem cells composition include, once daily, or once weekly, or once monthly, or once every other month. The composition can be administered twice per week or twice per month, or once every two, three or four weeks. The composition can be administered as two, three, or more sub-doses at appropriate intervals throughout the day or even using continuous infusion or delivery through a controlled release formulation. In that case, the therapeutic agent contained in each sub-dose may be correspondingly smaller in order to achieve the total daily dosage. The dosage can also be compounded for delivery over several days, e.g., using a conventional sustained release formulation, which provides sustained release of the agent over a several day period. Sustained release formulations are well known in the art and are particularly useful for delivery of agents at a particular site.

The present invention features methods in which a value or values that is a function of the level of gene expression for (each of) a plurality of genes can be acquired at the time of or after diagnosis of cancer in a subject. The acquisition of the value or values that is a function of the level of gene expression can be acquired at a predetermined interval, e.g., a first point in time and at least at a subsequent point in time. The cancer can include cancers characterized as comprising cancer stem cells, cancer associated mesenchymal cells, or tumor initiating cancer cells. The cancer can include cancers that have been characterized as being enriched with cancer stem cells, cancer associated mesenchymal cells, or tumor initiating cancer cells. Exemplary cancers include epithelial cancers, breast, lung, pancreatic, colorectal, prostate, head and neck, melanoma, acute myelogenous leukemia, and glioblastoma. Exemplary breast cancers include triple negative breast cancer, basal-like breast cancer, claudin-low breast cancer, invasive, inflammatory, metaplastic, and advanced Her-2 positive or ER-positive cancers resistant to therapy. Other cancers include but are not limited to, brain, abdominal, esophagus, gastrointestinal, glioma, liver, tongue, neuroblastoma, osteosarcoma, ovarian, retinoblastoma, Wilm's tumor, multiple myeloma, skin, lymphoma, blood, retinal, acute lymphoblastic leukemia, bladder, cervical, kidney, endometrial, meningioma, lymphoma, skin, uterine, lung, non small cell lung, nasopharyngeal carcinoma, neuroblastoma, solid tumor, hematologic malignancy, leukemia, squamous cell carcinoma, testicular, thyroid, mesothelioma, brain vulval, sarcoma, intestine, oral, T cell leukemia, endocrine, salivary, spermatocytic seminoma, sporadic medulalry thyroid carcinoma, non-proliferating testes cells, cancers related to malignant mast cells, non-Hodgkin's lymphoma, and diffuse large B cell lymphoma.

The cancer can be a primary tumor, i.e., located at the anatomical site of tumor growth initiation. The cancer can also be metastatic, i.e., appearing at least a second anatomical site other than the anatomical site of tumor growth initiation. The cancer can be a recurrent cancer, i.e., cancer that returns following treatment, and after a period of time in which the cancer was undetectable. The recurrent cancer can be anatomically located locally to the original tumor, e.g., anatomically near the original tumor; regionally to the original tumor, e.g., in a lymph node located near the original tumor; or distantly to the original tumor, e.g., anatomically in a region remote from the original tumor.

The acquisition of a value or values that is a function of the level of gene expression described herein, can be acquired prior to, during, or after administration of a treatment to a subject. The treatment can include an agent that inhibits or kills cancer associated mesenchymal cells, tumor initiating cancer cells, or cancer stem cells therapy. The treatment can include a chemotherapeutic agent, antiemetic, analgesic, or anti-inflammatory agent. Suitable chemotherapeutic agents are any chemical substances or compounds, such as cytotoxic or cytostatic agent, that is used to treat a condition, particularly cancer, including, but not limited to: alkylating agents (e.g., nitrogen mustards such as chlorambucil, cyclophosphamide, isofamide, mechlorethamine, melphalan, and uracil mustard; aziridines such as thiotepa; methanesulphonate esters such as busulfan; nitroso ureas such as carmustine, lomustine, and streptozocin; platinum complexes such as cisplatin and carboplatin; bioreductive alkylators such as mitomycin, procarbazine, dacarbazine and altretamine); DNA strand-breakage agents (e.g., bleomycin); topoisomerase II inhibitors (e.g., amsacrine, dactinomycin, daunorubicin, idarubicin, mitoxantrone, doxorubicin, etoposide, and teniposide); DNA minor groove binding agents (e.g., plicamydin); antimetabolites (e.g., folate antagonists such as methotrexate and trimetrexate; pyrimidine antagonists such as fluorouracil, fluorodeoxyuridine, CB3717, azacitidine, cytarabine, and floxuridine; purine antagonists such as mercaptopurine, 6-thioguanine, fludarabine, pentostatin; asparginase; and ribonucleotide reductase inhibitors such as hydroxyurea); tubulin interactive agents (e.g., vincristine, vinblastine, and paclitaxel (Taxol)); hormonal agents (e.g., estrogens; conjugated estrogens; ethinyl estradiol; diethylstilbesterol; chlortrianisen; idenestrol; progestins such as hydroxyprogesterone caproate, medroxyprogesterone, and megestrol; and androgens such as testosterone, testosterone propionate, fluoxymesterone, and methyltestosterone); adrenal corticosteroids (e.g., prednisone, dexamethasone, methylprednisolone, and prednisolone); leutinizing hormone releasing agents or gonadotropin-releasing hormone antagonists (e.g., leuprolide acetate and goserelin acetate); and antihormonal antigens (e.g., tamoxifen, antiandrogen agents such as flutamide; and antiadrenal agents such as mitotane and aminoglutethimide). Exemplary chemotherapeutic agents include, Capecitabine, Carboplatin, Cisplatin, Cyclophosphamide, Docetaxel, Doxorubicin, Epirubicin, Eribulin, mesylate5-Fluorouracil, Gemcitabine, Ixabepilone, Liposomal doxorubicin, Methotrexate, Paclitaxel, or Vinorelbine; or any combination thereof.

The subject can be a responder or non-responder to the current or prior treatment. The agent that inhibits or kills cancer associated mesenchymal cells, tumor initiating cancer cells, or cancer stem cells; can be administered as an additional therapeutic agent, e.g., an agent that inhibits or kills cancer associated mesenchymal cells, tumor initiating cancer cells, or cancer stem cells in addition to a current therapeutic regimen, or in addition to a new therapeutic regimen. The current treatment of the subject can be stopped and replaced with treatment an agent that inhibits or kills cancer associated mesenchymal cells, tumor initiating cancer cells, or cancer stem cells. The current treatment regimen can also be altered with the addition of an agent that inhibits or kills cancer associated mesenchymal cells, tumor initiating cancer cells, or cancer stem cells as an additional therapeutic agent. Therapeutic agents administered in combination with an agent that inhibits or kills cancer associated mesenchymal cells, tumor initiating cancer cells, or cancer stem cells; can kill or inhibit the growth of non-cancer stem cells, non-cancer associated mesenchymal cells, or non-tumor initiating cells in the subject.

### Kits or Products

The present invention features a kit or product that includes a means to assay the level of gene expression of a gene or plurality of genes in Table 1. For example, the kit or product can include an agent capable of interacting with a gene expression product of a gene from the genes in Table 1; and can further contain a second agent capable of interacting with a different gene expression product from a gene in Table 1. The kit can contain a plurality of different agents capable of interacting with a plurality of genes expression products from a gene in Table 1. The kit can contain a plurality of different agents capable of interacting with a plurality of genes expression products from a plurality of genes in Table 1. The agent can include, but is not limited to, an antibody, a plurality of antibodies, an oligonucleotide, or a plurality of oligonucleotides. The kit or product can further comprise an agent capable of interacting with a gene expression product of a gene not in Table 1. The kit or product can contain a plurality of agents capable of interacting with a plurality of gene expression product of a plurality of genes not in Table 1. The gene expression product can include, but is not limited to, a RNA product of the associated gene, or a protein product of the associated gene.

The kit or product can further optionally include reagents for performing the level of gene expression assays described herein. For example, the kit can include buffers, solvents, stabilizers, preservatives, purification columns, detection reagents, and enzymes, which may be necessary for isolating nucleic acids from a subject sample, amplifying the samples, e.g., by qRT-PCR, and applying the samples to the agent described above; or for isolating proteins from a subject sample, and applying the samples to the agent described above; or reagents for directly applying the subject sample to the agent described above. A kit can also include positive and negative control samples, e.g., control nucleic acid samples (e.g., nucleic acid sample from a non-cancer subject, or a non-tumor tissue sample, or a subject who has not received treatment for cancer, or other test samples for testing at the same time as subject samples. A kit can also include instructional material, which may provide guidance for collecting and processing patient samples, applying the samples to the level of gene expression assay, and for interpreting assay results.

The components of the kit can be provided in any form, e.g., liquid, dried, semi-dried, or in lyophilized form, or in a form for storage in a frozen condition. Typically, the components of the kit are provided in a form that is sterile. When reagents are provided in a liquid solution, the liquid solution generally is an aqueous solution, e.g., a sterile aqueous solution. When reagents are provided in a dried form, reconstitution generally is accomplished by the addition of a suitable solvent. The solvent, e.g., sterile buffer, can optionally be provided in the kit.

The kit can include one or more containers for the kit components in a concentration suitable for use in the level of gene expression assays or with instructions for dilution for use in the assay. The kit can contain separate containers, dividers or compartments for the assay components, and the informational material. For example, the positive and negative control samples can be contained in a bottle or vial, the clinically compatible classifier can be sealed in a sterile plastic wrapping, and the informational material can be contained in a plastic sleeve or packet. The kit can include a plurality (e.g., a pack) of individual containers, each containing one or more unit forms (e.g., for use with one assay) of an agent. The containers of the kits can be air tight and/or waterproof. The container can be labeled for use.

The kit can include informational material for performing and interpreting the assay. The kit can also provide guidance as to where to report the results of the assay, e.g., to a treatment center or healthcare provider. The kit can include forms for reporting the results of a gene activity assay described herein, and address and contact information regarding where to send such forms or other related information; or a URL (Uniform Resource Locator) address for reporting the results in an online database or an online application (e.g., an app). In another embodiment, the informational material can include guidance regarding whether a patient should receive treatment with an ant-cancer stem cell agent, depending on the results of the assay.

The informational material of the kits is not limited in its form. In many cases, the informational material, e.g., instructions, is provided in printed matter, e.g., a printed text, drawing, and/or photograph, e.g., a label or printed sheet. However, the informational material can also be provided in other formats, such as computer readable material, video recording, or audio recording. The informational material of the kit can be contact information, e.g., a physical address, email address, website, or telephone number, where a user of the kit can obtain substantive information about the gene activity assay and/or its use in the methods described herein. The informational material can also be provided in any combination of formats.

A subject sample can be provided to an assay provider, e.g., a service provider (such as a third party facility) or a healthcare provider that evaluates the sample in an assay and provides a read out. For example, an assay provider can receive a sample from a subject, such as a tissue sample, or a plasma, blood or serum sample, and evaluate the sample using an assay described herein, and determines that the subject is a candidate to receive an agent that inhibits or kills cancer associated mesenchymal cells, tumor initiating cancer cells, or cancer stem cells.

The assay provider can inform a healthcare provider that the subject is a candidate for treatment with an agent that inhibits or kills cancer associated mesenchymal cells, tumor initiating cancer cells, or cancer stem cells, and the candidate is administered the agent that inhibits or kills cancer associated mesenchymal cells, tumor initiating cancer cells, or cancer stem cells. The assay provider can provide the results of the evaluation, and optionally, conclusions regarding one or more of diagnosis, prognosis, or appropriate therapy options to, for example, a healthcare provider, or patient, or an insurance company, in any suitable format, such as by mail or electronically, or through an online database. The information collected and provided by the assay provider can be stored in a database.

### Reports

The present invention features optionally providing a report. The report can include a prediction of the likelihood that a subject will respond positively or will not respond positively to treatment with an agent that inhibits or kills cancer associated mesenchymal cells, tumor initiating cancer cells, or cancer stem cells, e.g., salinomycin; a gamma secretase inhibitor; a DLL4 inhibitor, e.g., a therapeutic antibody targeting DLL4; a TRAIL inhibitor, e.g., a therapeutic antibody targeting TRAIL; a Hedgehog inhibitor, e.g., a therapeutic antibody targeting Hedgehog; a NOTCH3 inhibitor, e.g., a therapeutic antibody targeting NOTCH3; a NOTCH4 inhibitor, e.g., a therapeutic antibody targeting NOTCH4; a panNOTCH inhibitor, e.g., a therapeutic antibody targeting panNOTCH; a FGFR1 inhibitor, e.g., a therapeutic antibody targeting FGR1; a FGFR2 inhibitor, e.g., a therapeutic antibody targeting FGR2; a FGFR3 inhibitor, e.g., a therapeutic antibody targeting FGR3; a FGFR4 inhibitor, e.g., a therapeutic antibody targeting FGR4; a RON inhibitor, e.g., a therapeutic antibody targeting RON; Wnt pathway inhibitor, e.g., therapeutic antibodies targeting the Wnt pathway; a PI3Kinase inhibitor; a mTOR inhibitor; sodium meta arsenite; verapail; reserpine; a perifosen inhibitor of FAK1; a FAK inhibitor; a p38 inhibitor. The report can include a prediction of the likelihood a subject will respond positively or not to treatment with an agent that inhibits or kills cancer associated mesenchymal cells, tumor initiating cancer cells, or cancer stem cells. The report can also include a proposal including any one of or combination of the following: whether a subject is a candidate for treatment with an agent that inhibits or kills cancer associated mesenchymal cells, tumor initiating cancer cells, or cancer stem cells; whether a subject should be treated with a preselected drug, e.g. an agent that inhibits or kills cancer associated mesenchymal cells, tumor initiating cancer cells, or cancer stem cells; or whether treatment with a preselected drug, e.g., an agent that inhibits or kills cancer associated mesenchymal cells, tumor initiating cancer cells, or cancer stem cells, should be withheld.

The report can be provided by an assay service provider (such as a third party facility) that evaluates the sample in an assay and provides a report, or a healthcare provider. In the former case, the assay service provider can inform a healthcare provider that the subject is a candidate for treatment with an agent that inhibits or kills cancer associated mesenchymal cells, tumor initiating cancer cells, or cancer stem cells, and the candidate is administered the agent that inhibits or kills cancer associated mesenchymal cells, tumor initiating cancer cells, or cancer stem cells. The assay provider can provide the results of the evaluation, and optionally, conclusions regarding one or more of diagnosis, prognosis, or appropriate therapy options to, for example, a healthcare provider, or subject, or an insurance company, in any suitable format, such as by mail or electronically, or through an online database. The information collected and provided by the assay provider can be stored in a database. The report can be reported back to the healthcare provider, such as through a form, which can be submitted by mail or electronically (e.g., through facsimile or e-mail) or through an on-line database or on-line application (e.g., through an “app”). The results of the assay (including the level of gene expression) can be stored in a database and can be accessed by a healthcare provider, such as through the worldwide web.

### EXAMPLES

### Example 1

### Set of Genes Derived from Cancer Stem Cell and EMT Identifiers Found in More than One Statistical Measure for Gene Ranking

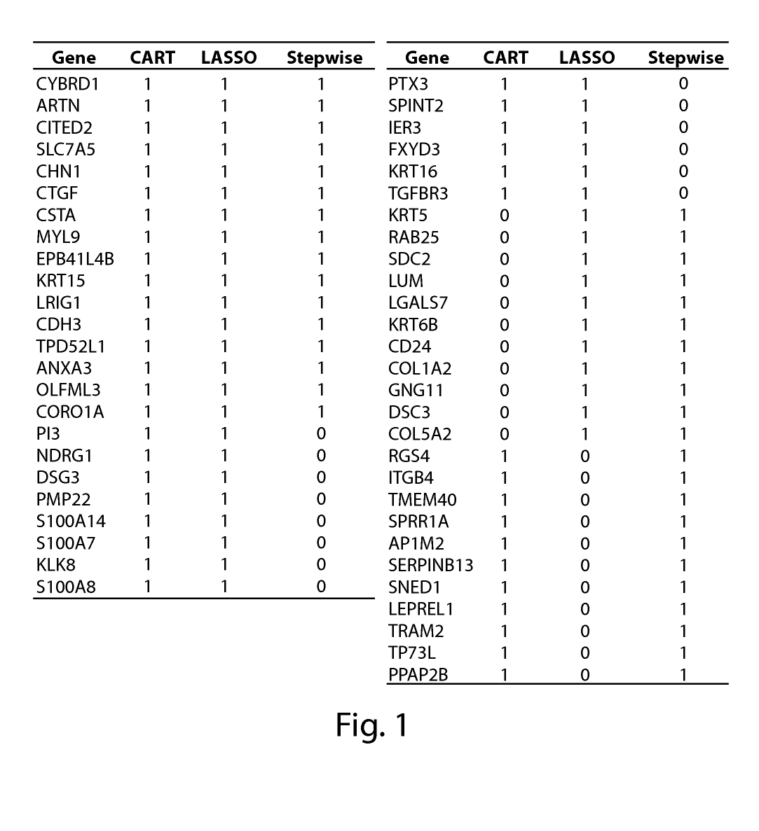

Three statistical processes of gene sampling methods were utilized to rank genes that contribute to tumor initiating cancer cells, cancer stem cells, and cancer associated mesenchymal cells (FIG. 1). The three partitioning and ranking methods used were LASSO [LASSO Variable Selection; Tibshirani, R. (1996)], Recursive Partitioning (or CART), and Stepwise selection. Patient samples used to train the models were classified as likely to recur using an arbitrary threshold as follows, Rscore <0.7 as ‘predicted recurrence’. In the LASSO method, the process is to fit outcomes using all genes, with the added restriction that the sum of model parameters must be less than an arbitrary threshold. While increasing the threshold in small steps and re-fitting the outcomes model; the determined gene ranking is the order that the genes model parameter exceeds threshold. In the Stepwise method, the process is to fit outcomes using all genes, and successively dropping the least significant gene from the model, and re-fitting the outcomes model until model performance does not improve. AIC criterion is used as a measure of fit, which balances the number of parameters against the improvement in performance. In the CART method, the process is to successively choose from the list of all genes, a single gene that splits samples into groups with the smallest impurity of outcomes. This process splits each subgroup using a single gene, until the minimum group size is reached, or the impurity of outcomes cannot be improved.

In the LASSO gene ranking method, there is a selection of the first 20 genes to enter a model using a 1e−10 threshold, and opening up using regression status. The next step involves fitting a logit model using all 20 genes, and repeating a logit fit using the top 10 genes, and a logit fit using restriction to increased statistical significance in additional cycles.

In the STEPWISE gene ranking method, sixty (60) genes are chosen randomly from a gene list. A model is fit that predicts outcome with all 60 genes using all samples. The least significant genes (highest AIC) are dropped from the set and refit until predictive ability of the model is affected (AIC increases). The final list of genes is kept and their statistical significance in the final model (p-values) is determined. The procedure is repeated 100 times to capture variety of starting genes with 60 ‘different’ or random genes. The ‘best’ genes are chosen based on average significance level within all models.

In the CART gene ranking method, 5000 iterations are performed, sampling from a patient dataset to be evaluated, that is equally split between patients having had recurrences or non-recurrences. Random sets of 20 genes from the analysis group are selected and fit to outcome in a decision tree composed of 5 samples per leaf. Trimming is undertaken to reveal the most important branches (cp=0.01). Between 1 and 5 genes are kept for next step (median of 3 genes). In the counting strategy, the more often a gene is included in a model is indicative of a higher statistical importance of that gene.

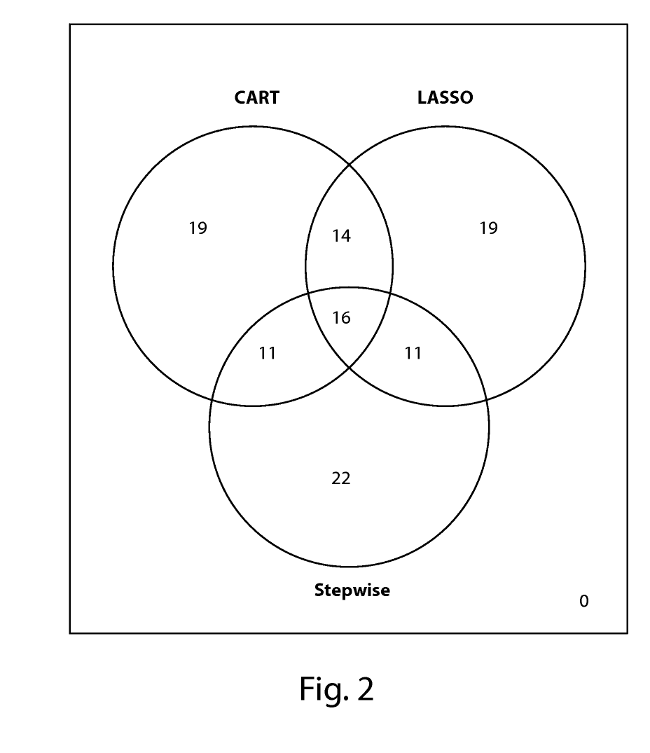

A group of 52 genes were identified that were highly ranked by two of the three methods. A concordance of the genes from these three methods was observed, where 16 genes were found by all of the methods (FIG. 1 and FIG. 2), such as the genes: CYBRD1, ARTN, CITED2, SLC7A5, CHN1, CTGF, CSTA, MYL9, EPB41L4B, KRT15, LRIG1, CDH3, TPD52L1, ANXA3, OLFML3, and CORO1A.

Triple negative breast cancer patients (n=178) were evaluated by standard histopathology criteria and determined to be of the triple negative breast cancer subtype by examining ER, PR, and HER2 by immunohistochemistry and pathology of tumor biopsy specimen. Following their diagnosis, patients were treated with standard chemotherapy and their survival was evaluated over the next seven years, so that outcome data was available for the patient cohort. In one test, gene subsets identified by the above rank ordering strategies were evaluated in breast cancer patient specimens with RNA expression data formed from profiling of patient tumors. In addition, recurrence and/or survival data from the patients was utilized.

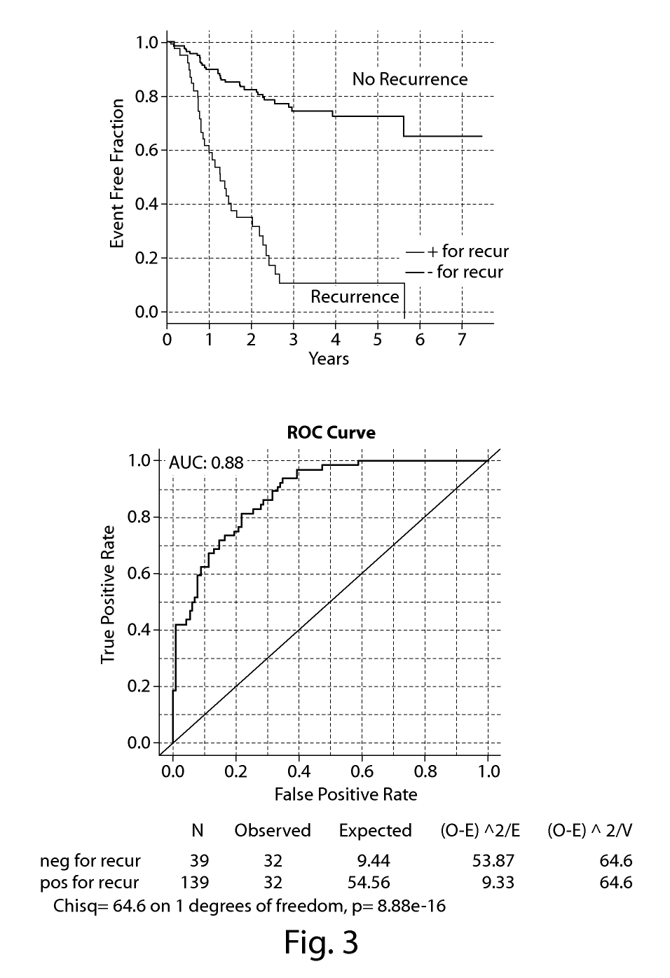

Using the top 20 genes of the LASSO method, namely genes: CYBRD1, ARTN, KRT15, ITGB4, RGS4, SPRR1B, SLC7A5, ANXA3, IL18, AP1M2, CHN1, OLFML3, ZBED2, LEPREL1, ABLIM1, CDH3, DNAJB4, TRAM2, UCHL1, and CTGF; analysis of patient and RNA data for these genes in Kaplan-Meier recurrence plots showed, the Event Free Recurrence subgroup of patients was significantly distinguished from the patient subset suffering recurrence by this 20-gene test (FIG. 3). In addition, the 20-gene model had a significant True Positive Rate over a False Positive Rate as exemplified by Area-Under the Curve (AUC) statistical analysis, with an AUC=0.88 (FIG. 3). The p-value separating the Event Free Recurrence subgroup from patients with Recurrence was determined to be p=8.88e−16. The top five ranked genes by this method displayed a separation of Even Free Recurrences with an AUC=0.73 and p=0.000423. Permutation analysis on LASSO method in this exercise was evaluated with a p=0.01 for 99.1 percentile for the top 20 gene model with regard to calculated p values, and a p=0.003 for 99.7 percentile for the top 20 gene model with regard to calculated AUCs.

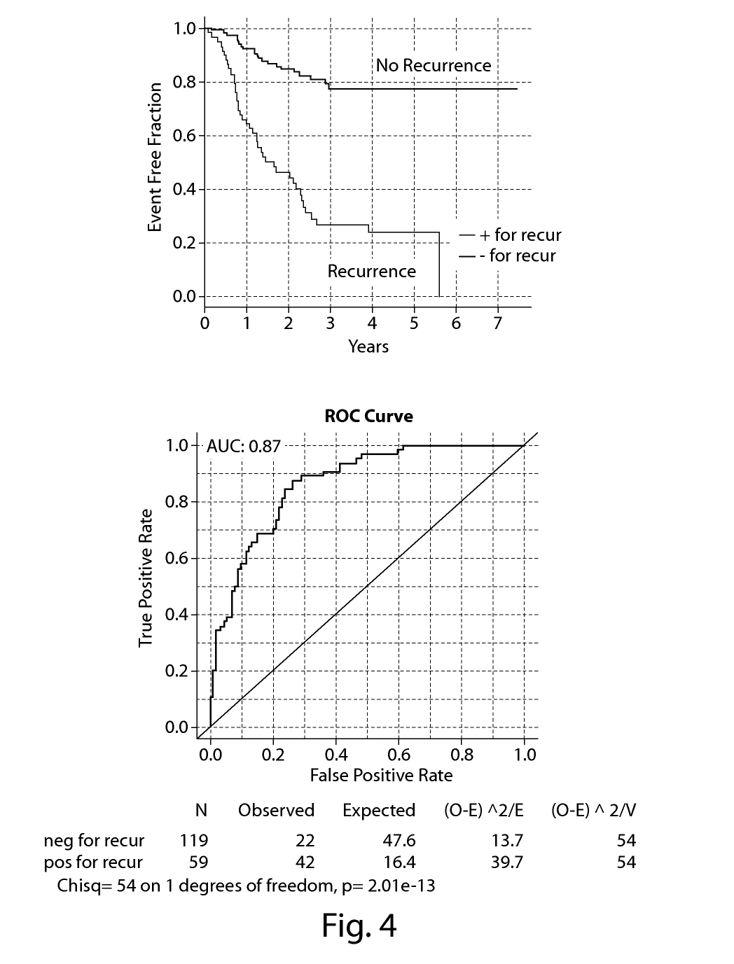

Using the top 20 genes of the STEPWISE method, namely genes: CYBRD1, ARTN, RGS4, CITED2, SLC7A5, ITGB4, CHN1, PI3, TMEM40, NDRG1, SPRR1A, PCDH9, CTGF, MAP1B, DSG3, AP1M2, FBLN5, SERPINB13, PMP22, and CSTA; analysis of patient and RNA data for these genes in Kaplan-Meier recurrence plots showed, the Event Free Recurrence subgroup of patients was significantly distinguished from the patient subset suffering recurrence by this 20-gene test, with a highly significant p-value distinguishing the groups of p=2.01e−13 (FIG. 4). In addition, the 20-gene model had a significant True Positive Rate over a False Positive Rate as exemplified by Area-Under the Curve (AUC) statistical analysis, with an AUC=0.87 (FIG. 4).

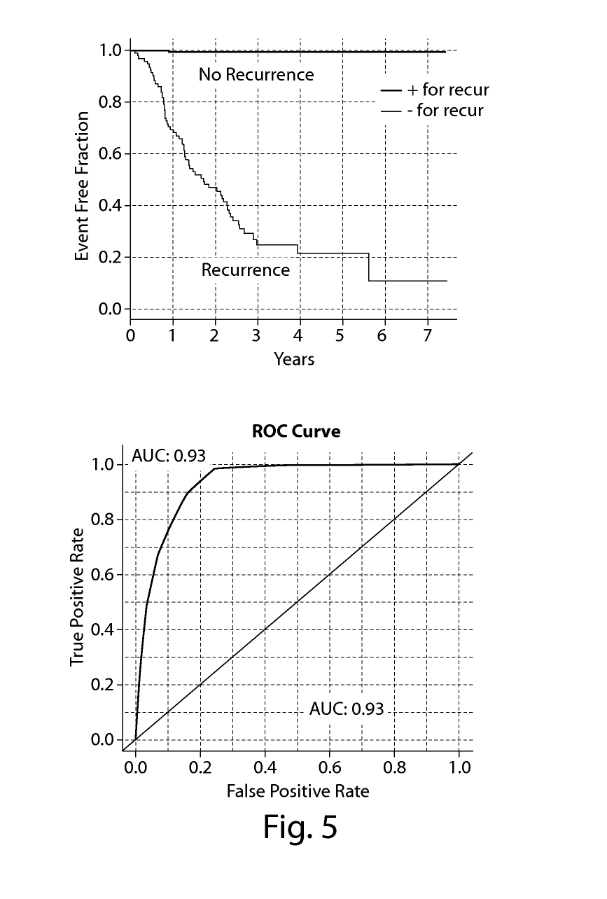

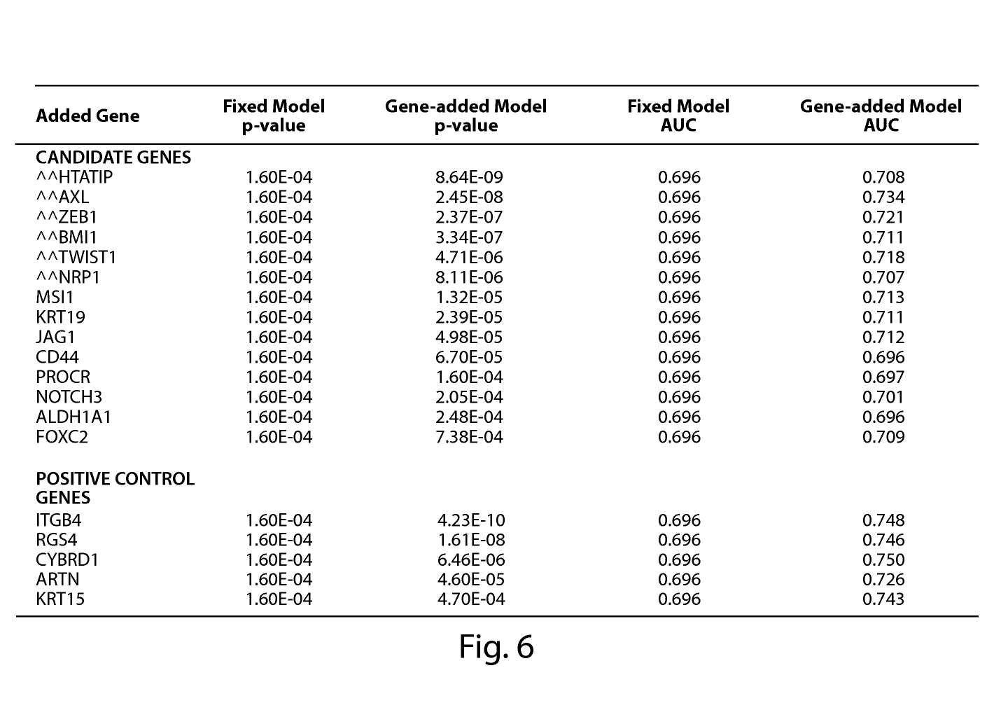

Using the top 20 genes of the CART method, namely genes: CYBRD1, RGS4, CDH3, CHN1, SLC7A5, PRRX1, CITED2, COL5A2, ARTN, ZBED2, SNED1, ITGB4, LUM, PVRL3, OLFML3, BIN1, CCND2, DAB2, ANXA3, and IL18; analysis of patient and RNA data for these genes in Kaplan-Meier recurrence plots showed, the Event Free Recurrence subgroup of patients was significantly distinguished from the patient subset suffering recurrence by this 20-gene test (FIG. 5). In addition, the 20-gene model has a significant True Positive Rate over a False Positive Rate as exemplified by Area-Under the Curve (AUC) statistical analysis, with an AUC=0.93 (FIG. 5).

Utilizing these methods, gene sets were determined that identify recurrence-free patient subgroups from Kaplan-Meier analysis. Importantly, using genes that identify cancer stem cells and the epithelial-mesenchymal transition, associated with their gene expression, key models were discovered that may be utilized in a diagnostic platform for patient discrimination. As this example is displayed for breast cancer, and particularly for triple negative breast cancer, the method is of importance in these cancers. In all likelihood, additional value may be extended to predicting outcome for other cancer types, particularly other solid tumor types.

### Example 2

### Determination of Candidate Cancer Stem Cell Genes that Increase the Performance of Gene Set Models for Identifying the Recurrence-Free Patient Subset in Breast Cancer

In another example, genes that significantly contribute to the ability of gene expression signatures to predict recurrence of cancer were discovered by a process of combining genes with other gene sets. In this exercise, a similar evaluation of breast cancers was explored as an example of the method of discovery and identification of important genes. These genes may describe properties of cancer stem cells, tumor initiating cancer cells, cancer stem cells, and cancer associated mesenchymal cells. This example was conducted on triple negative breast cancer, but it is likely not to be limited to this cancer type or disease. In the present example triple negative breast cancer patients (n=178) were evaluated by standard histopathology criteria and determined to be of the triple negative breast cancer subtype by examining ER, PR, and HER2 by immunohistochemistry and pathology of tumor biopsy specimen. Following their diagnosis, patients were treated with standard chemotherapy and their survival was evaluated over the next seven years, so that outcome data was available for the patient cohort. In one test, gene subsets identified by the above described rank ordering strategies were evaluated in breast cancer patient specimens with RNA expression data formed from profiling patient tumors. In addition, recurrence and/or survival data from the patients was utilized.

Genes used in the addition test were at least one from the group of: AXL, NOTCH3, GPR30, PROCR, PAX2, PI3KA, CD44, CD133, ALDH1, BMI1, KRT19, NRP1, MSI1, MTOR, JAG1, p38, ESA1, ZEB1, TWIST1, FOXC2, CDH2, ZEB2, FOXC1, SNAI1, SNAI2, TWIST2, GSC, TCF3, CD44, VIM, FN1, MLPH1, and ARHGEF11. In the procedure to discover the significance of these genes towards a recurrence prediction, the following test was executed. First, a set of genes was selected from the gene list (i); also shown in the Table 1. In this example, the base model from this group was identified by the LASSO procedure. Eight of the genes that were moderately ranked [Rank 6-13] were selected [SPRR1B, SLC7A5, ANXA3, IL18, AP1M2, CHN1, OLFML3, and ZBED2] after trimming of the top five genes on the LASSO output list. In the analysis, gene addition benefit is demonstrated by comparison of a ‘positive control’ gene dictated by the highest ranking genes of the LASSO set. In this analysis, any one of the following genes was used [CYBRD1, ARTN, KRT15, ITGB4, RGS4; Rank order 1-5]. To evaluate the importance of a new gene, a model was assembled with the Added gene or with a Positive Control gene. Both 5-gene models were then assessed to predict recurrence/non-recurrence patient subgroup identification with genes (logit model, 0.5 probability threshold for assignment). The method used the difference in recurrence curves of predicted classification as a performance measure.

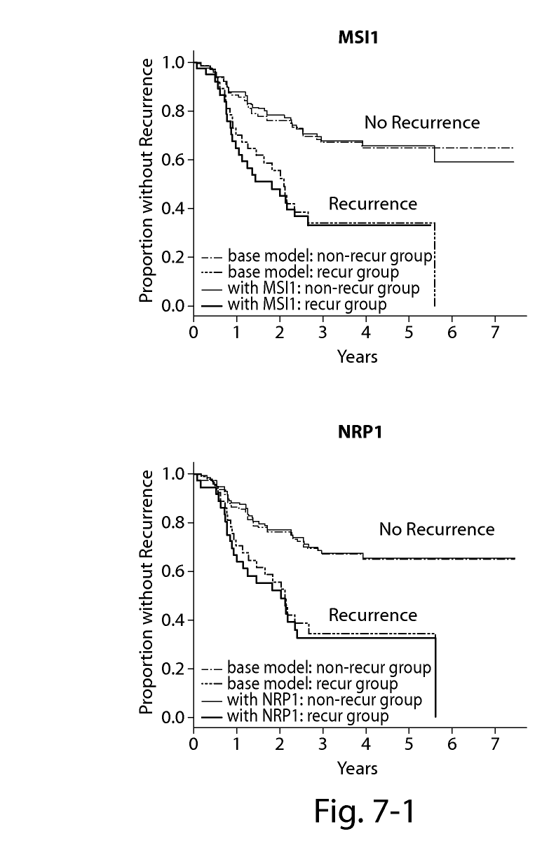

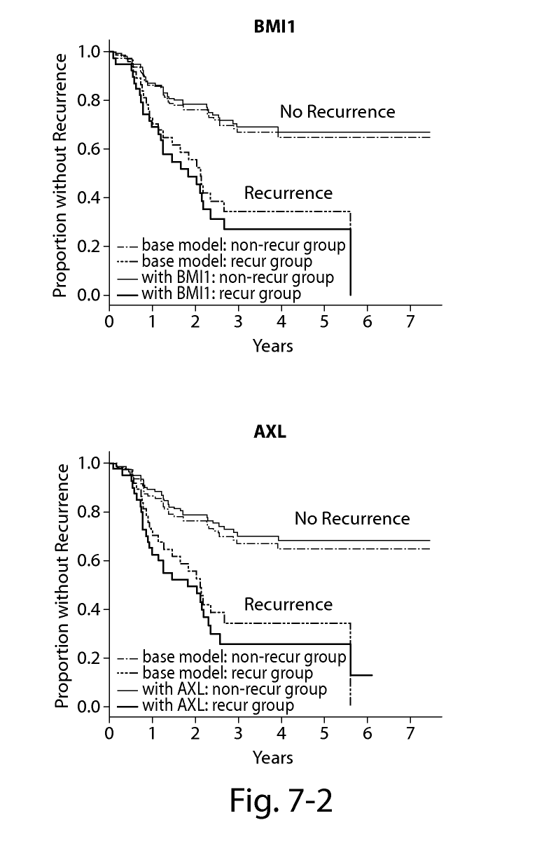

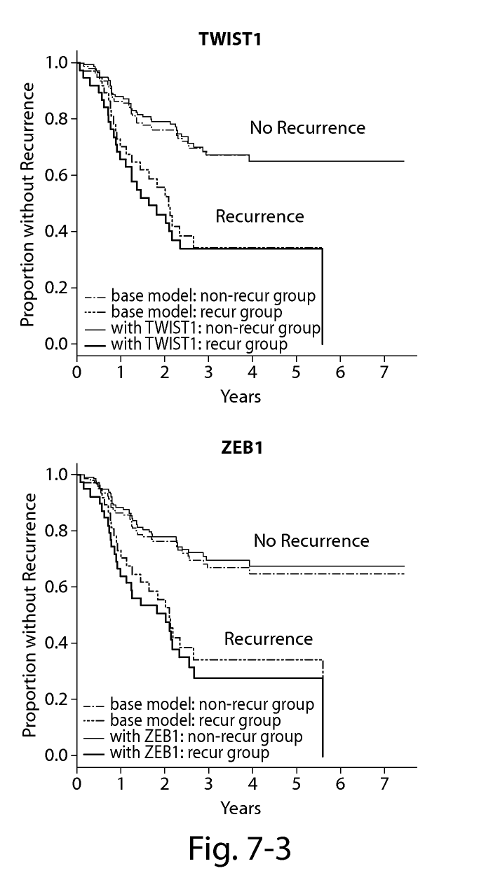

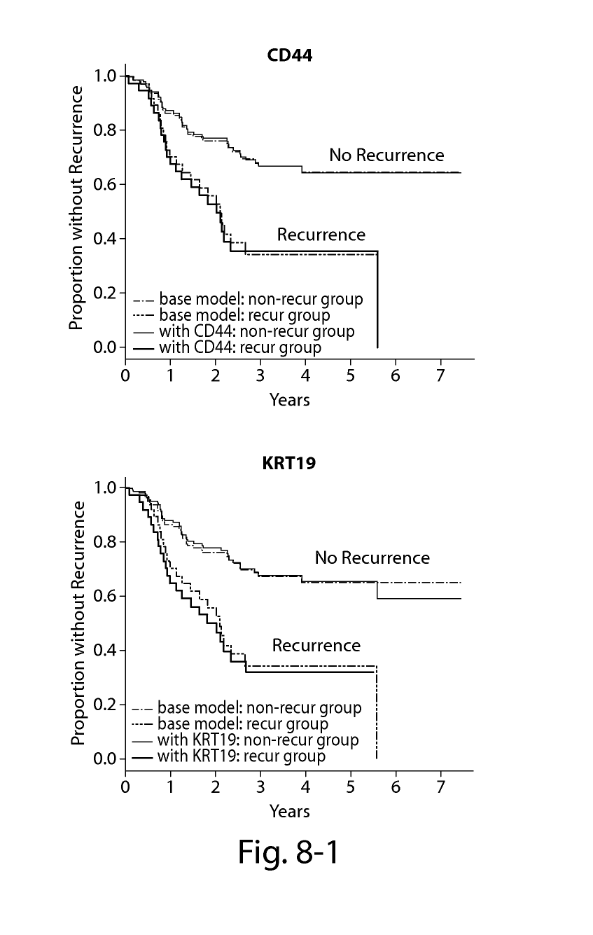

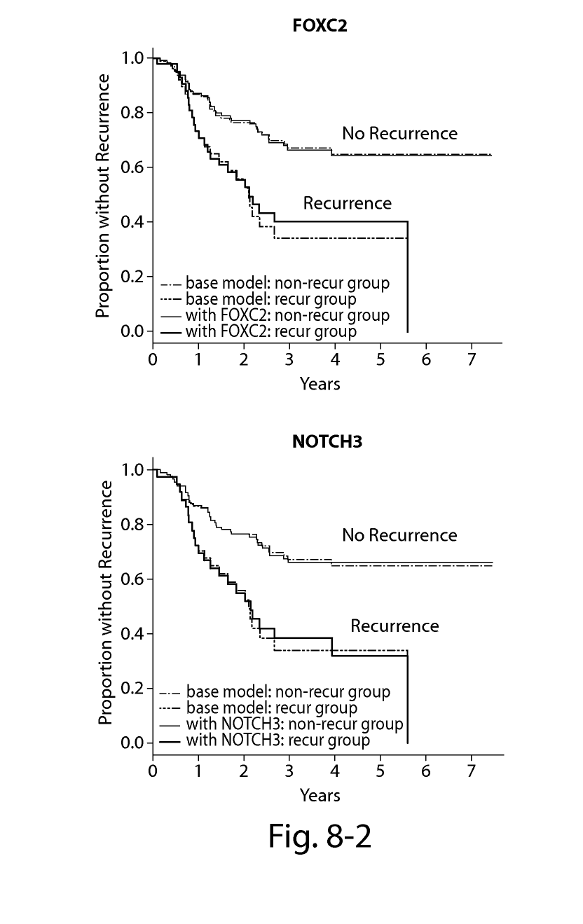

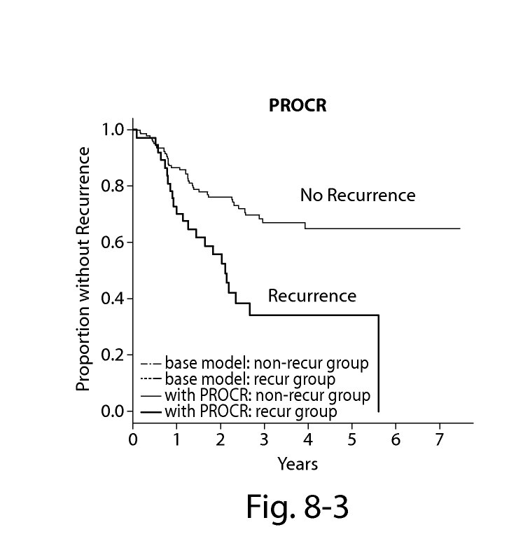

As illustrated in FIG. 7 and FIG. 8, a number of candidate genes were identified to improve the statistical values of base models. These improvements were computed according to each of the genes designated in FIG. 7 and FIG. 8. The p-values of the addition of the candidate genes ranged from p=2.45e−8 to p=7.38e−4. For purposes of comparison, the p-values of addition of the Positive Control Gene ranged from p=4.23e−10 to p=4.70e−04. It is noteworthy that a very high percentage of cancer stem cell/epithelial-mesenchymal transition genes were shown to improve 5-gene models containing the new gene. According to the output of this method, important genes are interrogated for significance when combined in 5-gene models from the base 4-gene models. The method demonstrates the importance of the added genes. Gene models containing the new gene are illustrated to identify recurrence subgroups of triple negative breast cancer patients.

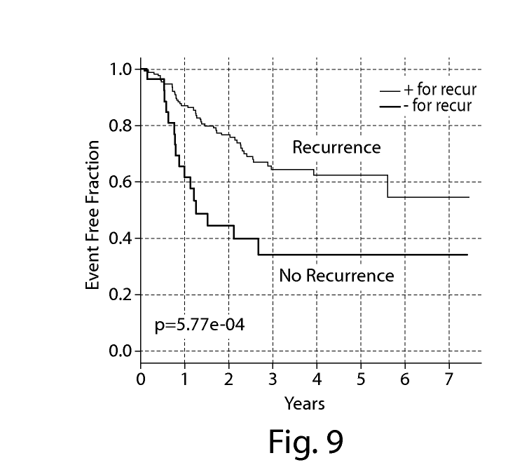

In addition to demonstrating individual contributions of driver genes, the same genes were evaluated as a group in Kaplan-Meier recurrence plots (FIG. 9). In one example, the genes of the candidate CSC genes [NOTCH3, PROCR, BMI1, KRT19, MSI1, NRP1, JAG1, ALDH1A1, and HTATIP] were used in a multi-gene model and in a Kaplan-Meier recurrence plot. The 9-gene model gave separation of triple negative breast cancer patients into the Recurrence and No Recurrence groups with a p=5.77e−04 (FIG. 9).

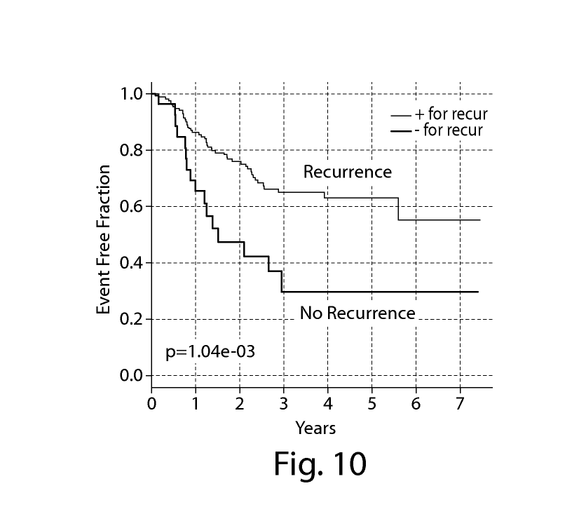

In another example, the role of candidate CSC genes was delineated by evaluation of a 5-gene model (FIG. 10). The genes NRP1, BMI1, JAG1, MSI1, and HTATIP were used in a Kaplan-Meier recurrence plot. The 5-gene model gave separation of triple negative breast cancer patients into the Recurrence and No Recurrence groups with a p=1.04e−03 (FIG. 10). Whereas the 5-gene model exhibited high significance, none of the individual genes demonstrated a p<0.05 individually.

### Example 3

### Identification of Genes Expressed from Tumorspheres that Increase the Performance of Gene Set Models for Identifying the Recurrence-Free Patient Subset in Breast Cancer

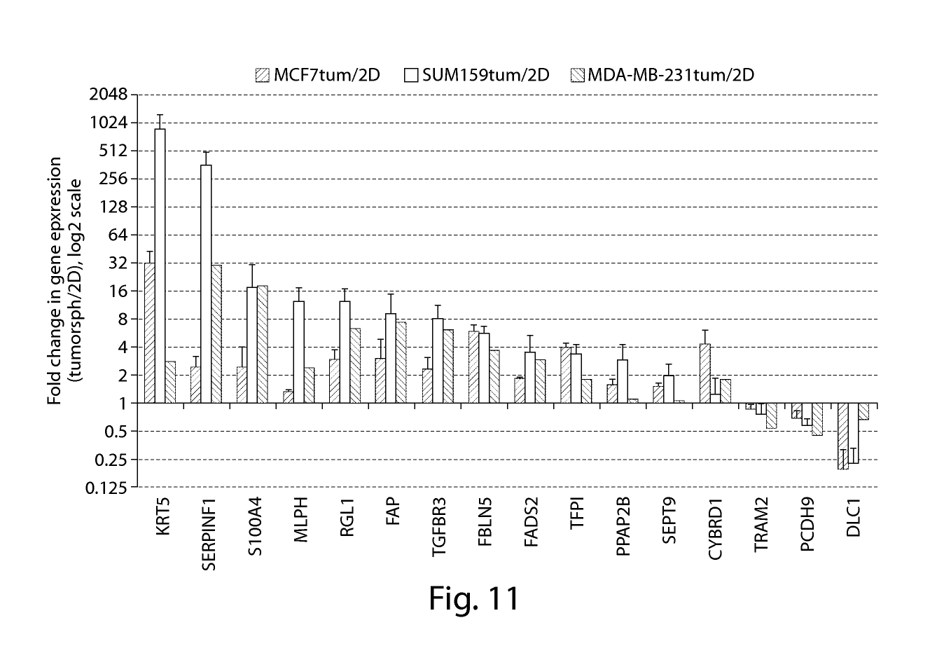

The identification of critical genes from cancer stem cells and tumor initiating cancer cells is exemplified by analysis of in vitro formation of tumorspheres from human cancer cell lines. In this example, tumorspheres were derived from four human breast cancer cell lines: MDA-MB-231, SUM-159, MCF7, and Hs578T. Notably, two of these cell lines are derived from breast cancer patients with basal-like morphology. The method illustrates a means to determine the relative expression of candidate cancer stem cell and tumor initiating cancer cell genes by comparing tumorspheres with two-dimensional cell culture. The fold-change in gene expression was measured by Q-PCR for RNA samples isolated from Tumorspheres or from two-dimensional cell culture of human breast cancer cell lines. The genes KRT5, SERPINF1, S100A4, RGL1, FAP, TGFBR3, FBLN5, MLPH, FADS2, TFPI, PPAP2B, SEPT9, CYBRD1, UGDH, TRAM2, PCDH9, and DLC1 were evaluated in this analysis. The plots show the fold change of Tumorsphere/2D culture on a log 2 scale for the three cell lines for several of the genes (FIG. 11). Genes from tumor initiating cancer cell, EMT, and cancer stem cell discriminators (Table 1) that were changed in 3 out of the 4 human breast cancer cell lines between tumorspheres and 2D cultures were: KRT5, MMP1, RGL1, FAP, TGFBR3, FBLN5, STC1, FADS2, IGFBP4, VIM, IBGFBP3, UGDH, ENPP2, IER3, SNAIL2, MYL9, DOCK10, DAB2, ROR1, FERMT2, MAP1B, PVRL3, PTGER4, CDH2, CTGF, and TAGLN. Genes that were upregulated or downregulated in a consistent manner between all four of the human breast cancer cells lines between tumorspheres and 2D culture were: CYBRD1, DLC1, MLPH, MYL1, PCDH9, S100A4, SERPINF1, TFPI, and TRAM2. The genes KRT5, SERPINF1, S100A4, RGL1, FAP, TGFBR3, FBLN5, MLPH, FADS2, TFPI, PPAP2B, SEPT9, CYBRD1, and UGDH were determined to be upregulated. Likewise, the genes TRAM2, PCDH9, and DLC1 were downregulated. There were several genes that were upregulated >2 fold in tumorspheres of all 3 cell lines compared to 2D, including: KRT5, SERPINF1, S100A4, RGL1, FAP, TGFBR3, FBLN5, PPAP2B, SEPT9, CYBRD1 and UGDH. Genes that were upregulated in tumorspheres of all 3 cell lines compared to 2D (at least changed in the same direction): MLPH, FADS2, TFPI, DLC1, TRAM2 and PCDH9. Similarly, with genes from tumor initiating property discriminators (Table 1) that were changed in 2 out of the 3 human breast cancer cell lines [MCF7, SUM159, Hs578T] were: ARID5B, MALAT1, NT5E, CNR1, HIPK2, FOXO1, NUP188, FXYDS, FKBP5, and DDB2. Also, genes from tumor initiating property discriminators (Table 1) that were changed in all 3 of the human breast cancer cell lines were: BASP1, FKBP5, FLII, POLR2A, PTGS1, SH3BGRL3, and SSBP2.

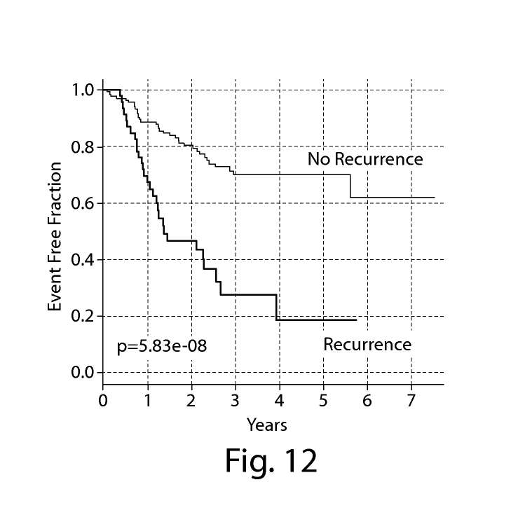

Evaluation of the role of these genes in predicting breast cancer recurrence was evaluated in triple negative breast cancer (TNBC) patients. In this study, the tumor biopsies of 178 TNBC patients were examined for RNA profiling levels of different genes on the Affymetrics U133 microarray chip. Expression values for genes of interest were computed. A Kaplan-Meier display was outputted for the study, in which patient medical records were used to determine the ‘event-free fraction’ from 0-7 years, for the group of genes [KRT5, SERPINF1, S100A4, RGL1, FAP, TGFBR3, FBLN5, MLPH, FADS2, TFPI, PPAP2B, SEPT9, CYBRD1, UGDH, TRAM2, PCDH9 and DLC1, 16 gene model] and other similar models were determined. In one example, using a 16 gene model from the group [CYBRD1, FAP, TRAM2, PPAP2B, TFPI, PCDH90, RGL1, FADS2, SERPINF1, DLC1, SEPT9, MLPH, S100A4, FBLN5, and TGFBR3], it was determined that these genes fit a model to separate Likely to Recur from Not Likely to Recur patients with high statistical significance, p=5.83e−08 (FIG. 12). The AUC for this separation of patient outcome was determined to be AUC=0.72. A Relative Risk was calculated to be 2.4 with a 95% Confidence Interval range from 1.7-3.4.

In another determination of multigene models, a second 5-gene group consisting of CYBRD1, FAP, PPAP2B, TRAM2 and TFPI genes was examined on the same triple negative breast cancer patient group. A Kaplan-Meier display was outputted for the study where patient medical records were used to determine the ‘event-free fraction’ from 0-7 years. It was determined that these genes fit a model to separate Likely to Recur from Not Likely to recur patients, with a p-value between the ‘Recurrence’ and ‘No recurrence’ subgroups of patients of p=2.19e−08 (FIG. 13). The AUC for this separation of patient outcome was determined to be AUC=0.69 (FIG. 13). A Relative Risk was calculated to be 2.4 with a 95% Confidence Interval range from 1.7-3.4.

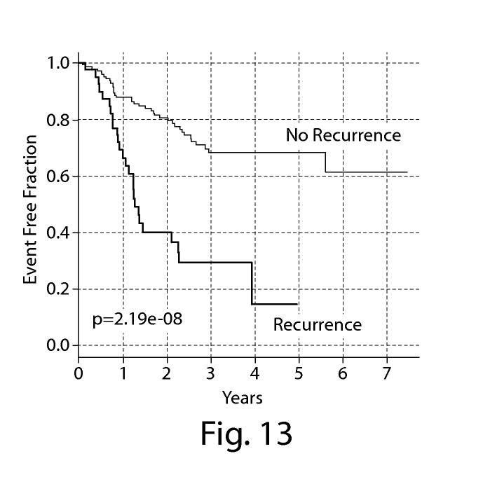

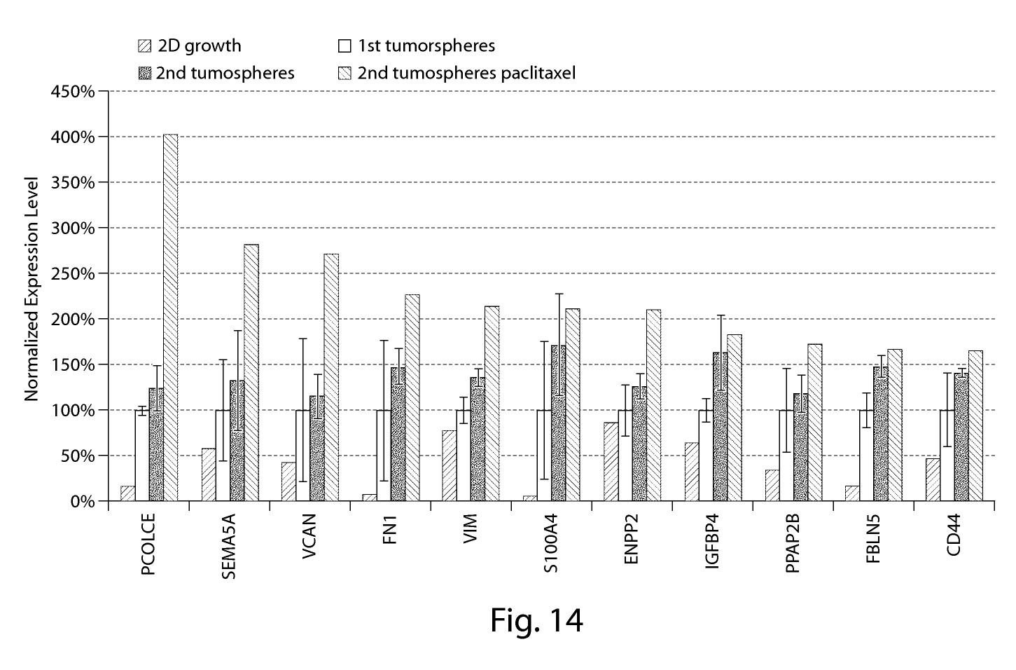

In addition, tumorsphere cultures were utilized to screen for the upregulation or downregulation of candidate genes under conditions in which the tumorspheres were treated with chemotherapeutic agents, cytotoxic agents, cytostatic agents and the like. Particular cells may be enhanced or eliminated by these treatments leading to an increased change in expression of key genes in the remaining tumorspheres in culture (secondary tumorspheres). Information about which genes are upregulated or downregulated or unchanged can be informative in the development of clinical diagnostics, and may be useful as drug response biomarkers. For example, sequential passaging of tumorspheres of SUM159 cells led to upregulation of 11 out of 126 CSC-associated genes (FIG. 14). These genes that are changed upon secondary culture of tumorspheres include: CD44, ENPP2, FBLN5, FN1, IGFBP4, PCOLCE, PPAP2B, S100A4, SEMA5A, VCAN, and VIM. Furthermore, expression of upregulated genes was enhanced further in paclitaxel-treated tumorspheres. Other culture conditions such as Matrigel are also relevant to the discrimination of gene sets that show changed regulation under such culture conditions. Correlations observed between the presence of certain expressed genes and specific drug treatment, such as with agents that target cancer stem cells, are expected to change the expression levels of these genes in the drug response.

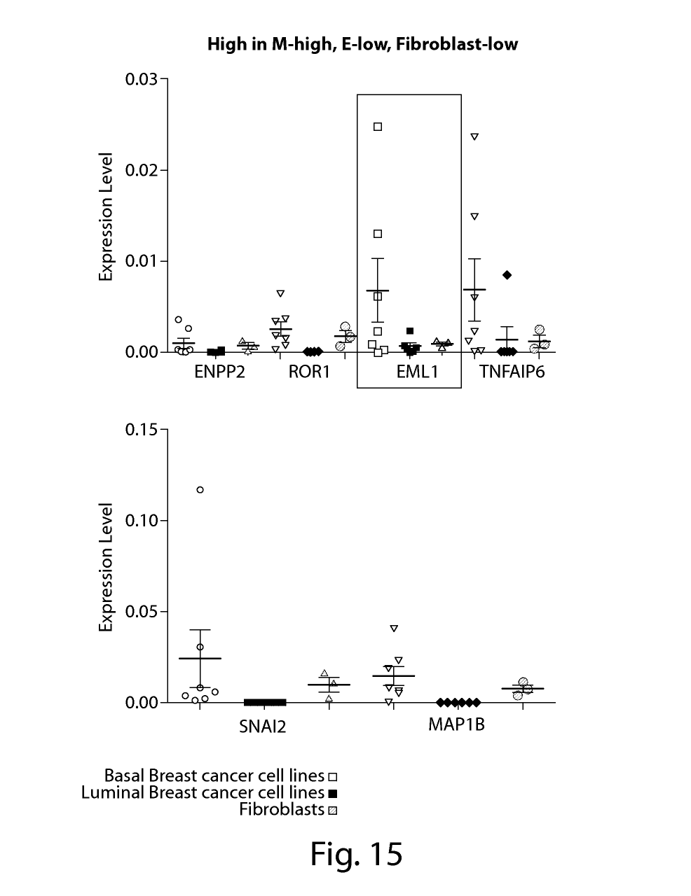

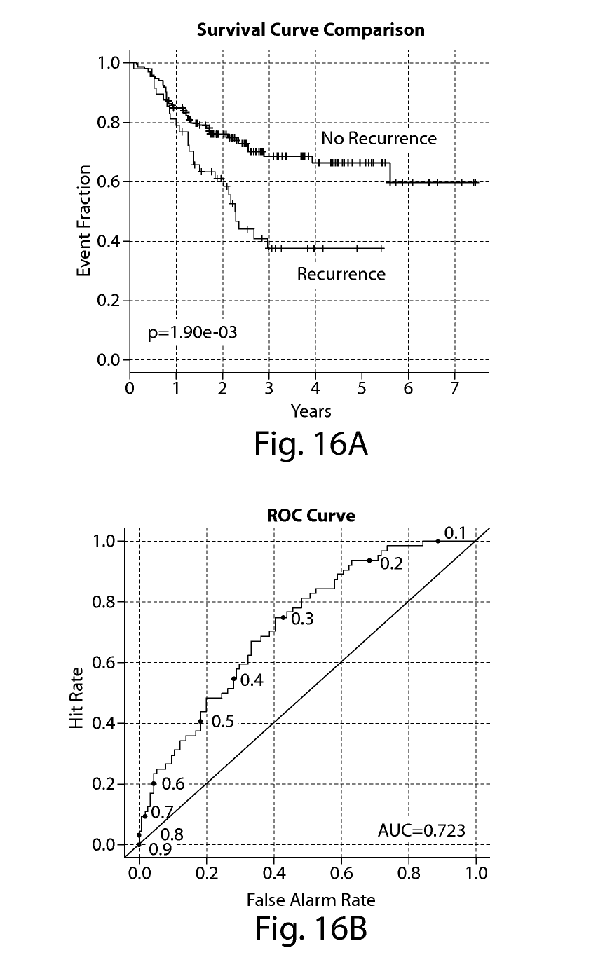

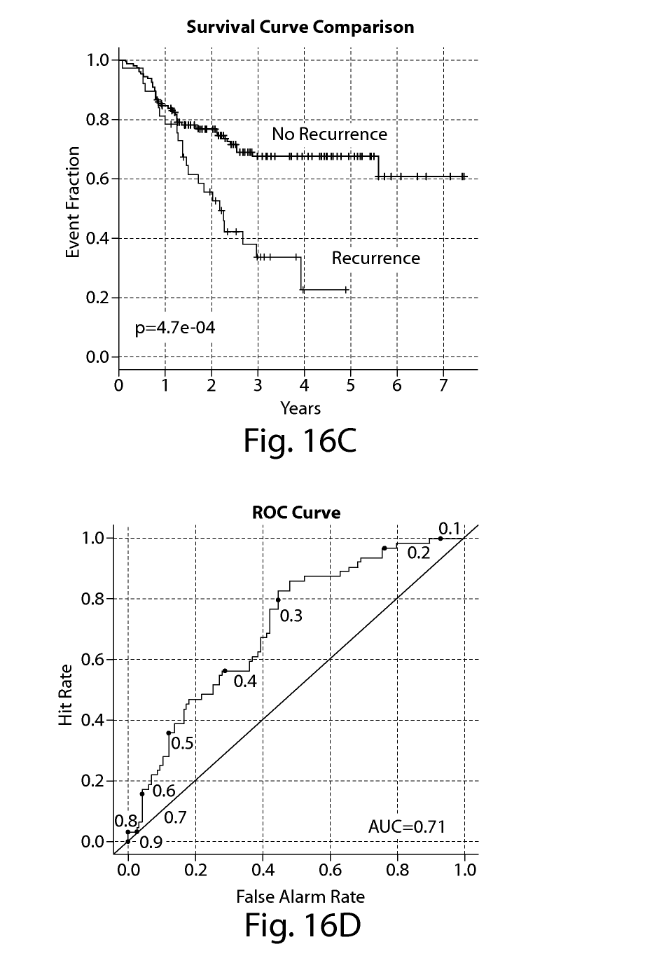

In an additional refinement, genes were evaluated with regard to differential expression in particular cell types. In this mode, we evaluate the genes that are expressed in mesenchymal (M) tumor cells, and low or not expressed in epithelial (E) tumor cells, and low or not expressed in fibroblast (F) non-tumor cells [M-high, E-low, Fibroblast-low] (FIG. 15). In human tumor specimens, fibroblast or stromal cells are generally not of cancer cell origin, and are not expected to be derived from cancer stem cells. Therefore, screens that exclude the fibroblast or stromal cells, may aid in the definition of cancer stem cells, tumor initiating cancer cells, and mesenchymal associated cancer cells. Examples of M-high, E-low, Fibroblast-low genes include: CDH2, CHN1, CTGF, CYP1B1, DNAJB4, EML1, ENPP2, HAS2, MAP1B, NID1, PDGFC, PRR16, PRRX1, PVRL3, ROR1, SDC2, SNAI1, SNAI2, TNFAIP6, and VCAN. Other genes may have similar properties where different cut-off values will be utilized for discriminating preferential expression between mesenchymal cancer cell and fibroblast-type cells. These genes and gene groups showing differential properties between mesenchymal-type and fibroblast-type cells are considered to describe properties of cancer stem cells, tumor initiating cancer cells, and cancer associated mesenchymal cells in human primary cancer specimens. It is important to determine whether the overexpression and underexpression of genes in this group is able to identify patients that may have a likelihood to recur following chemotherapy treatment, or may have a likelihood to respond and not have evident clinical recurrence after chemotherapy treatment. To evaluate whether the M-High, E-Low, Fibroblast-Low gene subgroup may be sufficient to determine reoccurrence outcome, a cohort of 178 Triple negative breast cancer patient tumor specimens were examined for RNA profiling levels of different genes on the Affymetrics U133 microarray chip. Expression values for genes of interest were computed. A Kaplan-Meier display was outputted for the study where patient medical records were used to determine the ‘event-free fraction’ from 0-7 years. The M-High, E-Low, Fibroblast-Low group of CDH2, CHN1, CTGF, CYP1B1, DNAJB4, EML1, ENPP2, HAS2, MAP1B, NID1, PDGFC, PRR16, PRRX1, PVRL3, ROR1, SDC2, SNAI1, SNAI2, TNFAIP6, and VCAN [19-gene model]), determined that these genes fit a model to separate Likely to Recur (Recurrence) from Not Likely to Recur (No Recurrence) patients with high statistical significance of p=1.9e−03, with an AUC of 0.723 (FIG. 16 A, B). Discrimination of subgroups of these genes with high statistical significance was outputted. This subgroup of the genes that were M-High, E-Low, Fibroblast-Low was derived by drop-one-out iterations with statistical performance assessments, to yield the 6 gene set including genes: CHN1, CTGF, DNAJB4, PRRX1, PVRL3, and VCAN having high statistical significance of a p=4.7e−04 separating the No Recurrence versus Recurrence patients, and with an AUC of 0.71 (FIG. 16 C, D) for the model test.

It is striking that candidate genes derived from either human tumor cell line culture measurements in tumorspheres or from discrimination between mesenchymal and fibroblast gene expression levels and patterns, are both a means to rank and derive multi-gene models that identify patients separating No Recurrence and Recurrence groups.

### Example 4

### Gene Ranking Based on Gene Expression Variance and Polarity

Genes from the CSC, EMT, and Tumor Initiating origin (Table 1) were evaluated for variance in expression by analyzing the range in expression values amongst a group of samples. The samples used in this evaluation may be from multiple sources, including human cancers, or from models of cancers, such as patient-derived xenografts, or mouse xenografts of human tumor cells, or from comparisons between human cancer cell lines. In the present evaluation, 178 human breast cancer specimens, and associated gene expression microarray data were compared for each gene in the array. The mean expression value and the variance distribution amongst the gene members of the set, relative to all the genes on the array, were processed. In this determination, genes were ranked by increased [cv category A, Table 2], mid-level, or decreased variances. Thus, genes of interest would be determined to have a variance rank (cv.rank), a percentage of variance relative to all genes on the array (percent.cv), and a mean expression, allowing an assessment of relative value. CV category A is indicative of genes that are more variable amongst the sample specimens. CV category B and C indicate genes where the gene expression is not as variable as other genes in the set, and for the specimens tested.

Genes from the CSC, EMT, and Tumor Initiating origin (Table 1) were also inspected for their likelihood to change between increased versus decreased expression, termed Polarity (Table 2). Simulations of 92-143 cycles resulting from 1000 model iterations were processed to output a median.polarity value, a Polarity Percentile, and a median.p (P value of the likelihood of significance based on Highly Significant being a gene that had a consistent Polarity of increased or decreased expression level (Table 2)). In order to discriminate high value of Polarity amongst the genes in the group, an arbitrary cut-off of p<0.01 was established that indicated the genes with <20% or >80% value in Polarity.

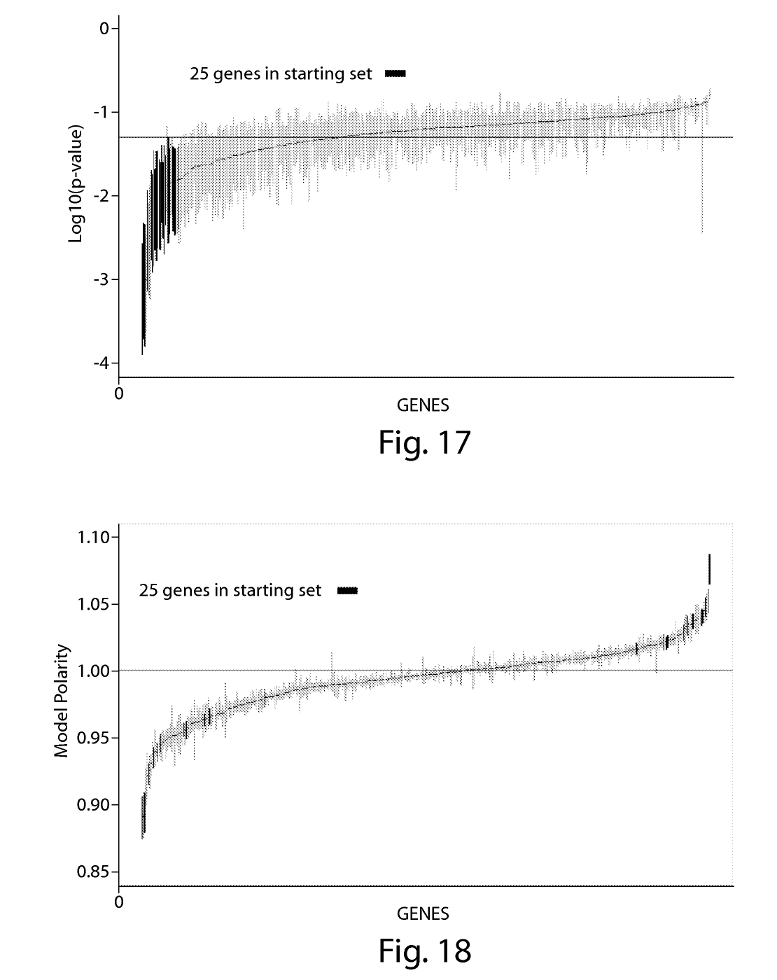

Accordingly, subgrouping of significant genes based on Variance and Polarity amongst a large group of human breast cancer specimens, was processed. FIG. 17, shows that the 25 genes that are highly ranked by the Variance criteria, are more variable than all genes in the most Variance group (below the horizontal line, p<0.01), and significantly more variable than the All Genes in the array. FIG. 18, shows that the 25 most highly ranked genes have Polarity values that are consistently of one polarity or the other relative to all the genes in the dataset. Genes that have a Polarity value of 1.00 or near 1.00 have approximately equal probability of being increased or decreased expression when used in a statistical model.

As shown above, selection of Variance and Polarity criteria may be assembled to determine gene ranking prioritization of membership in statistical models for response criteria and clinical outcome data, such as with patient recurrence with the example of human breast cancers. The example was conducted on triple negative breast cancer, but it is not likely to be limited to this cancer type or disease. In the present example, triple negative breast cancer patients (n=178) were evaluated by standard histopathology criteria and determined to be of the triple negative breast cancer subtype by examining ER, PR, and HER2 by immunohistochemistry and pathology of tumor biopsy specimens. Following their diagnosis, patients were treated with standard chemotherapy and their survival was evaluated over the next seven years, so that outcome data was available for the patient cohort. In one test, gene subsets identified by the above rank ordering strategies were evaluated in breast cancer patient specimens with RNA expression data formed from profiling of patient tumors. In addition, recurrence and/or survival data from the patients was utilized.

Genes used in the addition test were at least one from the group of the 25 highest ranked genes based on Variance and Polarity: CYBRD1, INTS8, RGS4, FBXO21, PRRX1, POLS, ID3, OLFML3, PRSS16, CIRBP, CHN1, SERPINE1, SH2B3, ZBTB16, TWIST1, EIF3S9, DPF2, CDH11, CTGF, CCNB1, VIL2, UBE2S, APLP2, MGP, and NOL8. However, more genes may populate these lists based on the criteria of Variance and/or Polarity. Alternatively, genes may be mixed based on Good Ranking Status as one criteria (Table 2) and on Appearance in Tumorsphere cultures as an independent criteria.

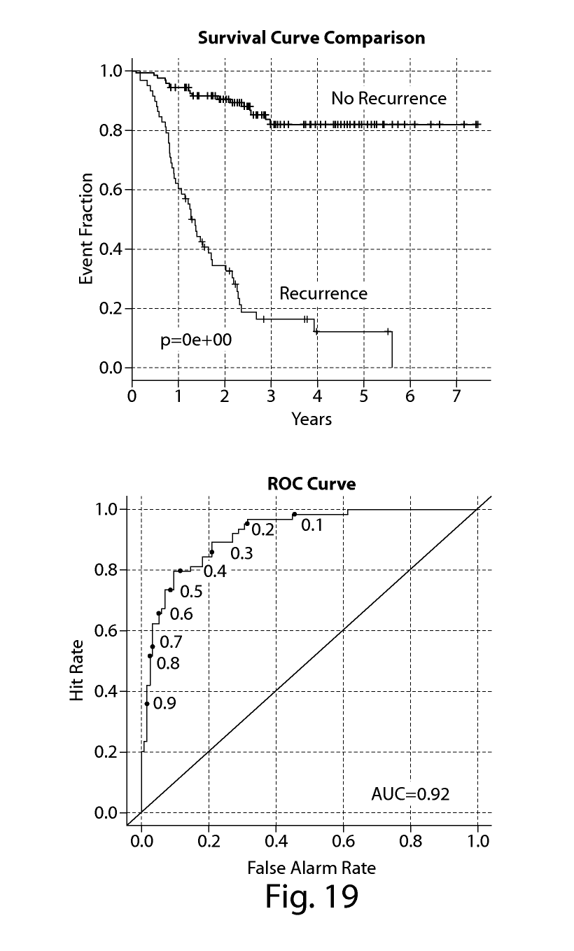

In one execution of statistical models, the 25 highest ranked genes based on Variance and Polarity [CYBRD1, INTS8, RGS4, FBXO21, PRRX1, POLS, ID3, OLFML3, PRSS16, CIRBP, CHN1, SERPINE1, SH2B3, ZBTB16, TWIST1, EIF3S9, DPF2, CDH11, CTGF, CCNB1, VIL2, UBE2S, APLP2, MGP, and NOL8] were iteratively sampled by logistic regression and leave-one-out sampling to identify the 18 top ranked genes. The top 18 ranked genes (CYBRD1, INTS8, RGS4, FBXO21, PRRX1, POLS, ID3, OLFML3, PRSS16, CIRBP, CHN1, SERPINE1, SH2B3, ZBTB16, TWIST1, EIF3S9, DPF2, CDH11, CTGF, CCNB1, VIL2, and UBE2S) were highly ranked, where the models containing these genes showed a p<0.05 significance for 13 of the genes. When analyzed in Kaplan-Meier Time to Recurrence plots, the top 18 gene model (multigene model) showed a separation of triple negative breast cancer patients into the Recurrence and No Recurrence groups with a p<0e+00 (FIG. 19). In comparison, with the 25 gene model, the AUC computed in Receiver Operator Curve analysis 0.92.

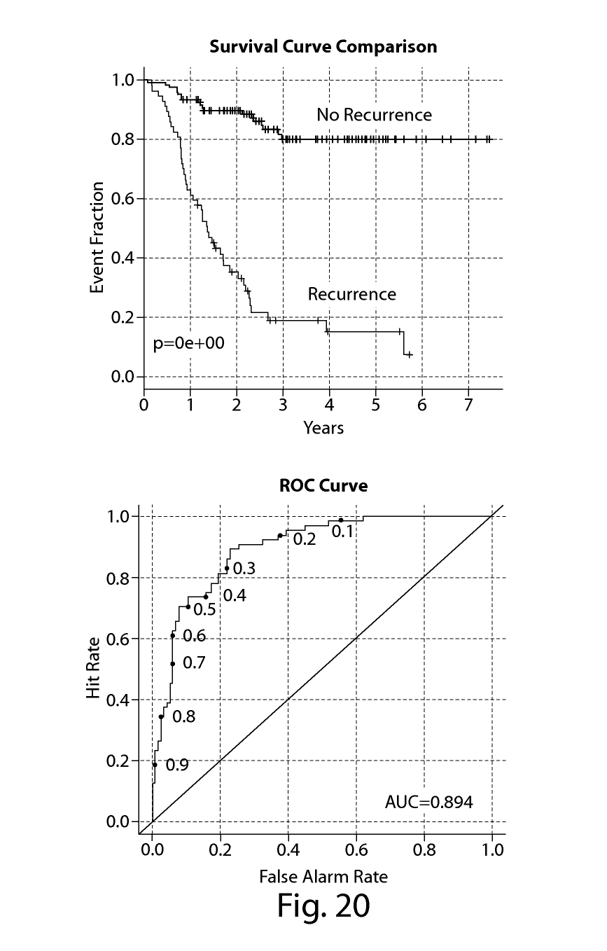

Extension of the analysis to reduce the number of genes in the model, and to discriminate the driver genes in the model was also conducted with these gene lists. Starting from the top 18 genes determined by Variance, Polarity, and Leave-one-out, statistical output in Kaplan-Meier Recurrence plots determined that a refinement to a 12-gene model (CYBRD1, INTS8, RGS4, FBXO21, PRRX1, ID3, CHN1, SERPINE1, ZBTB16, TWIST1, CCNB1, UBE2S) had a highly significant statistical value for the separation of triple negative breast cancer patients into the Recurrence and No Recurrence groups with a p<0e+00 (FIG. 20). In comparison, for this 12 gene model, the computed AUC was 0.894 in Receiver Operator Curve analysis. In the top 12 gene model [CYBRD1, INTS8, RGS4, FBXO21, PRRX1, ID3, CHN1, SERPINE1, ZBTB16, TWIST1, CCNB1, UBE2S], all 12 genes had a p<0.05.

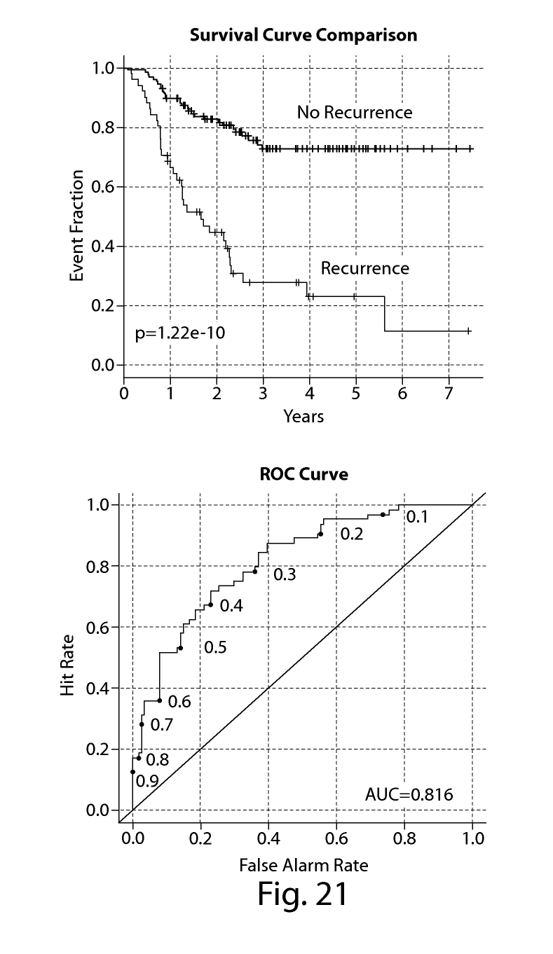

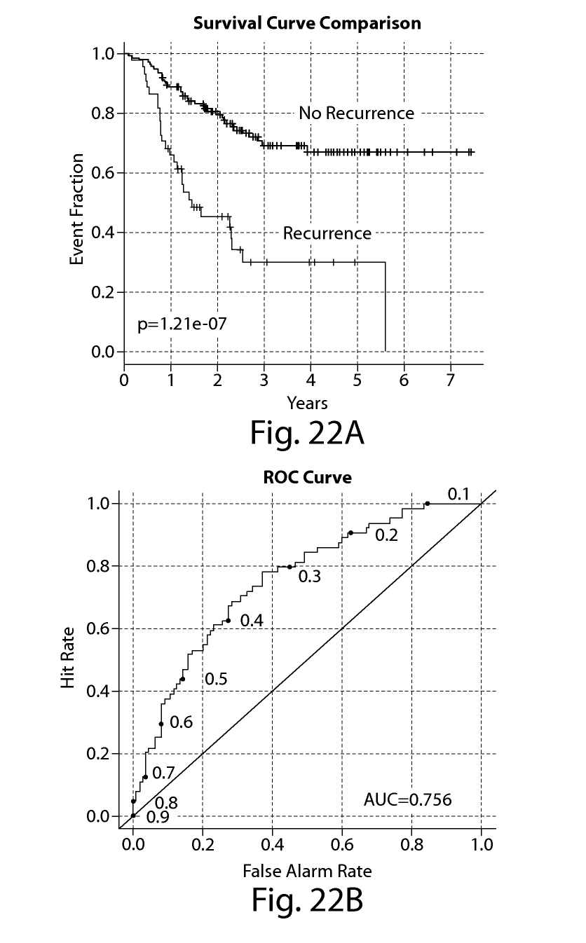

Likewise, starting from the top 12 genes by Variance, Polarity, and Leave-one-out statistical output in a Kaplan-Meier Recurrence plot was executed. It was determined that a refinement to give a 6-gene model (CYBRD1, RGS4, PRRX1, ID3, SERPINE1, and ZBTB16) had a highly significant statistical value for the separation of triple negative breast cancer patients into the Recurrence and No Recurrence groups with a p<1.22e−10 was observed (FIG. 21). In comparison, for this 6 gene model, the AUC was computed to 0.816 in Receiver Operator Curve analysis. In the top 6 gene model [CYBRD1, RGS4, PRRX1, ID3, SERPINE1, and ZBTB16], all 6 genes had a p<0.05. Genes that are either upregulated or downregulated may contribute to these models.

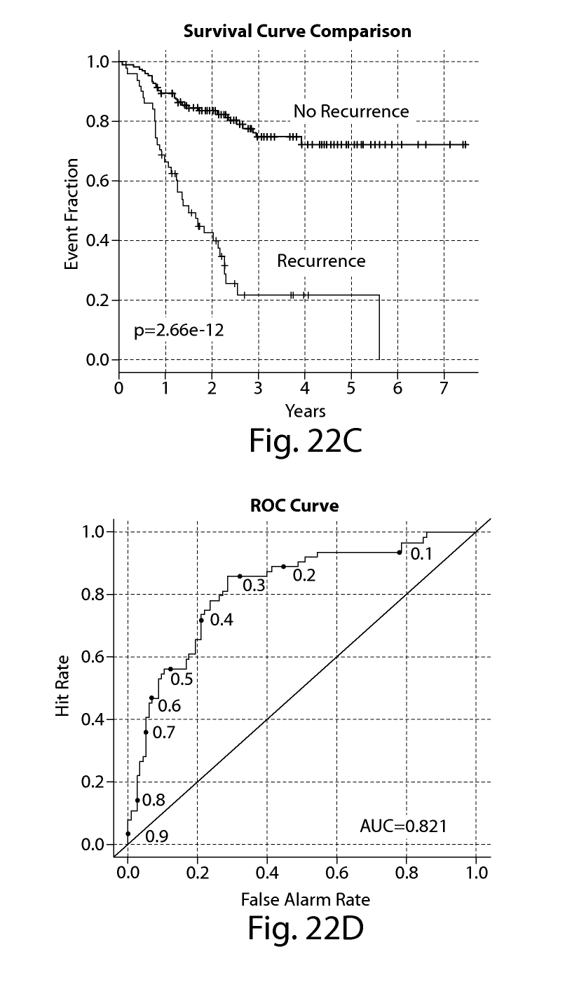

Genes that were candidate genes based on laboratory Q-PCR measurements, such as with Tumorsphere culture analysis were also co-evaluated in gene ranking strategies based on Variance and Polarity. In one example, the four genes CYBRD1, SERPINF1, FAP, and PPAP2B, individually indicated that increased expression is found in tumorsphere cultures, possibly possessing increased levels of cancer stem cells, and statistical output in Kaplan-Meier Recurrence plots were executed. Further refinements also gave several multi-gene models that had highly significant statistical value for the separation of triple negative breast cancer patients into the Recurrence and No Recurrence groups. For example, CYBRD1, SERPINF1, FAP, PPAP2B with RGS4 and PRRX1 (Tumorsphere 6-gene model) yielded a p=1.21e−07, and an AUC of 0.756 (FIG. 22). In comparison, an 8-gene model combining CYBRD1, SERPINF1, FAP, PPAP2B with RGS4, PRRX1, FBXO21, and ID3 was a highly significant model with a p=2.66e−12, and the computed AUC of 0.821 in Receiver Operator Curve analysis. Genes that are either upregulated or downregulated may contribute to these models. In addition, the weighting of each gene may be individually determined.

These examples demonstrate that combinations of genes may be fit to combinatorial models defining an ability to separate patients into No Recurrence and Recurrence groups. Definition of these patient subgroups may contribute to and represent functionally relevant subgroups for future treatments with agents that inhibit or kill cancer stem cell, tumor initiating cancer cell, and tumor associated mesenchymal.

TABLE 2
Rank Order of Genes based on Variance and Polarity

| | | | | | | | cv | times used | | |
|---|---|---|---|---|---|---|---|---|---|---|
| | | median. | cv. | cv. | percent. | Mean. | cate- | in a | polarity, | polarity, |
| gene | median.p | polarity | rank | percentile | cv | expression | gory | simulation | percentile | category |
| CYBRD1 | 0.000447844 | 0.923472345 | 11663 | 87.41567981 | 10.0269784 | 5.971931781 | A | 110 | 1% | Good |
| INTS8 | 0.000616548 | 1.046942757 | 10715 | 80.31029831 | 8.527228238 | 7.72922699 | A | 118 | 99% | Good |
| RGS4 | 0.000822959 | 1.039597077 | 9718 | 72.83765552 | 7.507724639 | 5.633178734 | A | 109 | 99% | Good |
| FBXO21 | 0.002553602 | 0.955077734 | 9377 | 70.28181682 | 7.189143411 | 6.666660827 | A | 98 | 7% | Good |
| PRRX1 | 0.002666276 | 0.955583314 | 12330 | 92.4149303 | 11.78954373 | 5.545155189 | A | 115 | 8% | Good |
| POLS | 0.004302918 | 1.040256137 | 9156 | 68.62539349 | 7.022201602 | 8.08865953 | A | 110 | 99% | Good |
| ID3 | 0.004354395 | 1.051773745 | 12848 | 96.29740669 | 14.70610253 | 5.535638216 | A | 105 | 100% | Good |
| OLFML3 | 0.006954545 | 0.940273485 | 12594 | 94.39364413 | 12.96726543 | 7.370975636 | A | 110 | 3% | Good |
| PRSS16 | 0.007902452 | 0.966097766 | 11165 | 83.68310598 | 9.159501564 | 5.330501554 | A | 101 | 12% | Good |
| CIRBP | 0.008174492 | 0.937708742 | 11088 | 83.10598111 | 9.041599393 | 8.081095769 | A | 127 | 2% | Good |
| CHN1 | 0.008563674 | 1.022740484 | 9985 | 74.83885474 | 7.757662112 | 6.898214366 | A | 125 | 93% | Good |
| SERPINE1 | 0.008883279 | 1.036139646 | 10308 | 77.25978114 | 8.086722676 | 5.571009865 | A | 120 | 97% | Good |
| SH2B3 | 0.009050152 | 1.015900731 | 9231 | 69.18752811 | 7.078451596 | 6.474525203 | A | 109 | 87% | Good |
| ZBTB16 | 0.010047024 | 0.94722274 | 12784 | 95.81771848 | 14.1791896 | 5.075007435 | A | 96 | 4% | Good |
| TWIST1 | 0.011391626 | 1.075579454 | 13014 | 97.54159796 | 16.25944106 | 4.434120542 | A | 131 | 100% | Good |
| EIF3S9 | 0.012128966 | 1.022312183 | 7411 | 55.54639484 | 6.001613725 | 8.702829032 | A | 107 | 92% | Good |
| DPF2 | 0.013138673 | 0.964324655 | 8899 | 66.69914556 | 6.840009078 | 7.097320284 | A | 137 | 11% | Good |
| CDH11 | 0.01348098 | 0.947875852 | 13115 | 98.29860591 | 17.80084339 | 6.042363365 | A | 110 | 4% | Good |
| CTGF | 0.013752874 | 0.891138612 | 13252 | 99.32543846 | 21.7169806 | 6.984676613 | A | 99 | 0% | Good |
| CCNB1 | 0.013821791 | 1.03419766 | 12498 | 93.67411183 | 12.4850567 | 6.668403951 | A | 143 | 96% | Good |
| VIL2 | 0.014892512 | 1.021687837 | 10633 | 79.6956978 | 8.428163329 | 7.216439561 | A | 122 | 92% | Good |
| UBE2S | 0.01528562 | 0.979664602 | 12199 | 91.43306851 | 11.39222508 | 8.019233778 | A | 120 | 22% | Good |
| APLP2 | 0.015343287 | 0.945443626 | 11216 | 84.06535752 | 9.245491905 | 8.232277003 | A | 122 | 3% | Good |
| MGP | 0.015353658 | 0.897058445 | 13037 | 97.71398591 | 16.50926949 | 11.06831798 | A | 116 | 0% | Good |
| NOL8 | 0.015537274 | 1.029697179 | 9964 | 74.68145705 | 7.740372192 | 6.31811628 | A | 116 | 95% | Good |
| IGFBP7 | 0.015696722 | 1.001137554 | 13261 | 99.39289462 | 22.23554394 | 7.560811784 | A | 97 | 58% | Poor |
| CTNNB1 | 0.015988802 | 0.951844112 | 10396 | 77.91935242 | 8.16482316 | 8.201877814 | A | 123 | 6% | Good |
| CLDN4 | 0.017338142 | 1.027368568 | 11703 | 87.71548493 | 10.13444062 | 7.266147398 | A | 120 | 95% | Good |
| LUM | 0.018167638 | 0.969794331 | 13334 | 99.94003897 | 31.39303237 | 6.718396361 | A | 109 | 15% | Good |
| POSTN | 0.018770994 | 0.996917249 | 13150 | 98.56093539 | 18.36687176 | 5.726984481 | A | 100 | 50% | Poor |
| MYL9 | 0.019542106 | 0.912226471 | 13172 | 98.72582821 | 18.91298463 | 6.971630847 | A | 91 | 1% | Good |
| MARCH8 | 0.021280706 | 1.045476492 | 12267 | 91.94273722 | 11.60879333 | 6.13962269 | A | 116 | 99% | Good |
| SFPQ | 0.022377897 | 0.979004471 | 8483 | 63.58117224 | 6.580693249 | 7.083770203 | A | 92 | 21% | Good |
| RBM15 | 0.022727478 | 0.976622098 | 9464 | 70.93389297 | 7.268488277 | 6.931840351 | A | 105 | 19% | Good |
| ERBB3 | 0.022850985 | 0.973024232 | 8807 | 66.00959376 | 6.778733253 | 6.14068606 | A | 131 | 16% | Good |
| BTG1 | 0.023096581 | 0.950500449 | 10829 | 81.16474292 | 8.672832814 | 8.570409728 | A | 114 | 5% | Good |
| TPD52 | 0.023234514 | 1.010603283 | 12791 | 95.87018438 | 14.23442693 | 7.516610524 | A | 114 | 81% | Good |
| NUP62 | 0.023300327 | 0.979867486 | 7366 | 55.20911408 | 5.982017633 | 6.942777818 | A | 107 | 22% | Good |
| FAP | 0.023506772 | 1.004061302 | 12595 | 94.40113926 | 12.96841883 | 6.024875876 | A | 98 | 66% | Poor |
| PPAP2B | 0.023748859 | 0.958007438 | 11497 | 86.17148853 | 9.732243317 | 7.364726161 | A | 117 | 8% | Good |
| DNAPTP6 | 0.023803226 | 1.019898578 | 10924 | 81.87678009 | 8.826829556 | 8.065916216 | A | 109 | 91% | Good |
| FYN | 0.023827838 | 1.011799028 | 10351 | 77.58207165 | 8.123534234 | 5.285960467 | A | 116 | 82% | Good |
| LRP2 | 0.024274966 | 1.047306842 | 11282 | 84.56003598 | 9.350029818 | 5.10881537 | A | 113 | 100% | Good |
| PTGS1 | 0.024541016 | 1.036329331 | 8941 | 67.01394094 | 6.86973762 | 6.488618599 | A | 101 | 97% | Good |
| NID2 | 0.026681166 | 1.004849338 | 12654 | 94.84335182 | 13.34147014 | 5.638125179 | A | 118 | 67% | Good |
| TUBB3 | 0.026780898 | 1.015886784 | 10213 | 76.54774397 | 7.969793643 | 8.770726503 | A | 112 | 87% | Good |
| GSK3B | 0.026815921 | 0.972999591 | 10647 | 79.80062959 | 8.445282837 | 6.67339815 | A | 117 | 16% | Good |
| TGFB3 | 0.027153519 | 0.988004808 | 7602 | 56.97796432 | 6.111650196 | 7.149941128 | A | 117 | 30% | Good |
| ECHDC2 | 0.027187608 | 0.962803694 | 10435 | 78.21166242 | 8.201884038 | 7.930889688 | A | 112 | 10% | Good |
| CD97 | 0.027551005 | 1.034406195 | 10260 | 76.90001499 | 8.027118571 | 6.814423675 | A | 120 | 97% | Good |
| PSORS1C2 | 0.027621367 | 1.039509647 | 8787 | 65.8596912 | 6.764158437 | 5.918339127 | A | 132 | 98% | Good |
| SEMA5A | 0.028511613 | 0.992572064 | 8588 | 64.3681607 | 6.645242779 | 6.353350689 | A | 124 | 40% | Poor |
| ANXA5 | 0.028615878 | 0.968071623 | 7480 | 56.06355869 | 6.043746331 | 9.219588653 | A | 117 | 13% | Good |
| DUSP10 | 0.028825844 | 1.016882409 | 11222 | 84.11032829 | 9.25433409 | 5.724554818 | A | 107 | 88% | Good |
| SPARC | 0.02885106 | 0.995086707 | 13194 | 98.89072103 | 19.51614725 | 8.08817232 | A | 117 | 46% | Poor |
| ITGBL1 | 0.03009402 | 0.97201951 | 11693 | 87.64053365 | 10.12036996 | 4.757603281 | A | 127 | 16% | Good |
| PROCR | 0.030248086 | 0.98139105 | 9946 | 74.54654475 | 7.727404282 | 3.756769316 | A | 111 | 23% | Good |
| SH3BGRL | 0.030545781 | 0.961674856 | 11894 | 89.14705441 | 10.54424222 | 7.827599607 | A | 100 | 10% | Good |
| STARD13 | 0.030604916 | 0.982192308 | 7706 | 57.75745765 | 6.158437666 | 6.393370904 | A | 112 | 25% | Good |
| TGFB1I1 | 0.030610931 | 0.950910009 | 12834 | 96.19247489 | 14.57446646 | 5.700066466 | A | 106 | 5% | Good |
| CCDC92 | 0.030971772 | 0.974551506 | 7063 | 52.93809024 | 5.845490151 | 7.344843621 | A | 113 | 17% | Good |
| REEP5 | 0.032462419 | 0.961120433 | 11061 | 82.90361265 | 9.011099714 | 8.221658311 | A | 103 | 9% | Good |
| SKIV2L2 | 0.033171044 | 0.971929622 | 8915 | 66.81906761 | 6.849853258 | 7.005727204 | A | 103 | 16% | Good |
| CTBS | 0.033180484 | 1.009276888 | 8499 | 63.70109429 | 6.590587498 | 5.022598712 | A | 123 | 78% | Good |
| CD59 | 0.033380327 | 0.945253168 | 11724 | 87.87288263 | 10.17727689 | 9.163708042 | A | 125 | 3% | Good |
| DCN | 0.034121792 | 0.939694 | 13254 | 99.34042872 | 21.89477494 | 7.775674163 | A | 110 | 2% | Good |
| CITED2 | 0.0343109 | 0.94685154 | 12543 | 94.01139259 | 12.69664229 | 5.516936887 | A | 106 | 4% | Good |
| FN1 | 0.034487776 | 1.016406804 | 12870 | 96.46229951 | 14.92352135 | 9.972283396 | A | 123 | 88% | Good |
| SERPINF1 | 0.034497864 | 0.953997594 | 13007 | 97.48913206 | 16.23023863 | 7.832252688 | A | 128 | 7% | Good |
| IGFBP3 | 0.034699744 | 1.039429523 | 12801 | 95.94513566 | 14.3166115 | 7.569712229 | A | 125 | 98% | Good |
| SPOCK1 | 0.035214101 | 1.009987543 | 11581 | 86.8010793 | 9.883514348 | 6.096964305 | A | 98 | 79% | Good |
| MYO10 | 0.035286043 | 1.024906164 | 11025 | 82.63378804 | 8.962687791 | 6.388851103 | A | 103 | 94% | Good |
| SSBP2 | 0.035521187 | 0.961467549 | 9918 | 74.33668116 | 7.703550364 | 5.518643423 | A | 133 | 9% | Good |
| NIFUN | 0.035619214 | 0.966546046 | 9340 | 70.00449708 | 7.162211196 | 8.349568156 | A | 109 | 12% | Good |
| LOC388397 | 0.0364212 | 0.981205093 | 8983 | 67.32873632 | 6.894703408 | 4.703300964 | A | 106 | 23% | Good |
| PVRL3 | 0.036718216 | 1.019618758 | 7223 | 54.13731075 | 5.919449686 | 4.987661462 | A | 139 | 91% | Good |
| FSTL1 | 0.036811565 | 0.98276311 | 12923 | 96.8595413 | 15.30596317 | 8.82415883 | A | 107 | 25% | Good |
| CLDN3 | 0.03719767 | 0.963887258 | 12945 | 97.02443412 | 15.53709116 | 7.186124553 | A | 119 | 11% | Good |
| PSMD8 | 0.037260823 | 1.01636378 | 9559 | 71.64593015 | 7.353649877 | 9.122068456 | A | 117 | 87% | Good |
| AGPS | 0.037341896 | 1.037880541 | 11082 | 83.06101034 | 9.035022529 | 5.401390365 | A | 113 | 98% | Good |
| IL13RA1 | 0.037527615 | 0.998913896 | 11398 | 85.42947084 | 9.551024598 | 6.550059063 | A | 122 | 54% | Poor |
| DAB2 | 0.037570858 | 0.971910665 | 11779 | 88.28511468 | 10.30606776 | 6.667650435 | A | 99 | 15% | Good |
| GNG11 | 0.037926758 | 1.001886527 | 12878 | 96.52226053 | 14.99439355 | 5.427606628 | A | 121 | 61% | Poor |
| FKBP5 | 0.038677717 | 0.939940183 | 12957 | 97.11437566 | 15.64621134 | 5.197440247 | A | 99 | 3% | Good |
| SLC38A2 | 0.03907335 | 0.963817018 | 10171 | 76.23294858 | 7.92910891 | 8.820154703 | A | 109 | 11% | Good |
| UGDH | 0.039881238 | 0.974540813 | 11701 | 87.70049468 | 10.133079 | 6.283186837 | A | 132 | 17% | Good |
| DBR1 | 0.040269416 | 0.995654341 | 7764 | 58.19217509 | 6.184331265 | 4.51888702 | A | 113 | 47% | Poor |
| SNRPN | 0.040441891 | 0.950901014 | 12178 | 91.27567081 | 11.31125892 | 7.586200142 | A | 126 | 5% | Good |
| LDHA | 0.040556405 | 0.996967123 | 7059 | 52.90810973 | 5.84445503 | 11.06438928 | A | 123 | 50% | Poor |
| FLNB | 0.041865103 | 1.007390443 | 9479 | 71.04631989 | 7.285309575 | 7.657875997 | A | 105 | 74% | Good |
| KLF13 | 0.042315813 | 0.988769976 | 9971 | 74.73392295 | 7.743982203 | 5.548923778 | A | 119 | 32% | Good |
| KDELR2 | 0.043186242 | 0.981348028 | 10563 | 79.17103882 | 8.360291047 | 8.528602961 | A | 104 | 23% | Good |
| TMED5 | 0.043392451 | 1.007080236 | 9532 | 71.44356168 | 7.325146241 | 6.67780651 | A | 117 | 73% | Good |
| TGIF | 0.043625131 | 1.006912658 | 11045 | 82.7836906 | 8.984191126 | 6.772798007 | A | 109 | 72% | Good |
| CHPT1 | 0.044294713 | 0.952973286 | 11711 | 87.77544596 | 10.15630195 | 7.869692753 | A | 109 | 6% | Good |
| ETS1 | 0.044353829 | 1.018617495 | 7734 | 57.96732124 | 6.171734411 | 5.639135859 | A | 116 | 89% | Good |
| SSR1 | 0.044677648 | 0.976786917 | 10065 | 75.438465 | 7.8232335 | 7.239957828 | A | 102 | 19% | Good |
| DDIT4 | 0.04481242 | 1.039489206 | 12520 | 93.83900465 | 12.58433447 | 8.085099068 | A | 106 | 98% | Good |
| ZEB1 | 0.044974662 | 0.995883789 | 9042 | 67.77094888 | 6.936086814 | 4.55858065 | A | 97 | 47% | Poor |
| CHST2 | 0.045018767 | 0.957579565 | 12713 | 95.28556438 | 13.68689954 | 4.804627223 | A | 125 | 8% | Good |
| TUBB | 0.04517446 | 1.000077573 | 7484 | 56.0935392 | 6.044508267 | 10.61181493 | A | 118 | 56% | Poor |
| GREM1 | 0.045477406 | 0.989901322 | 13088 | 98.09623745 | 17.18917409 | 4.548108995 | A | 108 | 34% | Poor |
| ARHGAP24 | 0.046706069 | 1.023356549 | 7716 | 57.83240893 | 6.162376786 | 4.750632859 | A | 109 | 93% | Good |
| LOC283824 | 0.047299197 | 0.974994664 | 9943 | 74.52405936 | 7.725810909 | 5.674670787 | A | 122 | 18% | Good |
| ID4 | 0.047333161 | 0.926290837 | 13175 | 98.7483136 | 19.04594117 | 5.36801418 | A | 116 | 1% | Good |
| CALD1 | 0.047335546 | 0.975672609 | 11349 | 85.06220956 | 9.469529157 | 5.752658554 | A | 111 | 18% | Good |
| SETD5 | 0.047365706 | 0.97984763 | 8565 | 64.19577275 | 6.634601298 | 7.613185373 | A | 130 | 22% | Good |
| BGN | 0.048339531 | 0.997665346 | 12113 | 90.78848748 | 11.12728011 | 6.262085281 | A | 111 | 52% | Poor |
| HSPA2 | 0.048826033 | 0.931279326 | 13013 | 97.53410283 | 16.25384777 | 5.74573716 | A | 105 | 2% | Good |
| PREPL | 0.048920223 | 0.968369698 | 9528 | 71.41358117 | 7.3227757 | 6.227496378 | A | 112 | 14% | Good |
| PCOLCE | 0.049394724 | 0.989680586 | 12569 | 94.20626593 | 12.80969501 | 6.161098888 | A | 111 | 34% | Poor |
| H2AFZ | 0.049792598 | 0.985456902 | 8993 | 67.4036876 | 6.900564544 | 9.620431347 | A | 118 | 27% | Good |
| FTL | 0.050542822 | 0.982057704 | 7698 | 57.69749663 | 6.152683998 | 11.24754452 | A | 115 | 24% | Good |
| MMP2 | 0.050599232 | 0.977082837 | 12576 | 94.25873182 | 12.86264339 | 7.851665299 | A | 116 | 19% | Good |
| TNFAIP6 | 0.050693153 | 1.037703169 | 12844 | 96.26742617 | 14.6630354 | 5.769113086 | A | 117 | 97% | Good |
| FBN1 | 0.050864318 | 0.964885169 | 12917 | 96.81457053 | 15.24599852 | 6.382559387 | A | 132 | 11% | Good |
| CDKN2C | 0.051563247 | 0.971522551 | 9958 | 74.63648628 | 7.737459293 | 5.54430355 | A | 122 | 15% | Good |
| TGFBR3 | 0.05190111 | 0.953558514 | 13144 | 98.51596462 | 18.28297346 | 5.651828325 | A | 115 | 7% | Good |
| TACSTD1 | 0.052113157 | 0.973656827 | 12362 | 92.6547744 | 11.90521366 | 8.666395185 | A | 130 | 17% | Good |
| APOBEC3G | 0.052130686 | 0.995981903 | 7937 | 59.48883226 | 6.27897091 | 6.518417958 | A | 110 | 47% | Poor |
| MLPH | 0.052329976 | 0.990255975 | 12512 | 93.77904362 | 12.53458876 | 8.238630904 | A | 142 | 35% | Poor |
| THY1 | 0.053261819 | 0.991225808 | 11726 | 87.88787288 | 10.17882942 | 6.873596074 | A | 122 | 38% | Poor |
| PTGER4 | 0.053909735 | 0.976041605 | 11357 | 85.12217059 | 9.487759113 | 5.55424783 | A | 120 | 19% | Good |
| CD44 | 0.054299143 | 1.018693434 | 12061 | 90.39874082 | 10.95846408 | 7.958652143 | A | 116 | 89% | Good |
| RYBP | 0.054526411 | 0.982445682 | 6847 | 51.31914256 | 5.753487942 | 7.151997263 | A | 106 | 25% | Good |
| HIF1A | 0.054595768 | 0.991093187 | 11460 | 85.89416879 | 9.669752382 | 8.316753302 | A | 119 | 37% | Poor |
| PLOD2 | 0.054711258 | 0.978663474 | 12893 | 96.63468745 | 15.14814977 | 6.535608639 | A | 99 | 21% | Good |
| CD24 | 0.05479293 | 1.031812692 | 12081 | 90.54864338 | 11.03343396 | 9.645361683 | A | 118 | 96% | Good |
| MAPT | 0.054829639 | 0.986985328 | 10640 | 79.74816369 | 8.434202522 | 6.175932422 | A | 102 | 29% | Good |
| ZMYM2 | 0.054890756 | 0.977908822 | 8984 | 67.33623145 | 6.894733811 | 4.045907178 | A | 111 | 20% | Good |
| FKSG49 | 0.054912365 | 1.019174242 | 7435 | 55.72627792 | 6.017473205 | 8.740764899 | A | 94 | 89% | Good |
| KLF10 | 0.055936512 | 1.009127749 | 11685 | 87.58057263 | 10.08895166 | 6.739359769 | A | 119 | 77% | Good |
| SMYD3 | 0.056458642 | 1.012037521 | 12029 | 90.15889672 | 10.87096281 | 7.021742714 | A | 107 | 82% | Good |
| PGK1 | 0.056867134 | 0.992071554 | 10570 | 79.22350472 | 8.364592863 | 8.9091619 | A | 130 | 39% | Poor |
| NAT10 | 0.057027928 | 0.994938582 | 8072 | 60.50067456 | 6.341315286 | 7.937177411 | A | 124 | 45% | Poor |
| TNS3 | 0.057440585 | 0.960931473 | 12219 | 91.58297107 | 11.43623106 | 7.526211843 | A | 113 | 9% | Good |
| ALG5 | 0.057593273 | 0.99255466 | 10540 | 78.99865088 | 8.324688197 | 7.87469492 | A | 118 | 40% | Poor |
| CXCL2 | 0.05793095 | 1.013077618 | 12926 | 96.88202668 | 15.3566365 | 5.060339355 | A | 116 | 84% | Good |
| EMP3 | 0.058019782 | 0.989955188 | 11904 | 89.2220057 | 10.56790767 | 7.564953976 | A | 118 | 35% | Poor |
| METTL7A | 0.058125645 | 1.000924279 | 8913 | 66.80407735 | 6.847593838 | 6.032787886 | A | 108 | 58% | Poor |
| ECOP | 0.058250035 | 0.988456949 | 10913 | 81.79433368 | 8.807370769 | 8.156107951 | A | 120 | 31% | Good |
| DNAJB1 | 0.058387278 | 0.99463299 | 9030 | 67.68100735 | 6.929318486 | 7.824524157 | A | 88 | 44% | Poor |
| SEPT6 | 0.058499125 | 1.001453977 | 10447 | 78.30160396 | 8.213386956 | 5.611716712 | A | 110 | 60% | Poor |
| FOXO1A | 0.058684039 | 0.991662529 | 7269 | 54.48208664 | 5.938461181 | 6.427190252 | A | 122 | 39% | Poor |
| KNTC2 | 0.05875687 | 1.019577799 | 13062 | 97.90136411 | 16.83772312 | 4.876486199 | A | 113 | 91% | Good |
| SDF4 | 0.058901527 | 0.996027205 | 9563 | 71.67591066 | 7.355976181 | 7.244552352 | A | 119 | 47% | Poor |
| LTF | 0.058991843 | 0.891657126 | 13282 | 99.55029231 | 22.88262723 | 9.078743245 | A | 111 | 0% | Good |
| PMP22 | 0.059411595 | 0.953937134 | 12782 | 95.80272823 | 14.17441451 | 8.430933931 | A | 115 | 7% | Good |
| CEBPD | 0.059658661 | 1.001791786 | 9140 | 68.50547144 | 7.010599231 | 6.572230051 | A | 106 | 61% | Poor |
| THBS1 | 0.059998233 | 0.994531318 | 11946 | 89.53680108 | 10.66998094 | 5.646315594 | A | 124 | 44% | Poor |
| TGIF2 | 0.060179816 | 1.004051263 | 9055 | 67.86838555 | 6.941024143 | 6.378831142 | A | 101 | 65% | Poor |
| PSMB7 | 0.061335145 | 1.01191009 | 8348 | 62.56932994 | 6.48988523 | 9.130438587 | A | 118 | 82% | Good |
| MMP1 | 0.062035976 | 0.922115184 | 13339 | 99.97751462 | 35.83680532 | 4.419777459 | A | 116 | 1% | Good |
| CYP1B1 | 0.062435749 | 0.964387374 | 13308 | 99.74516564 | 24.66200778 | 6.280096841 | A | 107 | 11% | Good |
| SCGN | 0.062736617 | 1.009032736 | 7024 | 52.64578024 | 5.82868347 | 6.679948767 | A | 99 | 77% | Good |
| MEF2A | 0.062915503 | 0.99079724 | 10416 | 78.06925498 | 8.180122908 | 4.507490413 | A | 118 | 36% | Poor |
| NRP1 | 0.062947993 | 1.005506589 | 12642 | 94.75341028 | 13.24482768 | 4.919936938 | A | 130 | 69% | Good |
| MMP9 | 0.063140539 | 0.95536991 | 12743 | 95.51041823 | 13.85506057 | 8.579089506 | A | 112 | 8% | Good |
| PPP3CA | 0.063429532 | 0.968182261 | 11224 | 84.12531854 | 9.25664422 | 7.268082335 | A | 121 | 14% | Good |
| TGFBI | 0.063449576 | 1.017707534 | 12162 | 91.15574876 | 11.26661692 | 8.60122753 | A | 142 | 88% | Good |
| ISGF3G | 0.063691853 | 0.99289378 | 10546 | 79.04362165 | 8.332493251 | 8.601769853 | A | 127 | 41% | Poor |
| STAM | 0.063955366 | 0.993307853 | 10968 | 82.20656573 | 8.885919946 | 6.485197543 | A | 137 | 42% | Poor |
| CDH1 | 0.063990642 | 0.986256849 | 12438 | 93.22440414 | 12.22373315 | 6.919764517 | A | 114 | 28% | Good |
| SEPT9 | 0.064233513 | 0.981691306 | 9086 | 68.10073452 | 6.963536599 | 6.461530497 | A | 126 | 24% | Good |
| KDELR3 | 0.064321613 | 0.989231356 | 11717 | 87.82041673 | 10.1645103 | 7.313461377 | A | 100 | 33% | Good |
| ARID5B | 0.064431404 | 0.973368765 | 11337 | 84.97226803 | 9.447693266 | 7.790965233 | A | 125 | 17% | Good |
| RGL1 | 0.064552908 | 0.994087901 | 10265 | 76.93749063 | 8.03376252 | 7.540015464 | A | 109 | 44% | Poor |
| COL1A1 | 0.064582159 | 0.993702236 | 12042 | 90.25633338 | 10.91231407 | 7.001737454 | A | 117 | 43% | Poor |
| ATP2B1 | 0.065112625 | 0.991672492 | 11195 | 83.90795983 | 9.212229629 | 4.081174457 | A | 102 | 39% | Poor |
| CDKN1A | 0.065400067 | 1.012062062 | 8839 | 66.24943787 | 6.800113431 | 7.792606502 | A | 102 | 83% | Good |
| LRIG1 | 0.065419254 | 0.936986999 | 12494 | 93.64413131 | 12.46306291 | 5.693379708 | A | 128 | 2% | Good |
| CCND2 | 0.065595281 | 0.984122713 | 8107 | 60.76300405 | 6.359827224 | 5.566803935 | A | 117 | 26% | Good |
| MSX1 | 0.065792082 | 0.997608989 | 10048 | 75.31104782 | 7.810901502 | 4.388789279 | A | 127 | 51% | Poor |
| IER3 | 0.065942671 | 1.011484853 | 12218 | 91.57547594 | 11.43448028 | 8.273296205 | A | 101 | 81% | Good |
| FGFR2 | 0.066022239 | 1.00281586 | 10523 | 78.8712337 | 8.303116129 | 5.273177578 | A | 102 | 64% | Poor |
| FRAS1 | 0.066232058 | 1.018215725 | 8104 | 60.74051866 | 6.359422969 | 5.571262561 | A | 106 | 89% | Good |
| BUB1 | 0.066374098 | 1.010229466 | 8094 | 60.66556738 | 6.356090093 | 5.008199211 | A | 111 | 80% | Good |
| TAGLN | 0.066753768 | 0.945502863 | 13240 | 99.23549693 | 21.34313683 | 8.023282275 | A | 112 | 3% | Good |
| FADS2 | 0.066894084 | 1.00928935 | 12092 | 90.63108979 | 11.0644294 | 7.025023687 | A | 118 | 78% | Good |
| TPM1 | 0.06769776 | 0.951736201 | 12608 | 94.49857593 | 13.03768935 | 8.198073725 | A | 133 | 5% | Good |
| PLAA | 0.068144961 | 1.006889525 | 7456 | 55.88367561 | 6.030267694 | 4.624008733 | A | 119 | 72% | Good |
| FBLN5 | 0.068145172 | 0.966143729 | 11007 | 82.49887573 | 8.94296832 | 6.828876438 | A | 124 | 12% | Good |
| VIM | 0.068301136 | 0.990511107 | 9956 | 74.62149603 | 7.735132821 | 11.32438972 | A | 101 | 36% | Poor |
| DPYSL3 | 0.068327827 | 0.999031789 | 12043 | 90.26382851 | 10.91821131 | 6.179769285 | A | 107 | 55% | Poor |
| TP53 | 0.068890725 | 1.002054383 | 12540 | 93.98890721 | 12.69380463 | 5.450920278 | A | 104 | 62% | Poor |
| SH3BGRL3 | 0.068989975 | 0.998889705 | 8519 | 63.85099685 | 6.604720209 | 8.473259078 | A | 141 | 54% | Poor |
| PDGFC | 0.069010155 | 0.96995612 | 13076 | 98.00629591 | 17.04374598 | 6.827112736 | A | 126 | 15% | Good |
| VCAN | 0.069297615 | 1.000936397 | 13136 | 98.4560036 | 18.17202162 | 5.291336585 | A | 111 | 58% | Poor |
| IGFBP4 | 0.069437136 | 0.968345316 | 12817 | 96.06505771 | 14.42612452 | 7.456892762 | A | 107 | 14% | Good |
| METTL2B | 0.069484582 | 1.017701034 | 8334 | 62.46439814 | 6.48229014 | 3.487954209 | A | 115 | 88% | Good |
| BAT3 | 0.069508605 | 0.998700011 | 7285 | 54.60200869 | 5.944249708 | 8.654222218 | A | 103 | 54% | Poor |
| ZBTB38 | 0.069949149 | 0.966746157 | 11310 | 84.76989957 | 9.403118607 | 6.946326518 | A | 118 | 13% | Good |
| EIF4ENIF1 | 0.070126638 | 0.989140522 | 7777 | 58.28961175 | 6.191371812 | 6.104279535 | A | 127 | 33% | Good |
| PPM1D | 0.070191618 | 0.987304421 | 9155 | 68.61789837 | 7.021412099 | 4.886494829 | A | 117 | 29% | Good |
| TUBA3 | 0.070205258 | 0.981819985 | 11966 | 89.68670364 | 10.71903946 | 9.146891529 | A | 133 | 24% | Good |
| CSTF1 | 0.070220098 | 0.991464442 | 6869 | 51.48403538 | 5.762287305 | 5.4732504 | A | 114 | 38% | Poor |
| ATP13A3 | 0.070749942 | 0.993536962 | 7089 | 53.13296357 | 5.856718599 | 6.019358571 | A | 124 | 42% | Poor |
| STC1 | 0.070875003 | 1.026596717 | 12493 | 93.63663619 | 12.45013807 | 5.828474049 | A | 117 | 94% | Good |
| MYCBP2 | 0.070973157 | 0.981188131 | 11001 | 82.45390496 | 8.930354974 | 7.56106142 | A | 107 | 22% | Good |
| LMCD1 | 0.070998528 | 0.959786862 | 10786 | 80.84245241 | 8.610524808 | 6.885739155 | A | 117 | 8% | Good |
| S100A4 | 0.071013984 | 0.98603156 | 12234 | 91.69539799 | 11.49326185 | 8.514013401 | A | 115 | 27% | Good |
| SDC2 | 0.071226367 | 0.949185923 | 12849 | 96.30490181 | 14.70925771 | 7.304027171 | A | 121 | 4% | Good |
| NUCKS1 | 0.071342918 | 0.988673035 | 7914 | 59.31644431 | 6.265676817 | 8.208101691 | A | 120 | 31% | Good |
| KRT5 | 0.071586657 | 0.961563148 | 13226 | 99.13056513 | 20.60166991 | 8.123189295 | A | 112 | 10% | Good |
| USP22 | 0.071976141 | 1.020473168 | 7115 | 53.32783691 | 5.87046935 | 4.829598306 | A | 112 | 91% | Good |
| MYL1 | 0.072597769 | 1.018112974 | 7693 | 57.66002099 | 6.150561576 | 5.445345463 | A | 116 | 89% | Good |
| FXYD3 | 0.072830343 | 0.985294431 | 10235 | 76.71263679 | 8.005600752 | 8.756347961 | A | 119 | 26% | Good |
| AKAP2 | 0.072864843 | 0.986609499 | 12437 | 93.21690901 | 12.22222858 | 6.19271358 | A | 129 | 28% | Good |
| ENPP2 | 0.072910324 | 0.951935449 | 13190 | 98.86074052 | 19.40597917 | 5.846518734 | A | 125 | 6% | Good |
| MAST4 | 0.074053136 | 1.00872382 | 7510 | 56.28841253 | 6.061291271 | 6.683026188 | A | 121 | 76% | Good |
| LRPAP1 | 0.07418212 | 0.988766696 | 7837 | 58.73931944 | 6.223163164 | 7.410948634 | A | 102 | 32% | Good |
| ECM1 | 0.074561655 | 0.992787288 | 7717 | 57.83990406 | 6.164484692 | 7.046216208 | A | 112 | 41% | Poor |
| STAU1 | 0.074831662 | 0.989242699 | 7908 | 59.27147354 | 6.263780625 | 9.021766442 | A | 125 | 33% | Poor |
| GAS7 | 0.074852579 | 0.989957448 | 8577 | 64.28571429 | 6.640121271 | 5.827292079 | A | 127 | 35% | Poor |
| MAP4K4 | 0.075044686 | 0.987534308 | 9962 | 74.6664668 | 7.740300545 | 5.896665079 | A | 109 | 29% | Good |
| HS3ST2 | 0.075415011 | 1.013784175 | 6960 | 52.16609204 | 5.800160915 | 6.232013975 | A | 112 | 85% | Good |
| PLEKHC1 | 0.075632005 | 0.965296409 | 12368 | 92.69974517 | 11.9137684 | 5.30124757 | A | 115 | 12% | Good |
| NSF | 0.076431045 | 0.988756452 | 7948 | 59.57127867 | 6.283194251 | 7.482771953 | A | 118 | 31% | Good |
| MT2A | 0.076533192 | 0.978656007 | 8735 | 65.46994454 | 6.728458889 | 10.93221467 | A | 96 | 21% | Good |
| MMP14 | 0.076548535 | 1.006404677 | 6730 | 50.44221256 | 5.703151797 | 6.774690793 | A | 115 | 70% | Good |
| MLF1 | 0.076721003 | 0.999914598 | 12412 | 93.0295308 | 12.12445146 | 5.966685543 | A | 108 | 56% | Poor |
| NUP37 | 0.076823725 | 0.998581101 | 9775 | 73.26487783 | 7.5586067 | 6.985063829 | A | 109 | 53% | Poor |
| BDH2 | 0.076885147 | 0.952537387 | 11735 | 87.95532904 | 10.19544234 | 6.189184891 | A | 120 | 6% | Good |
| MRPL42 | 0.077137429 | 0.988894716 | 9201 | 68.96267426 | 7.053594083 | 7.812151962 | A | 94 | 32% | Good |
| ETNK1 | 0.077272351 | 0.968555234 | 11794 | 88.3975416 | 10.3308613 | 4.768183348 | A | 123 | 14% | Good |
| C10ORF7 | 0.077852801 | 1.006595942 | 10188 | 76.36036576 | 7.944578698 | 8.478487642 | A | 149 | 71% | Good |
| WEE1 | 0.077876722 | 0.988024797 | 7868 | 58.97166842 | 6.238141723 | 7.164204426 | A | 117 | 31% | Good |
| LTBP2 | 0.07791878 | 0.986831097 | 11283 | 84.5675311 | 9.350076099 | 6.999420183 | A | 104 | 28% | Good |
| KCNMA1 | 0.077966763 | 0.993302798 | 11198 | 83.93044521 | 9.218242947 | 5.152484658 | A | 138 | 42% | Poor |
| SORBS2 | 0.079306562 | 0.990643114 | 9580 | 71.80332784 | 7.370629882 | 6.524991842 | A | 118 | 36% | Poor |
| NR2F1 | 0.079595536 | 0.966615968 | 12086 | 90.58611902 | 11.05286316 | 5.066994165 | A | 131 | 13% | Good |
| DPT | 0.079735764 | 0.952182936 | 12967 | 97.18932694 | 15.71852287 | 5.047312052 | A | 107 | 6% | Good |
| HNMT | 0.080033585 | 0.99127788 | 7084 | 53.09548793 | 5.855770954 | 4.780660847 | A | 101 | 38% | Poor |
| DNAJC13 | 0.080338554 | 1.002215106 | 6821 | 51.12426923 | 5.739985685 | 5.195115649 | A | 105 | 62% | Poor |
| NEBL | 0.080499686 | 0.977443415 | 8240 | 61.75985609 | 6.432740239 | 6.039167608 | A | 100 | 20% | Good |
| KPNA3 | 0.080518202 | 1.003541345 | 7372 | 55.25408484 | 5.98392567 | 7.118066712 | A | 123 | 65% | Poor |
| SCCPDH | 0.08111085 | 0.994540059 | 10085 | 75.58836756 | 7.849323848 | 6.954962555 | A | 110 | 44% | Poor |
| TBX3 | 0.081153545 | 0.96702269 | 12143 | 91.01334133 | 11.20941391 | 5.639524448 | A | 109 | 13% | Good |
| CDH2 | 0.081459821 | 1.009182615 | 7268 | 54.47459152 | 5.937989375 | 4.968761682 | A | 111 | 78% | Good |
| AMMECR1 | 0.0823953 | 1.033372614 | 12395 | 92.90211363 | 12.06524327 | 6.173349279 | A | 109 | 96% | Good |
| PLP2 | 0.082539275 | 0.992919803 | 10271 | 76.9824614 | 8.041833925 | 8.615654634 | A | 107 | 41% | Poor |
| HS2ST1 | 0.082735053 | 1.015298178 | 9067 | 67.95832709 | 6.949328432 | 4.213077405 | A | 118 | 86% | Good |
| CNIH4 | 0.082912768 | 1.019177709 | 12626 | 94.63348823 | 13.1654349 | 6.813580509 | A | 132 | 90% | Good |
| KIAA1217 | 0.083305849 | 1.015571208 | 6802 | 50.98186179 | 5.731883811 | 7.811091686 | A | 120 | 86% | Good |
| EIF2C2 | 0.08349556 | 1.034393949 | 10900 | 81.69689702 | 8.780506716 | 6.065294901 | A | 118 | 96% | Good |
| ICMT | 0.084397089 | 1.002920288 | 8879 | 66.54924299 | 6.825259531 | 6.307082341 | A | 107 | 64% | Poor |
| IER5 | 0.085452591 | 1.005831521 | 12704 | 95.21810823 | 13.62496933 | 6.878858514 | A | 125 | 69% | Good |
| NOTCH2 | 0.085543756 | 0.974260289 | 8875 | 66.51926248 | 6.823958309 | 7.514383233 | A | 109 | 17% | Good |
| VTCN1 | 0.08578649 | 0.981477259 | 12921 | 96.84455104 | 15.29110333 | 8.054099209 | A | 91 | 23% | Good |
| MAFF | 0.086497949 | 1.007013768 | 12479 | 93.53170439 | 12.39351987 | 6.009550547 | A | 114 | 73% | Good |
| SNAI2 | 0.086574535 | 0.962420986 | 13201 | 98.94318693 | 19.74765063 | 5.322643176 | A | 117 | 10% | Good |
| FMNL2 | 0.086841007 | 1.028262012 | 7678 | 57.54759406 | 6.144369796 | 5.447286508 | A | 122 | 95% | Good |
| WFDC2 | 0.087257206 | 0.975271754 | 12388 | 92.84964773 | 12.02598283 | 7.118478554 | A | 109 | 18% | Good |
| GABARAPL1 | 0.087277933 | 0.995266597 | 8892 | 66.64667966 | 6.833603312 | 5.847708059 | A | 124 | 46% | Poor |
| CASP8 | 0.087421397 | 1.002688822 | 7957 | 59.63873482 | 6.286765402 | 5.897421355 | A | 104 | 64% | Poor |
| NID1 | 0.087843474 | 0.989749576 | 12306 | 92.23504722 | 11.72130736 | 5.171256868 | A | 110 | 34% | Poor |
| RAB23 | 0.087857858 | 0.992586751 | 9113 | 68.30310298 | 6.984974883 | 4.712004618 | A | 121 | 40% | Poor |
| STC2 | 0.087904728 | 0.985726133 | 10262 | 76.91500525 | 8.031336724 | 6.919758563 | A | 116 | 27% | Good |
| AIM1 | 0.088211291 | 0.981017219 | 12633 | 94.68595413 | 13.18773458 | 8.094941948 | A | 108 | 22% | Good |
| ZBTB20 | 0.088478936 | 0.976149944 | 9230 | 69.18003298 | 7.078161877 | 6.102837084 | A | 120 | 19% | Good |
| PAK2 | 0.088737701 | 1.001933025 | 11298 | 84.67995803 | 9.381673543 | 5.839209371 | A | 124 | 62% | Poor |
| ANK3 | 0.088801077 | 0.987388726 | 11407 | 85.496927 | 9.566173955 | 6.157982418 | A | 122 | 29% | Good |
| WNT5A | 0.088962096 | 0.99843273 | 11659 | 87.3856993 | 10.01262977 | 4.690399636 | A | 113 | 53% | Poor |
| EXT1 | 0.089319348 | 1.008762726 | 7389 | 55.38150202 | 5.990970898 | 6.381146541 | A | 119 | 76% | Good |
| COL11A2 | 0.091309029 | 1.010180563 | 6725 | 50.40473692 | 5.700008942 | 6.460264931 | A | 105 | 80% | Good |
| RIN2 | 0.091892713 | 0.989724047 | 11680 | 87.54309699 | 10.0710586 | 6.867399743 | A | 118 | 34% | Poor |
| SGK3 | 0.092100841 | 0.986226616 | 13078 | 98.02128616 | 17.08613324 | 4.076982724 | A | 120 | 28% | Good |
| CEP350 | 0.092399536 | 1.005254166 | 9349 | 70.07195323 | 7.168109756 | 6.272724389 | A | 115 | 68% | Good |
| BASP1 | 0.093953473 | 1.004659362 | 12424 | 93.11947234 | 12.17698969 | 8.634079152 | A | 126 | 67% | Good |
| STK39 | 0.094477437 | 0.977826945 | 12255 | 91.85279568 | 11.55444502 | 5.290319946 | A | 106 | 20% | Good |
| PTX3 | 0.094693519 | 0.961407318 | 13323 | 99.85759256 | 28.1324859 | 4.311307552 | A | 88 | 9% | Good |
| NFYA | 0.095713033 | 0.99611783 | 7144 | 53.54519562 | 5.882806875 | 6.129571257 | A | 110 | 47% | Poor |
| MME | 0.096026621 | 0.996843604 | 12051 | 90.32378954 | 10.94090406 | 6.208853699 | A | 109 | 49% | Poor |
| SPP1 | 0.0973073 | 1.034622206 | 13089 | 98.10373257 | 17.20035112 | 9.602476381 | A | 108 | 97% | Good |
| CPS1 | 0.097351825 | 0.984522832 | 12708 | 95.24808874 | 13.65547073 | 4.654868227 | A | 120 | 26% | Good |
| TFPI | 0.097929983 | 0.999249541 | 12427 | 93.14195773 | 12.18453708 | 3.95334446 | A | 115 | 55% | Poor |
| ATIC | 0.098124163 | 0.992285019 | 9002 | 67.47114376 | 6.907580925 | 8.193399336 | A | 88 | 40% | Poor |
| PTGFR | 0.098956076 | 0.990940671 | 7852 | 58.85174636 | 6.231009874 | 3.58087155 | A | 111 | 37% | Poor |
| TOB2 | 0.099371499 | 0.997273579 | 8416 | 63.07899865 | 6.540253029 | 6.04147893 | A | 113 | 50% | Poor |
| MARS | 0.100664359 | 0.991546405 | 11440 | 85.74426623 | 9.638801634 | 6.697500891 | A | 111 | 39% | Poor |
| SRP54 | 0.101071913 | 0.977955491 | 9320 | 69.85459451 | 7.14508165 | 7.668295957 | A | 102 | 21% | Good |
| KRT6B | 0.101564496 | 1.004845522 | 13059 | 97.87887873 | 16.77676281 | 7.477978183 | A | 94 | 67% | Good |
| EPPK1 | 0.103045521 | 0.997754807 | 10739 | 80.49018138 | 8.553905214 | 7.057086919 | A | 112 | 52% | Poor |
| CES1 | 0.103365221 | 0.993725109 | 11159 | 83.63813521 | 9.152249574 | 5.861298358 | A | 103 | 43% | Poor |
| THEM2 | 0.104189298 | 0.997303091 | 10852 | 81.33713086 | 8.714432399 | 7.038182753 | A | 122 | 51% | Poor |
| PFKFB3 | 0.104988685 | 1.005322731 | 11482 | 86.05906161 | 9.710066398 | 6.755897972 | A | 100 | 69% | Good |
| RAD23B | 0.104990727 | 0.997097047 | 10412 | 78.03927447 | 8.175735873 | 7.563156654 | A | 111 | 50% | Poor |
| EMP1 | 0.107749455 | 0.989455201 | 13242 | 99.25048718 | 21.3926789 | 6.131865312 | A | 107 | 33% | Poor |
| MMP7 | 0.110763102 | 1.00661322 | 13280 | 99.53530205 | 22.77381948 | 8.379755002 | A | 121 | 71% | Good |
| PILRB | 0.113239479 | 0.994640009 | 11425 | 85.6318393 | 9.6085534 | 6.967198571 | A | 125 | 45% | Poor |
| LAMA2 | 0.113712178 | 0.986215791 | 10475 | 78.51146755 | 8.257917231 | 4.37876049 | A | 112 | 28% | Good |
| CHST11 | 0.11401461 | 1.006465565 | 10070 | 75.47594064 | 7.833199439 | 5.091184862 | A | 114 | 71% | Good |
| ANXA6 | 0.116727976 | 0.987943215 | 10398 | 77.93434268 | 8.167392935 | 7.920064873 | A | 107 | 30% | Good |
| GTF3C3 | 0.116876154 | 0.992607052 | 9233 | 69.20251836 | 7.079273961 | 6.423745468 | A | 96 | 41% | Poor |
| LTBP1 | 0.118087056 | 1.001437177 | 9031 | 67.68850247 | 6.931433998 | 7.06272816 | A | 127 | 59% | Poor |
| PTGER2 | 0.11986875 | 0.997094826 | 7944 | 59.54129816 | 6.282635374 | 4.598143852 | A | 125 | 50% | Poor |
| FBLN1 | 0.121359663 | 0.998315961 | 8781 | 65.81472043 | 6.759868092 | 6.842070183 | A | 102 | 53% | Poor |
| GPRC5A | 0.122961107 | 0.987611101 | 12765 | 95.67531105 | 14.05034413 | 7.555993599 | A | 101 | 30% | Good |
| GNPDA1 | 0.12509288 | 0.999533044 | 8302 | 62.22455404 | 6.467482305 | 6.879013896 | A | 99 | 56% | Poor |
| SWAP70 | 0.130474933 | 0.981547602 | 10117 | 75.82821166 | 7.875858511 | 5.830347266 | A | 111 | 24% | Good |
| ATP1B3 | 0.131830106 | 0.999420865 | 10091 | 75.63333833 | 7.851367399 | 9.545326018 | A | 111 | 56% | Poor |
| MMP10 | 0.164779488 | 0.998396792 | 10920 | 81.84679958 | 8.822068421 | 3.745111984 | A | 112 | 53% | Poor |
| JTV1 | 0.003204972 | 1.022919703 | 5826 | 43.6666167 | 5.352863101 | 8.174106486 | B | 109 | 93% | Good |
| GBX2 | 0.014449505 | 1.026339068 | 5505 | 41.26068056 | 5.226970939 | 4.051663642 | B | 115 | 94% | Good |
| XPNPEP1 | 0.015396931 | 0.968931722 | 6297 | 47.19682207 | 5.529644192 | 6.996324653 | B | 131 | 14% | Good |
| SNED1 | 0.018307564 | 1.013080486 | 4187 | 31.38210163 | 4.813588383 | 4.998283657 | B | 109 | 84% | Good |
| SLC11A1 | 0.018929015 | 1.02313728 | 4569 | 34.24524059 | 4.927450102 | 6.763001666 | B | 127 | 93% | Good |
| ADAMTS7 | 0.023034398 | 1.014112283 | 6444 | 48.29860591 | 5.583706838 | 5.138618655 | B | 128 | 85% | Good |
| GTPBP1 | 0.023807917 | 1.020920722 | 3745 | 28.06925498 | 4.672224091 | 5.919511762 | B | 114 | 92% | Good |
| TRAM2 | 0.023969061 | 0.977420929 | 5905 | 44.25873182 | 5.380820292 | 6.604805949 | B | 100 | 20% | Good |
| MAPK14 | 0.02547542 | 1.014325417 | 4002 | 29.99550292 | 4.758622153 | 6.580294539 | B | 116 | 85% | Good |
| LRP1 | 0.027488558 | 0.991611677 | 3340 | 25.03372808 | 4.543243403 | 6.835485128 | B | 131 | 39% | Poor |
| GNB2L1 | 0.030890249 | 0.974775467 | 6459 | 48.41103283 | 5.590550504 | 7.910934413 | B | 110 | 18% | Good |
| CAMK2B | 0.031161237 | 0.993300817 | 5411 | 40.55613851 | 5.195039495 | 4.696629449 | B | 137 | 42% | Poor |
| TGFB1 | 0.032505161 | 0.996191955 | 5423 | 40.64608005 | 5.199865271 | 6.279307709 | B | 112 | 48% | Poor |
| TRIO | 0.035568563 | 1.019459509 | 3437 | 25.76075551 | 4.571591941 | 5.632634989 | B | 129 | 90% | Good |
| CLIC5 | 0.038302723 | 1.026880935 | 6620 | 49.61774846 | 5.653526997 | 4.250555849 | B | 126 | 95% | Good |
| DNAJB4 | 0.040587612 | 1.008282713 | 6322 | 47.38420027 | 5.538953258 | 4.353922559 | B | 118 | 75% | Good |
| TMEM63A | 0.042033336 | 1.008461021 | 5167 | 38.72732724 | 5.121087294 | 6.129186684 | B | 113 | 75% | Good |
| PSD | 0.042758143 | 1.01174101 | 6652 | 49.85759256 | 5.666413442 | 5.045674777 | B | 104 | 81% | Good |
| NT5E | 0.042867461 | 1.008493845 | 4214 | 31.58447009 | 4.821287185 | 5.740755289 | B | 100 | 75% | Good |
| PSMA5 | 0.042932042 | 1.001177544 | 6455 | 48.38105232 | 5.5883386 | 8.466188705 | B | 112 | 59% | Poor |
| FBXL18 | 0.043121041 | 1.023814184 | 5681 | 42.57982311 | 5.285119356 | 4.842451458 | B | 100 | 94% | Good |
| ST3GAL5 | 0.043414465 | 0.986198492 | 6259 | 46.9120072 | 5.514777943 | 7.189554894 | B | 120 | 27% | Good |
| SMPD1 | 0.046288268 | 1.021137066 | 4337 | 32.50637086 | 4.861915365 | 5.313541576 | B | 110 | 92% | Good |
| ARF3 | 0.04663004 | 0.98395595 | 3521 | 26.39034627 | 4.598461631 | 8.526647644 | B | 117 | 25% | Good |
| GAPDH | 0.047951792 | 0.996452041 | 3875 | 29.04362165 | 4.719251752 | 12.43369353 | B | 131 | 48% | Poor |
| COPZ2 | 0.048545006 | 1.002960187 | 6255 | 46.88202668 | 5.512841087 | 6.965871158 | B | 109 | 65% | Poor |
| IL8RA | 0.048654499 | 1.011601337 | 4331 | 32.46140009 | 4.860430883 | 7.247323975 | B | 125 | 81% | Good |
| ELL2 | 0.052024721 | 1.008268505 | 4768 | 35.7367711 | 4.985786697 | 5.312578136 | B | 97 | 75% | Good |
| FXYD5 | 0.052604063 | 1.021814942 | 6562 | 49.18303103 | 5.631635597 | 7.298651887 | B | 101 | 92% | Good |
| KLK3 | 0.053451536 | 1.004810946 | 3937 | 29.50831959 | 4.735273566 | 6.352365244 | B | 115 | 67% | Good |
| ATXN3 | 0.053853417 | 1.010645532 | 4884 | 36.60620597 | 5.028065588 | 3.94858219 | B | 103 | 81% | Good |
| C1ORF63 | 0.054714725 | 0.991147236 | 4311 | 32.31149753 | 4.853094096 | 6.43854867 | B | 122 | 37% | Poor |
| ELP4 | 0.056871199 | 1.008878238 | 5812 | 43.5616849 | 5.347421718 | 6.392940346 | B | 117 | 77% | Good |
| PCTK2 | 0.057264782 | 0.996619794 | 3355 | 25.146155 | 4.547917914 | 5.257992978 | B | 97 | 49% | Poor |
| CNOT4 | 0.058563281 | 1.015498212 | 4737 | 35.50442213 | 4.975482415 | 4.647202978 | B | 113 | 86% | Good |
| ARPC5 | 0.058812533 | 0.993132789 | 4151 | 31.11227702 | 4.802391759 | 9.551114774 | B | 113 | 42% | Poor |
| TNRC6B | 0.061585503 | 1.00493343 | 5387 | 40.37625543 | 5.187061915 | 5.876065572 | B | 121 | 68% | Good |
| CNR1 | 0.062502761 | 1.010340114 | 4953 | 37.12336981 | 5.053719929 | 5.27782066 | B | 104 | 80% | Good |
| WWOX | 0.064188007 | 1.011831147 | 3392 | 25.42347474 | 4.558316791 | 5.91322382 | B | 130 | 82% | Good |
| PRKCA | 0.064519713 | 1.006996779 | 5970 | 44.74591516 | 5.40503885 | 5.681840748 | B | 120 | 73% | Good |
| TGFBR1 | 0.065287493 | 1.004114214 | 5849 | 43.83900465 | 5.358698653 | 4.826721378 | B | 115 | 66% | Good |
| UGCGL1 | 0.066585663 | 1.019522416 | 5700 | 42.72223055 | 5.2904279 | 5.572911872 | B | 112 | 90% | Good |
| COPB2 | 0.068075935 | 0.993990059 | 4101 | 30.73752061 | 4.787062704 | 6.1498873 | B | 128 | 44% | Poor |
| TAS2R14 | 0.068194207 | 1.002110981 | 5716 | 42.8421526 | 5.299596368 | 5.423281781 | B | 104 | 62% | Poor |
| DOCK10 | 0.068227546 | 1.000096802 | 6347 | 47.57157847 | 5.548950174 | 3.932605508 | B | 125 | 57% | Poor |
| DCUN1D4 | 0.069000046 | 0.99480297 | 4209 | 31.54699445 | 4.820055146 | 5.788236954 | B | 118 | 45% | Poor |
| FAM53C | 0.070065685 | 1.0040693 | 3476 | 26.05306551 | 4.581495102 | 7.588488115 | B | 117 | 66% | Poor |
| ZFHX1B | 0.071091597 | 0.997750729 | 5177 | 38.80227852 | 5.122745904 | 3.463636081 | B | 112 | 52% | Poor |
| PTPRA | 0.071162198 | 0.987805878 | 4534 | 33.98291111 | 4.918221295 | 5.554080282 | B | 122 | 30% | Good |
| LPIN2 | 0.071184707 | 1.005147909 | 4593 | 34.42512367 | 4.936699448 | 6.303011207 | B | 122 | 68% | Good |
| NDEL1 | 0.071868675 | 1.001613164 | 6610 | 49.54279718 | 5.649116738 | 6.838990595 | B | 103 | 60% | Poor |
| DNM1 | 0.072039805 | 1.009705227 | 3367 | 25.23609654 | 4.552789571 | 6.022019507 | B | 122 | 79% | Good |
| BIN1 | 0.072858777 | 0.990182196 | 6170 | 46.24494079 | 5.478892673 | 7.159294163 | B | 116 | 35% | Poor |
| CREB3L1 | 0.073155202 | 1.007507766 | 4035 | 30.24284215 | 4.767435118 | 6.463534704 | B | 126 | 74% | Good |
| HAS2 | 0.073188069 | 1.002887889 | 5527 | 41.42557338 | 5.234724161 | 4.000166384 | B | 93 | 64% | Poor |
| SDHAL1 | 0.076750617 | 1.005968065 | 3422 | 25.64832859 | 4.567198875 | 8.763855971 | B | 108 | 70% | Good |
| ROR1 | 0.078740429 | 1.002352518 | 4871 | 36.5087693 | 5.023136838 | 4.997233037 | B | 108 | 63% | Poor |
| EIF4E2 | 0.081254067 | 1.000733692 | 6614 | 49.57277769 | 5.651590085 | 6.66540388 | B | 131 | 58% | Poor |
| EML1 | 0.081877176 | 0.995097599 | 5730 | 42.9470844 | 5.307363622 | 3.983998361 | B | 105 | 46% | Poor |
| FAM49B | 0.08353526 | 1.01236775 | 4153 | 31.12726728 | 4.802850912 | 5.016285835 | B | 110 | 83% | Good |
| CHST7 | 0.08568526 | 1.00863996 | 5293 | 39.67171339 | 5.158784112 | 5.447210971 | B | 119 | 76% | Good |
| QKI | 0.087219371 | 0.990747708 | 5687 | 42.62479388 | 5.28662618 | 5.984907217 | B | 113 | 36% | Poor |
| SNX6 | 0.087819189 | 0.993569263 | 4075 | 30.54264728 | 4.778104354 | 7.363871414 | B | 119 | 43% | Poor |
| LARP1 | 0.088542647 | 0.999517496 | 3500 | 26.23294858 | 4.589809462 | 8.103120611 | B | 126 | 56% | Poor |
| DDB2 | 0.09261684 | 0.983955802 | 6265 | 46.95697796 | 5.517217091 | 6.18229142 | B | 95 | 25% | Good |
| DDR2 | 0.098470039 | 0.988229502 | 5993 | 44.9183031 | 5.411531384 | 6.215293751 | B | 129 | 31% | Good |
| NOTCH3 | 0.099498625 | 1.00410418 | 6263 | 46.94198771 | 5.516037017 | 7.152488578 | B | 124 | 66% | Good |
| KLF7 | 0.101220269 | 1.008573894 | 5140 | 38.52495878 | 5.110202821 | 7.472285062 | B | 97 | 76% | Good |
| DHRS4 | 0.106248327 | 0.98468674 | 6451 | 48.3510718 | 5.586789265 | 7.014512199 | B | 127 | 26% | Good |
| ERBB4 | 0.109124088 | 1.000687663 | 3438 | 25.76825064 | 4.572291059 | 4.524602834 | B | 97 | 57% | Poor |
| DNMT3A | 0.109264487 | 1.006092904 | 3791 | 28.41403088 | 4.687603161 | 6.163119538 | B | 102 | 70% | Good |
| PCNX | 0.113247075 | 1.001876769 | 3414 | 25.58836756 | 4.565391125 | 4.147407073 | B | 121 | 61% | Poor |
| ZCCHC6 | 0.114181724 | 1.001416528 | 6669 | 49.98500974 | 5.671624773 | 7.650567691 | B | 104 | 59% | Poor |
| CSNK2A1 | 0.120905109 | 1.000904796 | 5796 | 43.44176285 | 5.338501705 | 7.355664723 | B | 120 | 58% | Poor |
| GGCX | 0.132200055 | 0.996237684 | 4727 | 35.42947084 | 4.973062314 | 6.097073153 | B | 111 | 48% | Poor |
| RNF2 | 0.138291474 | 1.000404524 | 3994 | 29.9355419 | 4.755613151 | 4.75417006 | B | 113 | 57% | Poor |
| BICD1 | 0.000997899 | 0.995479784 | 845 | 6.333383301 | 3.596604619 | 7.276725939 | C | 121 | 46% | Poor |
| POU2F2 | 0.015878559 | 1.019510995 | 1713 | 12.83915455 | 4.000005069 | 5.555314232 | C | 121 | 90% | Good |
| CG018 | 0.021754796 | 0.988878543 | 3195 | 23.94693449 | 4.491712915 | 5.011038491 | C | 111 | 32% | Good |
| ACTG1 | 0.027652893 | 0.990805209 | 817 | 6.123519712 | 3.579911364 | 12.49096002 | C | 112 | 37% | Poor |
| SLC25A16 | 0.031872768 | 1.024251496 | 1673 | 12.53934942 | 3.98374567 | 4.458671727 | C | 117 | 94% | Good |
| SYT11 | 0.039319097 | 0.997453577 | 3100 | 23.23489732 | 4.463242088 | 4.819834744 | C | 116 | 51% | Poor |
| FSTL4 | 0.04042613 | 1.015843697 | 2236 | 16.75910658 | 4.180153387 | 5.647671942 | C | 103 | 87% | Good |
| BCL2 | 0.042220431 | 0.991340249 | 2757 | 20.66406836 | 4.358849031 | 6.059490629 | C | 105 | 38% | Poor |
| NFRKB | 0.045890333 | 1.010114992 | 532 | 3.987408185 | 3.384743692 | 6.644913041 | C | 116 | 80% | Good |
| POLR2A | 0.046934535 | 1.009962241 | 2162 | 16.2044671 | 4.151777952 | 4.480060266 | C | 121 | 79% | Good |
| ERN1 | 0.048382257 | 1.007297966 | 2398 | 17.97331734 | 4.236720625 | 6.364227225 | C | 120 | 73% | Good |
| KLHL20 | 0.048456385 | 1.012940488 | 918 | 6.880527657 | 3.639701372 | 5.337923827 | C | 106 | 84% | Good |
| C7ORF25 | 0.049043508 | 1.012484816 | 408 | 3.058012292 | 3.287209075 | 4.84821256 | C | 128 | 83% | Good |
| ADRBK1 | 0.050711378 | 1.008428438 | 2625 | 19.67471144 | 4.310653823 | 7.010186885 | C | 111 | 75% | Good |
| LARS | 0.052978046 | 0.989025076 | 2533 | 18.98515965 | 4.282906087 | 8.118859388 | C | 108 | 33% | Good |
| MAP1B | 0.055360088 | 1.009544692 | 2542 | 19.0526158 | 4.287262187 | 4.653352601 | C | 109 | 78% | Good |
| KIAA0194 | 0.055451709 | 1.000342672 | 228 | 1.708889222 | 3.097201468 | 7.701227206 | C | 111 | 57% | Poor |
| LUZP1 | 0.057217627 | 1.002400713 | 14 | 0.104931794 | 2.393743761 | 7.491755018 | C | 120 | 63% | Poor |
| ALKBH1 | 0.058662383 | 0.998142886 | 2570 | 19.26247939 | 4.293919654 | 7.377745876 | C | 119 | 53% | Poor |
| NUP188 | 0.059353633 | 1.012540825 | 1558 | 11.67740968 | 3.937055029 | 5.178103055 | C | 113 | 83% | Good |
| SNAI1 | 0.060508864 | 1.012521793 | 1283 | 9.616249438 | 3.815857079 | 6.683897367 | C | 124 | 83% | Good |
| HIPK2 | 0.063008144 | 1.001895285 | 2778 | 20.82146605 | 4.366234811 | 6.265548604 | C | 119 | 61% | Poor |
| FLJ43663 | 0.064537856 | 1.007741024 | 201 | 1.506520762 | 3.048786797 | 3.814065669 | C | 124 | 74% | Good |
| PCDH9 | 0.0653874 | 1.014570227 | 1091 | 8.17718483 | 3.729993631 | 4.277113912 | C | 108 | 86% | Good |
| RERE | 0.065776079 | 1.006397817 | 552 | 4.137310748 | 3.40503803 | 4.361013509 | C | 102 | 70% | Good |
| PER1 | 0.068415183 | 1.002393399 | 104 | 0.779493329 | 2.872860021 | 6.980244659 | C | 125 | 63% | Poor |
| MR1 | 0.072032842 | 0.996628802 | 1185 | 8.881726878 | 3.773163213 | 5.982442371 | C | 96 | 49% | Poor |
| C11ORF17 | 0.073505038 | 0.990299378 | 2821 | 21.14375656 | 4.379304256 | 5.673302422 | C | 129 | 36% | Poor |
| HPS1 | 0.073548841 | 1.013649511 | 149 | 1.116774097 | 2.976950121 | 6.476592219 | C | 105 | 85% | Good |
| SCNM1 | 0.074244986 | 0.996396531 | 1547 | 11.59496327 | 3.932748595 | 9.004397408 | C | 128 | 48% | Poor |
| NDST2 | 0.07429773 | 1.008817643 | 769 | 5.76375356 | 3.547817017 | 6.939415527 | C | 123 | 77% | Good |
| RAD51L1 | 0.074460851 | 0.997754631 | 1579 | 11.83480738 | 3.944954476 | 4.097040759 | C | 129 | 52% | Poor |
| CACNA1G | 0.075211702 | 0.997563526 | 1384 | 10.37325738 | 3.858756224 | 4.800607103 | C | 101 | 51% | Poor |
| EIF1 | 0.076536456 | 0.994648703 | 2108 | 15.79973018 | 4.133156616 | 10.48885468 | C | 114 | 45% | Poor |
| CENTD2 | 0.080564655 | 1.006845736 | 717 | 5.374006896 | 3.519616268 | 6.93084978 | C | 116 | 72% | Good |
| PDE8A | 0.08097957 | 1.005686521 | 1365 | 10.23084995 | 3.85119531 | 5.819030061 | C | 122 | 69% | Good |
| DVL3 | 0.081543688 | 0.998622456 | 3126 | 23.42977065 | 4.469027263 | 6.455380783 | C | 124 | 54% | Poor |
| DYM | 0.081565656 | 1.009860818 | 2787 | 20.8889222 | 4.371258687 | 3.964215145 | C | 125 | 79% | Good |
| PDE4A | 0.083335146 | 1.013153044 | 3266 | 24.47908859 | 4.520067813 | 4.826309346 | C | 120 | 84% | Good |
| FGFR1 | 0.083804982 | 1.015614485 | 2506 | 18.78279119 | 4.274916322 | 6.861838821 | C | 124 | 86% | Good |
| PHF15 | 0.085726566 | 1.001440553 | 2157 | 16.16699146 | 4.150070456 | 6.182042197 | C | 98 | 59% | Poor |
| ST3GAL2 | 0.085927127 | 1.001725725 | 577 | 4.324688952 | 3.42893581 | 5.64176634 | C | 98 | 60% | Poor |
| FLII | 0.086013521 | 0.993969465 | 1932 | 14.48058762 | 4.072676222 | 7.079464198 | C | 117 | 43% | Poor |
| KLF12 | 0.08724397 | 1.006763617 | 970 | 7.270274322 | 3.672972828 | 5.523407118 | C | 107 | 72% | Good |
| IFNAR1 | 0.090763099 | 1.005154032 | 2673 | 20.03447759 | 4.330093106 | 5.620071998 | C | 123 | 68% | Good |
| PNPLA2 | 0.092560293 | 1.001734028 | 1016 | 7.615050217 | 3.69580935 | 8.421514347 | C | 113 | 60% | Poor |
| ZNF335 | 0.093172698 | 1.005406575 | 624 | 4.676959976 | 3.462618042 | 6.272871357 | C | 103 | 69% | Good |
| ABR | 0.094812705 | 0.999088198 | 1834 | 13.74606506 | 4.040402717 | 6.62483202 | C | 107 | 55% | Poor |
| CHRD | 0.096586451 | 1.006811688 | 3162 | 23.69959526 | 4.481484519 | 5.478358766 | C | 132 | 72% | Good |
| CLIC1 | 0.099728132 | 1.003507577 | 2984 | 22.36546245 | 4.42832086 | 9.648849067 | C | 124 | 65% | Poor |
| CLTC | 0.100536086 | 1.007783183 | 2185 | 16.37685504 | 4.159691969 | 3.48823819 | C | 135 | 74% | Good |
| HCFC1 | 0.104103183 | 1.002632451 | 30 | 0.224853845 | 2.602159713 | 7.280074735 | C | 109 | 63% | Poor |
| PDE4DIP | 0.104560793 | 1.006505039 | 1654 | 12.39694199 | 3.973761821 | 4.353246344 | C | 111 | 71% | Good |
| IRF5 | 0.113490731 | 1.004678089 | 770 | 5.771248688 | 3.548102358 | 6.202174462 | C | 118 | 67% | Good |
| DLC1 | 0.114091838 | 1.002775945 | 797 | 5.973617149 | 3.568438577 | 5.096913541 | C | 107 | 64% | Poor |
| ARHGEF2 | 0.117069343 | 1.001833226 | 2400 | 17.9883076 | 4.236806804 | 7.531437586 | C | 116 | 61% | Poor |
| KIAA0146 | 0.123537953 | 0.996667145 | 1456 | 10.91290661 | 3.892494472 | 5.304924979 | C | 94 | 49% | Poor |
| RNF8 | 0.125410979 | 1.009623546 | 2831 | 21.21870784 | 4.381387893 | 5.797011142 | C | 122 | 78% | Good |
| HOM_TES_103 | 0.065328707 | 0.999101055 | #N/A | #N/A | #N/A | #N/A | #N/A | 124 | 55% | Poor |

### EQUIVALENTS

Those skilled in the art will recognize, or be able to ascertain using no more than routine experimentation, many equivalents to the specific embodiments described herein. Such equivalents are intended to be encompassed by the following claims.

What is claimed:

1. A method of evaluating or treating a subject, comprising:
a) optionally, acquiring a subject sample;
b) acquiring a value or values that is a function of the level of gene expression for (each of) a plurality of genes selected from a first and/or second and/or third and/or fourth and or/fifth and/or sixth set of genes, wherein
(i) said first set of genes comprises or consists of:
S100A4, SDC2, CYP1B1, FERMT2, CYBRD1, SNAI2, CD44, BIN1, PMP22, CTGF, IGFBP3, ANXA6, MAP1B, TAGLN, ENPP2, FBLN5, PCOLCE, FN1, VIM, PPAP2B, SEM5A, VCAN, NID2, NID1, HAS2, PRRX1, TNFAIP6, CLDN4, FADS2, DDIT4, GNG11, PER1, ISCU, KPNA3, SLC44A1, FKBP5, NR2F1, MLPH, DOCK10, RGS4, IGFBP4; and
(ii) said second set of genes comprises or consists of:
S100A4, SDC2, CYP1B1, FERMT2, CYBRD1, SNAI2, CD44, BIN1, ENPP2, FBLN5, PCOLCE, FN1, VIM, PPAP2B, SEM5A, VCAN, CLDN4, FADS2, DDIT4, GNG11, PER1, ISCU, KPNA3, SLC44A1, FKBP5, IGFBP4; and
(iii) said third set of genes comprises or consists of:
SDC2, CYBRD1, SNAI2, PMP22, CTGF, IGFBP3, ANXA6, MAP1B, TAGLN, PPAP2B, HAS2, NRF2, MLPH, DOCK10, RGS4; and
(iv) said fourth set of genes comprises or consists of:
FERMT2, BIN1, PMP22, CHN1, CTGF, IGFBP3, NID2, CLDN4, CYBRD1, PRR16, ANXA6, PCOLCE, PPAP2B, HAS2, CDH11, MLPH, DOCK10, RGS4, PVRL3, NID2, SLC44A1, FKBP5; and
(v) said fifth set of genes comprises or consists of:
FERMT2, BIN1, PMP22, CHN1, CTGF, IGFBP3, NID2, CLDN4; and
(vi) said sixth set of genes comprises or consists of:
HAS2, BIN1, PCOLCE, FERMT2, CTGF, IGFBP3, NID2, SLC44A1, FKBP5, and MLPH; and
(c) responsive to said value or values:
(i) classifying said subject; or
(ii) administering treatment comprising an agent that inhibits or kills cancer associated mesenchymal cells, tumor initiating cancer cells, or cancer stem cells, to said subject; provided that, if (c)(ii) is not performed the acquisition in (a) or (b) comprises directly acquiring; thereby evaluating or treating said subject.

2-3. (canceled)

4. The method of claim 1, wherein c(i) and c(ii) are performed.

5-11. (canceled)

12. The method of claim 1, wherein step b) said plurality comprises, or consists of, a first gene and a second gene.

13. The method of claim 12, wherein step b) comprises acquiring a value that is a function of the level of gene expression of said first gene and the level of gene expression of said second gene.

14-17. (canceled)

18. The method of claim 13, wherein step b) comprises acquiring a first value that is a function of the level of gene expression of two or more genes of said plurality and a second value that is a function of the level of gene expression of one of the genes of the plurality.

19. The method of claim 1, further comprising acquiring a value or values that is a function of the level of gene expression of a gene not listed in claim 1.

20. (canceled)

21. The method of claim 13, wherein step b) comprises acquiring a value that is a function of the level of gene expression of said first gene, the level of gene expression of said second gene, and a weighting factor.

22. (canceled)

23. The method of claim 18, wherein said first value is a function of a first weighting factor and said second value is a function of a second weighting factor.

24. (canceled)

25. The method of claim 1, wherein said value or values is a function of a comparison with a reference criterion.

26. The method of claim 25, wherein said value or values is further a function of the determination of whether the level of gene expression has a preselected relationship with a reference criterion.

27. (canceled)

28. The method of claim 1, further comprising determining if said value or values has a preselected relationship with a reference criterion.

29-31. (canceled)

32. The method of claim 1, wherein said subject sample is a tumor sample, a tissue sample, a biopsy, blood sample, plasma sample, serum sample, interstitial fluid sample.

33. (canceled)

34. The method of claim 1, wherein a first value or values is acquired for a first location in said subject sample.

35. The method of claim 34, wherein a second value or values is acquired for a second location in said subject sample.

36. (canceled)

37. The method of claim 35, comprising determining if said first value or values and/or said second value or values has a preselected relationship with a reference criterion.

38-42. (canceled)

43. The method of claim 34, wherein said first value or values is a function of the level of gene expression of each gene of a plurality of genes at said first location.

44. (canceled)

45. The method of claim 1, comprising, responsive to the acquisition of said value or values one or more of:
(1) stratifying a patient population; and/or
(2) identifying or selecting the subject as likely or unlikely to respond positively to a treatment; and/or
(3) selecting a treatment; and/or
(4) prognosticating the time course of the disease in the subject and/or
(5) measuring the response at the end of therapy and predicting the long term outcome; and/or
(6) determining the cancer stem cell population as a predictor of response to a treatment or therapy.

46-61. (canceled)

62. A method of assaying in a subject sample the level of gene expression product of a plurality of genes selected from a first and/or second and/or third and/or fourth and/or fifth and/or sixth set of genes, wherein:
(i) said first set of genes comprises or consists of:
S100A4, SDC2, CYP1B1, FERMT2, CYBRD1, SNAI2, CD44, BIN1, PMP22, CTGF, IGFBP3, ANXA6, MAP1B, TAGLN, ENPP2, FBLN5, PCOLCE, FN1, VIM, PPAP2B, SEM5A, VCAN, NID2, NID1, HAS2, PRRX1, TNFAIP6, CLDN4, FADS2, DDIT4, GNG11, PER1, ISCU, KPNA3, SLC44A1, FKBP5, NR2F1, MLPH, DOCK10, RGS4, IGFBP4; and
(ii) said second set of genes comprises or consists of:
S100A4, SDC2, CYP1B1, FERMT2, CYBRD1, SNAI2, CD44, BIN1, ENPP2, FBLN5, PCOLCE, FN1, VIM, PPAP2B, SEM5A, VCAN, CLDN4, FADS2, DDIT4, GNG11, PER1, ISCU, KPNA3, SLC44A1, FKBP5, IGFBP4; and
(iii) said third set of genes comprises or consists of:
SDC2, CYBRD1, SNAI2, PMP22, CTGF, IGFBP3, ANXA6, MAP1B, TAGLN, PPAP2B, HAS2, NRF2, MLPH, DOCK10, RGS4; and
(iv) said fourth set of genes comprises or consists of:
FERMT2, BIN1, PMP22, CHN1, CTGF, IGFBP3, NID2, CLDN4, CYBRD1, PRR16, ANXA6, PCOLCE, PPAP2B, HAS2, CDH11, MLPH, DOCK10, RGS4, PVRL3, NID2, SLC44A1, FKBP5; and
(v) said fifth set of genes comprises or consists of:
FERMT2, BIN1, PMP22, CHN1, CTGF, IGFBP3, NID2, CLDN4; and
(vi) said sixth set of genes comprises or consists of:
HAS2, BIN1, PCOLCE, FERMT2, CTGF, IGFBP3, NID2, SLC44A1, FKBP5, and MLPH; comprising a first agent capable of interacting with a gene expression product of a plurality of genes selected from a first and/or second and/or third and/or fourth and/or fifth and/or sixth set of genes; and wherein the method comprises assaying the level of gene expression product of the plurality of genes.

63-77. (canceled)

78. A reaction mixture comprising:
a plurality of detection reagents; and
a plurality of target nucleic acid molecules derived from a subject, wherein each of the plurality of detection reagents comprises a plurality probes to measure the level of gene expression product of a plurality of genes selected from a first and/or second and/or third and/or fourth and/or fifth and/or sixth set of genes, wherein:
(i) said first set of genes comprises or consists of:
S100A4, SDC2, CYP1B1, FERMT2, CYBRD1, SNAI2, CD44, BIN1, PMP22, CTGF, IGFBP3, ANXA6, MAP1B, TAGLN, ENPP2, FBLN5, PCOLCE, FN1, VIM, PPAP2B, SEM5A, VCAN, NID2, NID1, HAS2, PRRX1, TNFAIP6, CLDN4, FADS2, DDIT4, GNG11, PER1, ISCU, KPNA3, SLC44A1, FKBP5, NR2F1, MLPH, DOCK10, RGS4, IGFBP4; and
(ii) said second set of genes comprises or consists of:
S100A4, SDC2, CYP1B1, FERMT2, CYBRD1, SNAI2, CD44, BIN1, ENPP2, FBLN5, PCOLCE, FN1, VIM, PPAP2B, SEM5A, VCAN, CLDN4, FADS2, DDIT4, GNG11, PER1, ISCU, KPNA3, SLC44A1, FKBP5, IGFBP4; and
(iii) said third set of genes comprises or consists of:
SDC2, CYBRD1, SNAI2, PMP22, CTGF, IGFBP3, ANXA6, MAP1B, TAGLN, PPAP2B, HAS2, NRF2, MLPH, DOCK10, RGS4; and
(iv) said fourth set of genes comprises or consists of:
FERMT2, BIN1, PMP22, CHN1, CTGF, IGFBP3, NID2, CLDN4, CYBRD1, PRR16, ANXA6, PCOLCE, PPAP2B, HAS2, CDH11, MLPH, DOCK10, RGS4, PVRL3, NID2, SLC44A1, FKBP5; and
(v) said fifth set of genes comprises or consists of:
FERMT2, BIN1, PMP22, CHN1, CTGF, IGFBP3, NID2, CLDN4; and
(vi) said sixth set of genes comprises or consists of:
HAS2, BIN1, PCOLCE, FERMT2, CTGF, IGFBP3, NID2, SLC44A1, FKBP5, and MLPH.

79-110. (canceled)
